(19) United States
(12) Patent Application Publication
(10) Pub. No.: US 2024/0408119 A1
(43) Pub. Date: Dec. 12, 2024

(54) DELIVERY OF THERAPEUTIC ALKALOID COMPOUNDS

(71) Applicant: Sensorium Therapeutics, Inc., Boston, MA (US)

(72) Inventors: Jacob M. Hooker, Winchester, MA (US); Michael S. Placzek, Tewksbury, MA (US)

(21) Appl. No.: 18/799,146

(22) Filed: Aug. 9, 2024

Related U.S. Application Data
(63) Continuation of application No. PCT/US23/28920, filed on Jul. 28, 2023.
(60) Provisional application No. 63/426,577, filed on Nov. 18, 2022.
(60) Provisional application No. 63/393,563, filed on Jul. 29, 2022.

(51) Int. Cl. A61K 31/675; A61K 9/08; A61K 31/404; A61K 31/665; C07D 209/12; C07F 9/6574; C07F 9/6584

(52) U.S. Cl. CPC A61K 31/675; A61K 9/08; A61K 31/404; A61K 31/665; C07D 209/12; C07F 9/65742; C07F 9/65744; C07F 9/6584

(57) ABSTRACT

Disclosed are compounds that can be converted to mesembrine under physiologically relevant conditions, and related methods of preparing and using the compounds.

### RELATED APPLICATIONS

This application claims the benefit of priority to U.S. Provisional Application No. 63/393,563, filed Jul. 29, 2022, and U.S. Provisional Application No. 63/426,577, filed Nov. 18, 2022; the contents of each of which are incorporated by reference in their entirety.

### TECHNICAL FIELD

The present disclosure relates to the field of medicine, including the discovery of alkaloid compounds useful for inhibiting the serotonin transporter protein (5-HTT).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine and mesembrenol are pharmacologically active alkaloids present in Sceletium tortuosum extracts used for treatment of anxiety, stress and mental health conditions.

Natural products obtained from plants of the genus Sceletium contain varying amounts of (−) mesembrine and (+)/(−) mesembrenone. The structure of mesembrine, also known as 3a-(3,4-dimethoxyphenyl)-octahydro-1-methyl-6H-indol-6-one, has been reported by Popelak et al., Naturwiss.47,156 (1960), and the configuration by P W Jeffs et al., J. Am. Chem. Soc. 91, 3831 (1969). Naturally occurring (−) mesembrine from Sceletium tortuosum has been reported as having serotonin (5-HT) uptake inhibitory activity useful in treating mental health conditions such as mild to moderate depression.

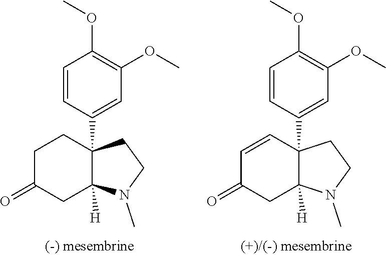 [SMILES: COc1ccc([C@@]23CCC(=O)C[C@@H]2N(C)CC3)cc1OC ; COc1ccc([C@@]23C=CC(=O)C[C@@H]2N(C)CC3)cc1OC]

An analysis of a standardized commercial extract of Sceletium tortuosum was reported in 2011 (obtained as a product under the tradename, Zembrin®) as having 0.35-0.45% total alkaloids, with mesembrenone and mesembrenol comprising ≥60%, and mesembrine contributing <20% (See Harvey et al., “Pharmacological actions of the South African medicinal and functional food plant Sceletium tortuosum and its principal alkaloids,” Journal of Ethnopharmacology 137 (2011) 1124-11292011 and Murbach et. al., “A toxicological safety assessment of a standardized extract of Sceletium tortuosum (Zembrin®) in rats,” Food and Chemical Toxicology 74 (2014) 190-199). The extract gave >80% inhibition at serotonin (5-HT) transporter with potency of the isolated alkaloids at the 5-HT transporter reported as shown in Table A below (Harvey et al., 2011). Referring to the data in Table A, concentration-dependent inhibition was found, with mesembrine being the more active compound (i.e., 20 times more potent than mesembrenone and 87 times more active than mesembrenol) in the 5-HT transporter assay. A toxicological safety assessment of this standardized extract was subsequently reported in 2014 (Murbach et al., 2014).

TABLE A
Summary of analysis of the concentration response curves of alkaloids
on binding to the 5-HT transporter (Harvey et al., 2011)

5-HT transporter (SERT)

| Compound | Ki (nM) | nH |
|---|---|---|
| Mesembrine | 1.4 | 1.0 |
| Mesembrenone | 27 | 1.0 |

However, bioactive plant extracts for therapeutic consumption can vary widely both seasonally and between different Sceletium tortuosum plants, and fail to provide a sufficiently reproducible and stable phytochemical profile of desired biologically active components. Plants of the genus Sceletium and extracts thereof can vary widely in terms of the total alkaloid content, as well as the chemistry and relative concentrations of individual Sceletium plant derived alkaloids. In addition, mesembrine is unstable under a variety of conditions that can occur during extraction from plant material, as well as during storage and formulation of the extract. For example, mesembrine has been reported to be unstable under conditions of fermentation, exposure to light, exposure to heat, and in an aqueous medium.

The therapeutic use of mesembrine has been limited by the variability and instability of these compounds content in natural extract products and the instability and pharmacokinetic profile of these compounds as obtained from natural products.

There remains an unmet need for pharmaceutical compositions comprising higher purity, predictable, stable and reproducible forms of therapeutic alkaloid compounds such as mesembrine. In addition, there is a need for oral pharmaceutical compositions providing pure therapeutic alkaloid compositions having desired pharmacokinetic properties upon administration.

### SUMMARY

Described are prodrug compounds that, when administered orally or intravenously to a subject, convert to mesembrine in vivo. Remarkably, the compounds allow for sustained release of mesembrine thereby extending exposure of mesembrine in the brain compared to a subject receiving an equivalent oral or intravenous dose of mesembrine itself. The sustained release and extended brain exposure to mesembrine will address recognized therapeutic shortcomings attributed to the pharmacokinetics of mesembrine. The prodrug compounds provide improved duration of action of mesembrine for enhanced therapeutic benefit.

Described herein are compounds of formula (I):

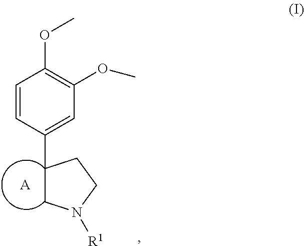

or a pharmaceutically acceptable salt thereof,
wherein

R¹ is H or C₁-C₇ alkyl;

ring A is

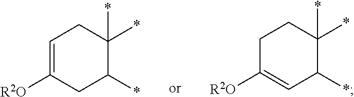

wherein * denotes the attachment points of ring A to the compound of formula (I),

R² is —C(O)NR³R⁴, —C(O)OR⁶, or —P(O)OR⁴OR⁵;

R³ is H, C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;

R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl; or R³ and R⁴ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁵ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R⁴ and R⁵ combine to form a 4-8 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and

n is 1 or 2.

In certain embodiments, the compound is of formula (I-A): is

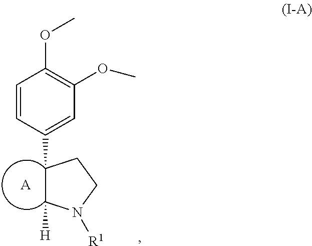

or a pharmaceutically acceptable salt thereof, wherein A and R¹ are as defined herein.

In certain embodiments, the compound is of formula (I-A):

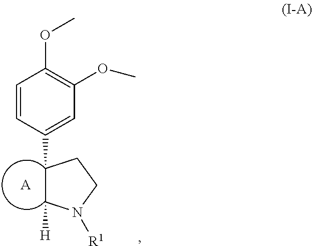

or a pharmaceutically acceptable salt thereof, wherein A and R¹ are as defined herein; and the compound has the absolute stereochemistry shown.

In certain embodiments, the compound is of formula (II):

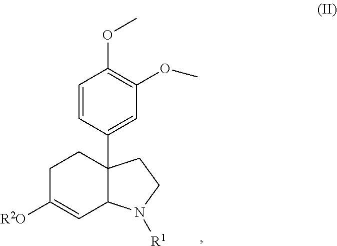

or a pharmaceutically acceptable salt thereof, wherein R¹ and R² are as defined herein.

In certain embodiments, the compound is of formula (II-A):

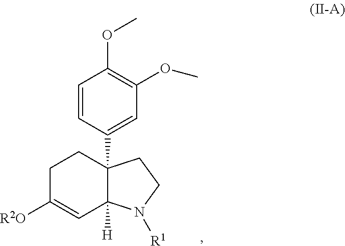

or a pharmaceutically acceptable salt thereof, wherein R¹ and R² are as defined herein.

In certain embodiments, the compound is of formula (II-A):

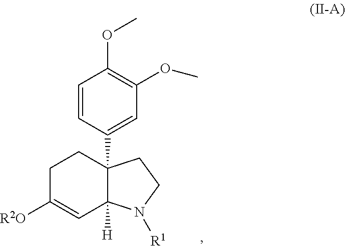

or a pharmaceutically acceptable salt thereof, wherein R¹ and R² are as defined herein; and the compound has the absolute stereochemistry shown.

In certain embodiments, the compound is selected from the group consisting of:

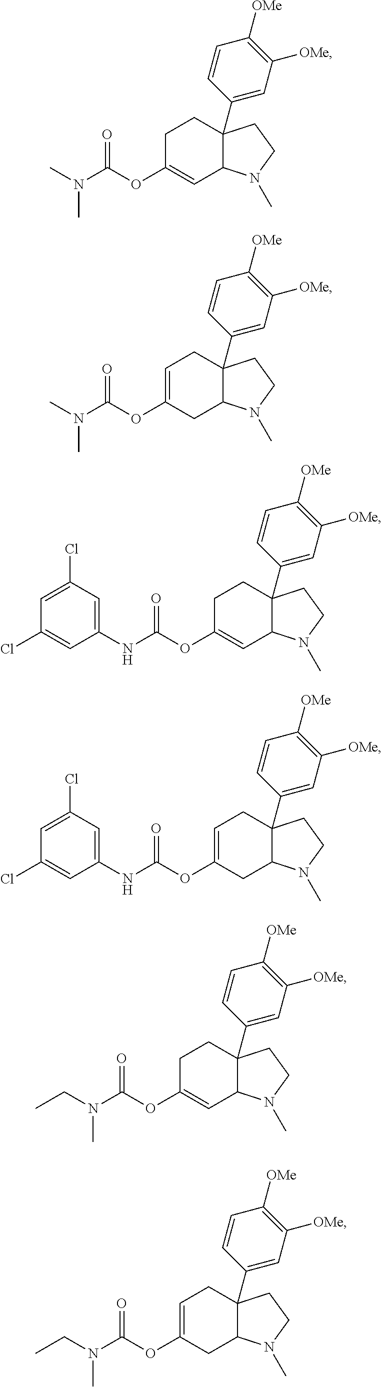

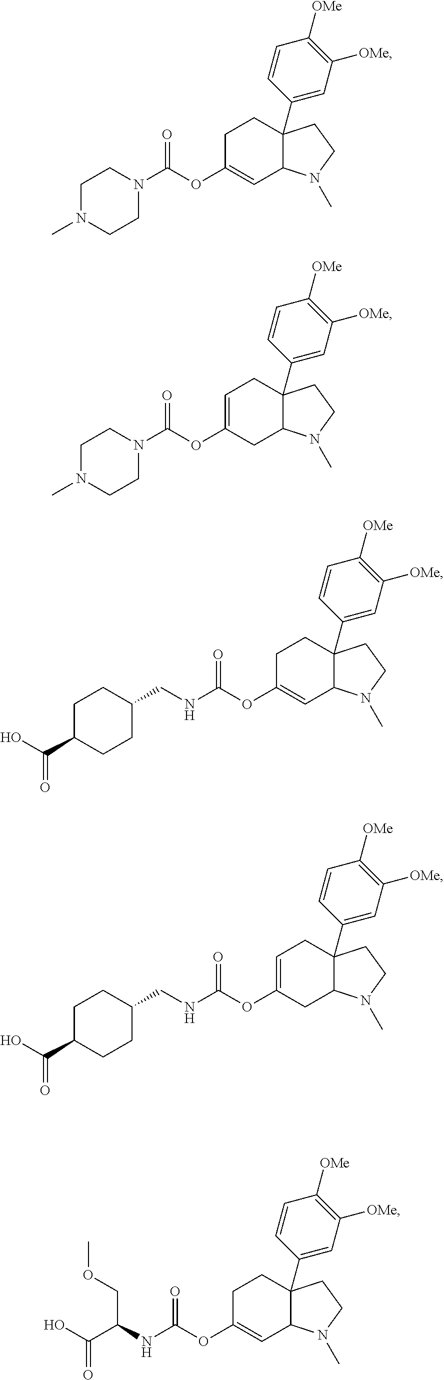 [SMILES: COc1ccc(C23CCC(OC(=O)N4CCN(C)CC4)=CC2N(C)CC3)cc1OC ; COc1ccc(C23CC=C(OC(=O)N4CCN(C)CC4)CC2N(C)CC3)cc1OC ; COc1ccc(C23CCC(OC(=O)NC[C@H]4CC[C@H](C(=O)O)CC4)=CC2N(C)CC3)cc1OC ; COc1ccc(C23CC=C(OC(=O)NC[C@H]4CC[C@H](C(=O)O)CC4)CC2N(C)CC3)cc1OC ; COC[C@@H](NC(=O)OC1=CC2N(C)CCC2(c2ccc(OC)c(OC)c2)CC1)C(=O)O]

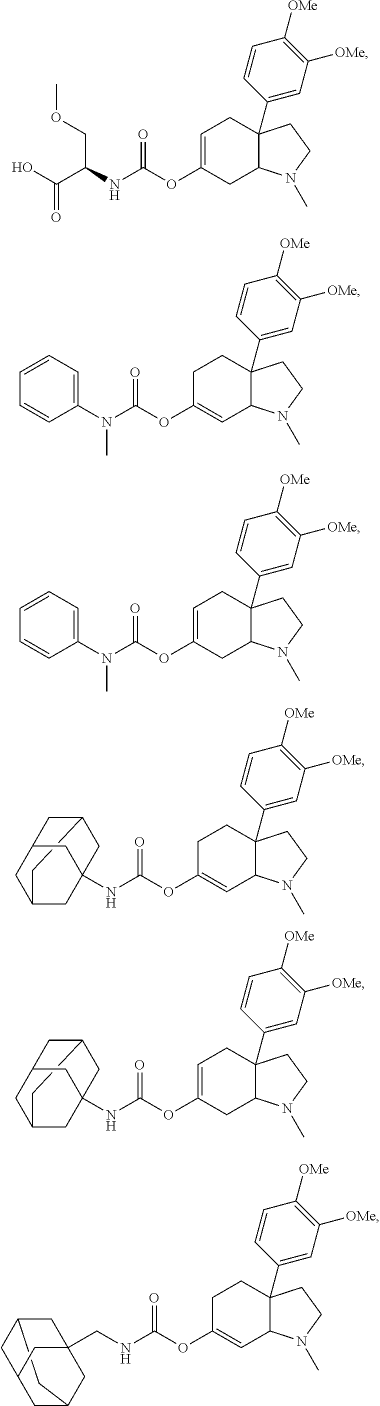

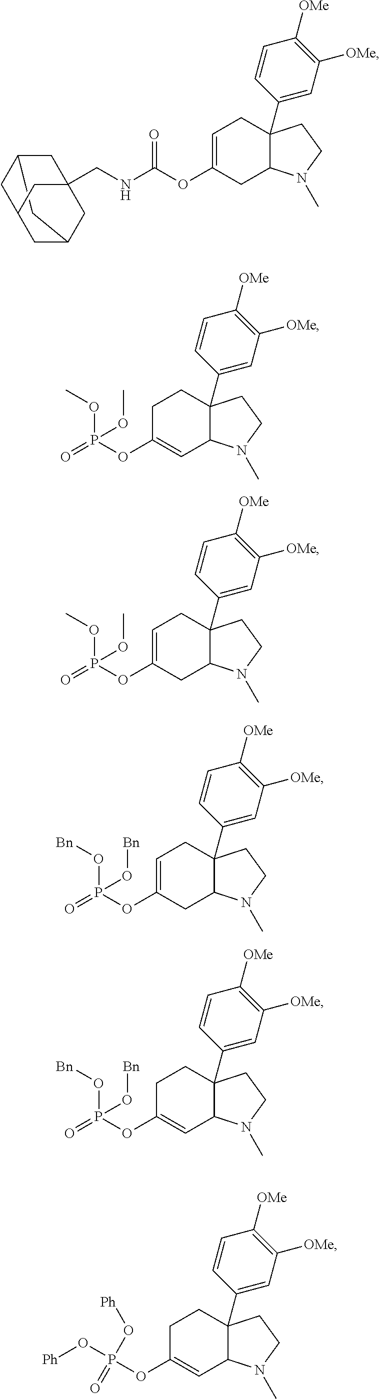

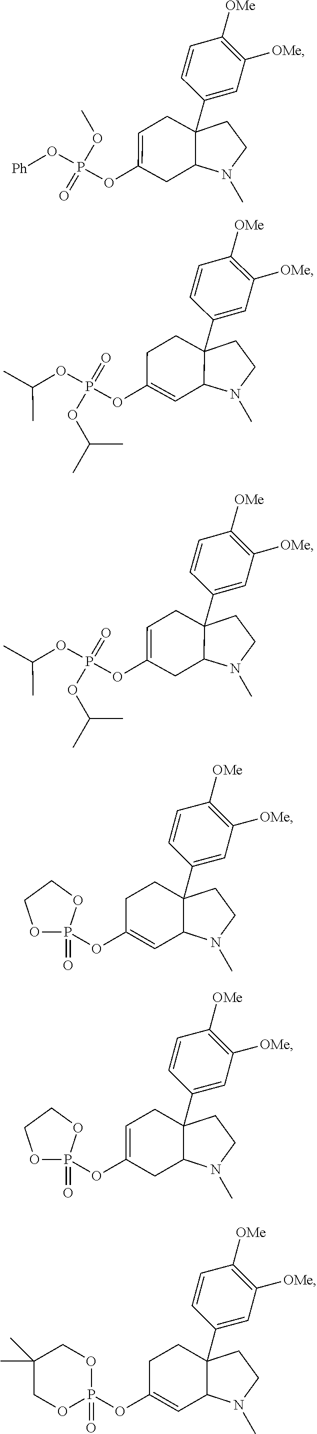

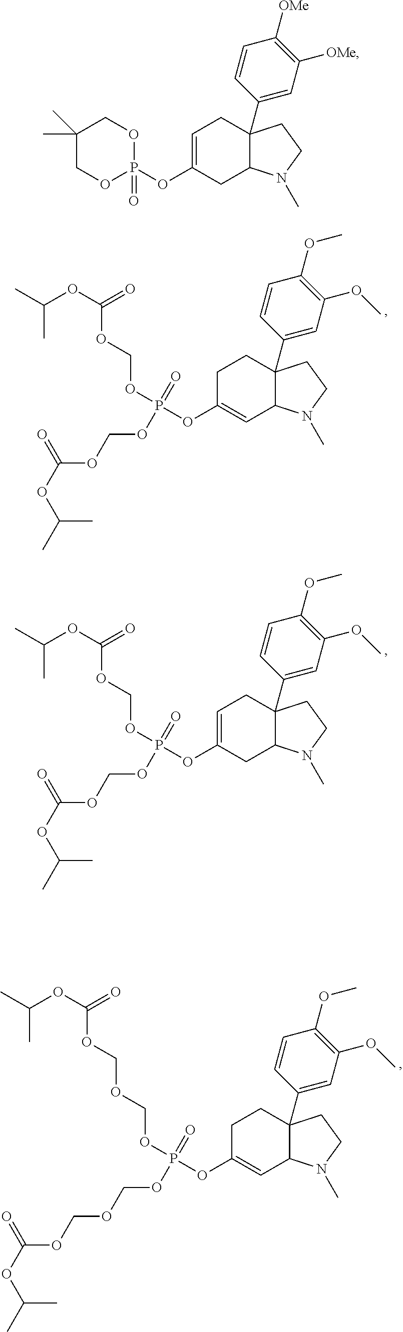

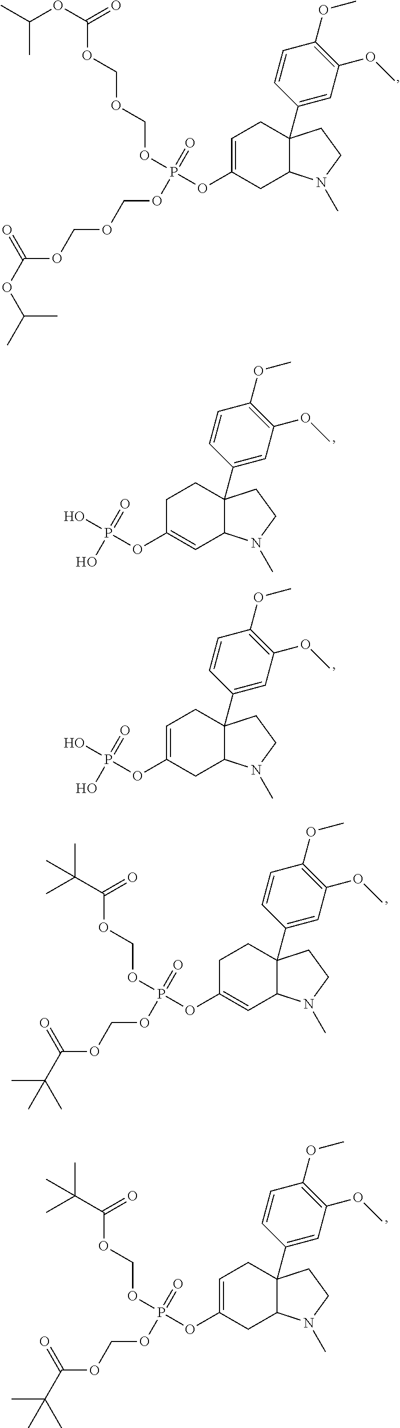

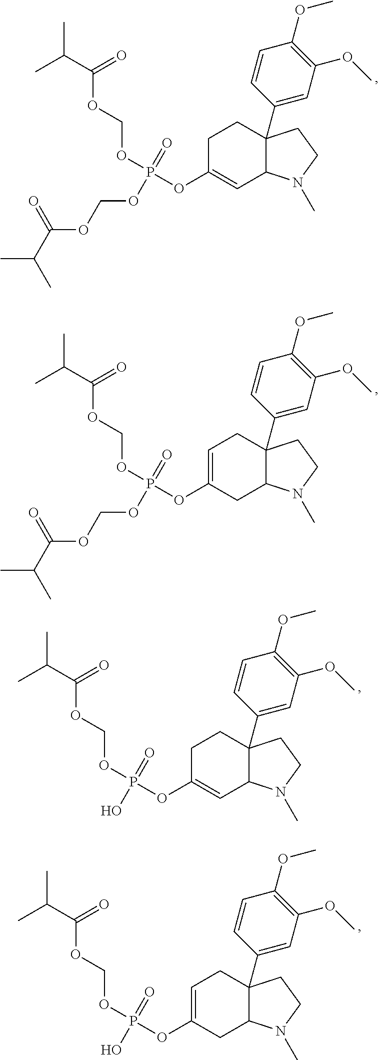

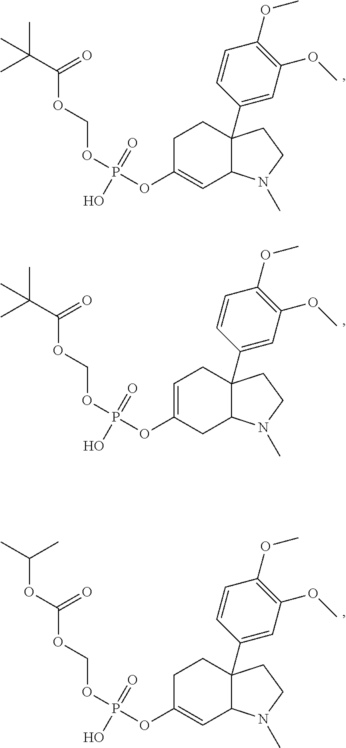

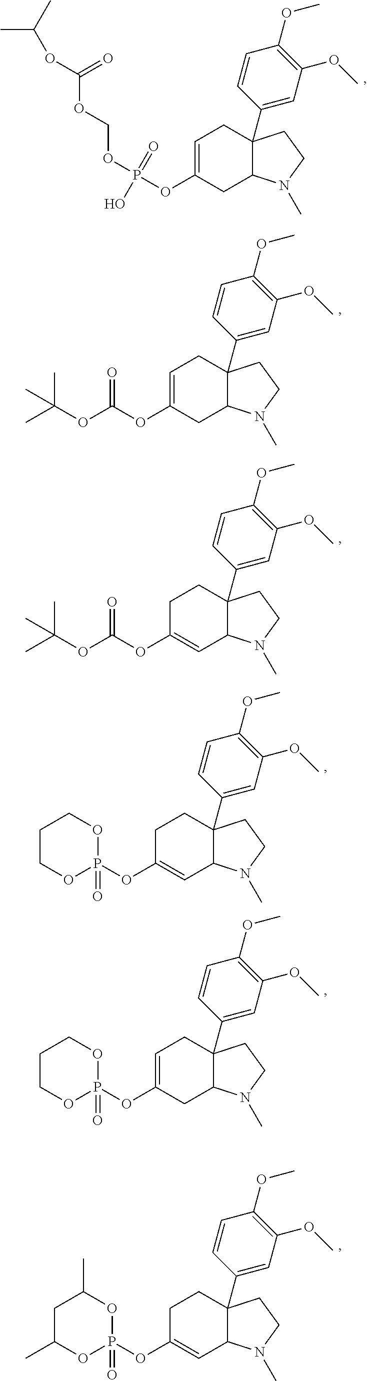

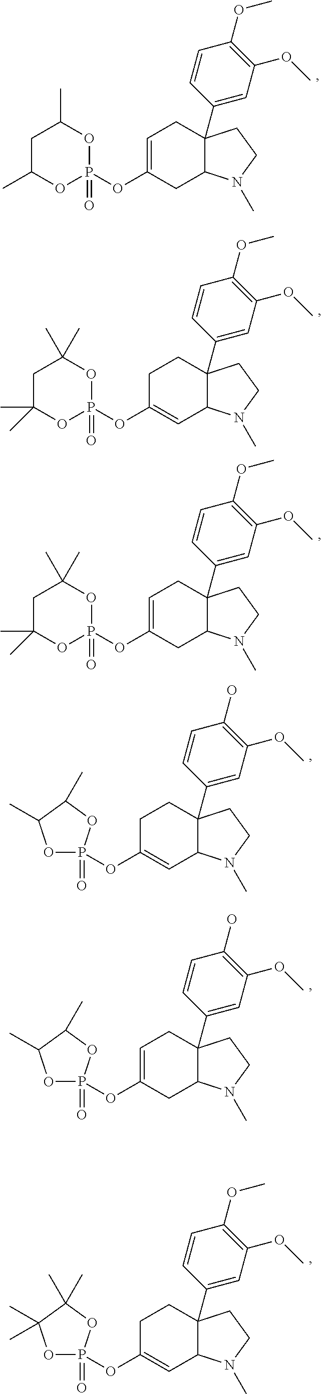

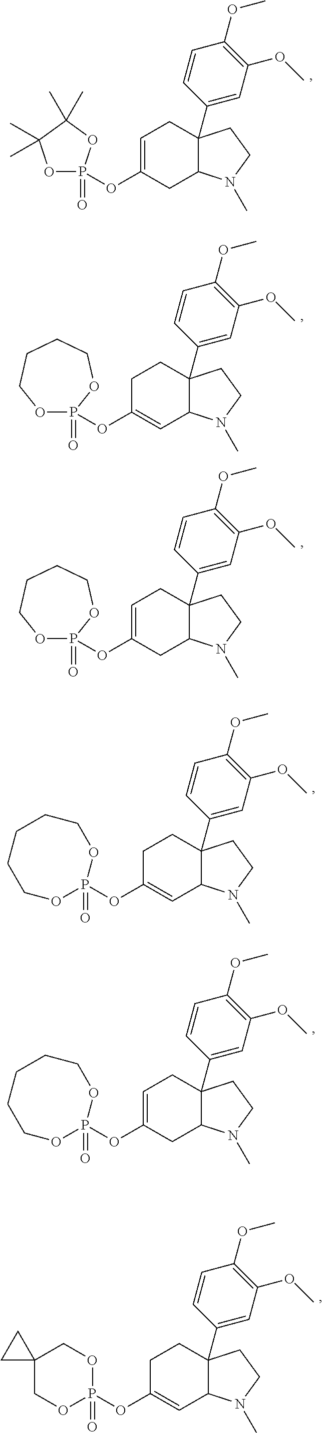

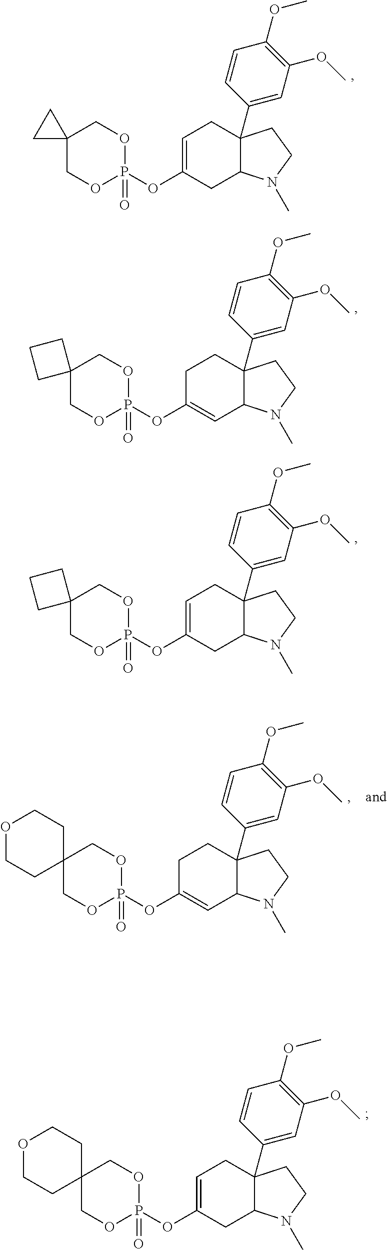

or a pharmaceutically acceptable salt thereof.

In some embodiments, the compound is selected from the group consisting of:

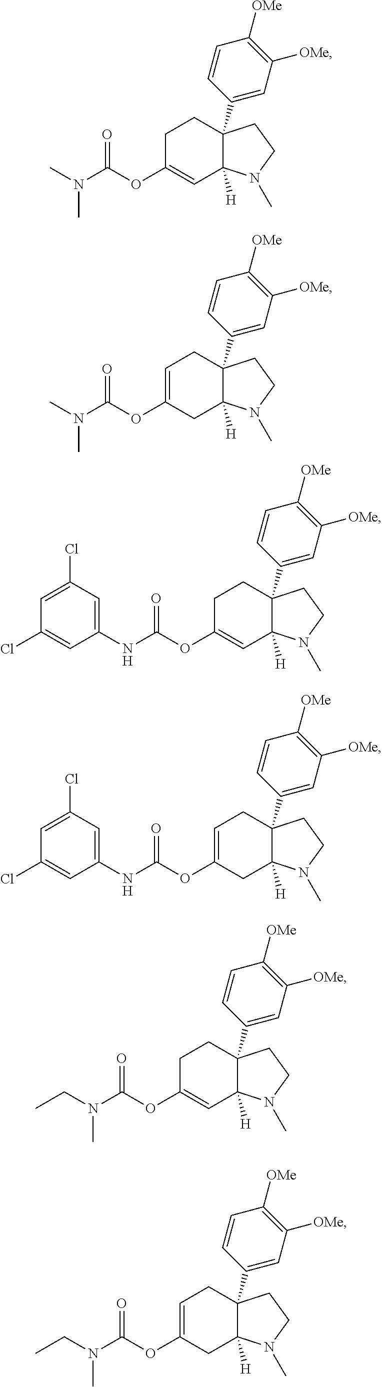

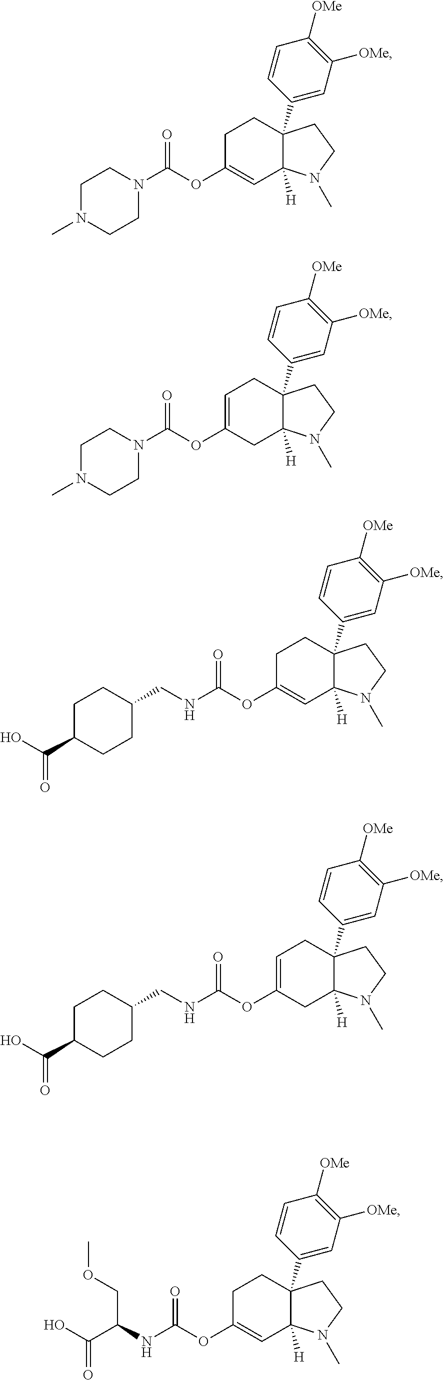 [SMILES: COc1ccc([C@@]23CCC(OC(=O)N4CCN(C)CC4)=C[C@@H]2N(C)CC3)cc1OC ; COc1ccc([C@@]23CC=C(OC(=O)N4CCN(C)CC4)C[C@@H]2N(C)CC3)cc1OC ; COc1ccc([C@@]23CCC(OC(=O)NC[C@H]4CC[C@H](C(=O)O)CC4)=C[C@@H]2N(C)CC3)cc1OC ; COc1ccc([C@@]23CC=C(OC(=O)NC[C@H]4CC[C@H](C(=O)O)CC4)C[C@@H]2N(C)CC3)cc1OC ; COC[C@@H](NC(=O)OC1=C[C@@H]2N(C)CC[C@]2(c2ccc(OC)c(OC)c2)CC1)C(=O)O]

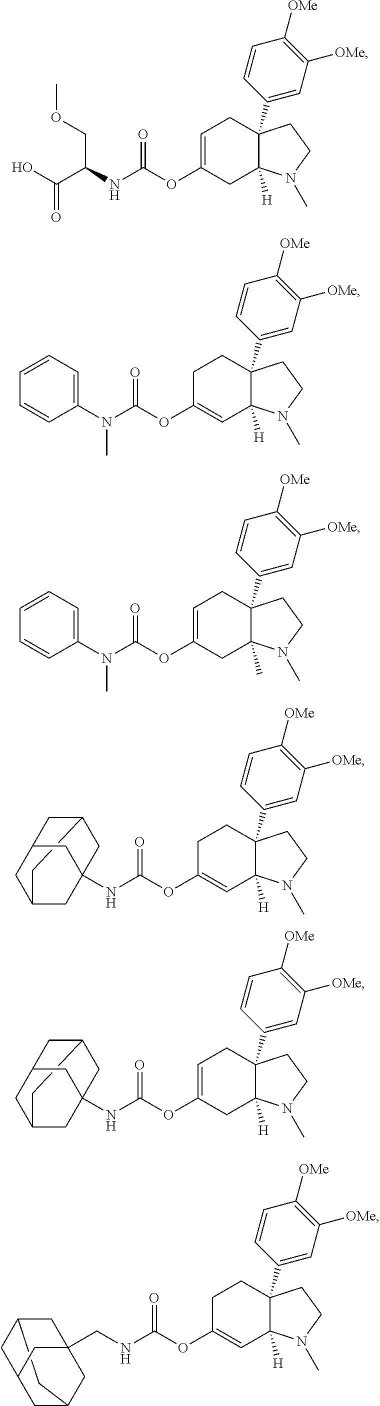

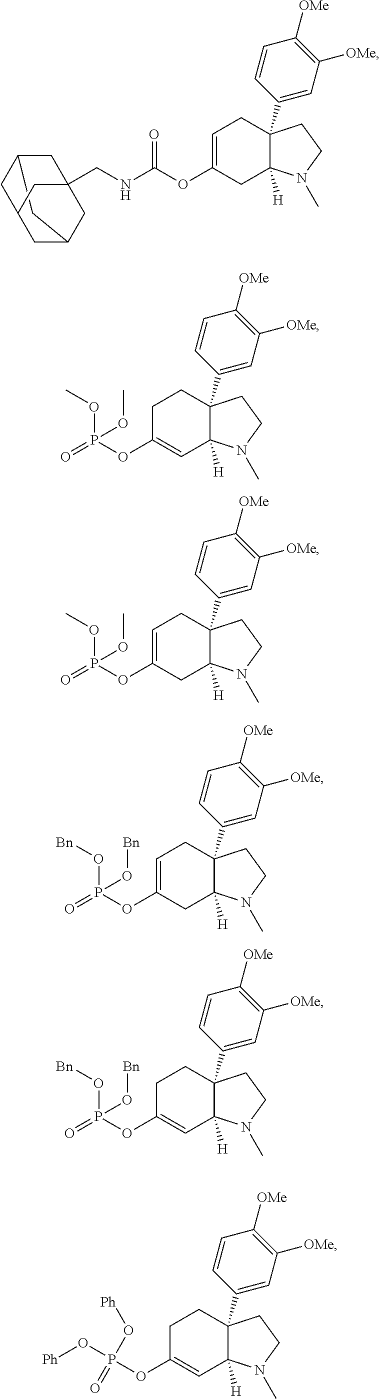

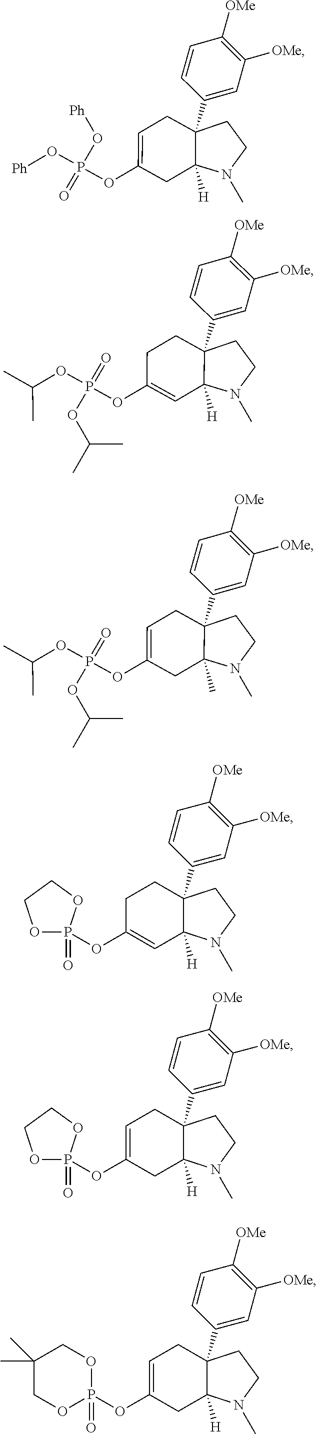

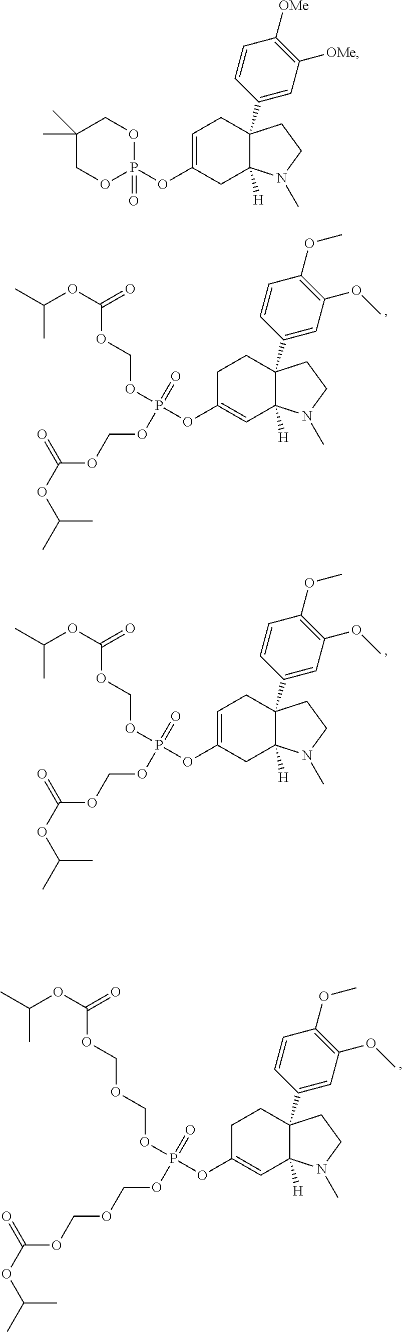

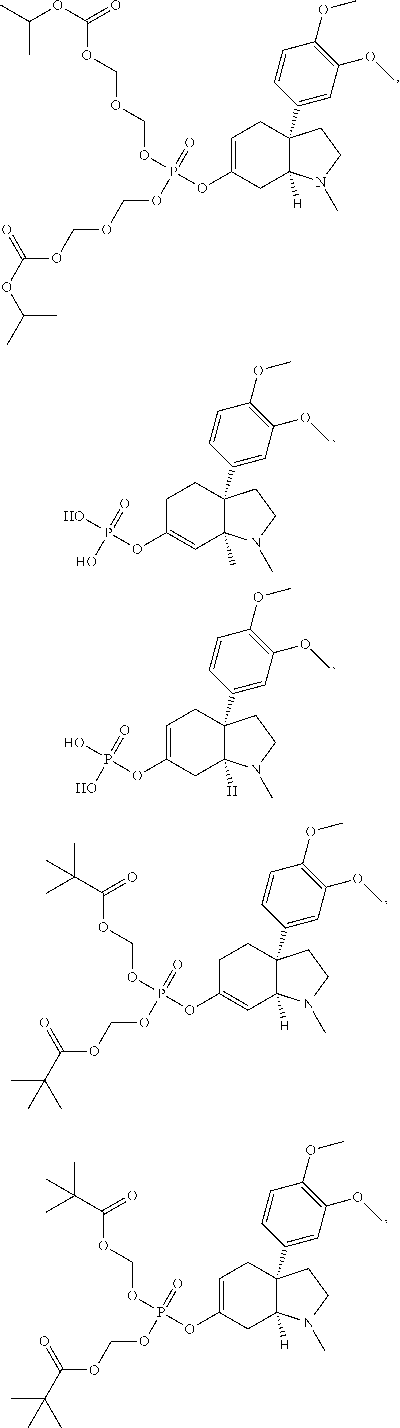

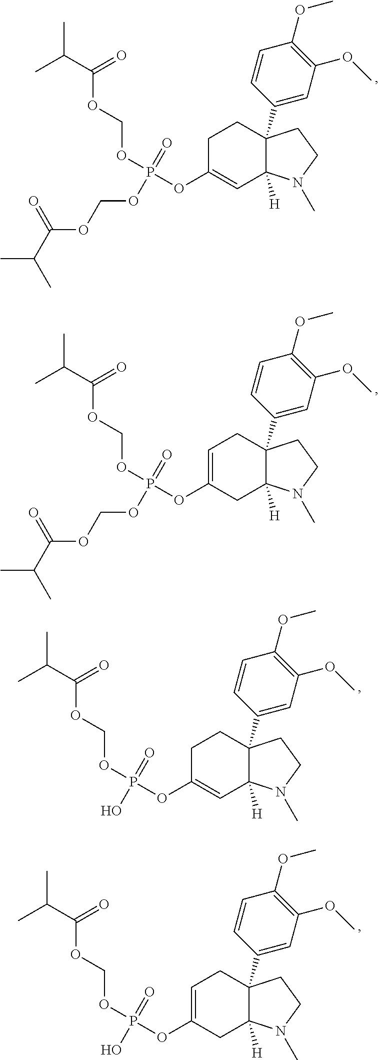

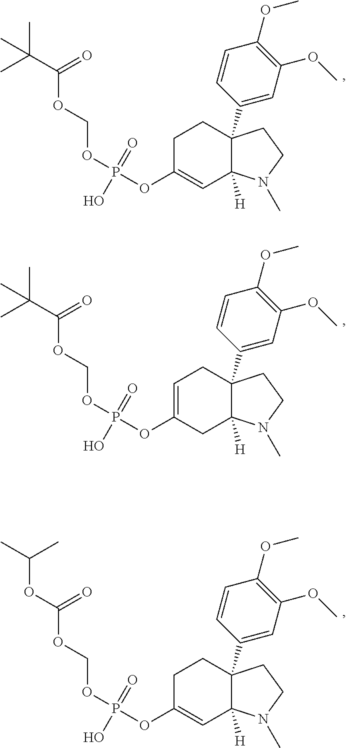

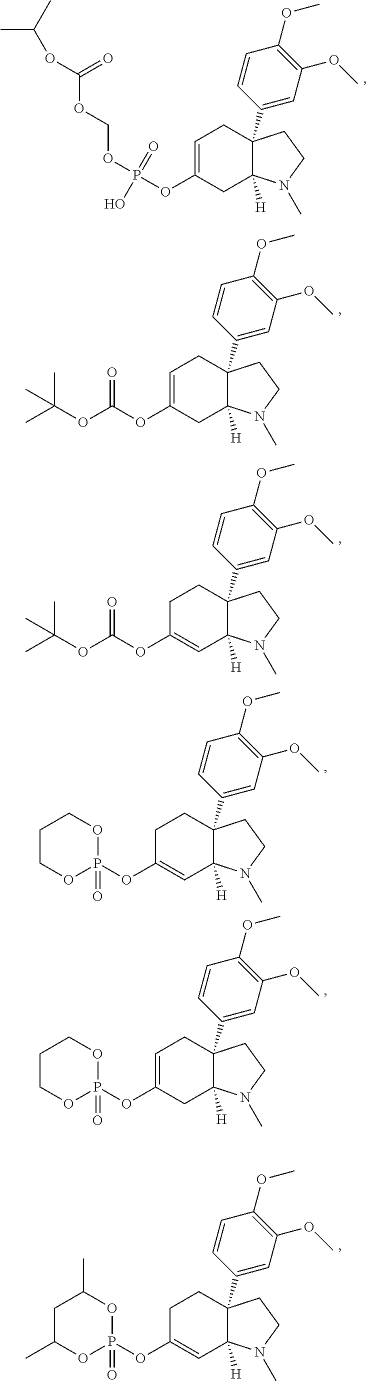

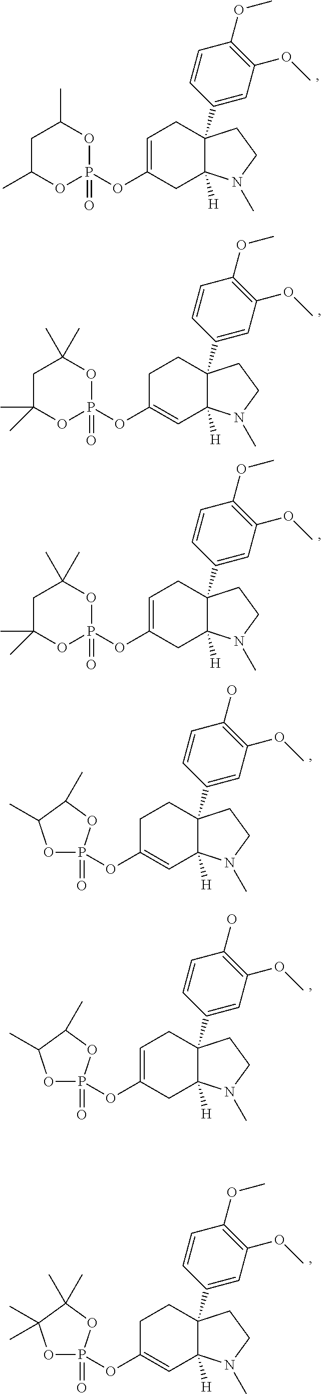

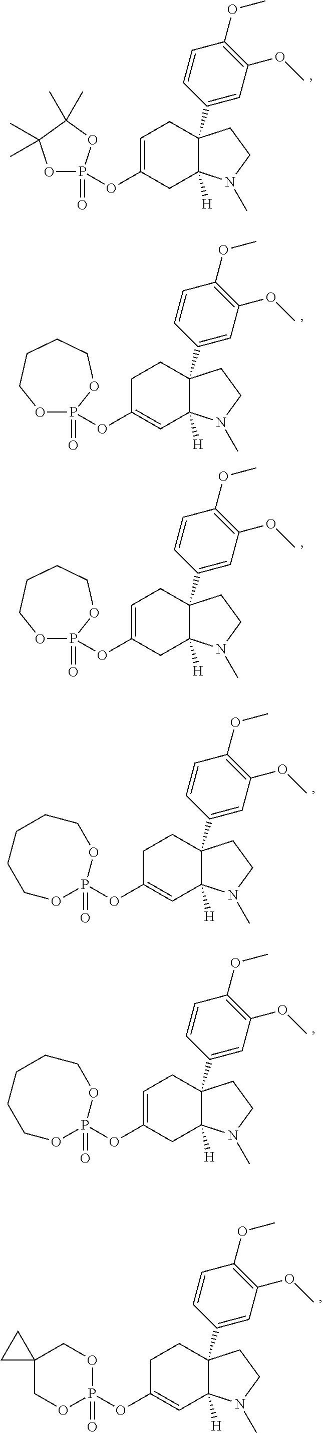

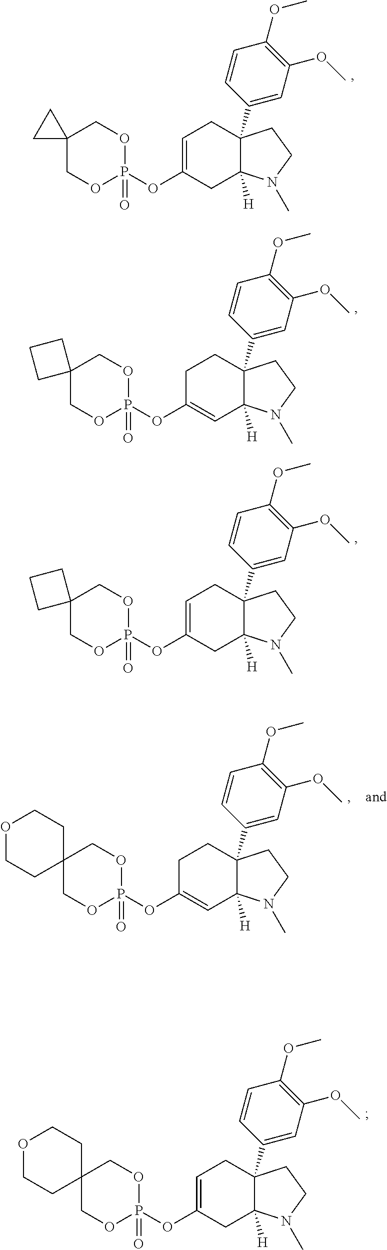

or a pharmaceutically acceptable salt thereof.

In some embodiments, the compound is selected from the group consisting of:

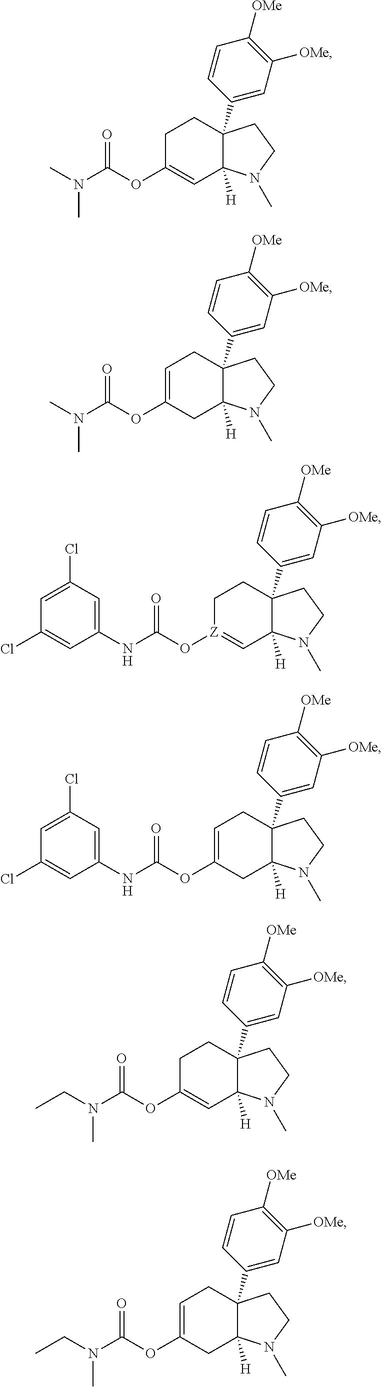

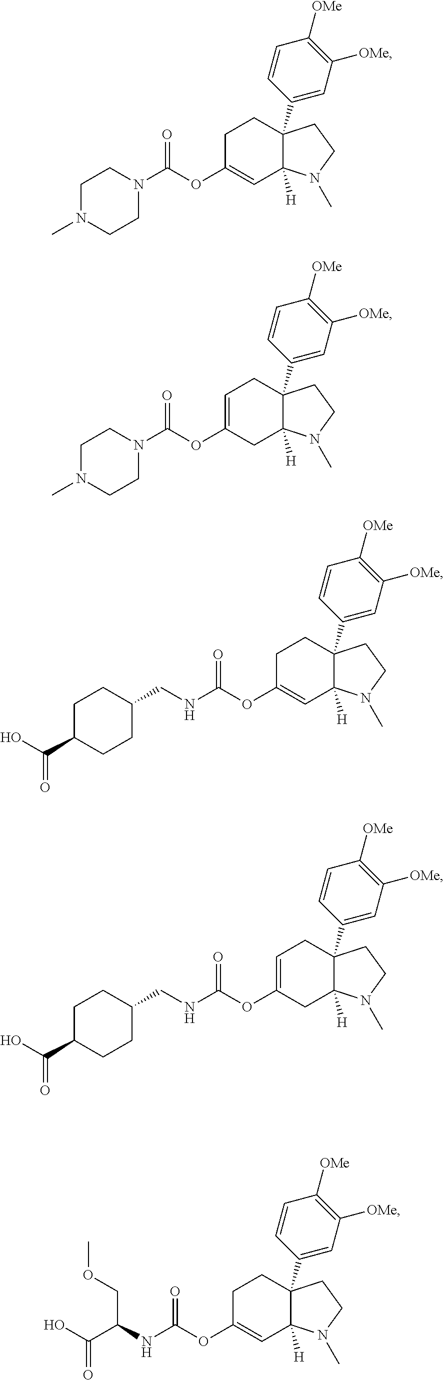 [SMILES: COc1ccc([C@@]23CCC(OC(=O)N4CCN(C)CC4)=C[C@@H]2N(C)CC3)cc1OC ; COc1ccc([C@@]23CC=C(OC(=O)N4CCN(C)CC4)C[C@@H]2N(C)CC3)cc1OC ; COc1ccc([C@@]23CCC(OC(=O)NC[C@H]4CC[C@H](C(=O)O)CC4)=C[C@@H]2N(C)CC3)cc1OC ; COc1ccc([C@@]23CC=C(OC(=O)NC[C@H]4CC[C@H](C(=O)O)CC4)C[C@@H]2N(C)CC3)cc1OC ; COC[C@@H](NC(=O)OC1=C[C@@H]2N(C)CC[C@]2(c2ccc(OC)c(OC)c2)CC1)C(=O)O]

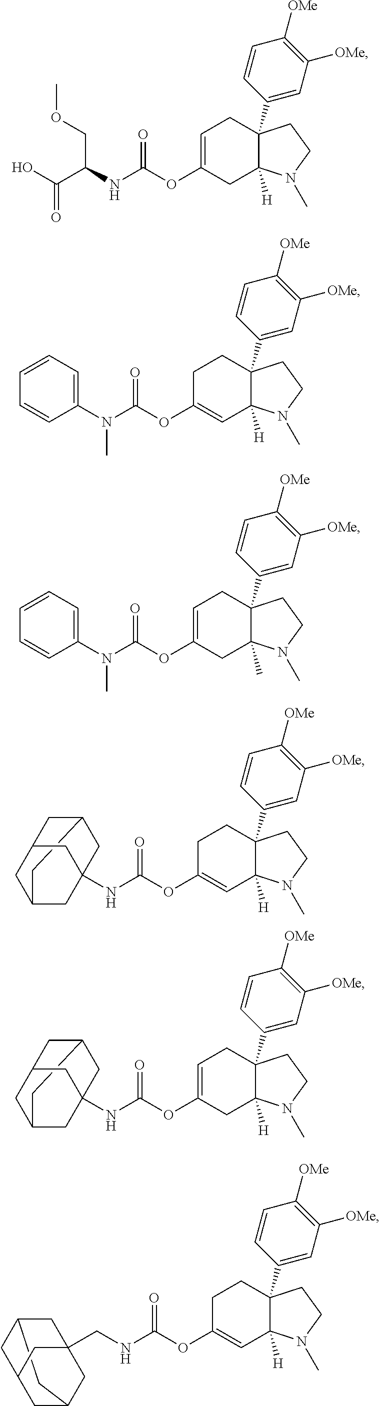

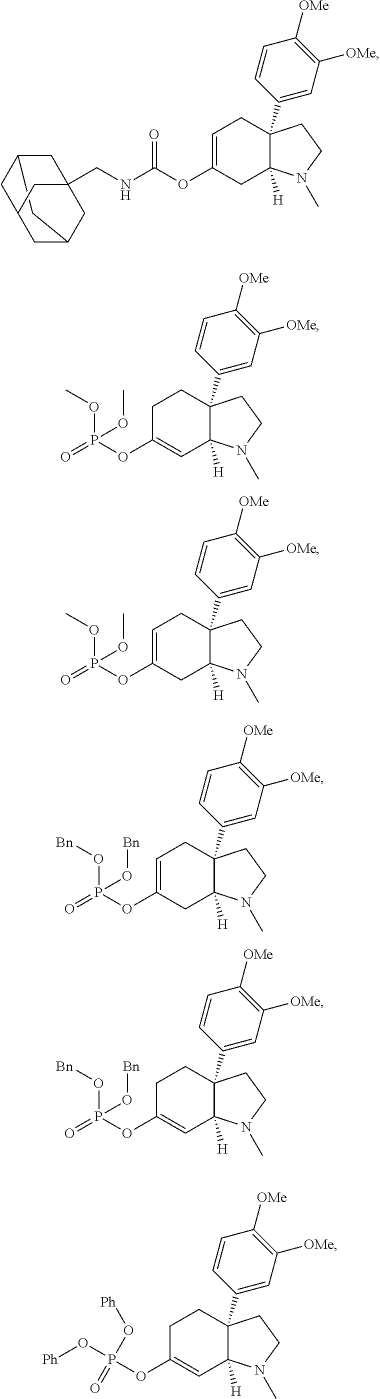

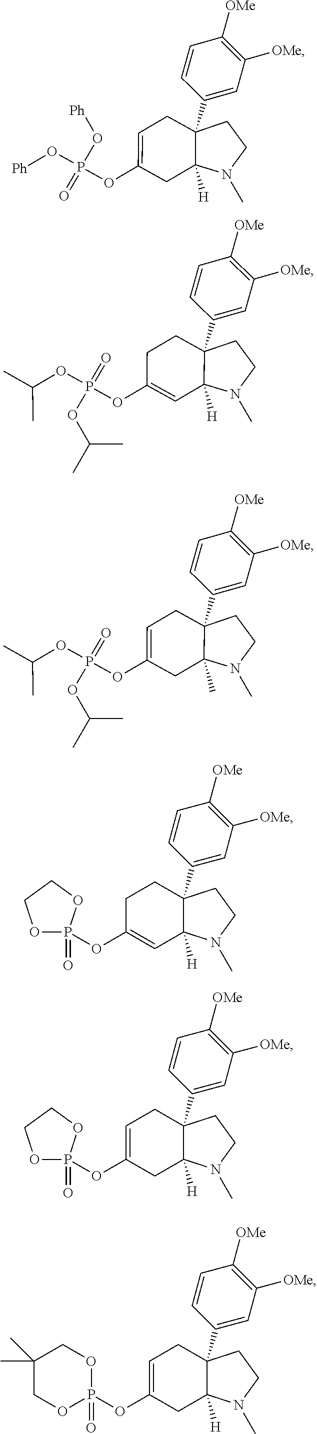

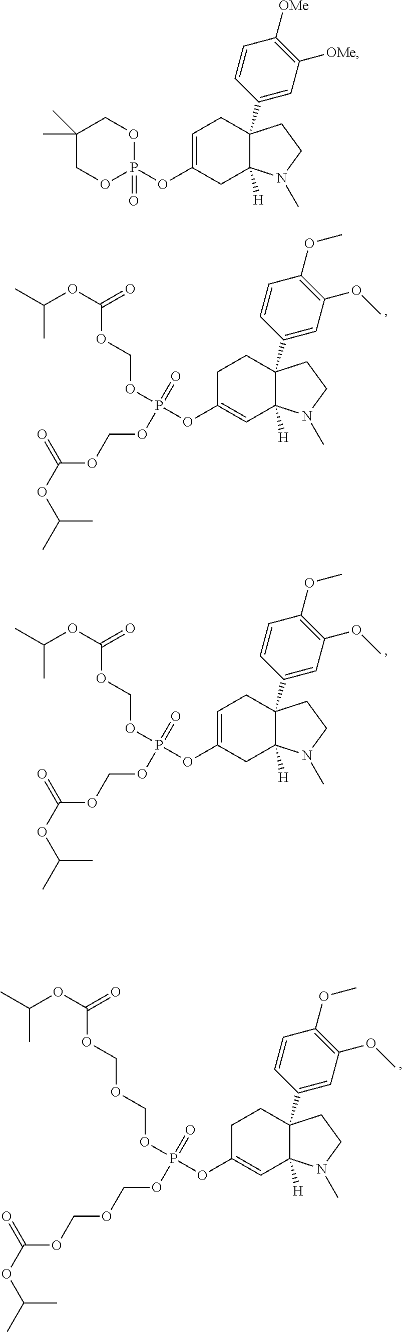

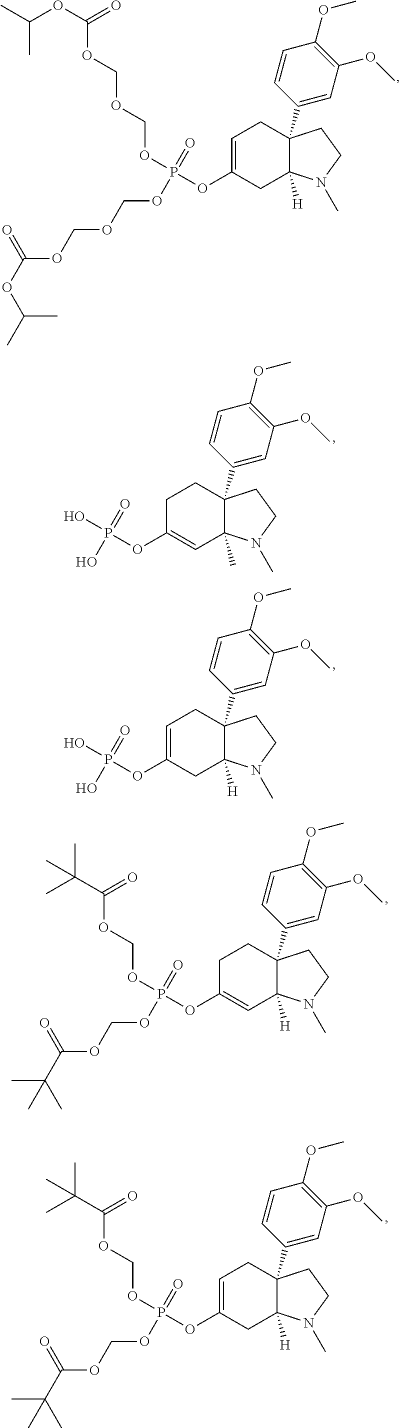

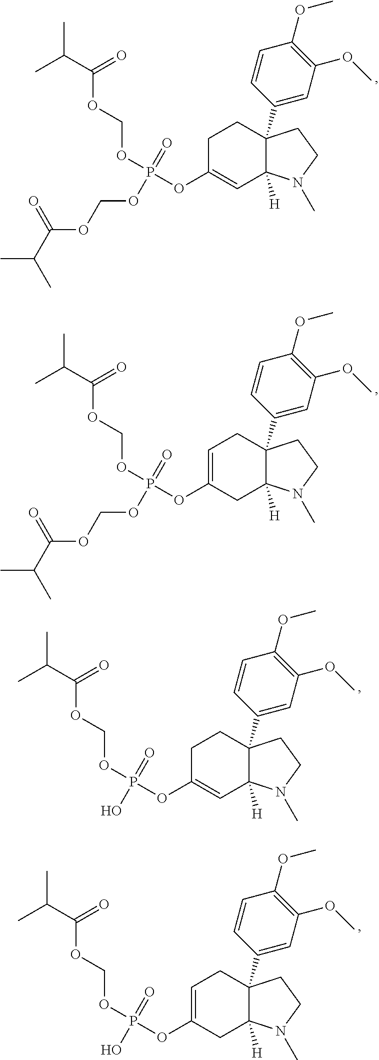

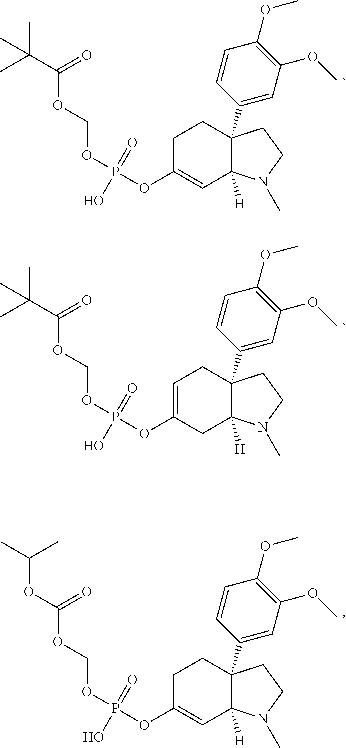

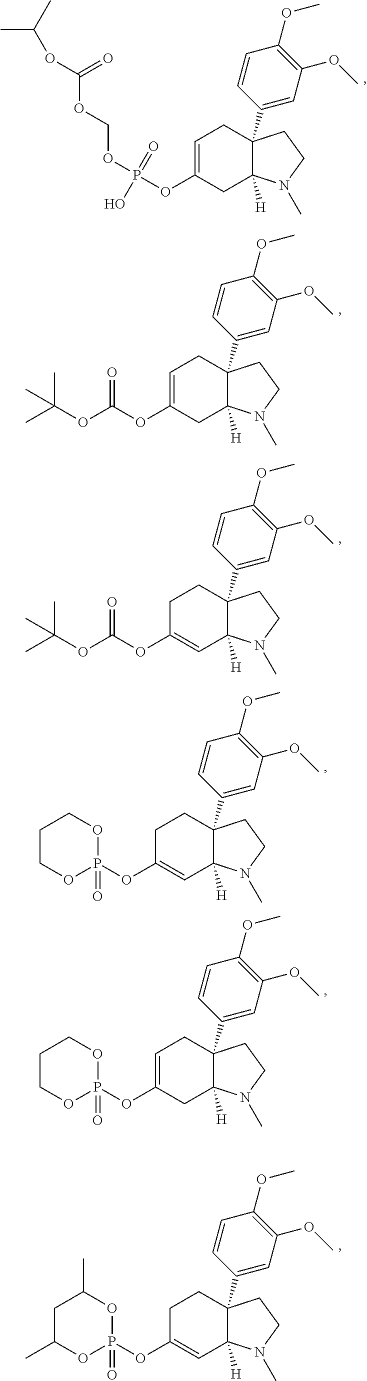

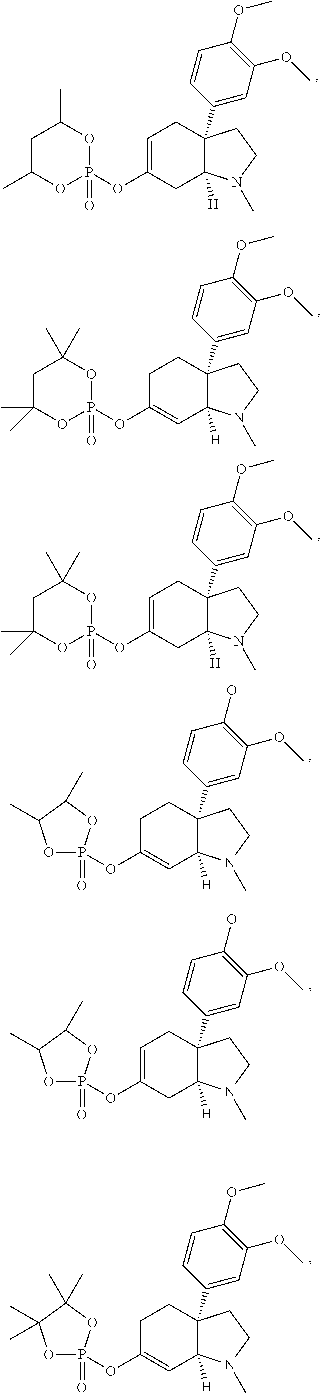

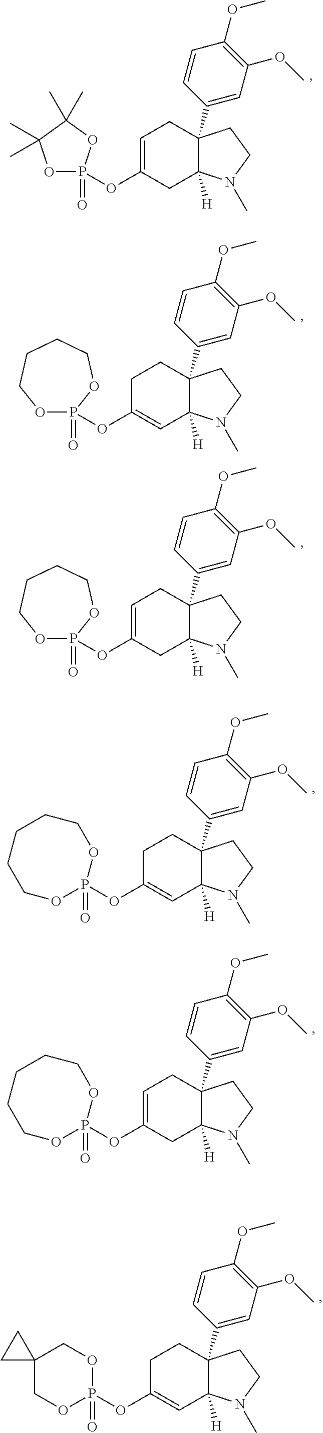

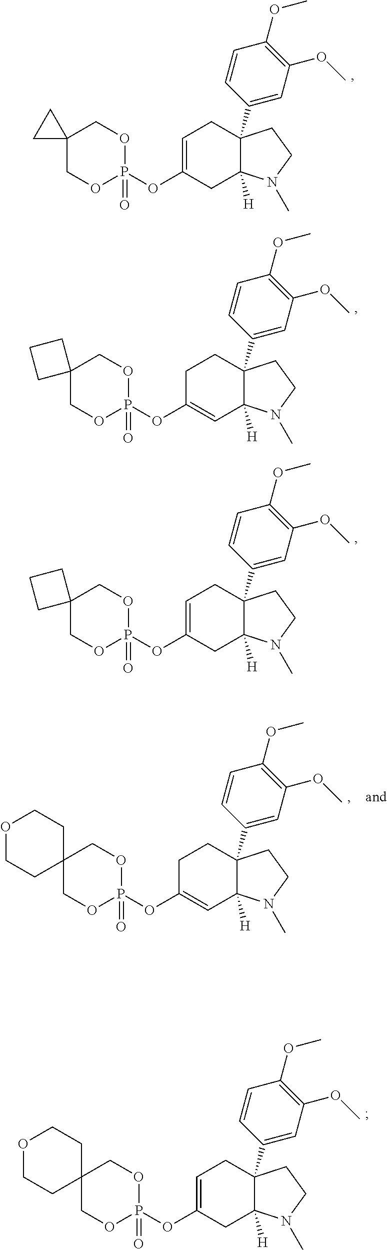

or a pharmaceutically acceptable salt thereof, and the compound has the absolute stereochemistry shown.

The invention is based in part on the discovery of compounds having useful and markedly different from naturally occurring mesembrine, but that can be converted to mesembrine under biologically relevant conditions. Certain compounds provided herein convert to mesembrine under physiologically relevant conditions.

In certain embodiments, the present disclosure provides a method of treating a mental disorder, comprising administering to the subject a compound of the present disclosure.

Numerous embodiments are further provided that can be applied to any aspect of the present invention described herein.

### BRIEF DESCRIPTION OF THE DRAWINGS

FIGS. 1A and 1B show plasma and brain concentrations-time profiles (mean) of 321 and 001 (metabolite) in male C57BL/6 mice following a single intravenous administration of 321 (Dose: 2 mg/kg, IV) on a linear scale (FIG. 1A) and log scale (FIG. 1B).

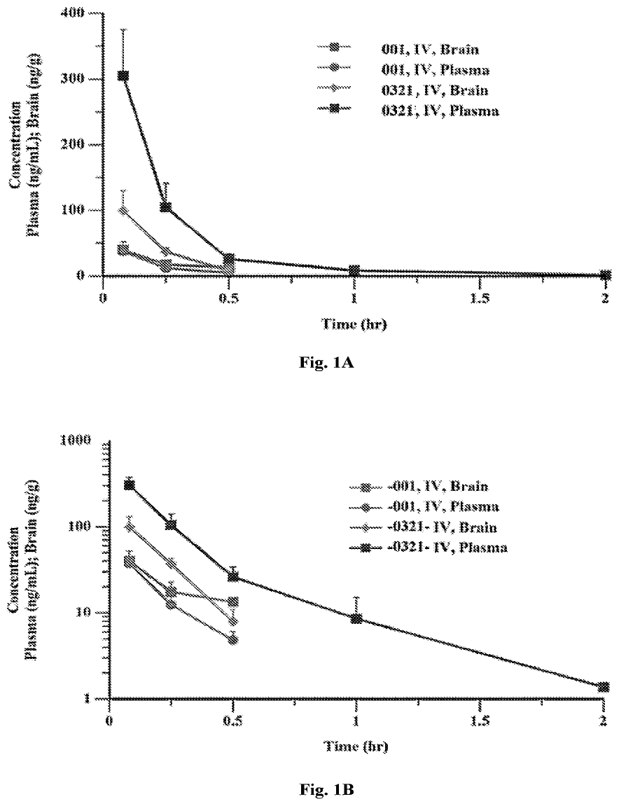

FIGS. 2A and 2B show plasma and brain concentrations-time profiles (mean) of 323 and 001 (metabolite) in male C57BL/6 mice following a single intravenous administration of 323 (Dose: 2 mg/kg, IV) on a linear scale (FIG. 2A) and log scale (FIG. 2B).

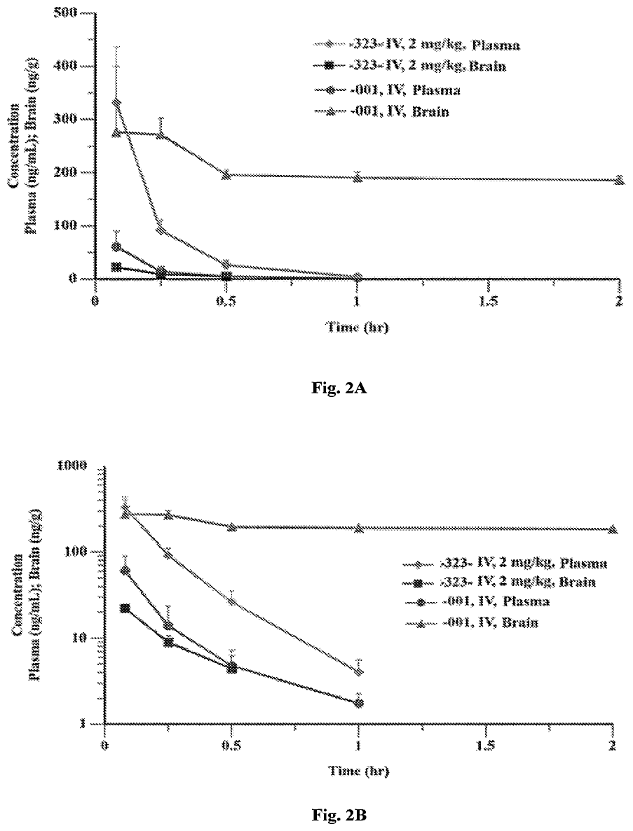

FIGS. 3A and 3B show plasma and brain concentrations-time profiles (mean) of 001 (metabolite) in male C57BL/6 mice following a single intravenous administration of 358 (Dose: 2 mg/kg, IV) on a linear scale (FIG. 3A) and log scale (FIG. 3B).

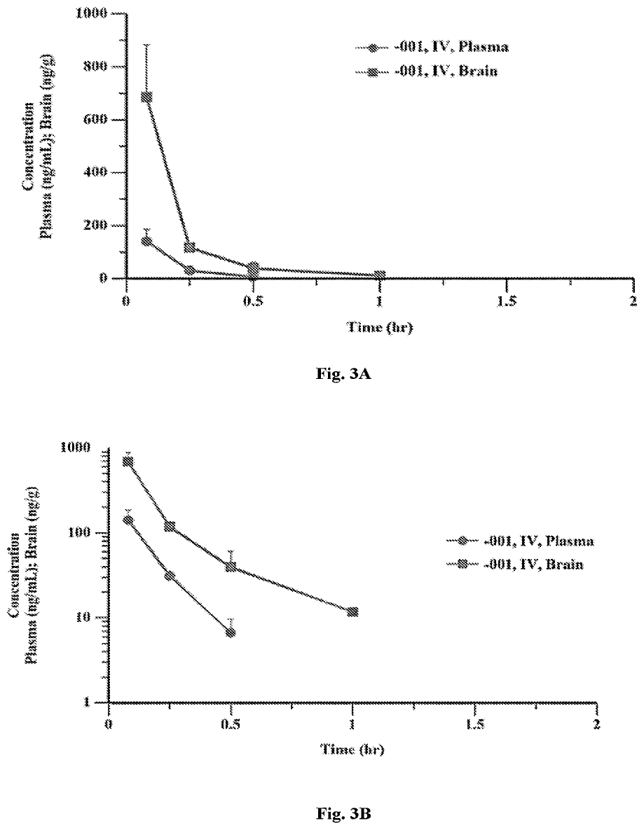

### DETAILED DESCRIPTION

The present invention is based, at least in part, on mesembrine and analogs thereof. Although (−) mesembrine is bioactive with certain desirable pharmacologic effects, certain other properties are less than ideal for use as a therapeutic. For example, the pharmacokinetics described for (−) mesembrine show rapid metabolism and excretion, which an undesirably low half-life in plasma of less than 2 hours. To take advantage of the desirable properties of (−) mesembrine and improve upon absorption, distribution, metabolism and excretion (ADME) that impact pharmacokinetics (PK), compounds have been developed and described here. At least some of the compounds have the shared properties characterized by one or more of the following: (1) they have a function group manipulation at, or related to, the ketone; (2) the modification to the structure impacts physiochemical properties; and (3) they are intended to tune the ADME/PK of mesembrine (e.g., (−) mesembrine) in vivo.

### EXEMPLARILY COMPOUNDS OF THE INVENTION

In certain embodiments, compounds described herein can form mesembrine (e.g., (−) mesembrine) under biologically relevant conditions. For example, in some embodiments, compounds of disclosed herein (e.g., compounds of Formula (I)) can hydrolyze at a rate that is advantageous for providing a desired bioabsorption (% F) following oral administration by a mammal and leading to a desired pharmacokinetic profile of mesembrine (e.g., (−) mesembrine) to the mammal. When a compound of Formula (I) is prepared from a single enantiomer (e.g., from (−) mesembrine), the compound of Formula (I) is itself considered to be a single enantiomer (ignoring possible stereocenters in the constituent R² moiety), absent evidence to the contrary.

In some embodiments, a compound according to the present disclosure is a compound of formula (I):

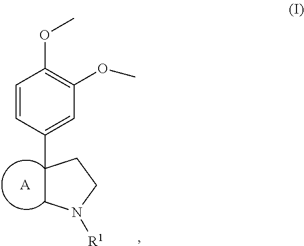

or a pharmaceutically acceptable salt thereof,
wherein

R¹ is H or C₁-C₇ alkyl;

ring A is

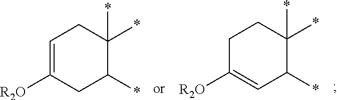

wherein * denotes the attachment points of ring A to the compound of formula (I),

R² is —C(O)NR³R⁴, —C(O)OR⁶, or —P(O)OR⁴OR⁵;

R³ is H, C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;

R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R³ and R⁴ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁵ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R⁴ and R⁵ combine to form a 4-8 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and

n is 1 or 2.

In some embodiments, a compound according to the present disclosure is of formula (I):

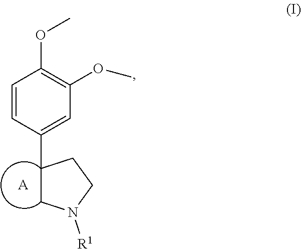

or a pharmaceutically acceptable salt thereof,
wherein

R¹ is H or C₁-C₇ alkyl;

ring A is

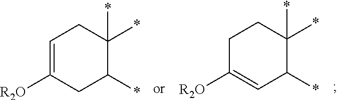

wherein * denotes the attachment points of ring A to the compound of formula (I),

R² is —C(O)NR³R⁴, —C(O)OR⁶, or —P(O)OR⁴OR⁵;

R³ is H, C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;

R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R³ and R⁴ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁵ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R⁴ and R⁵ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and

n is 1 or 2.

In certain embodiments, the compound is of formula (I-A):

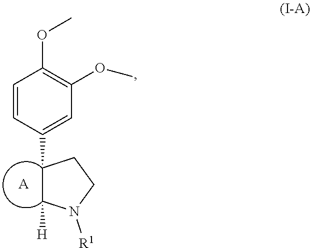

or a pharmaceutically acceptable salt thereof. In certain embodiments, the compound has the absolute stereochemistry shown.

In certain embodiments, the compound is of formula (II):

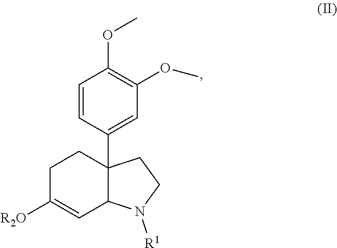

or a pharmaceutically acceptable salt thereof.

In certain embodiments, the compound is of formula (II-A):

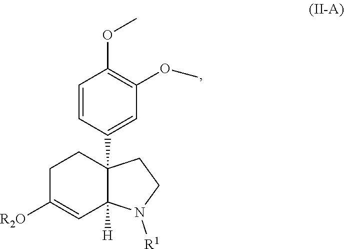

or a pharmaceutically acceptable salt thereof. In certain embodiments, the compound has the absolute stereochemistry shown.

In certain embodiments, the compound is of formula (III):

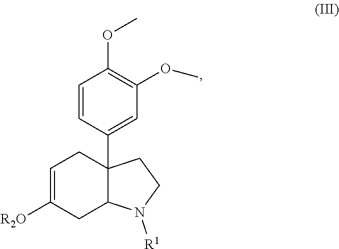

or a pharmaceutically acceptable salt thereof.

In certain embodiments, the compound is of formula (III-A):

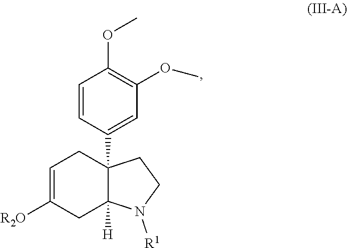

or a pharmaceutically acceptable salt thereof. In certain embodiments, the compound has the absolute stereochemistry shown.

In certain embodiments, R² is —C(O)NR³R⁴. In certain preferred embodiments, R³ is C₁-C₆ alkyl (e.g., methyl or ethyl), C₂-C₆ alkenyl, C₃-C₁₀ cycloalkyl (e.g., adamantyl), C₁-C₃ alkyl-C₃-C₁₀ cycloalkyl, phenyl (e.g., phenyl or dichlorphenyl), or 5- to 7-membered heteroaryl. In some embodiments, each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy (e.g., methoxy), nitro, —N(C₁-C₃ alkyl)₂, —NH₂, —N(H)C₁-C₃ alkyl, C₁-C₃ haloalkyl, —COOH, cyano, phenyl, or phenoxy. In certain preferred embodiments, R⁴ is C₁-C₆ alkyl, phenyl, —(CH₂O), —C(O)OC₁-C₆ alkyl, or —(CH₂O)ₙ—C(O)C₁-C₆ alkyl. In some embodiments, each hydrogen atom in the preceding C₁-C₆ alkyl is optionally substituted by phenyl. In certain embodiments, one of R³ and R⁴ is H. In certain preferred embodiments, both of R³ and R⁴ are not H.

In certain embodiments, R³ and R⁴ combine to form a 4-7 membered heterocycle. In some embodiments, each hydrogen atom in the 4-7 membered heterocycle is optionally substituted by halo, hydroxy, C₁-C₃ alkyl, C₁-C₃ alkoxy, C₃-C₁₀ cycloalkyl (e.g., adamantyl), phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy. For example, in certain embodiments R³ and R⁴ combine to form is

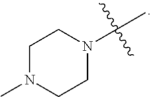 [SMILES: *N1CCN(C)CC1]

In certain embodiments, R² is —C(O)OR⁶. In some embodiments, R⁶ is C₁-C₆ alkyl (e.g., t-butyl), C₂-C₆ alkenyl, C₃-C₁₀ cycloalkyl (e.g., adamantyl), C₁-C₃ alkyl-C₃-C₁₀ cycloalkyl, phenyl, or 5- to 7-membered heteroaryl. In some embodiments, each hydrogen atom in C₁-C₆ alkyl, C₂-C₆ alkenyl, C₃-C₁₀ cycloalkyl, phenyl, and 5- to 7-membered heteroaryl is optionally substituted by halo, C₁-C₆ alkyl, C₁-C₃ alkoxy, nitro, —N(C₁-C₃ alkyl)₂, C₁-C₃ haloalkyl, —COOH, cyano, phenyl, or phenoxy.

In certain embodiments, R² is —P(O)OR⁴OR⁵. In certain preferred embodiments, each of R⁴ and R⁵ is independently C₁-C₆ alkyl (e.g., isopropyl or methyl), phenyl, —(CH₂O)ₙ—C(O)OC₁-C₆ alkyl (e.g., —CH₂O—C(O)C(CH₃)₃), or —(CH₂O)ₙ—C(O)C₁-C₆ alkyl. In some embodiments, each hydrogen atom in the C₁-C₆ alkyl in the preceding options is optionally substituted by halo, hydroxy, C₁-C₃ alkyl, C₁-C₃ alkoxy, C₃-C₁₀ cycloalkyl (e.g., adamantyl), phenyl (e.g., a substituted methyl to form benzyl), 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy. In some embodiments, one of R⁴ and R⁵ is C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy. In certain preferred embodiments, both of R⁴ and R⁵ are not H. In some embodiments, both of R⁴ and R⁵ are H. In some embodiments, one of R⁴ and R⁵ is H. In some embodiments, R⁵ is H.

In certain preferred embodiments, R⁴ and R⁵ combine to form a 4-7 membered heterocycle. In some embodiments, each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy. For example, in some embodiments, R⁴ and R⁵ can combine to form

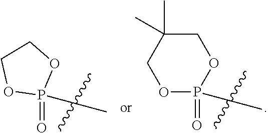

In some embodiments, the cyclic substituents (e.g., C₃-C₁₀ cycloalkyl, phenyl, 5-7 membered heterocycle, 5- to 7-membered heteroaryl), when present on the 4-7 membered heterocycle can combine to form a fused ring (e.g., aryl, heteroaryl, cycloalkyl, heterocycloalkyl), for example

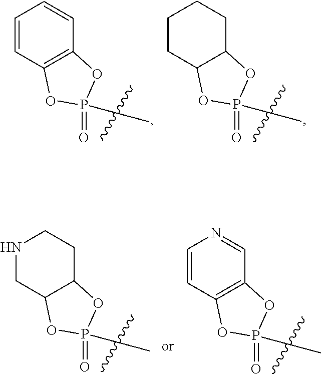 [SMILES: *P1(=O)Oc2ccccc2O1 ; *P1(=O)OC2CCCCC2O1 ; *P1(=O)OC2CCNCC2O1 ; *P1(=O)Oc2ccncc2O1]

or be attached to a single atom of the 4-7 membered heterocycle, for example

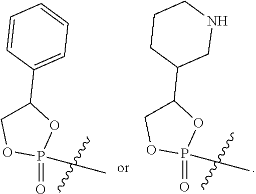 [SMILES: *P1(=O)OCC(c2ccccc2)O1 ; *P1(=O)OCC(C2CCCNC2)O1]

In certain preferred embodiments, R⁴ and R⁵ combine to form a 4-8 membered heterocycle. In some embodiments, each hydrogen atom in the 4-8 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy. In some embodiments, R⁴ and R⁵ can combine to form

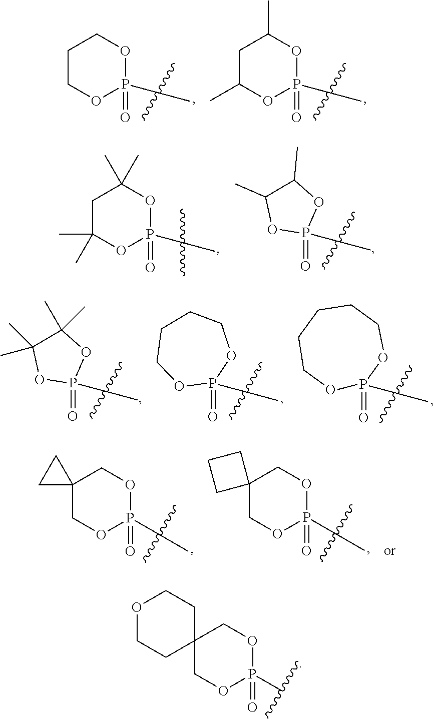

In some embodiments, the cyclic substituents (e.g., C₃-C₁₀ cycloalkyl, phenyl, 5-7 membered heterocycle, 5- to 7-membered heteroaryl), when present on the 4-8 membered heterocycle can combine to form a fused ring (e.g., aryl, heteroaryl, cycloalkyl, heterocycloalkyl), for example

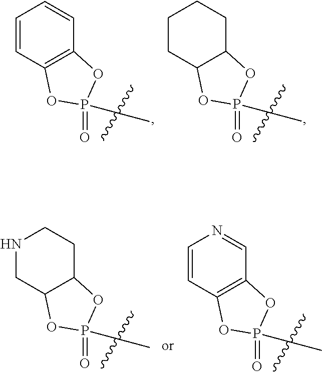 [SMILES: *P1(=O)Oc2ccccc2O1 ; *P1(=O)OC2CCCCC2O1 ; *P1(=O)OC2CCNCC2O1 ; *P1(=O)Oc2ccncc2O1]

or be attached to a single atom of the 4-7 membered heterocycle, for example

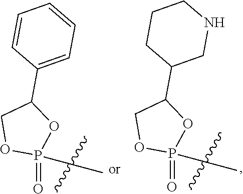 [SMILES: *P1(=O)OCC(c2ccccc2)O1 ; *P1(=O)OCC(C2CCCNC2)O1]

or form a spiro cycloalkyl or spiro heterocycle, for example

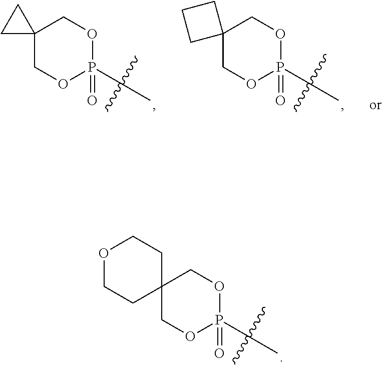 [SMILES: *P1(=O)OCC2(CC2)CO1 ; *P1(=O)OCC2(CCC2)CO1 ; *P1(=O)OCC2(CCOCC2)CO1]

In some embodiments, n is 1 or 2, and preferably n is 1.

In certain embodiments, wherein R¹ is C₁-C₇ alkyl (e.g., preferably methyl).

In certain embodiments, the compound is selected from:

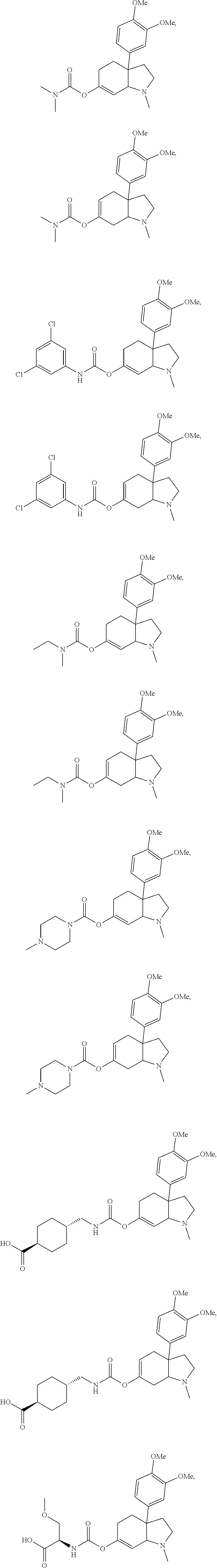

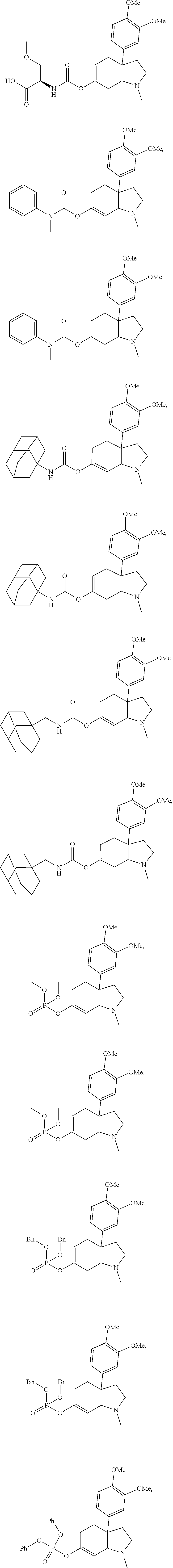

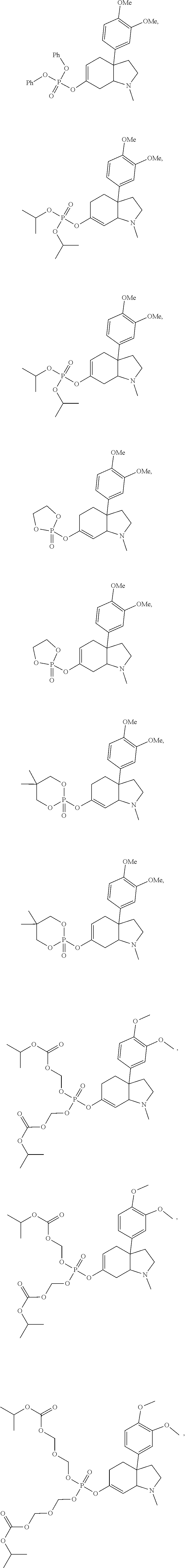

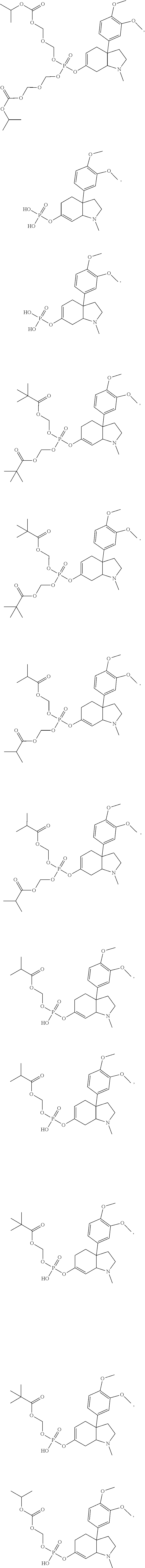

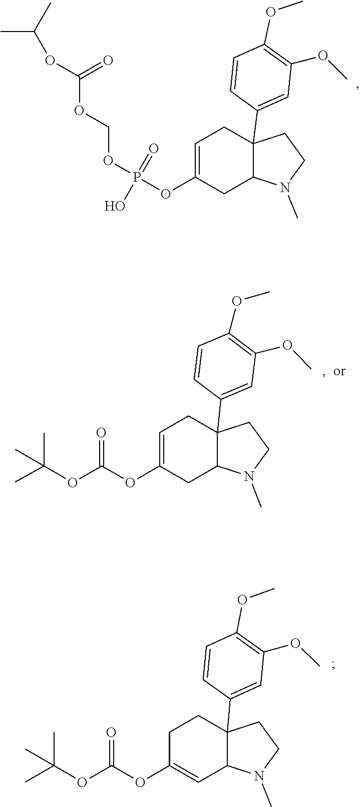

or a pharmaceutically acceptable salt thereof.

In certain embodiments, the compound is selected from:

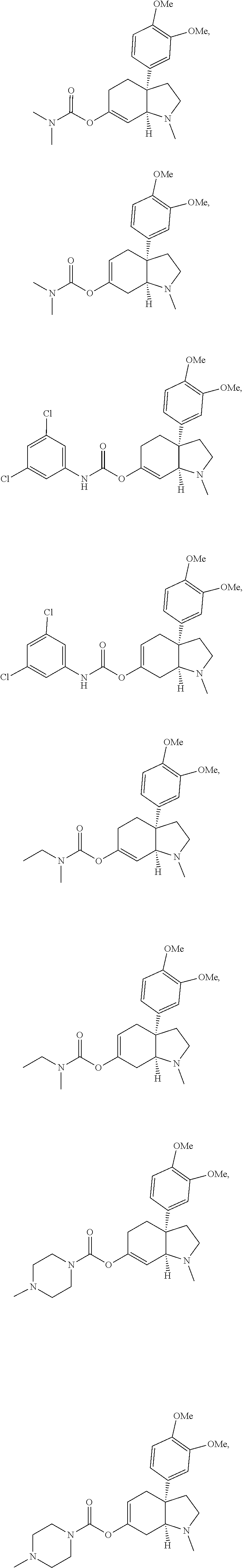

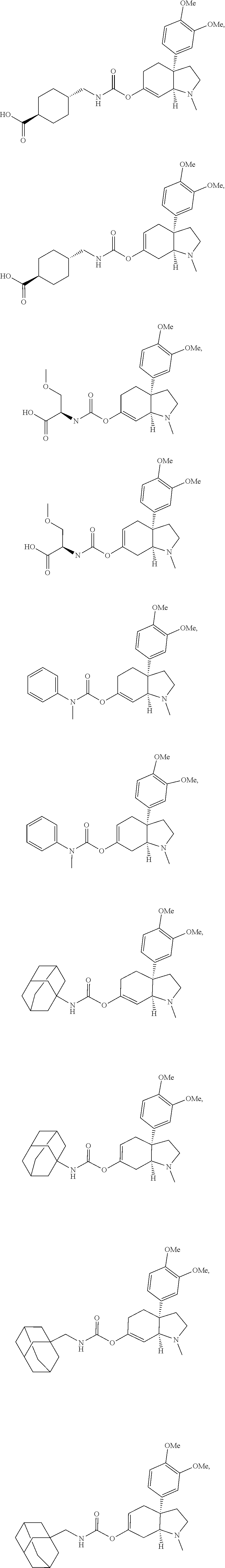

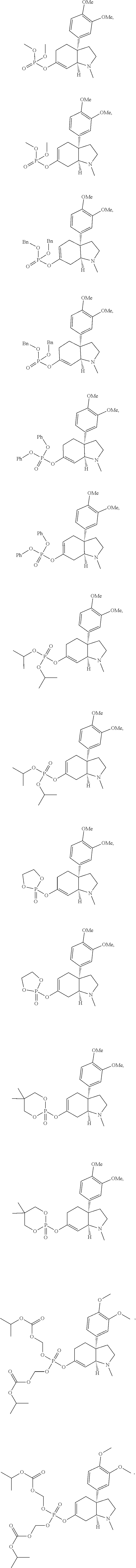

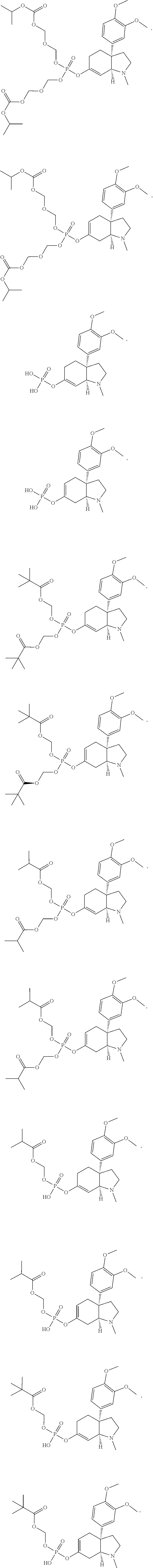

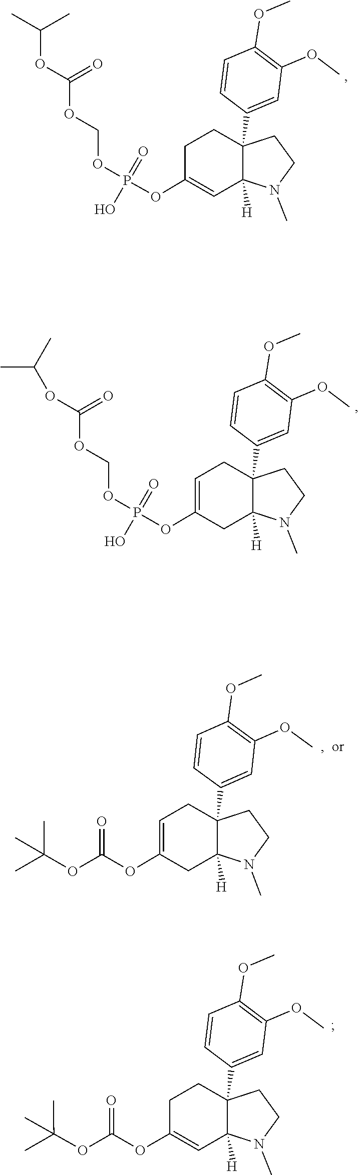

or a pharmaceutically acceptable salt thereof.

In certain embodiments, the compound is selected from:

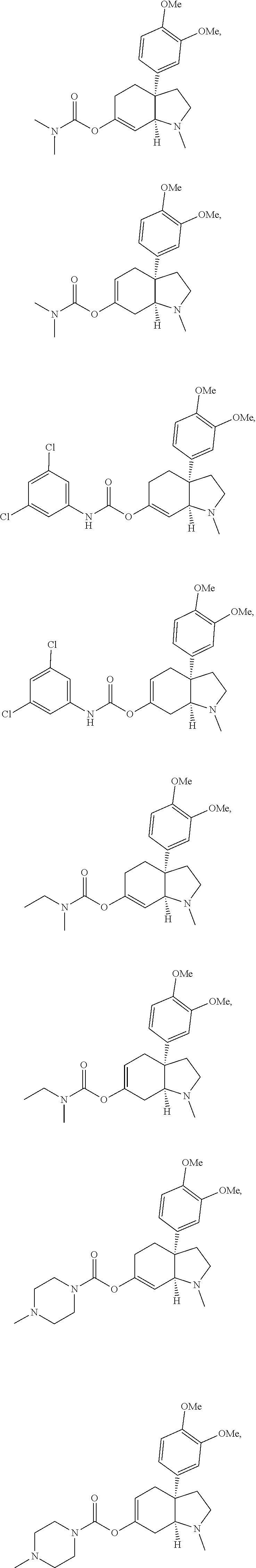

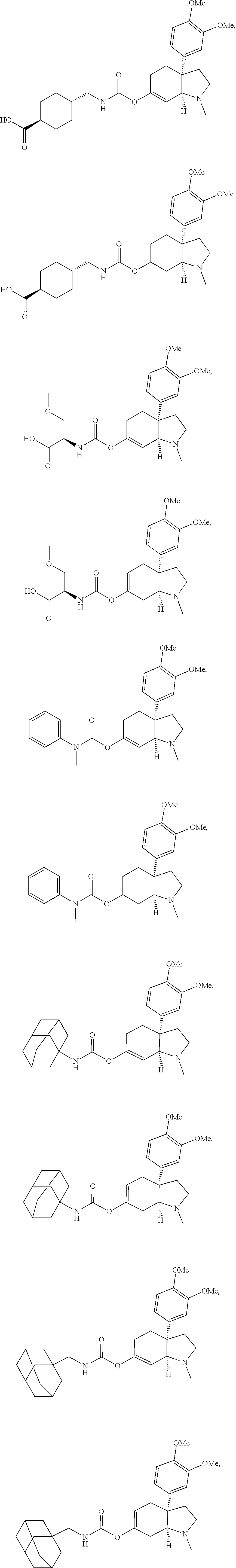

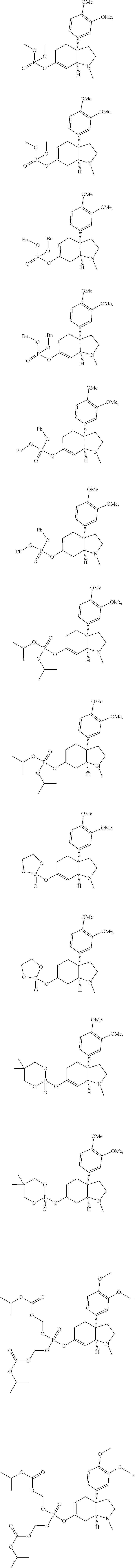

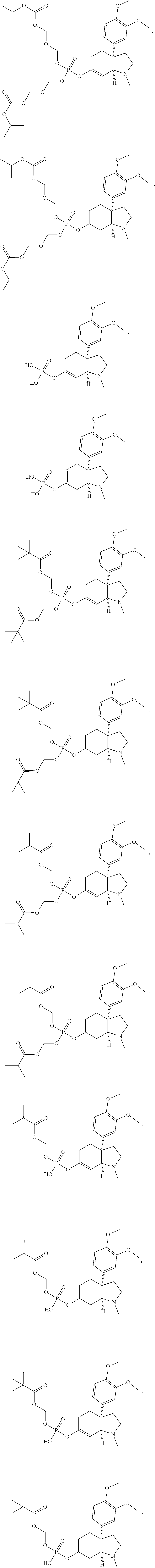

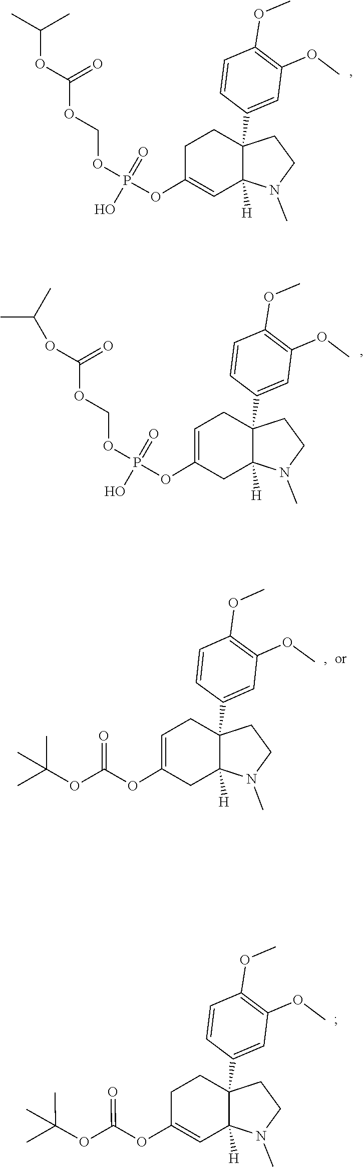

or a pharmaceutically acceptable salt thereof; and the compound has the absolute stereochemistry shown.

In certain embodiments, the compound is selected from:

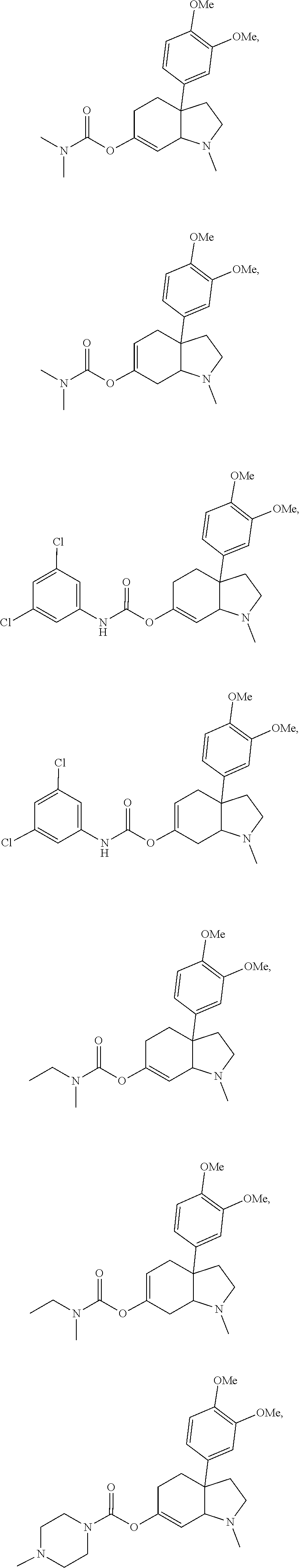

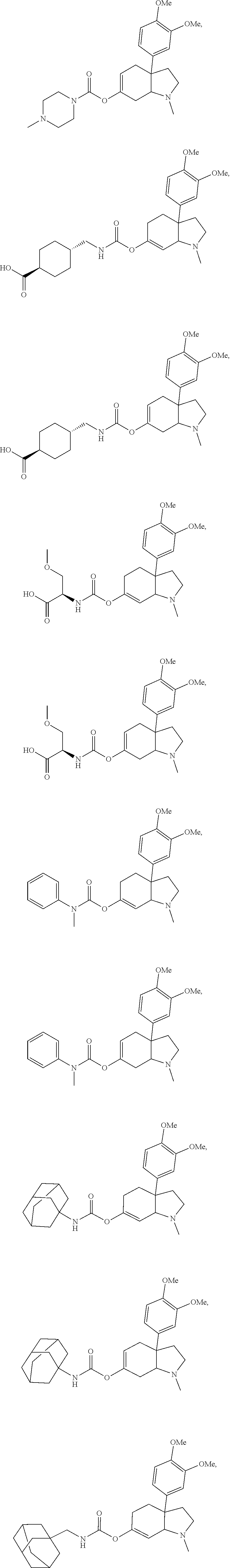

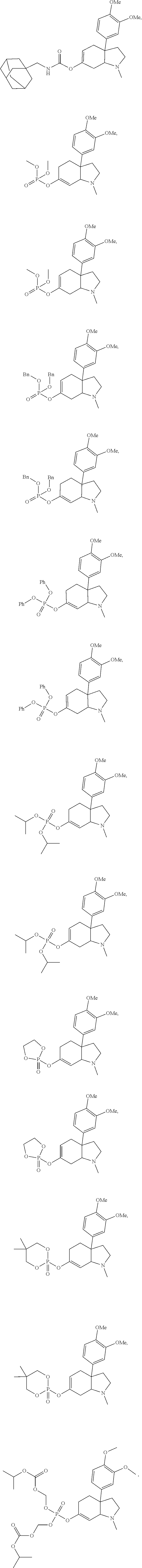

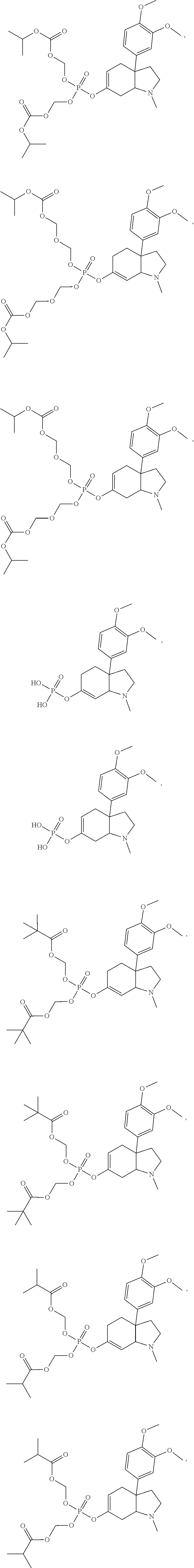

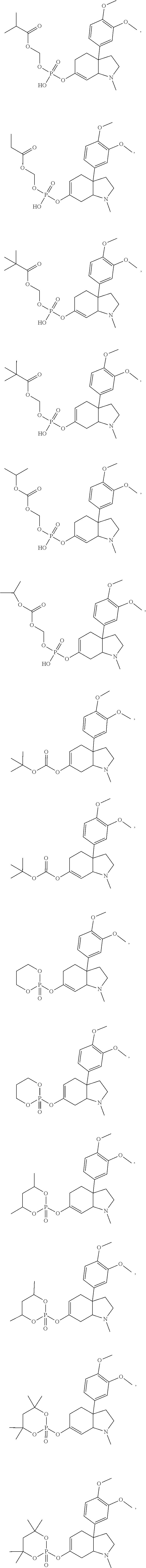

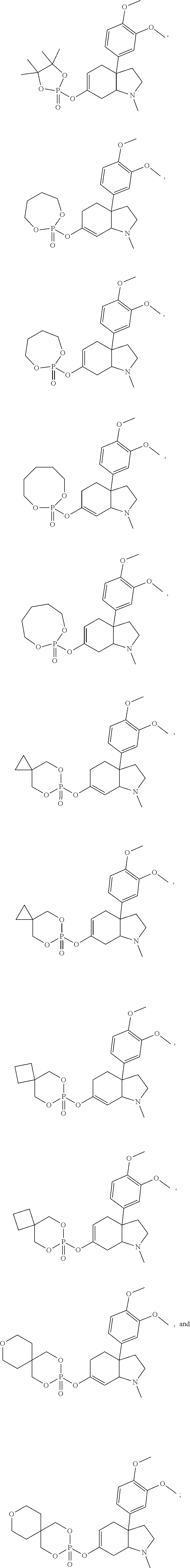

or a pharmaceutically acceptable salt thereof.

In certain embodiments, the compound is selected from:

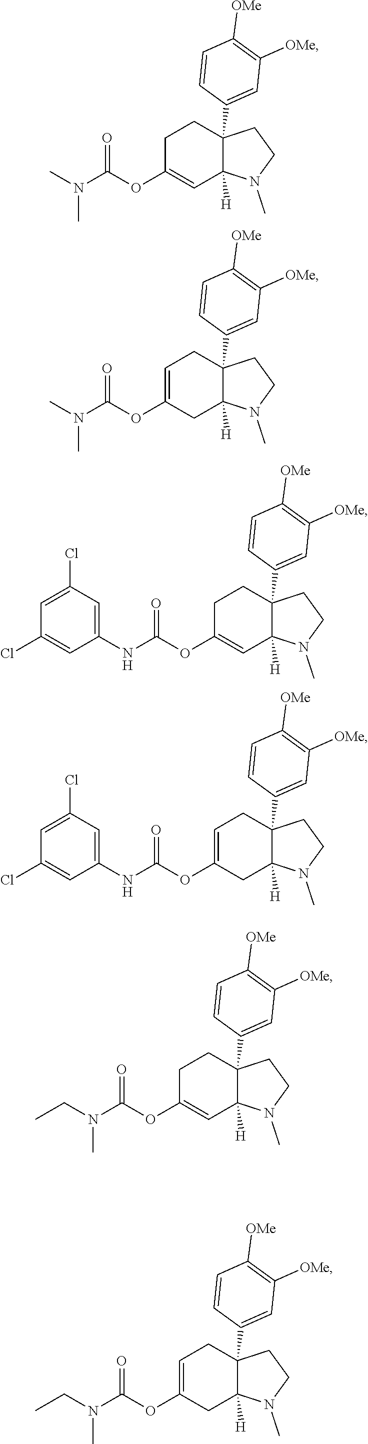

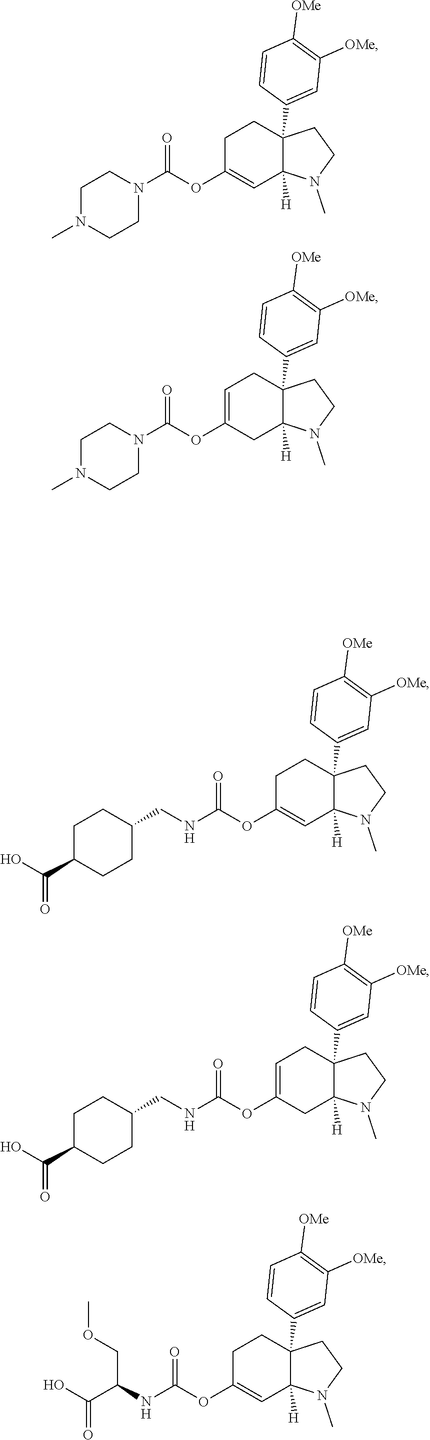 [SMILES: COc1ccc([C@@]23CCC(OC(=O)N4CCN(C)CC4)=C[C@@H]2N(C)CC3)cc1OC ; COc1ccc([C@@]23CC=C(OC(=O)N4CCN(C)CC4)C[C@@H]2N(C)CC3)cc1OC ; COc1ccc([C@@]23CCC(OC(=O)NC[C@H]4CC[C@H](C(=O)O)CC4)=C[C@@H]2N(C)CC3)cc1OC ; COc1ccc([C@@]23CC=C(OC(=O)NC[C@H]4CC[C@H](C(=O)O)CC4)C[C@@H]2N(C)CC3)cc1OC ; COC[C@@H](NC(=O)OC1=C[C@@H]2N(C)CC[C@]2(c2ccc(OC)c(OC)c2)CC1)C(=O)O]

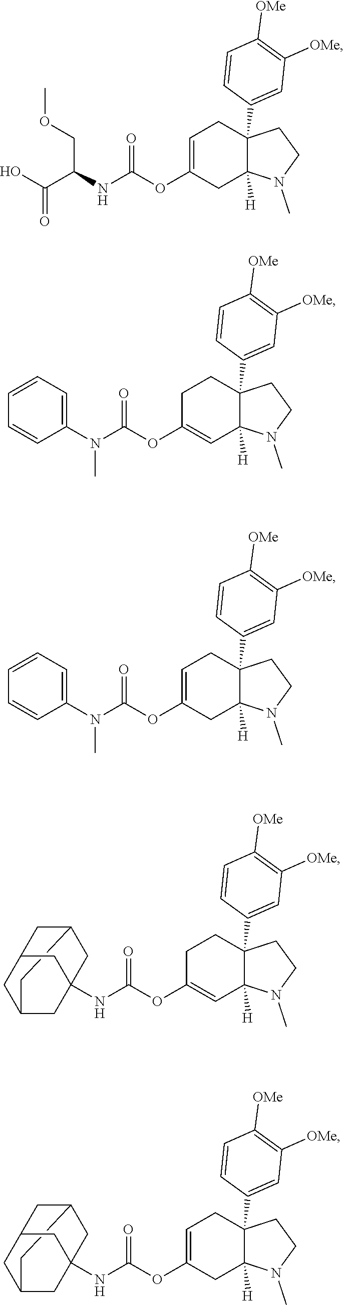 [SMILES: COC[C@@H](NC(=O)OC1=CC[C@@]2(c3ccc(OC)c(OC)c3)CCN(C)[C@H]2C1)C(=O)O ; COc1ccc([C@@]23CCC(OC(=O)N(C)c4ccccc4)=C[C@@H]2N(C)CC3)cc1OC ; COc1ccc([C@@]23CC=C(OC(=O)N(C)c4ccccc4)C[C@@H]2N(C)CC3)cc1OC ; COc1ccc([C@@]23CCC(OC(=O)NC45CC6CC(CC(C6)C4)C5)=C[C@@H]2N(C)CC3)cc1OC ; COc1ccc([C@@]23CC=C(OC(=O)NC45CC6CC(CC(C6)C4)C5)C[C@@H]2N(C)CC3)cc1OC]

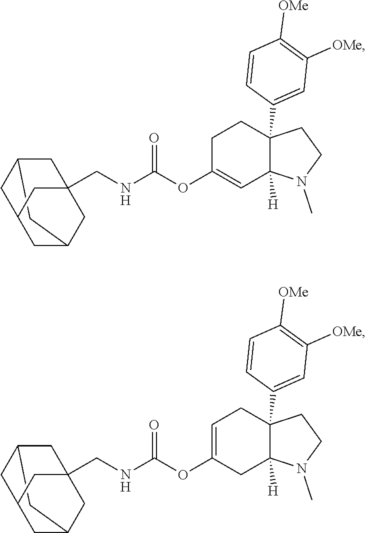 [SMILES: COc1ccc([C@@]23CCC(OC(=O)NCC45CC6CC(CC(C6)C4)C5)=C[C@@H]2N(C)CC3)cc1OC ; COc1ccc([C@@]23CC=C(OC(=O)NCC45CC6CC(CC(C6)C4)C5)C[C@@H]2N(C)CC3)cc1OC]

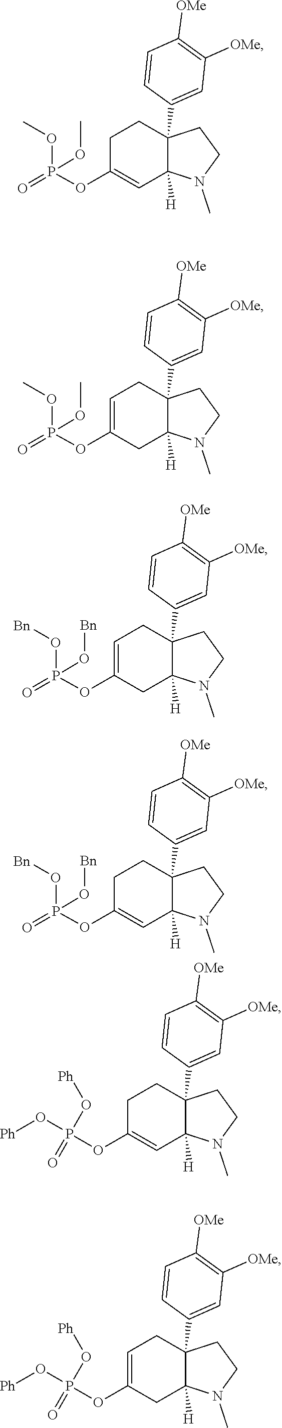

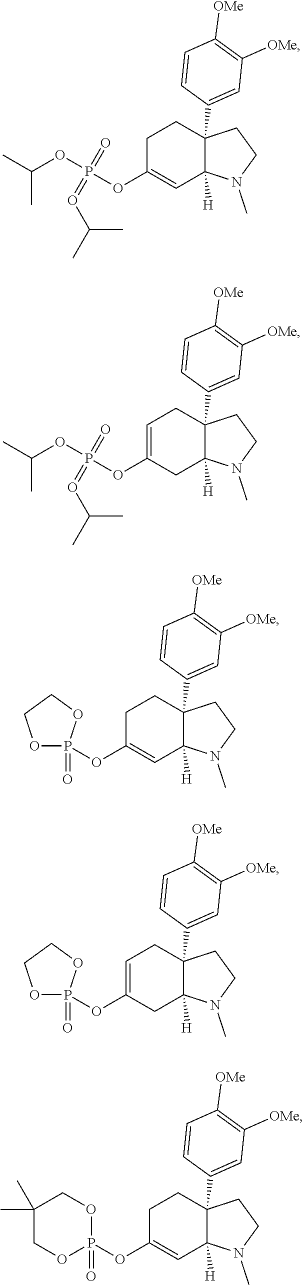

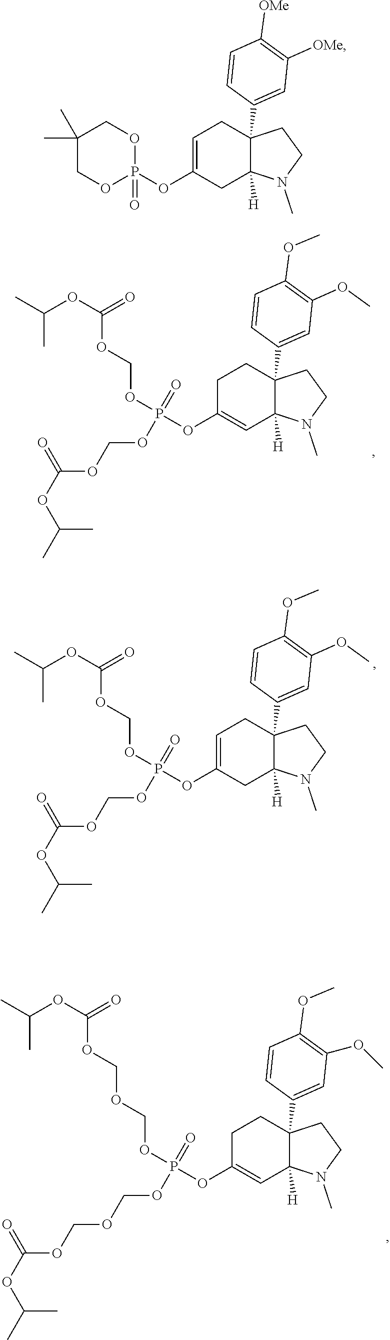

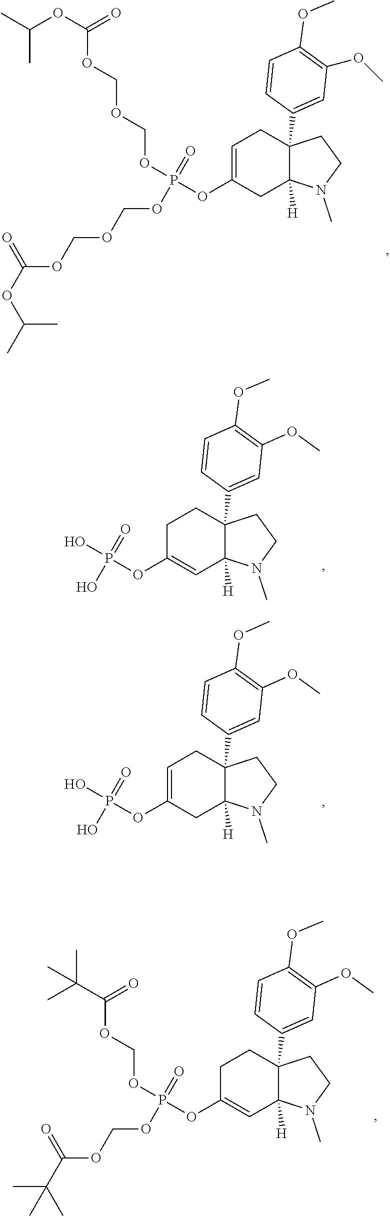

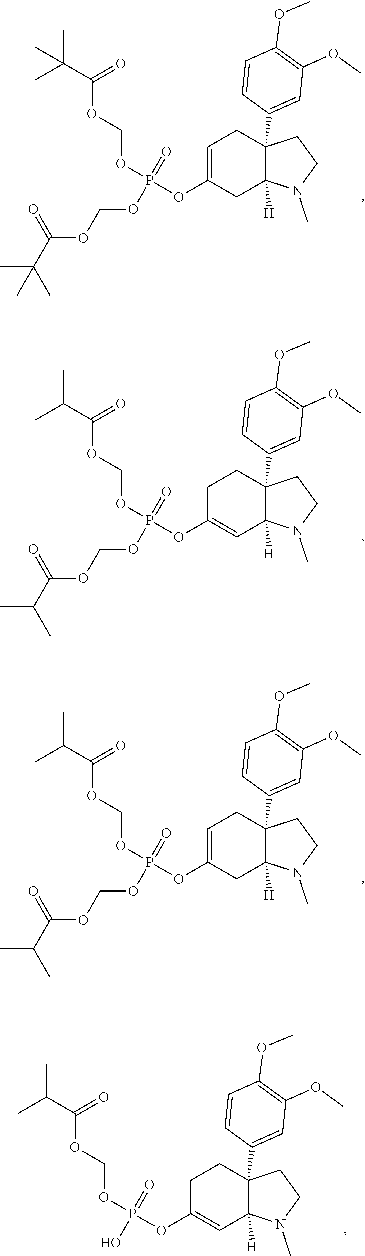

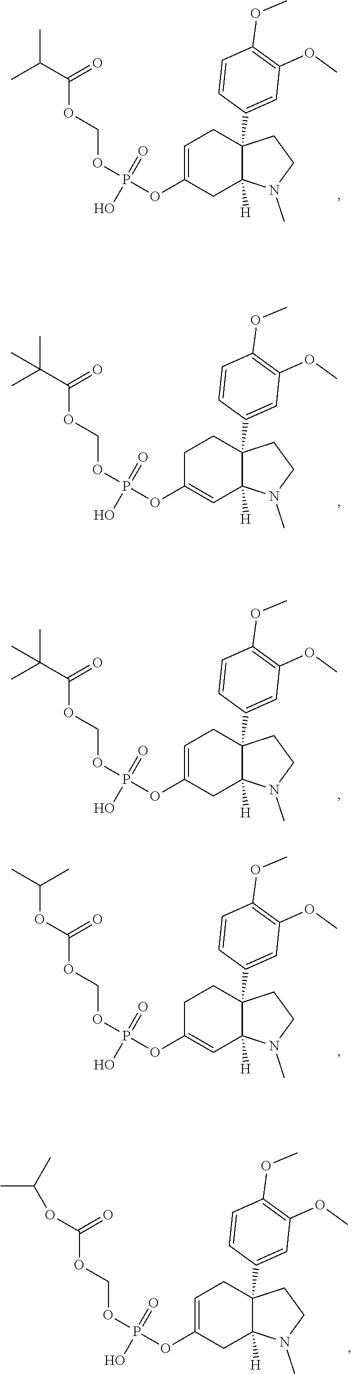

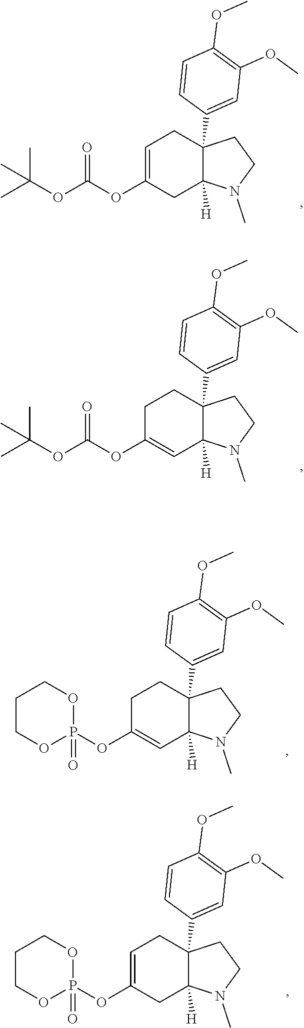

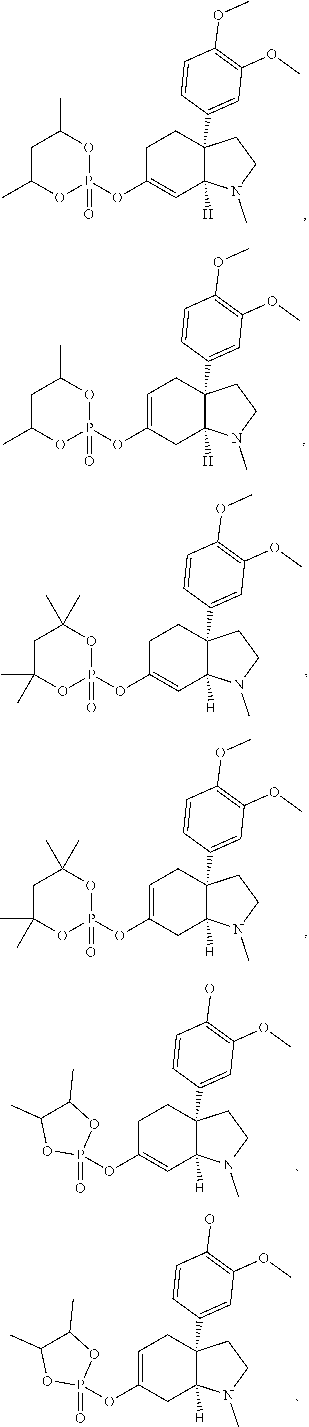

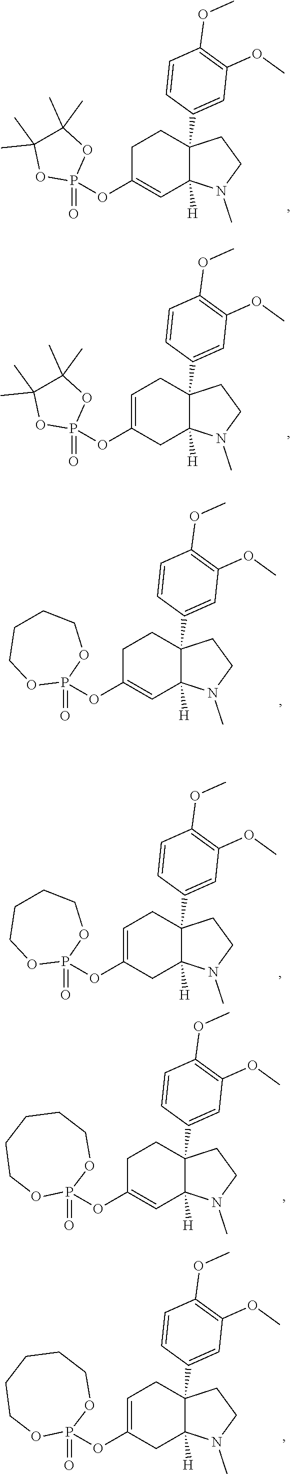

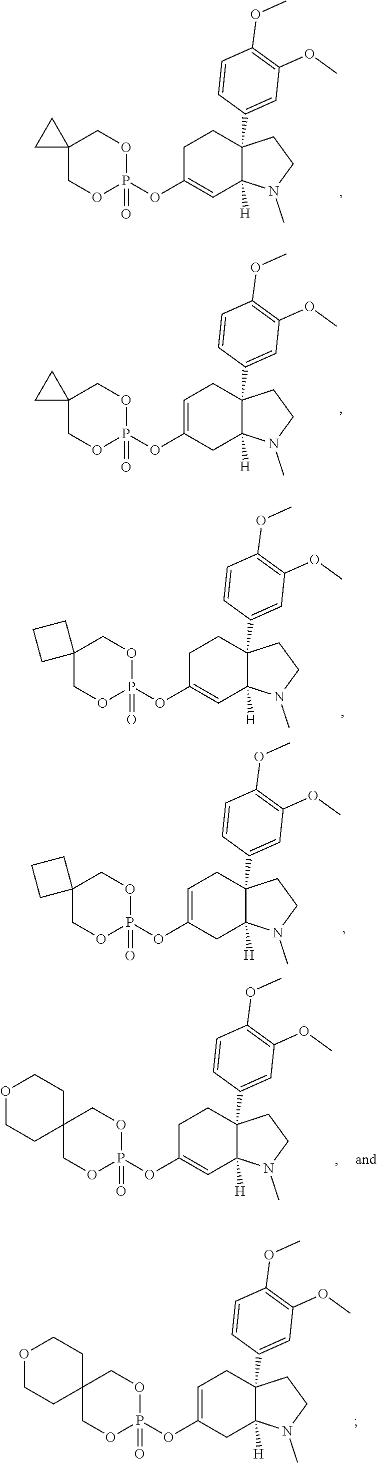

or a pharmaceutically acceptable salt thereof.

In certain embodiments, the compound is selected from:

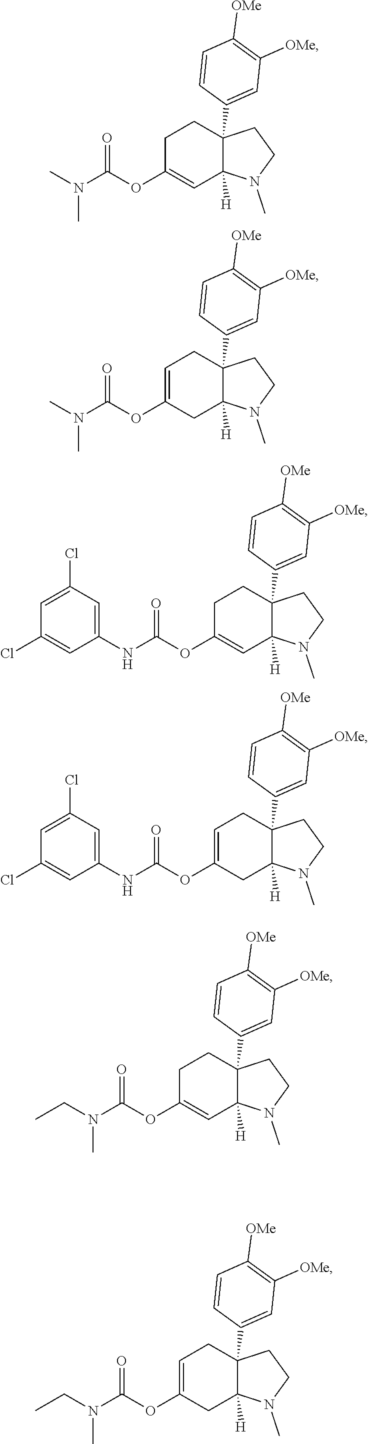

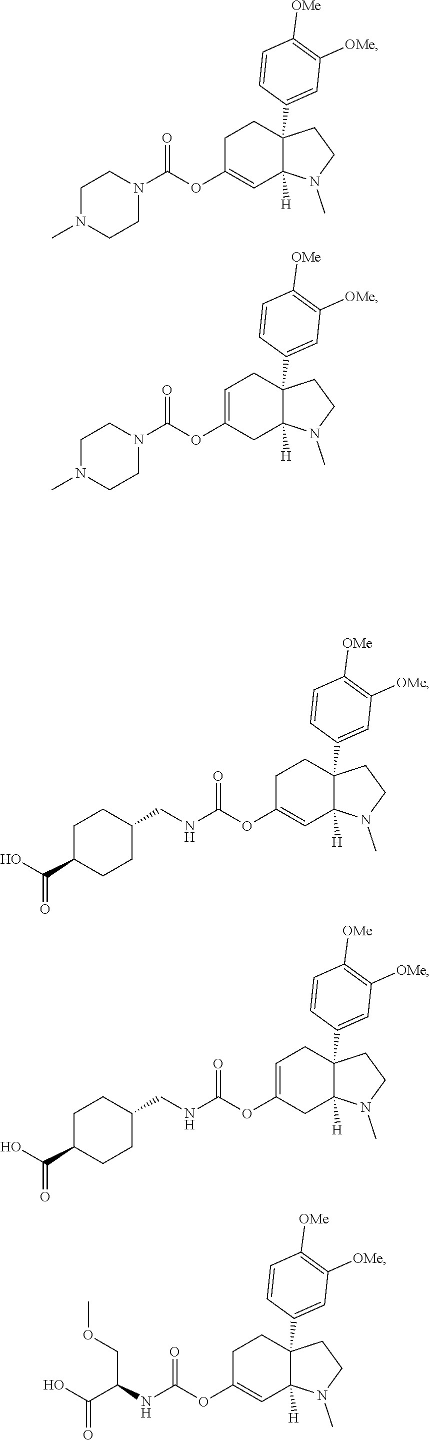 [SMILES: COc1ccc([C@@]23CCC(OC(=O)N4CCN(C)CC4)=C[C@@H]2N(C)CC3)cc1OC ; COc1ccc([C@@]23CC=C(OC(=O)N4CCN(C)CC4)C[C@@H]2N(C)CC3)cc1OC ; COc1ccc([C@@]23CCC(OC(=O)NC[C@H]4CC[C@H](C(=O)O)CC4)=C[C@@H]2N(C)CC3)cc1OC ; COc1ccc([C@@]23CC=C(OC(=O)NC[C@H]4CC[C@H](C(=O)O)CC4)C[C@@H]2N(C)CC3)cc1OC ; COC[C@@H](NC(=O)OC1=C[C@@H]2N(C)CC[C@]2(c2ccc(OC)c(OC)c2)CC1)C(=O)O]

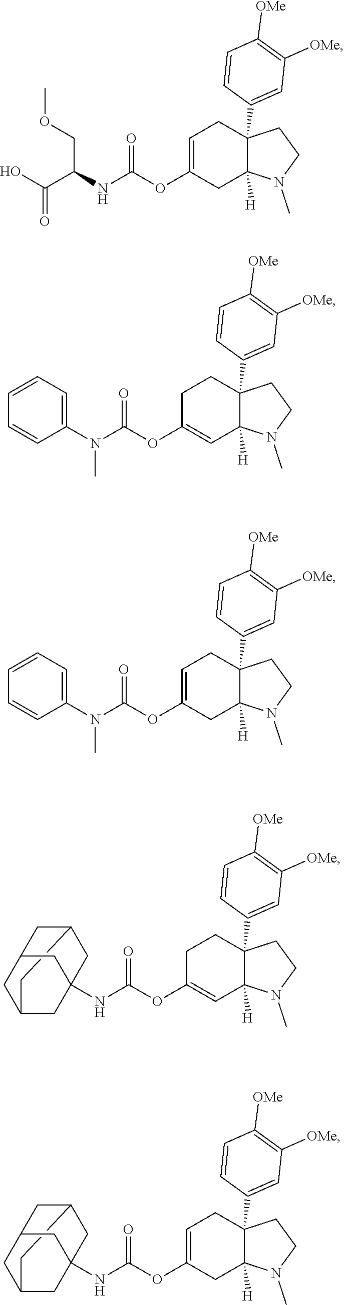 [SMILES: COC[C@@H](NC(=O)OC1=CC[C@@]2(c3ccc(OC)c(OC)c3)CCN(C)[C@H]2C1)C(=O)O ; COc1ccc([C@@]23CCC(OC(=O)N(C)c4ccccc4)=C[C@@H]2N(C)CC3)cc1OC ; COc1ccc([C@@]23CC=C(OC(=O)N(C)c4ccccc4)C[C@@H]2N(C)CC3)cc1OC ; COc1ccc([C@@]23CCC(OC(=O)NC45CC6CC(CC(C6)C4)C5)=C[C@@H]2N(C)CC3)cc1OC ; COc1ccc([C@@]23CC=C(OC(=O)NC45CC6CC(CC(C6)C4)C5)C[C@@H]2N(C)CC3)cc1OC]

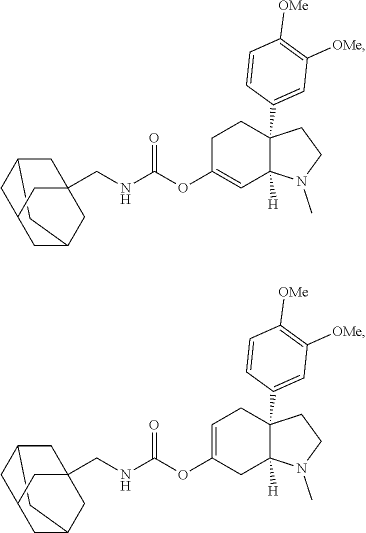 [SMILES: COc1ccc([C@@]23CCC(OC(=O)NCC45CC6CC(CC(C6)C4)C5)=C[C@@H]2N(C)CC3)cc1OC ; COc1ccc([C@@]23CC=C(OC(=O)NCC45CC6CC(CC(C6)C4)C5)C[C@@H]2N(C)CC3)cc1OC]

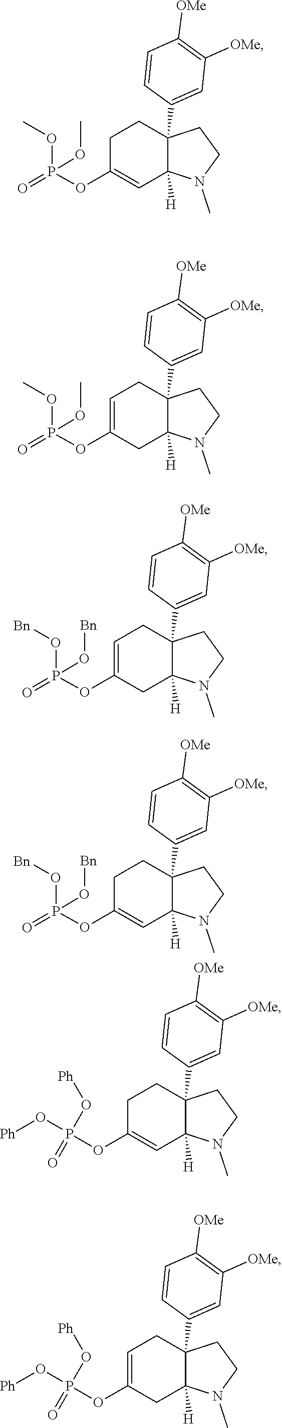

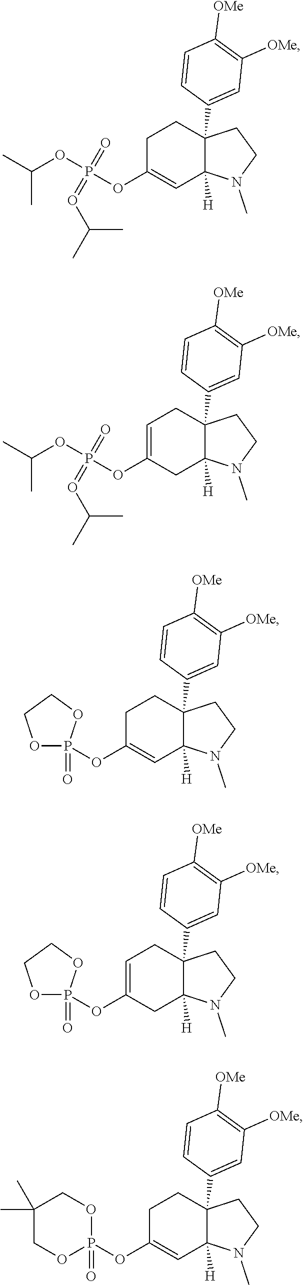

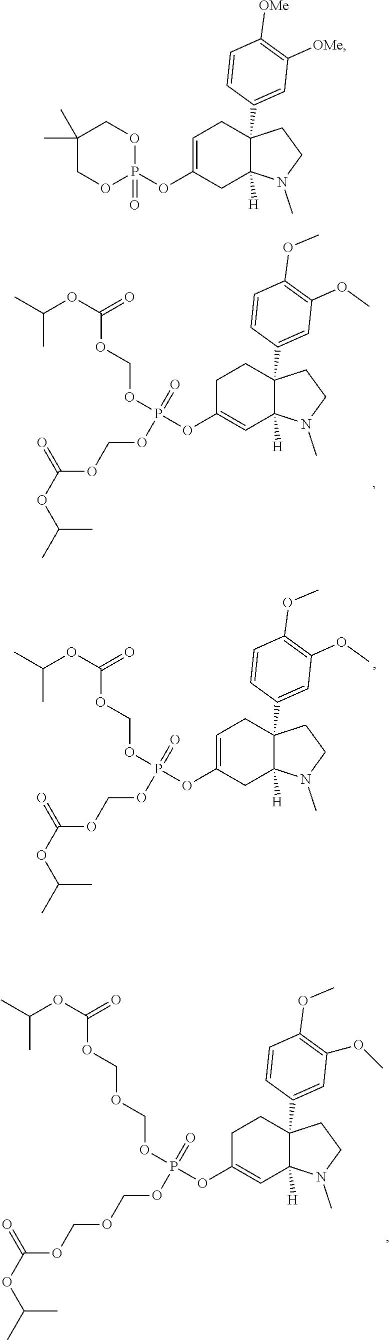

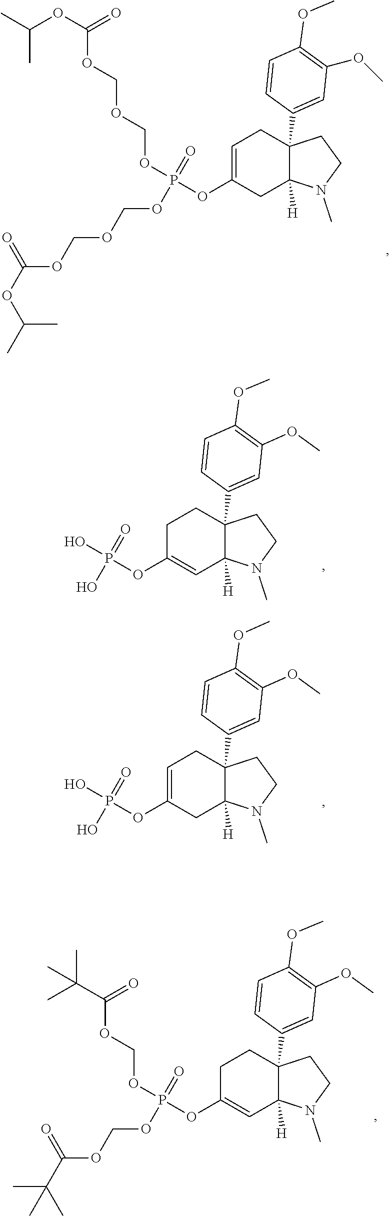

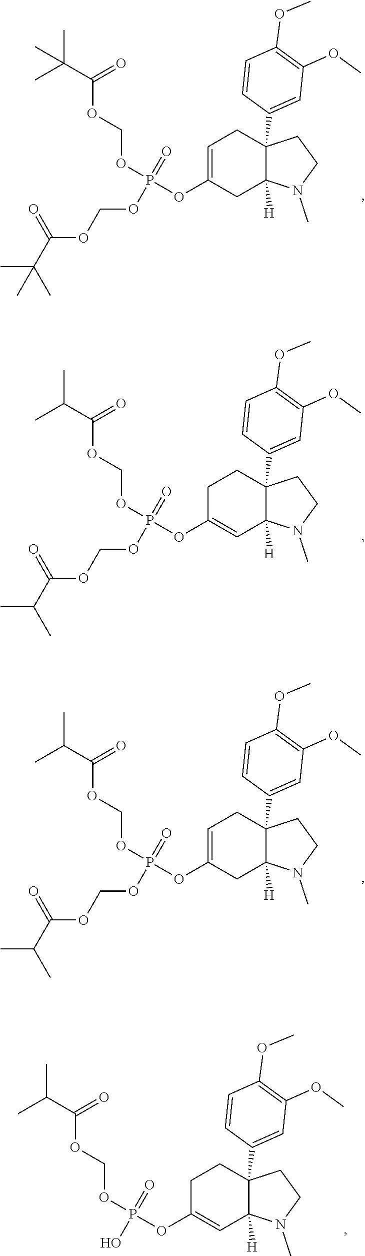

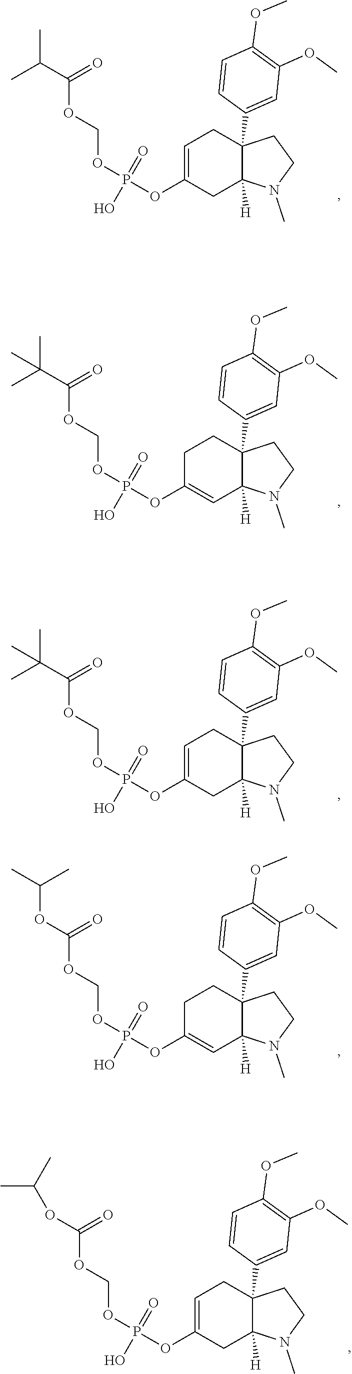

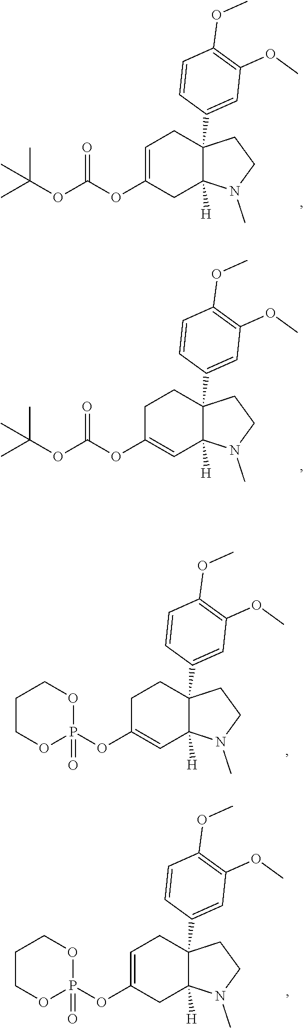

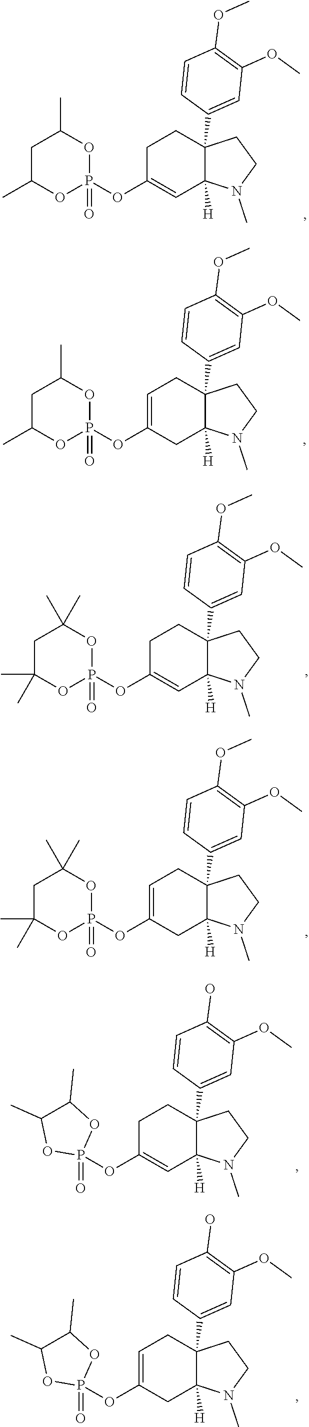

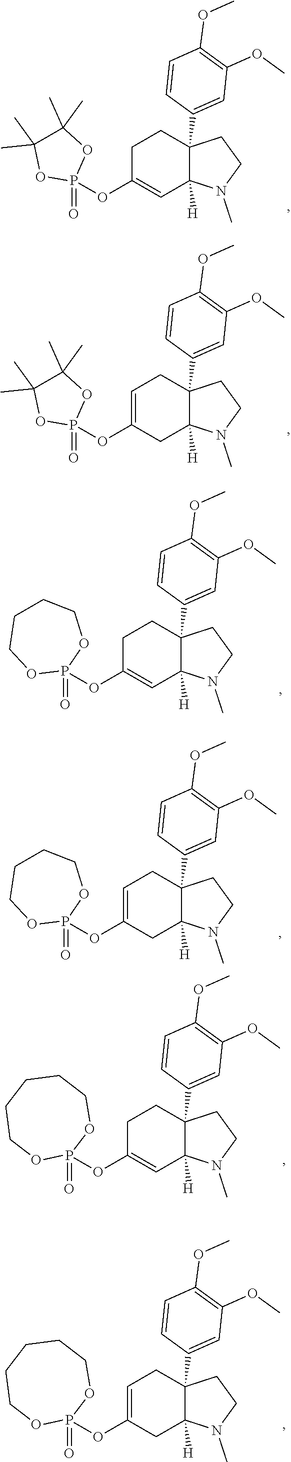

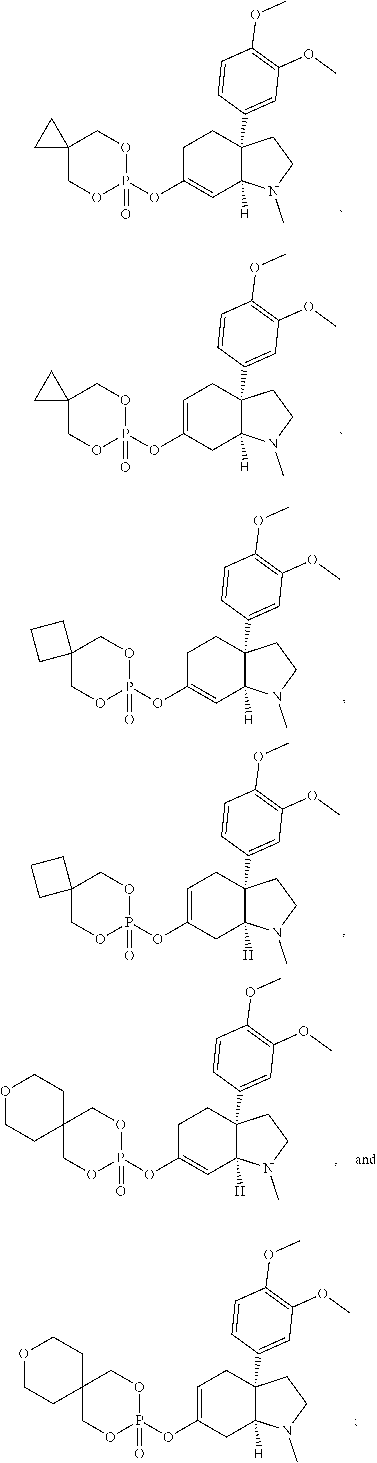

or a pharmaceutically acceptable salt thereof, and the compound has the absolute stereochemistry shown.

In certain embodiments, the compound is selected from:

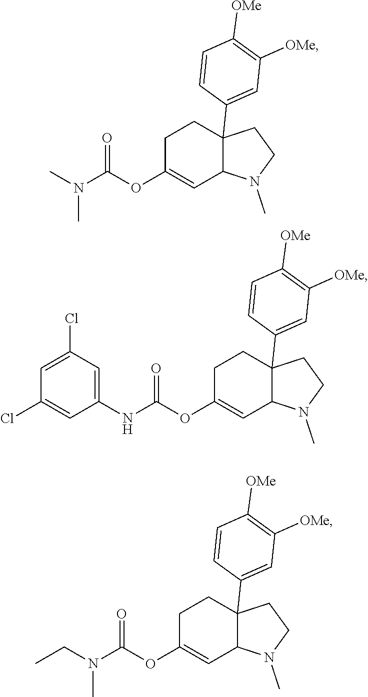

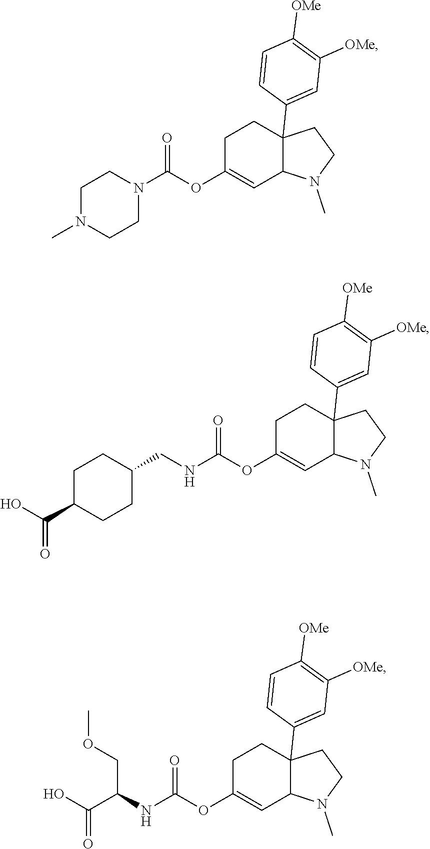 [SMILES: COc1ccc(C23CCC(OC(=O)N4CCN(C)CC4)=CC2N(C)CC3)cc1OC ; COc1ccc(C23CCC(OC(=O)NC[C@H]4CC[C@H](C(=O)O)CC4)=CC2N(C)CC3)cc1OC ; COC[C@@H](NC(=O)OC1=CC2N(C)CCC2(c2ccc(OC)c(OC)c2)CC1)C(=O)O]

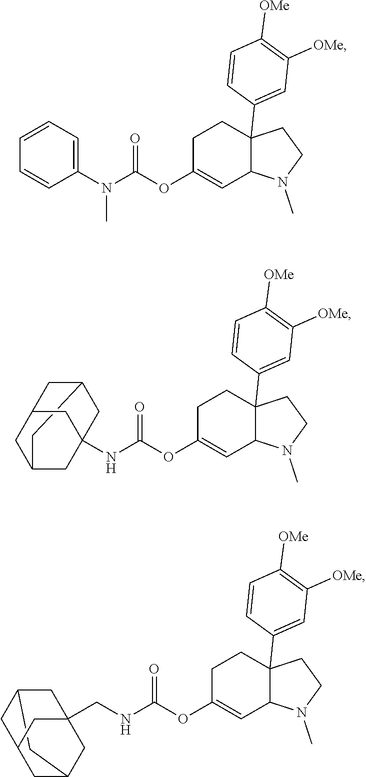 [SMILES: COc1ccc(C23CCC(OC(=O)N(C)c4ccccc4)=CC2N(C)CC3)cc1OC ; COc1ccc(C23CCC(OC(=O)NC45CC6CC(CC(C6)C4)C5)=CC2N(C)CC3)cc1OC ; COc1ccc(C23CCC(OC(=O)NCC45CC6CC(CC(C6)C4)C5)=CC2N(C)CC3)cc1OC]

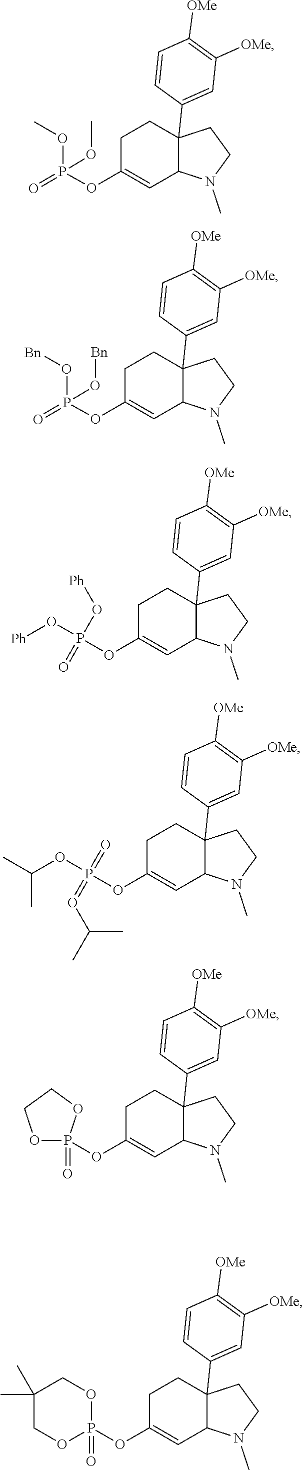

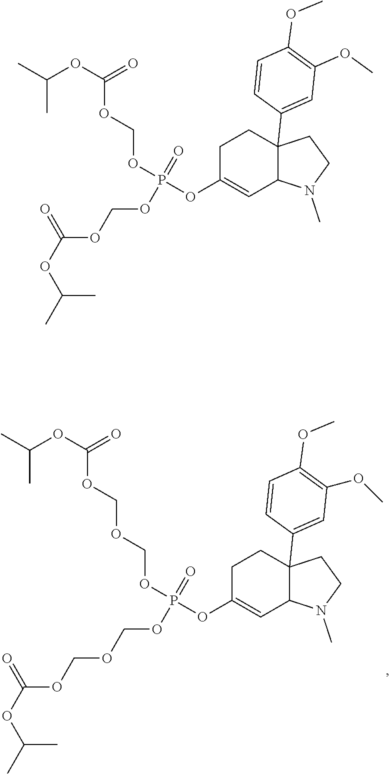

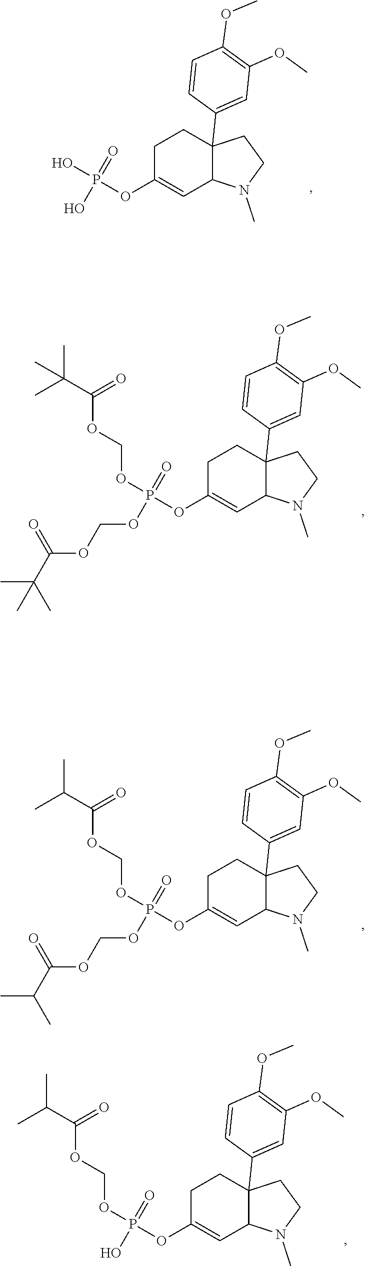

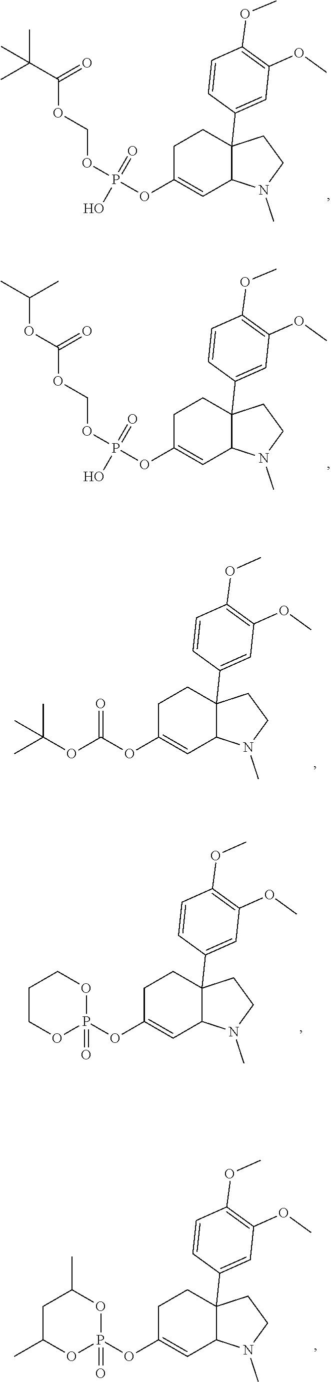

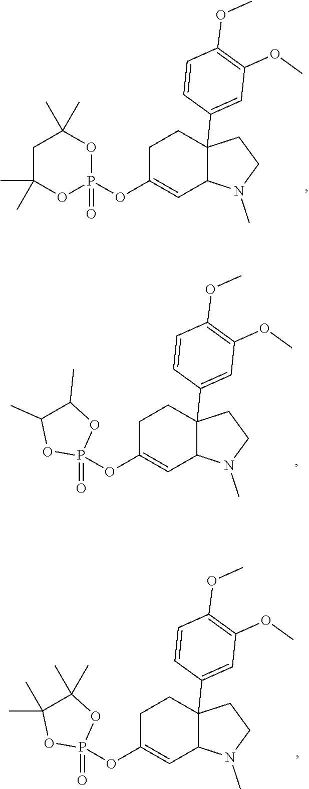

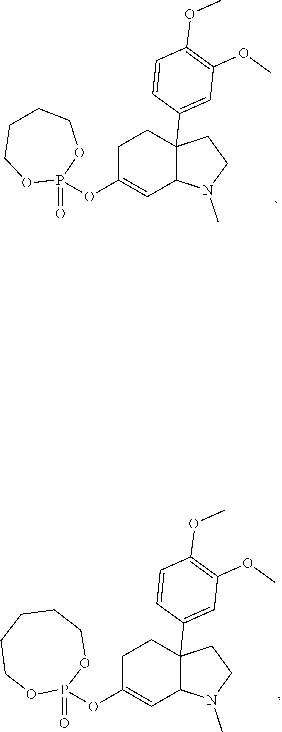 [SMILES: COc1ccc(C23CCC(OP4(=O)OCCCCO4)=CC2N(C)CC3)cc1OC ; COc1ccc(C23CCC(OP4(=O)OCCCCCO4)=CC2N(C)CC3)cc1OC]

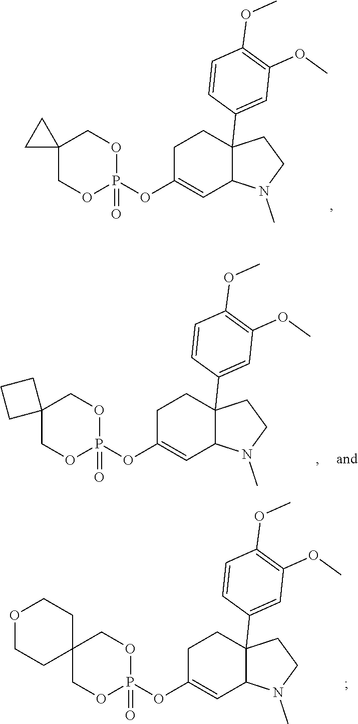

or a pharmaceutically acceptable salt thereof.

In certain embodiments, the compound is selected from:

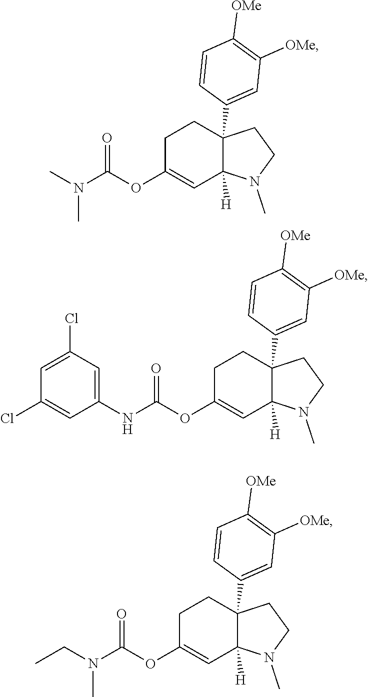

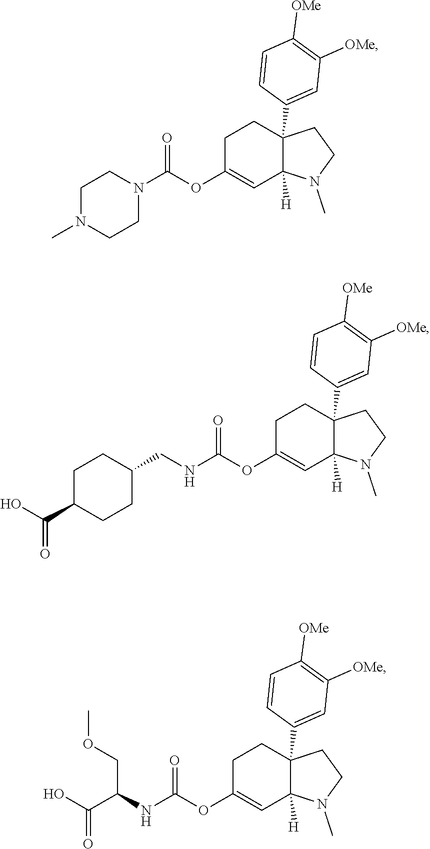 [SMILES: COc1ccc([C@@]23CCC(OC(=O)N4CCN(C)CC4)=C[C@@H]2N(C)CC3)cc1OC ; COc1ccc([C@@]23CCC(OC(=O)NC[C@H]4CC[C@H](C(=O)O)CC4)=C[C@@H]2N(C)CC3)cc1OC ; COC[C@@H](NC(=O)OC1=C[C@@H]2N(C)CC[C@]2(c2ccc(OC)c(OC)c2)CC1)C(=O)O]

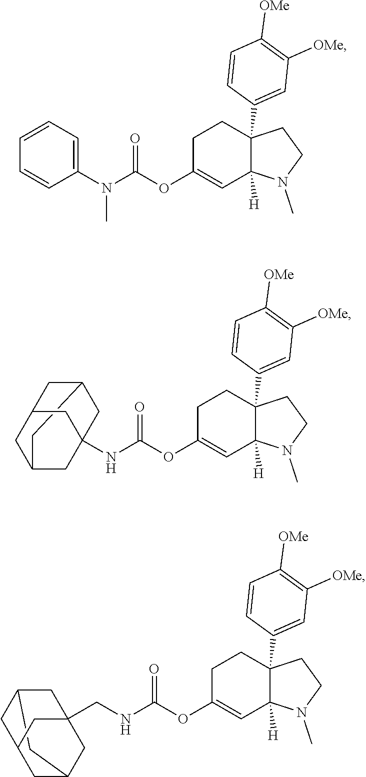 [SMILES: COc1ccc([C@@]23CCC(OC(=O)N(C)c4ccccc4)=C[C@@H]2N(C)CC3)cc1OC ; COc1ccc([C@@]23CCC(OC(=O)NC45CC6CC(CC(C6)C4)C5)=C[C@@H]2N(C)CC3)cc1OC ; COc1ccc([C@@]23CCC(OC(=O)NCC45CC6CC(CC(C6)C4)C5)=C[C@@H]2N(C)CC3)cc1OC]

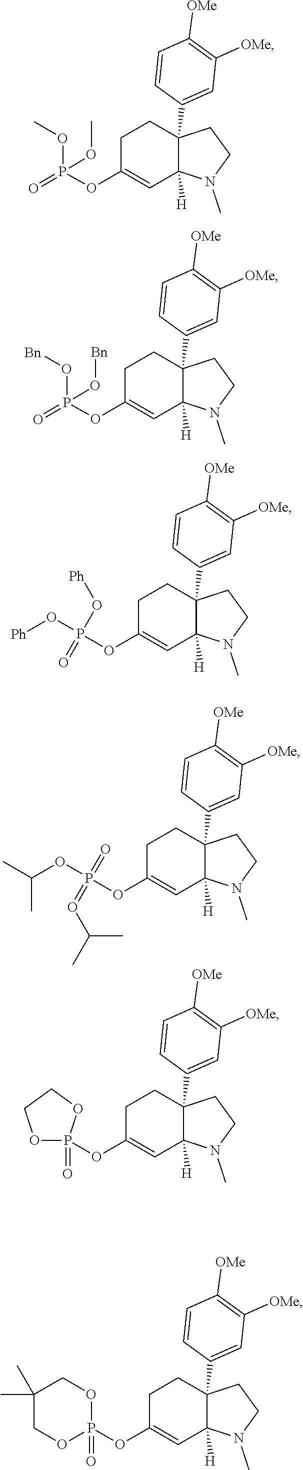

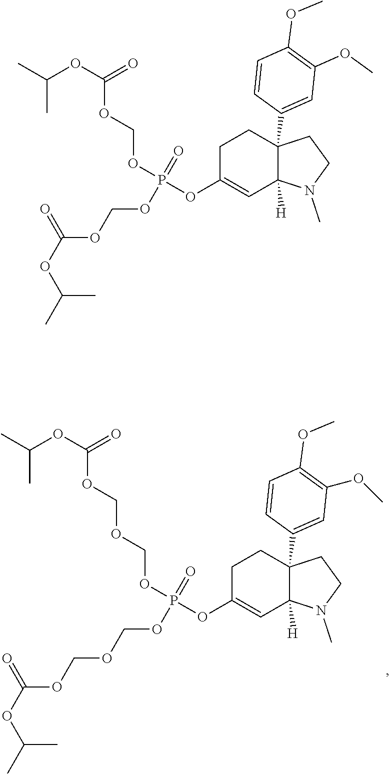

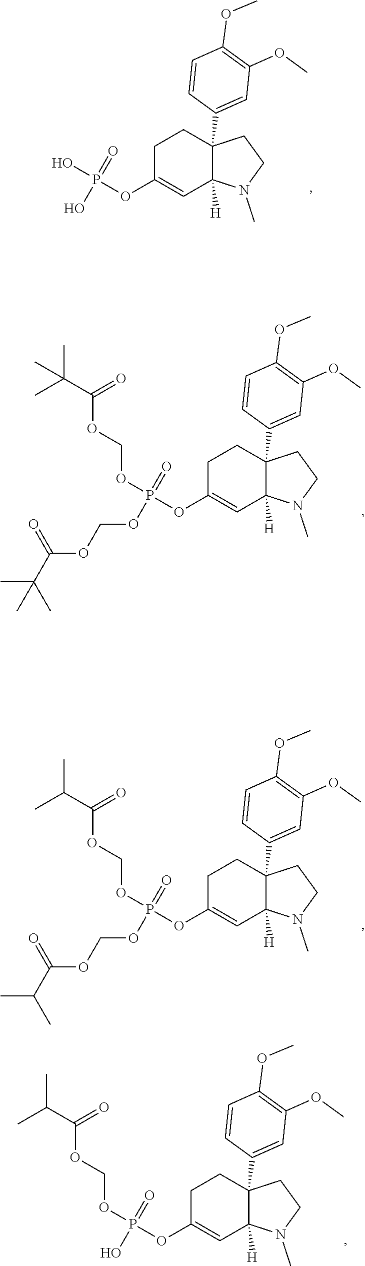

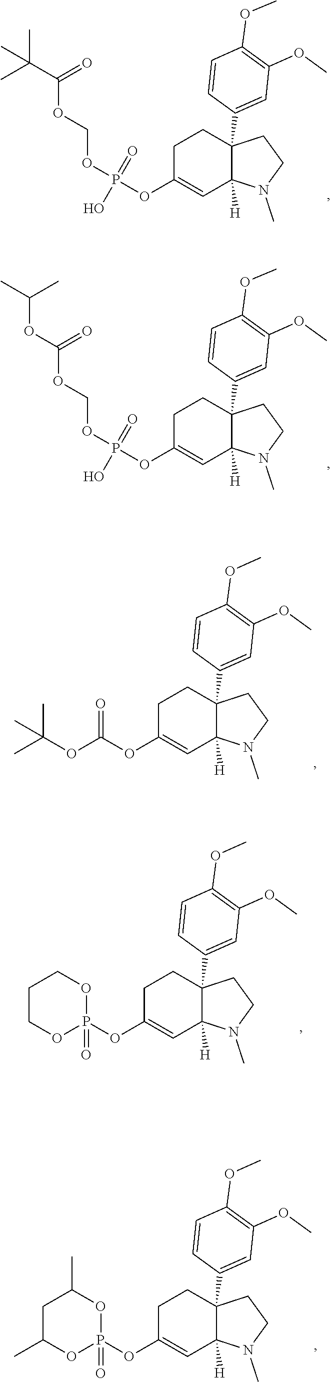

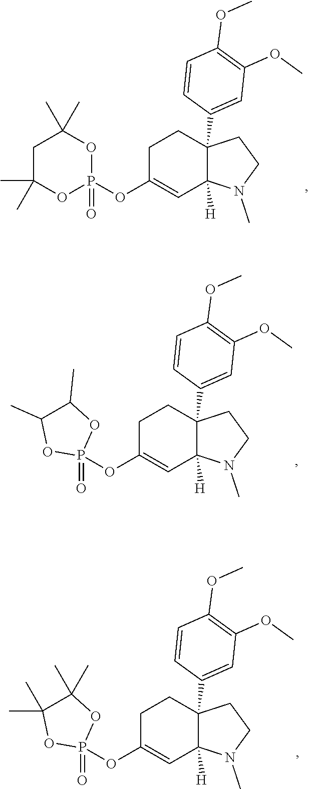

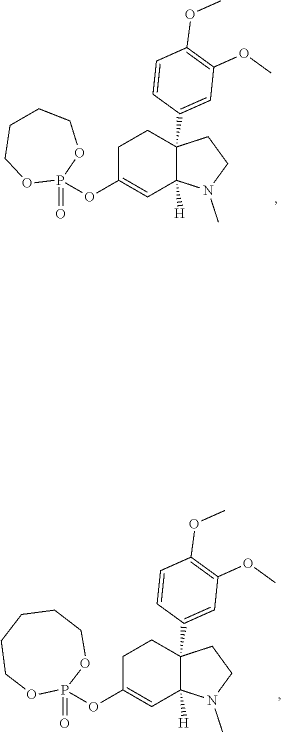 [SMILES: COc1ccc([C@@]23CCC(OP4(=O)OCCCCO4)=C[C@@H]2N(C)CC3)cc1OC ; COc1ccc([C@@]23CCC(OP4(=O)OCCCCCO4)=C[C@@H]2N(C)CC3)cc1OC]

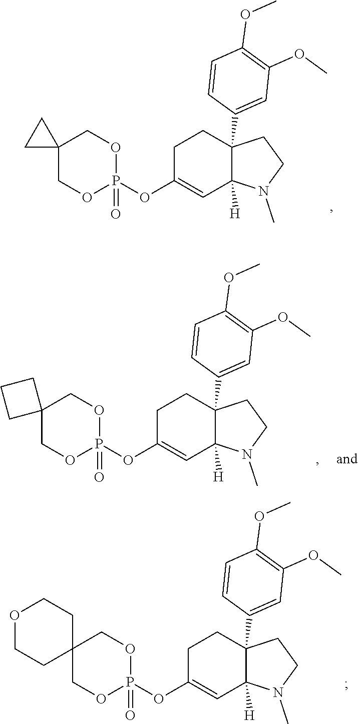

or a pharmaceutically acceptable salt thereof.

In certain embodiments, the compound is selected from:

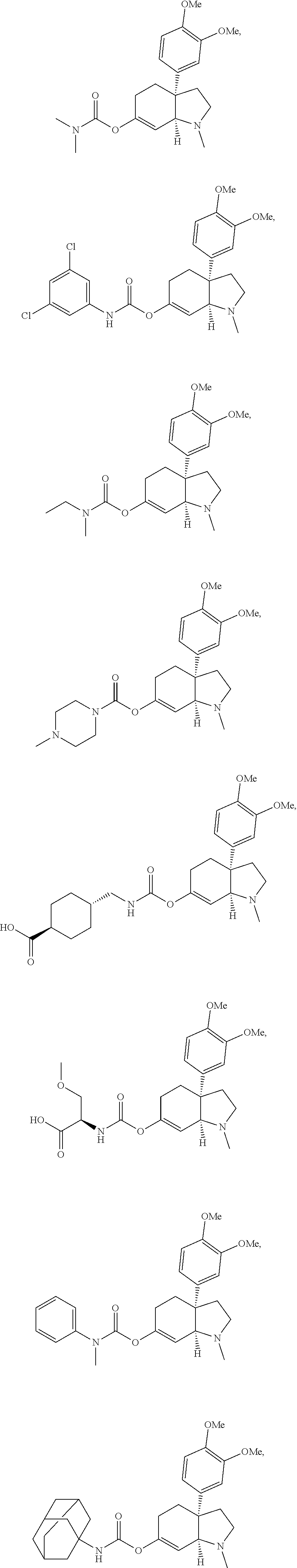

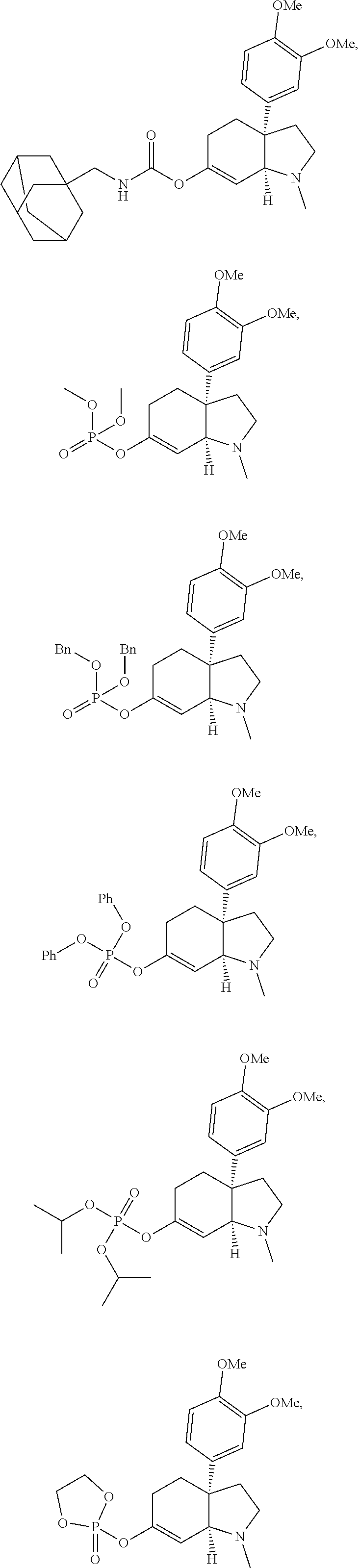 [SMILES: COc1ccc([C@@]23CCC(OC(=O)NCC45CC6CC(CC(C6)C4)C5)=C[C@@H]2N(C)CC3)cc1OC ; COc1ccc([C@@]23CCC(OP(=O)(OC)OC)=C[C@@H]2N(C)CC3)cc1OC ; COc1ccc([C@@]23CCC(OP(=O)(OCc4ccccc4)OCc4ccccc4)=C[C@@H]2N(C)CC3)cc1OC ; COc1ccc([C@@]23CCC(OP(=O)(Oc4ccccc4)Oc4ccccc4)=C[C@@H]2N(C)CC3)cc1OC ; COc1ccc([C@@]23CCC(OP(=O)(OC(C)C)OC(C)C)=C[C@@H]2N(C)CC3)cc1OC ; COc1ccc([C@@]23CCC(OP4(=O)OCCO4)=C[C@@H]2N(C)CC3)cc1OC]

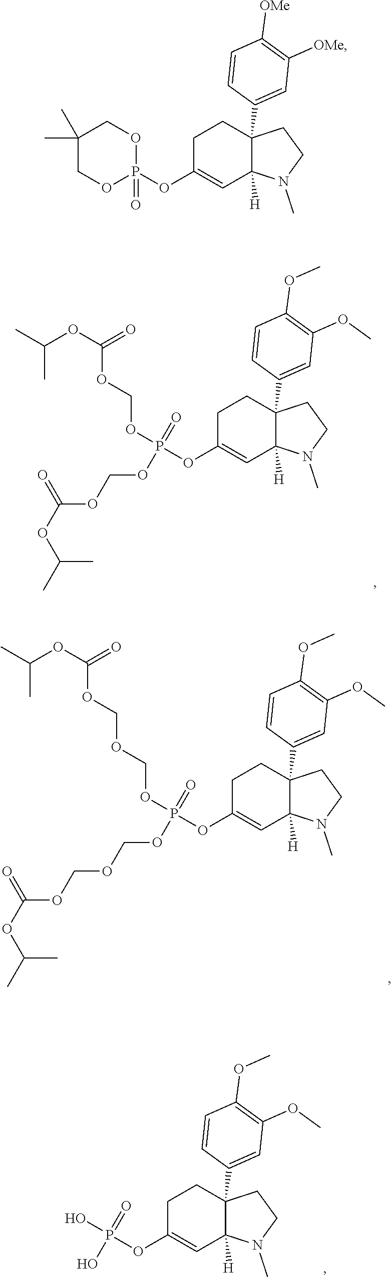

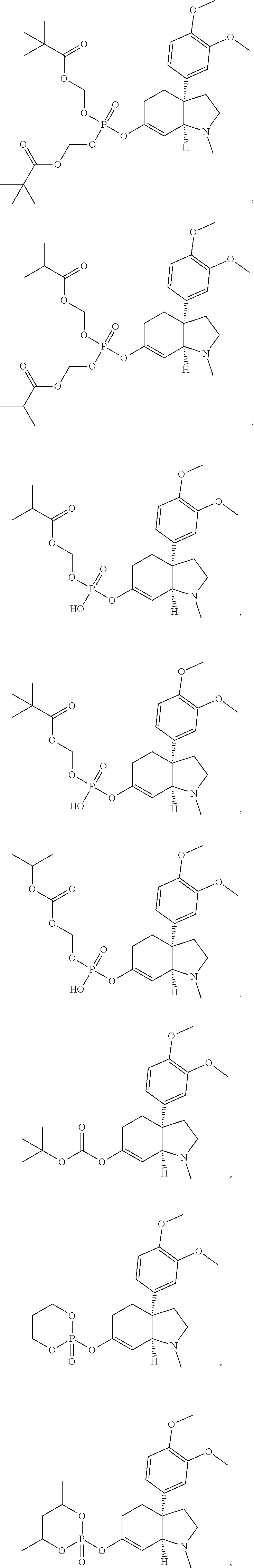

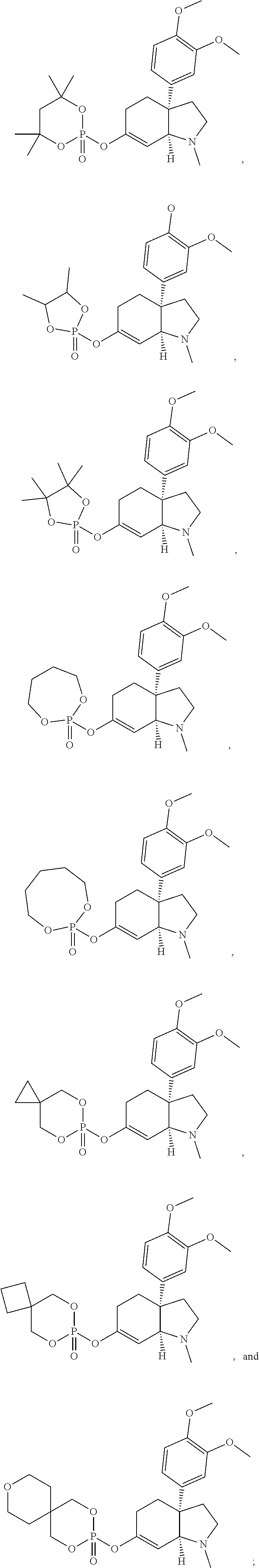

or a pharmaceutically acceptable salt thereof, and the compound has the absolute stereochemistry shown.

In some embodiments, the compounds are compounds of formula (I) or formula (I-A):

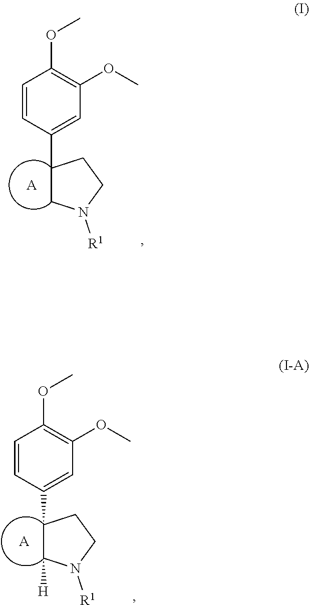

or a pharmaceutically acceptable salt thereof,
wherein

R¹ is methyl;

ring A is

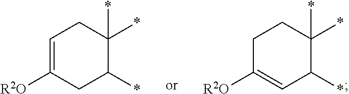

wherein * denotes the attachment points of ring A to the compound of formula (I),

R² is —C(O)NR³R⁴;

R³ is H, C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and

R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R³ and R⁴ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy; and

n is 1 or 2.

In some embodiments, the compounds are compounds of formula (I) or formula (I-A):

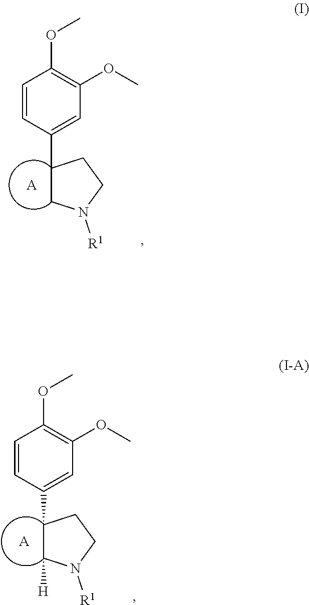

or a pharmaceutically acceptable salt thereof,
wherein

R¹ is methyl;

ring A is

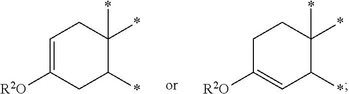

wherein * denotes the attachment points of ring A to the compound of formula (I),

R² is —C(O)NR³R⁴; and

R³ and R⁴ are each independently C₁-C₆ alkyl.

In some embodiments, the compounds are compounds of formula (I) or formula (I-A):

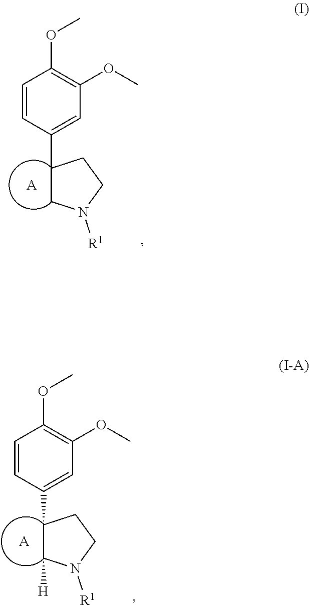

or a pharmaceutically acceptable salt thereof,
wherein

R¹ is methyl;

ring A is

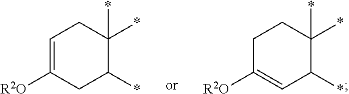

wherein * denotes the attachment points of ring A to the compound of formula (I),

R² is —C(O)NR³R⁴;

R³ is H or C₁-C₆ alkyl; and

R⁴ is phenyl optionally substituted with halogen.

In some embodiments, the compounds are compounds of formula (I) or formula (I-A):

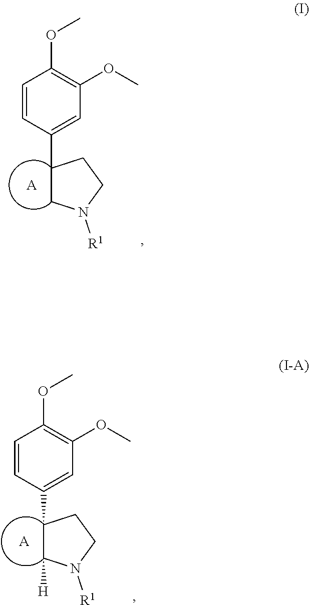

or a pharmaceutically acceptable salt thereof,
wherein

R¹ is methyl;

ring A is

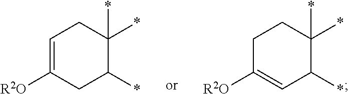

wherein * denotes the attachment points of ring A to the compound of formula (I),

R² is —C(O)NR³R⁴;

R³ is H or methyl; and

R⁴ is phenyl optionally substituted with halogen.

In some embodiments, the compounds are compounds of formula (I) or formula (I-A):

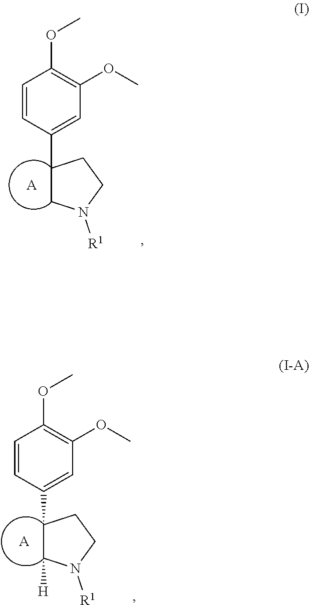

or a pharmaceutically acceptable salt thereof,
wherein

R¹ is methyl;

ring A is

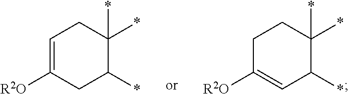

wherein * denotes the attachment points of ring A to the compound of formula (I),

R² is —C(O)NR³R⁴;

R³ is C₃-C₁₀ cycloalkyl, or C₁-C₃ alkyl-C₃-C₁₀ cycloalkyl, wherein each hydrogen atom in C₃-C₁₀ cycloalkyl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, or cyano; and

R⁴ is H.

In some embodiments, the compounds are compounds of formula (I) or formula (I-A):

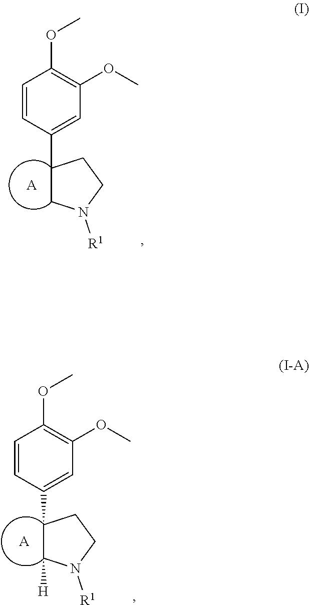

or a pharmaceutically acceptable salt thereof,
wherein

R¹ is methyl;

ring A is

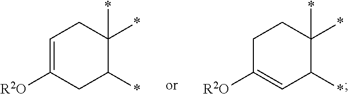

wherein * denotes the attachment points of ring A to the compound of formula (I),

R² is —C(O)NR³R⁴;

R³ is C₃-C₁₀ cycloalkyl, or C₁-C₃ alkyl-C₃-C₁₀ cycloalkyl, wherein each hydrogen atom in C₃-C₁₀ cycloalkyl is optionally substituted by —COOH; and

R⁴ is H.

In some embodiments, the compounds are compounds of formula (I) or formula (I-A):

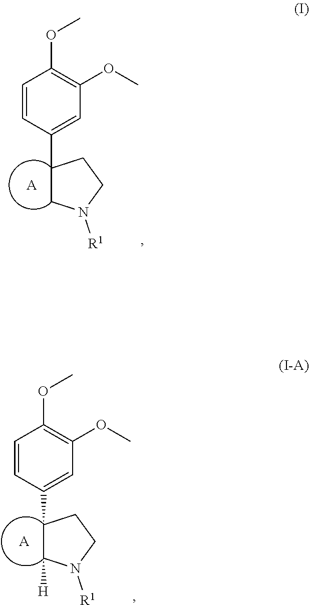

or a pharmaceutically acceptable salt thereof,
wherein

R¹ is methyl;

ring A is

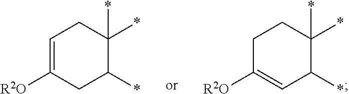

wherein * denotes the attachment points of ring A to the compound of formula (I),

R² is —C(O)NR³R⁴;

R³ is C₃-C₁₀ cycloalkyl, wherein each hydrogen atom in C₃-C₁₀ cycloalkyl is optionally substituted by —COOH; and

R⁴ is H.

In some embodiments, the compounds are compounds of formula (I) or formula (I-A):

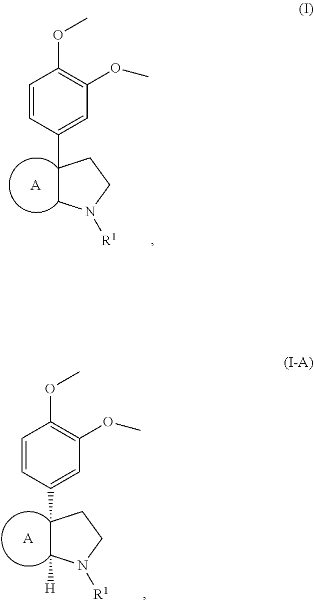

or a pharmaceutically acceptable salt thereof,
wherein

R¹ is methyl;

ring A is

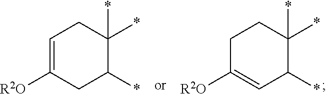

wherein * denotes the attachment points of ring A to the compound of formula (I),

R² is —C(O)NR³R⁴;

R³ is C₁-C₆ alkyl optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and

R⁴ is H.

In some embodiments, the compounds are compounds of formula (I) or formula (I-A):

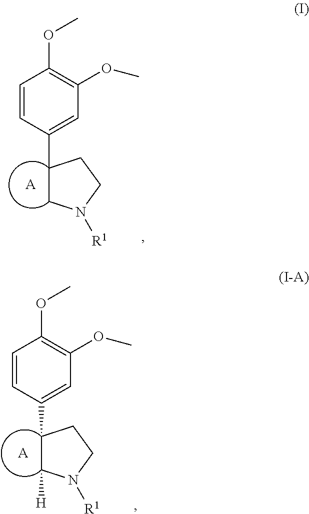

or a pharmaceutically acceptable salt thereof,
wherein

R¹ is methyl;

ring A is

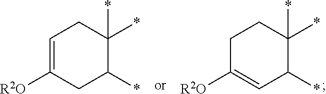

wherein * denotes the attachment points of ring A to the compound of formula (I),

R² is —C(O)NR³R⁴;

R³ is C₁-C₃ alkyl-C₃-C₁₀ cycloalkyl optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, or cyano; and

R⁴ is H.

In some embodiments, the compounds are compounds of formula (I) or formula (I-A):

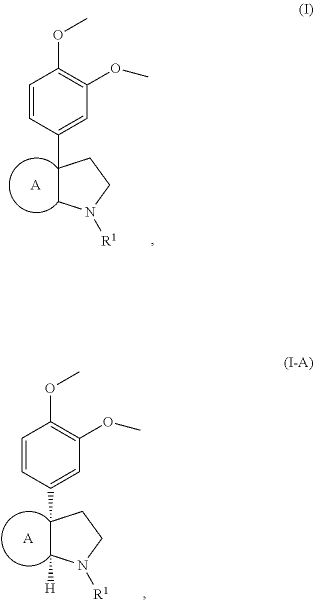

or a pharmaceutically acceptable salt thereof,
wherein

R¹ is methyl;

ring A is

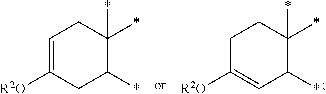

wherein * denotes the attachment points of ring A to the compound of formula (I),

R² is —C(O)NR³R⁴;

R³ is C₁-C₃ alkyl-C₃-C₁₀ cycloalkyl optionally substituted by —COOH; and

R⁴ is H.

In some embodiments, the compounds are compounds of formula (I) or formula (I-A):

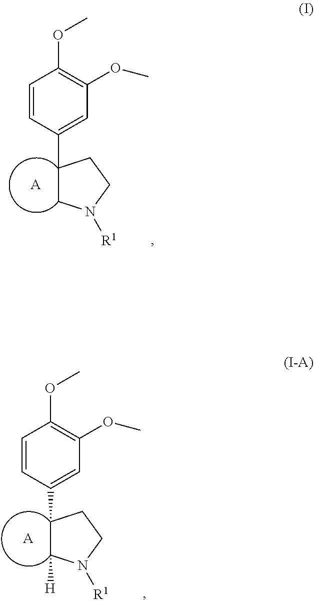

or a pharmaceutically acceptable salt thereof,
wherein

R¹ is methyl;

ring A is

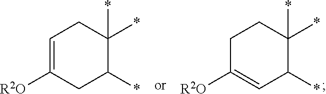

wherein * denotes the attachment points of ring A to the compound of formula (I),

R² is —C(O)NR³R⁴;

R³ is C₁-C₆ alkyl substituted by phenyl; and

R⁴ is H or C₁-C₆ alkyl.

In some embodiments, the compounds are compounds of formula (I) or formula (I-A):

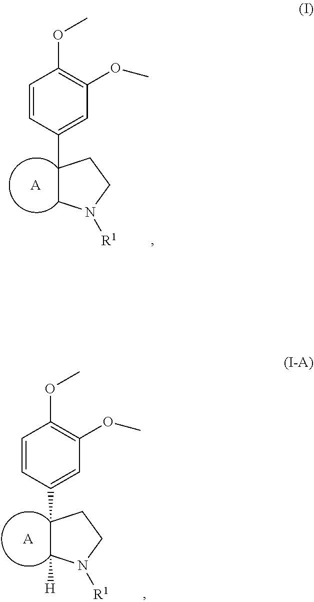

or a pharmaceutically acceptable salt thereof,
wherein

R¹ is methyl;

ring A is

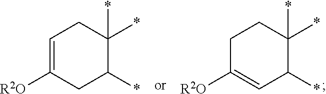

wherein * denotes the attachment points of ring A to the compound of formula (I),

R² is —C(O)NR³R⁴;

R³ is C₁-C₆ alkyl optionally substituted by —COOH; and

R⁴ is H.

In some embodiments, the compounds are compounds of formula (I) or formula (I-A):

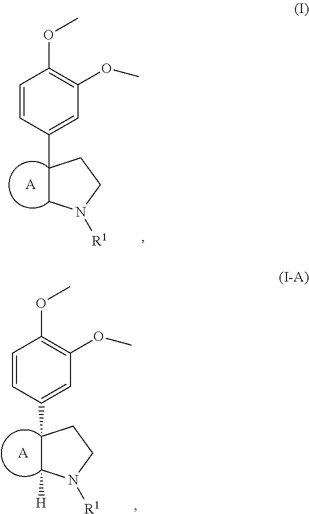

or a pharmaceutically acceptable salt thereof,
wherein

R¹ is methyl;

ring A is

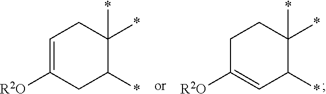

wherein * denotes the attachment points of ring A to the compound of formula (I),

R² is —C(O)NR³R⁴; and

R³ and R⁴ combine to form a 4-7 membered heterocycle, wherein each hydrogen atom in the 4-7 membered heterocycle is optionally substituted by halo, hydroxy, C₁-C₃ alkyl, C₁-C₃ alkoxy, nitro, —N(C₁-C₃ alkyl)₂, —NH₂, —N(H)C₁-C₃ alkyl, C₁-C₃ haloalkyl, —COOH, or cyano.

In some embodiments, the compounds are compounds of formula (I):

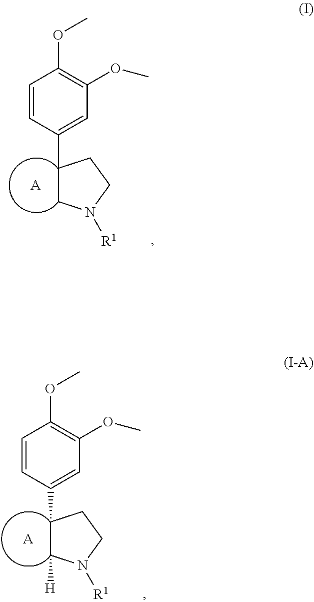

or a pharmaceutically acceptable salt thereof,
wherein

R¹ is methyl;

ring A is

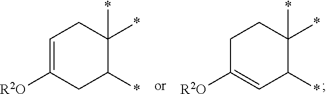

wherein * denotes the attachment points of ring A to the compound of formula (I),

R² is —C(O)NR³R⁴; and

R³ and R⁴ combine to form a 4-7 membered heterocycle, wherein each hydrogen atom in the 4-7 membered heterocycle is optionally substituted by C₁-C₃ alkyl.

In some embodiments, the compounds are compounds of formula (I) or formula (I-A):

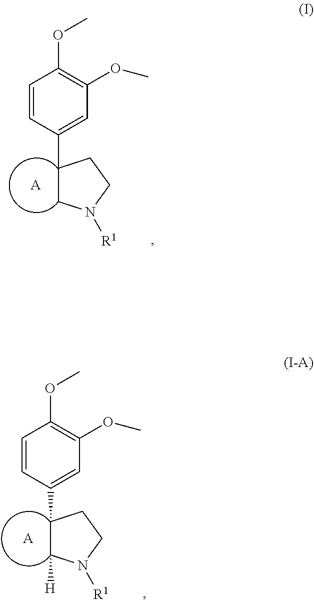

or a pharmaceutically acceptable salt thereof,
wherein

R¹ is methyl;

ring A is

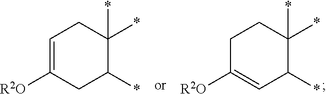

wherein * denotes the attachment points of ring A to the compound of formula (I),

R² is —C(O)OR⁶;

R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy.

In some embodiments, the compounds are compounds of formula (I) or formula (I-A):

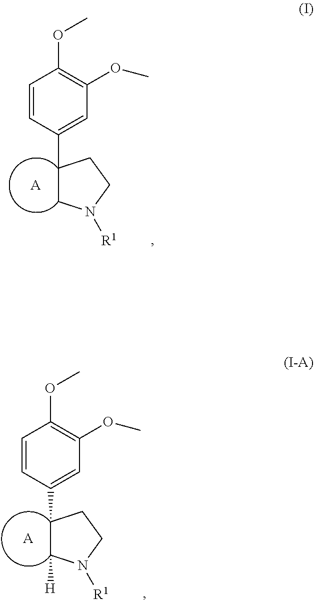

or a pharmaceutically acceptable salt thereof,
wherein

R¹ is methyl;

ring A is

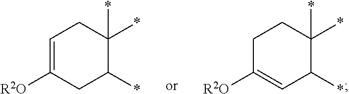

wherein * denotes the attachment points of ring A to the compound of formula (I),

R² is —C(O)OR⁶; and

R⁶ is C₁-C₆ alkyl.

In some embodiments, the compounds are compounds of formula (I) or formula (I-A):

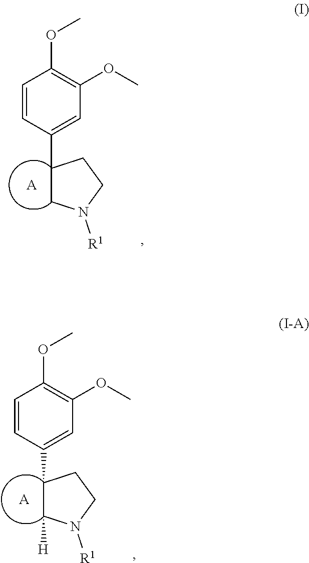

or a pharmaceutically acceptable salt thereof,
wherein

R¹ is methyl;

ring A is

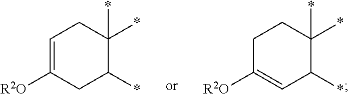

wherein * denotes the attachment points of ring A to the compound of formula (I),

R² is —P(O)OR⁴OR⁵;

R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

R⁵ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl; and

n is 1 or 2.

In some embodiments, the compounds are compounds of formula (I) or formula (I-A):

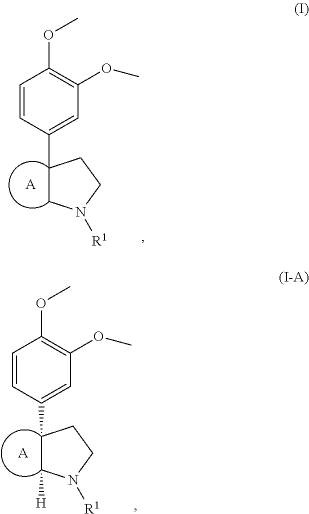

or a pharmaceutically acceptable salt thereof,
wherein

R¹ is methyl;

ring A is

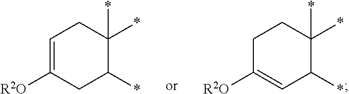

wherein * denotes the attachment points of ring A to the compound of formula (I),

R² is —P(O)OR⁴OR⁵;

R⁴ is C₁-C₆ alkyl; and

R⁵ is C₁-C₆ alkyl.

In some embodiments, the compounds are compounds of formula (I) or formula (I-A):

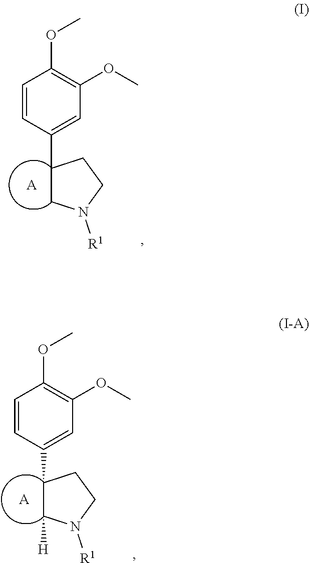

or a pharmaceutically acceptable salt thereof,
wherein

R¹ is methyl;

ring A is

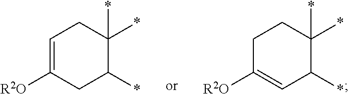

wherein * denotes the attachment points of ring A to the compound of formula (I),

R² is —P(O)OR⁴OR⁵; and

R⁴ is H or C₁-C₆ alkyl; and

R⁵ is H or C₁-C₆ alkyl.

In some embodiments, the compounds are compounds of formula (I) or formula (I-A):

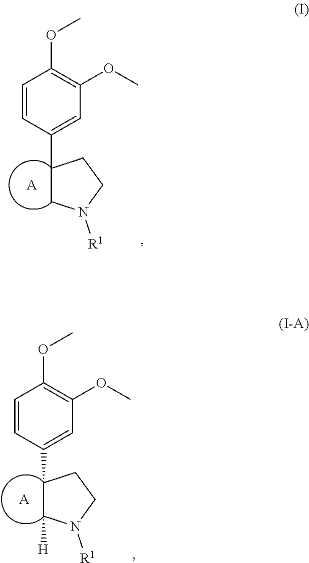

or a pharmaceutically acceptable salt thereof,
wherein

R¹ is methyl;

ring A is

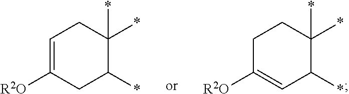

wherein * denotes the attachment points of ring A to the compound of formula (I),

R² is —P(O)OR⁴OR⁵; and

R⁴ and R⁵ are each hydrogen.

In some embodiments, the compounds are compounds of formula (I) or formula (I-A):

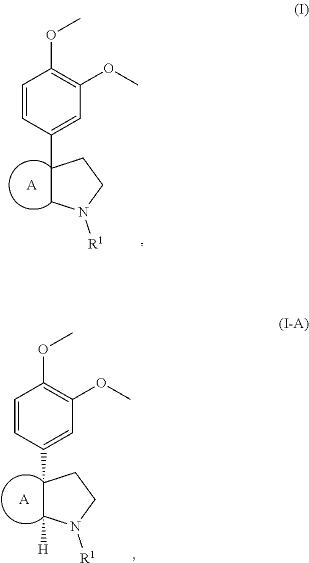

or a pharmaceutically acceptable salt thereof,
wherein

R¹ is methyl;

ring A is

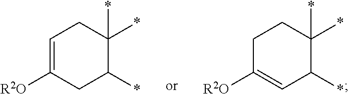

wherein * denotes the attachment points of ring A to the compound of formula (I),

R² is —P(O)OR⁴OR⁵;

R⁴ is —(CH₂O)ₙ—C(O)OC₁-C₆ alkyl or —(CH₂O)ₙ—C(O)C₁-C₆ alkyl;

R⁵ is —(CH₂O)ₙ—C(O)OC₁-C₆ alkyl or —(CH₂O)ₙ—C(O)C₁-C₆ alkyl; and

n is 1 or 2.

In some embodiments, the compounds are compounds of formula (I) or formula (I-A):

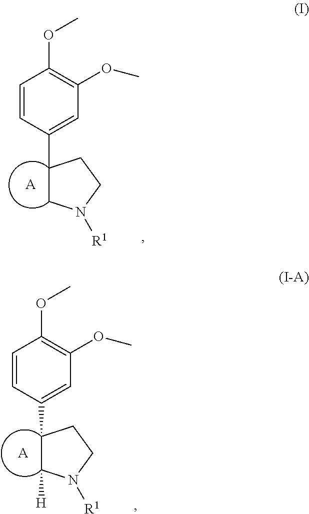

or a pharmaceutically acceptable salt thereof,
wherein

R¹ is methyl;

ring A is

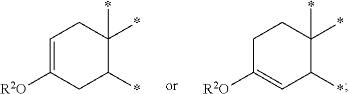

wherein * denotes the attachment points of ring A to the compound of formula (I),

R² is —P(O)OR⁴OR⁵; and

R⁴ and R⁵ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

In some embodiments, the compounds are compounds of formula (I) or formula (I-A):

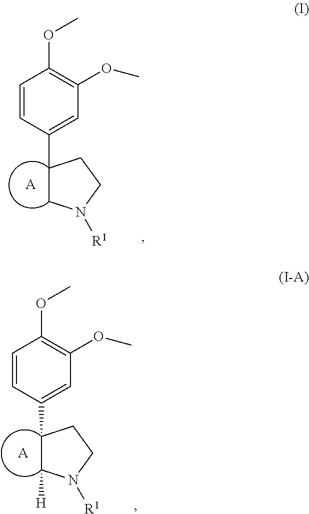

or a pharmaceutically acceptable salt thereof,
wherein

R¹ is methyl;

ring A is

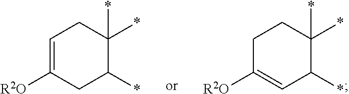

wherein * denotes the attachment points of ring A to the compound of formula (I),

R² is —P(O)OR⁴OR⁵; and

R⁴ and R⁵ combine to form a 4-7 membered heterocycle, wherein the 4-7 membered heterocycle is optionally substituted by a spirocyclic C₃-C₁₀ cycloalkyl.

In some embodiments, the compounds are compounds of formula (I) or formula (I-A):

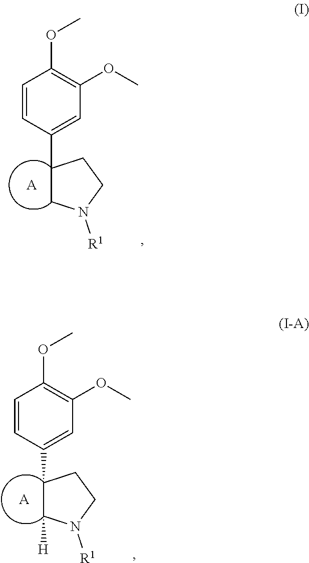

or a pharmaceutically acceptable salt thereof,
wherein

R¹ is methyl;

ring A is

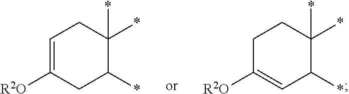

wherein * denotes the attachment points of ring A to the compound of formula (I),

R² is —P(O)OR⁴OR⁵; and

R⁴ and R⁵ combine to form a 4-7 membered heterocycle, wherein each hydrogen atom in the 4-7 membered heterocycle is optionally substituted by a C₁-C₃ alkyl; or the 4-7 membered heterocycle is optionally substituted by a spirocyclic C₃-C₆ cycloalkyl or a spirocyclic 5-7 member heterocycle.

In some embodiments, the compounds are compounds of formula (I) or formula (I-A):

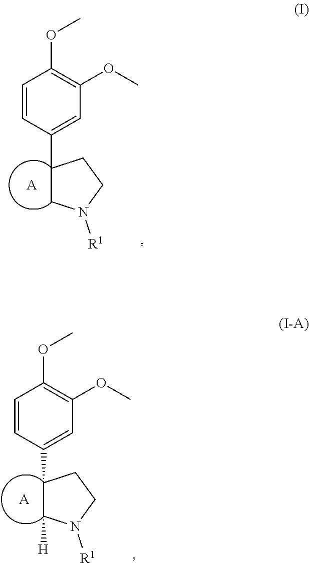

or a pharmaceutically acceptable salt thereof,
wherein

R¹ is methyl;

ring A is

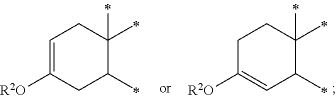

wherein * denotes the attachment points of ring A to the compound of formula (I),

R² is —P(O)OR⁴OR⁵; and

R⁴ and R⁵ combine to form a 4-7 membered heterocycle, wherein each hydrogen atom in the 4-7 membered heterocycle is optionally substituted by halo, hydroxy, C₁-C₃ alkyl, C₁-C₃ alkoxy, nitro, —N(C₁-C₃ alkyl)₂, —NH₂, —N(H)C₁-C₃ alkyl, C₁-C₃ haloalkyl, —COOH, or cyano.

In some embodiments, the compounds are compounds of formula (I) or formula (I-A):

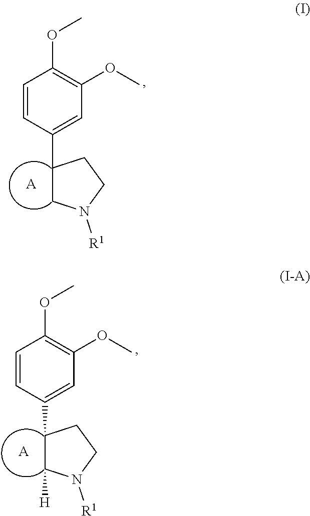

or a pharmaceutically acceptable salt thereof,
wherein

R¹ is methyl;

ring A is

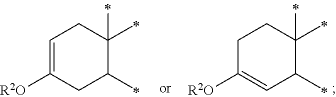

wherein * denotes the attachment points of ring A to the compound of formula (I),

R² is —P(O)OR⁴OR⁵; and

R⁴ and R⁵ combine to form a 4-7 membered heterocycle, wherein each hydrogen atom in the 4-7 membered heterocycle is optionally substituted by C₁-C₃ alkyl.

In some embodiments, the compounds are compounds of formula (I) or formula (I-A):

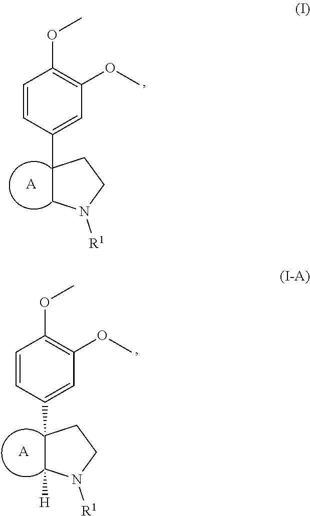

or a pharmaceutically acceptable salt thereof,
wherein

R¹ is methyl;

ring A is

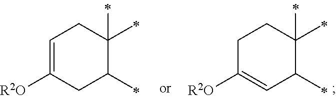

wherein * denotes the attachment points of ring A to the compound of formula (I),

R² is —P(O)OR⁴OR⁵;

R⁴ is phenyl; and

R⁵ is phenyl.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups, C₁-C₁₀ branched-chain alkyl groups, cycloalkyl (alicyclic) groups, alkyl-substituted cycloalkyl groups, and cycloalkyl-substituted alkyl groups. Preferably, the “alkyl” group refers to C₁-C₇ straight-chain alkyl groups or C₁-C₇ branched-chain alkyl groups. Most preferably, the “alkyl” group refers to C₁-C₃ straight-chain alkyl groups or C₁-C₃ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “haloalkyl” refers to an alkyl group substituted with at least one hydrogen atom on a carbon replaced by a halogen. Illustrative halogens include fluoro, chloro, bromo, and iodo. Illustrative haloalkyl groups include trifluoromethyl and 2,2,2-trifluoroethyl, etc.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

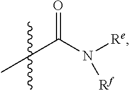

wherein R^{e} and R^{f} each independently represent a hydrogen or hydrocarbyl group, or R^{e} and R^{f} taken together with the N atom to which they are attached complete a heterocycle having from 4 to 8 atoms in the ring structure.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Preferably, the “alkoxy” group refers to C₁-C₇ straight-chain alkoxy groups or C₁-C₇ branched-chain alkoxy groups. Representative alkoxy groups include methoxy, ethoxy, propoxy, tert-butoxy and the like.

The term “aryloxy” refers to an aryl group having an oxygen attached thereto. Preferably, the “aryloxy” group refers to C₆-C₁₀ aryloxy groups or 5-7-membered heteroaryloxy groups. Representative aryloxy groups include phenoxy (C₆H₅—O—) and the like.

The terms “amine” and “amino” are art-recognized and refer to both unsubstituted and substituted amines and salts thereof, e.g., a moiety that can be represented by

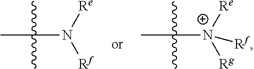

wherein R^{e}, R^{f}, and R^{g}, each independently represent a hydrogen or a hydrocarbyl group, or R^{e} and R^{f} taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring, for example a phenyl. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

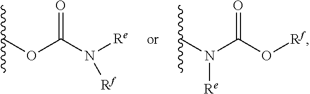

wherein R^{e} and R^{f} independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or =S substituent, and typically has at least one carbon-hydrogen bond and a primarily carbon backbone, but may optionally include heteroatoms.

Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains six or fewer carbon atoms, preferably four or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

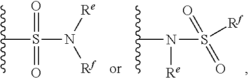

wherein R^{e} and R^{f} independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR^{e} or —SC(O)R^{e}

wherein R^{e} represents a hydrocarbyl.

The term “thioether”, as used herein, is equivalent to an ether, wherein the oxygen is replaced with a sulfur.

The term “urea” is art-recognized and may be represented by the general formula

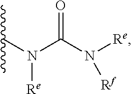

wherein R^{e} and R^{f} independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. The mono- or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form mesembrine. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs include using ester or phosphoramidate as biologically labile or cleavable (protecting) groups. The prodrugs of this disclosure are metabolized to produce mesembrine. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

Unless otherwise indicated in the tables of compounds herein, the abbreviation RAC or rac indicates a racemic mixture, and DIAST indicates a specific diastereomer. In illustrative embodiments, although a compound may be depicted with [symbol] or [symbol] bonds, such a depiction may be denoting relative stereochemistry based on elution peaks from a chiral separation.

### EXEMPLARY EMBODIMENTS

1. A compound of formula (I):

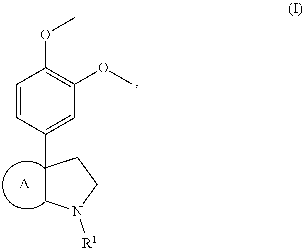

or a pharmaceutically acceptable salt thereof,
wherein

R¹ is H or C₁-C₇ alkyl;

ring A is

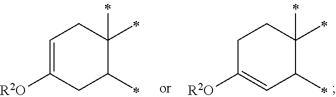

wherein * denotes the attachment points of ring A to the compound of formula (I),

R² is —C(O)NR³R⁴, —C(O)OR⁶, or —P(O)OR⁴OR⁵;

R³ is H, C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;

R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R³ and R⁴ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁵ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R⁴ and R⁵ combine to form a 4-8 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and

n is 1 or 2.

2. The compound of embodiment 1, wherein the compound is of formula (I):

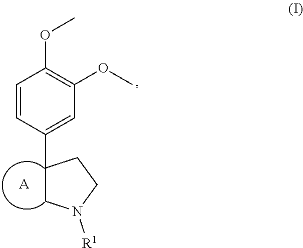

or a pharmaceutically acceptable salt thereof,
wherein

R¹ is H or C₁-C₇ alkyl;

ring A is

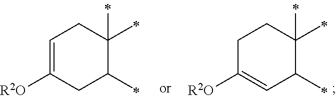

wherein * denotes the attachment points of ring A to the compound of formula (I),

R² is —C(O)NR³R⁴, —C(O)OR⁶, or —P(O)OR⁴OR⁵;

R³ is H, C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;

R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R³ and R⁴ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁵ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl;

or R⁴ and R⁵ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;

R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and

n is 1 or 2.

3. The compound of embodiment 1 or 2, wherein the compound is of formula (I-A):

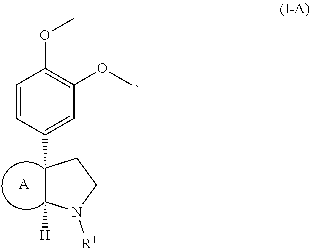

or a pharmaceutically acceptable salt thereof, wherein the compound has the absolute stereochemistry shown.

4. The compound of embodiment 1 or 2, wherein the compound is of formula (II):

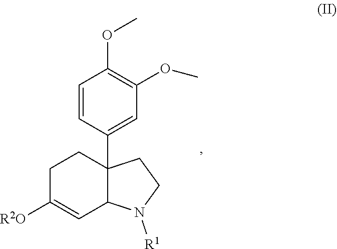

or a pharmaceutically acceptable salt thereof.

5. The compound of embodiment 4, wherein the compound is of formula (II-A):

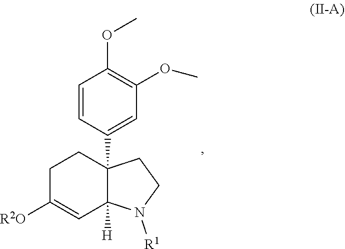

or a pharmaceutically acceptable salt thereof, wherein the compound has the absolute stereochemistry shown.

6. The compound of any one of embodiments 1-5, wherein R² is —C(O)NR³R⁴.

7. The compound of embodiment 6, wherein R³ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy.

8. The compound of embodiment 6 or 7, wherein R⁴ is C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by phenyl.

9. The compound of any one of embodiments 6-8, wherein one of R³ and R⁴ is H.

10. The compound of embodiment 6, wherein R³ and R⁴ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

11. The compound of any one of embodiments 1-5, wherein R² is —C(O)OR⁶.

12. The compound of embodiment 11, wherein R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, C₁-C₆ alkyl, C₁-C₃ alkoxy, nitro, —N(C₁-C₃ alkyl)₂, C₁-C₃ haloalkyl, —COOH, cyano, phenyl, or phenoxy.

13. The compound of any one of embodiments 1-5, wherein R² is —P(O)OR⁴OR⁵.

14. The compound of embodiment 13, wherein each of R⁴ and R⁵ is independently C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

15. The compound of embodiment 13, wherein one of R⁴ and R⁵ is C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

16. The compound of embodiment 15, wherein R⁵ is H.

17. The compound of any one of embodiments 13-16, wherein n is 1.

18. The compound of embodiment 13, wherein R⁴ and R⁵ combine to form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

19. The compound of embodiment 13, wherein R⁴ and R⁵ combine to form a 4-8 membered heterocycle.

20. The compound of embodiment 19, wherein R² is

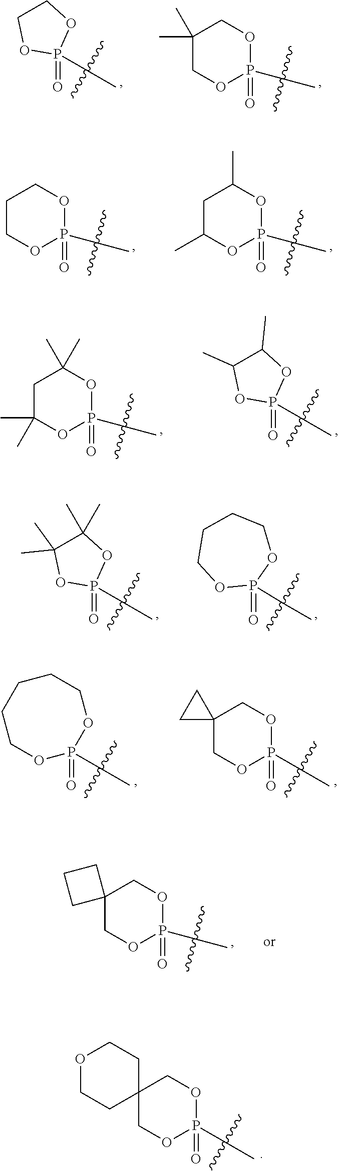

21. The compound of any one of embodiments 1-20, wherein R¹ is C₁-C₇ alkyl.

22. The compound of embodiment 21, wherein R¹ is methyl.

23. The compound of embodiment 1, selected from:

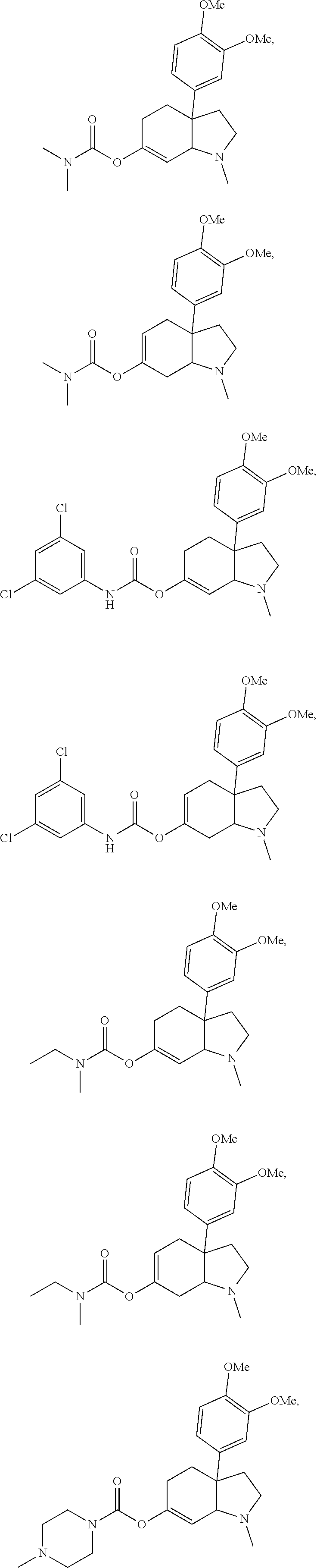

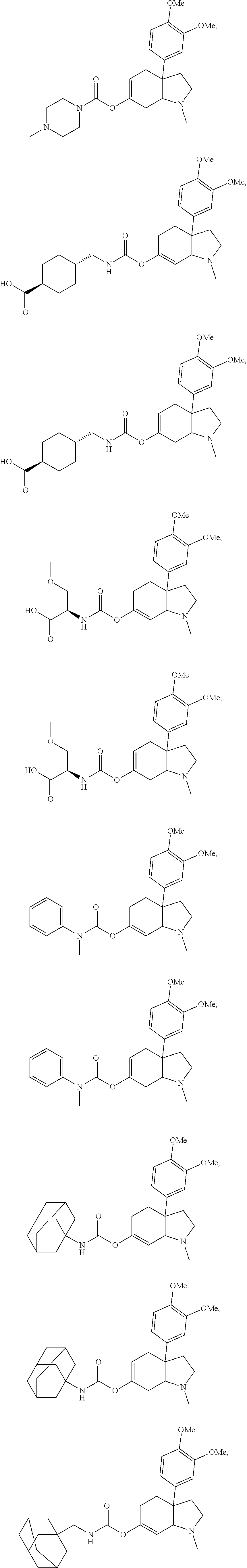

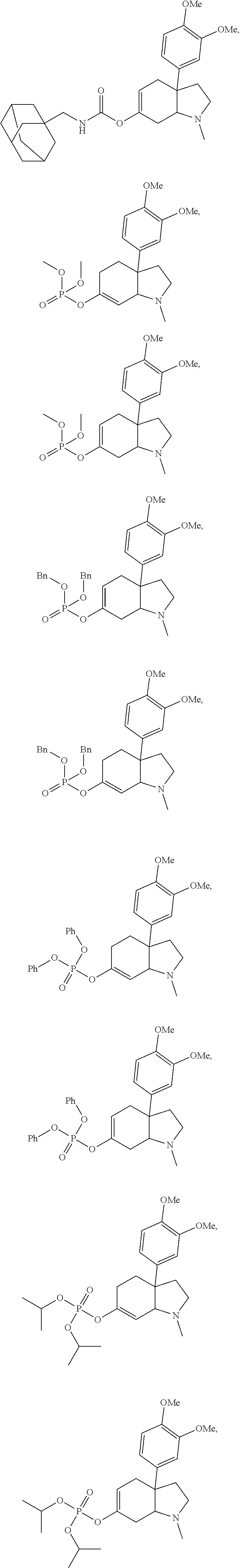

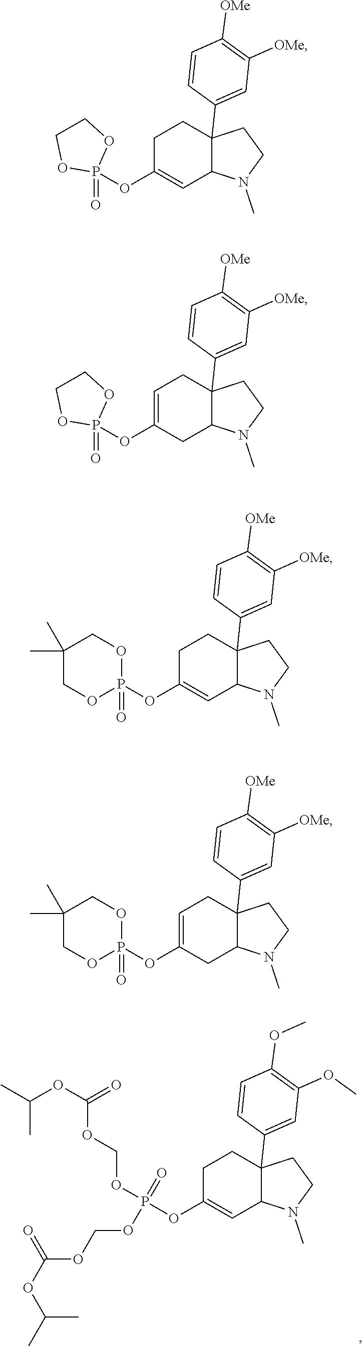

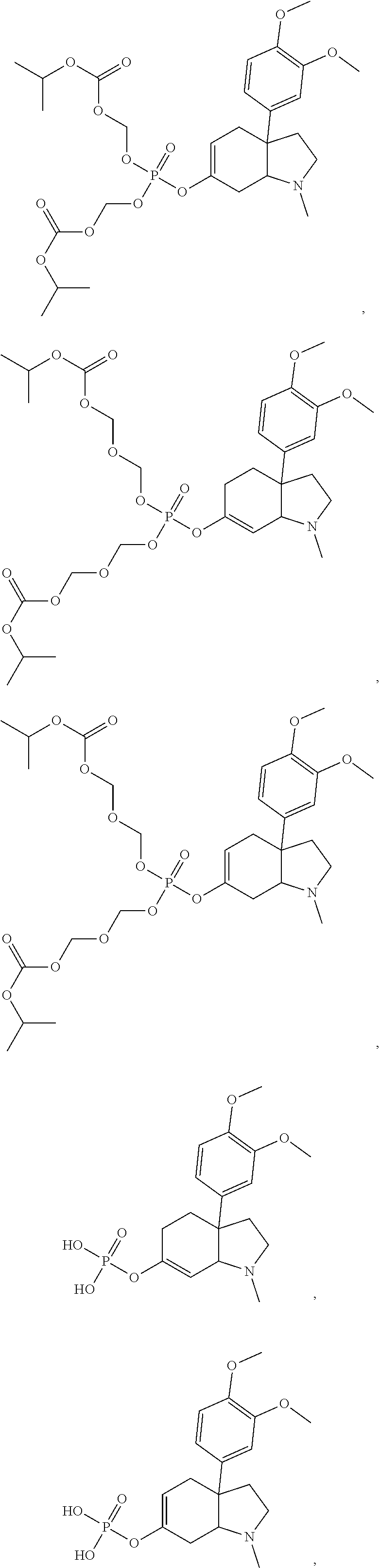

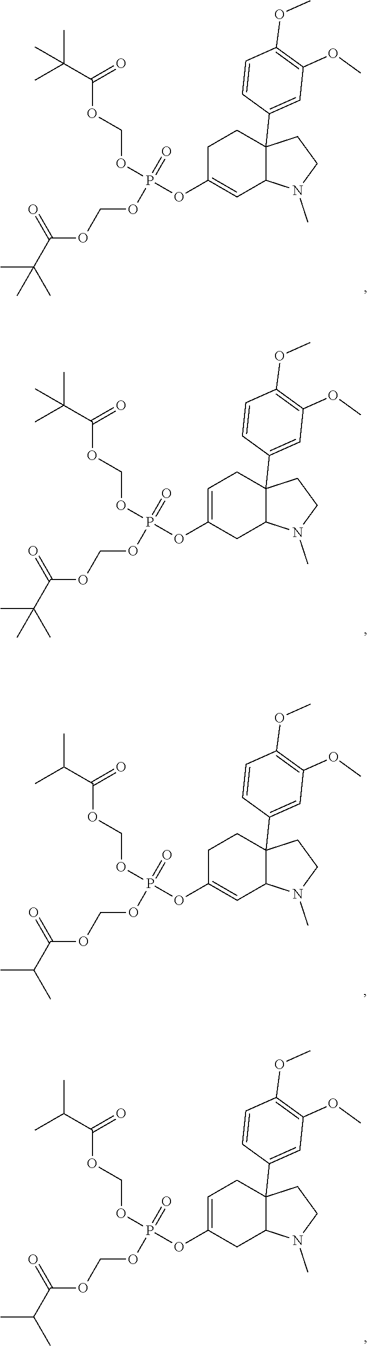

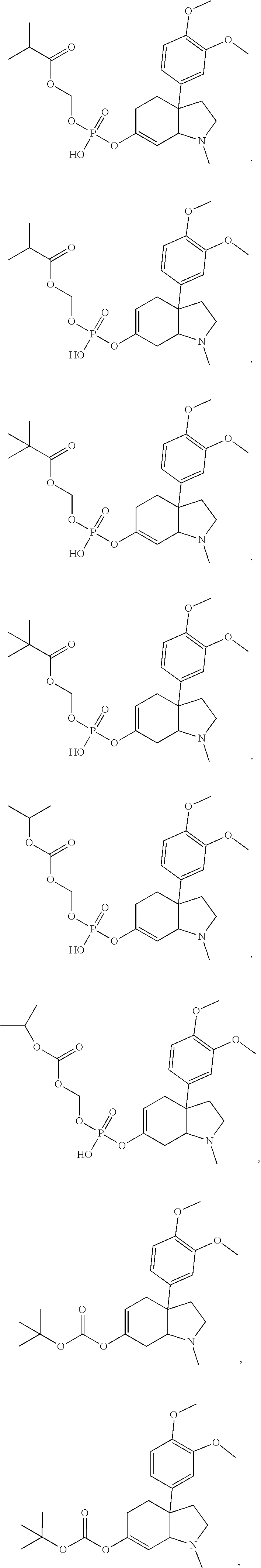

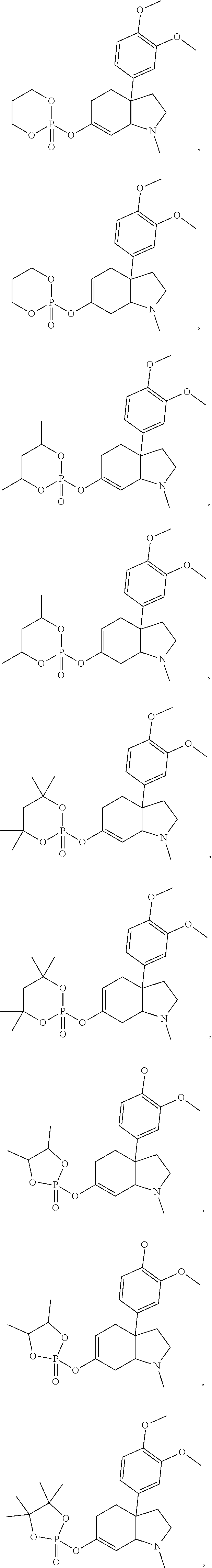

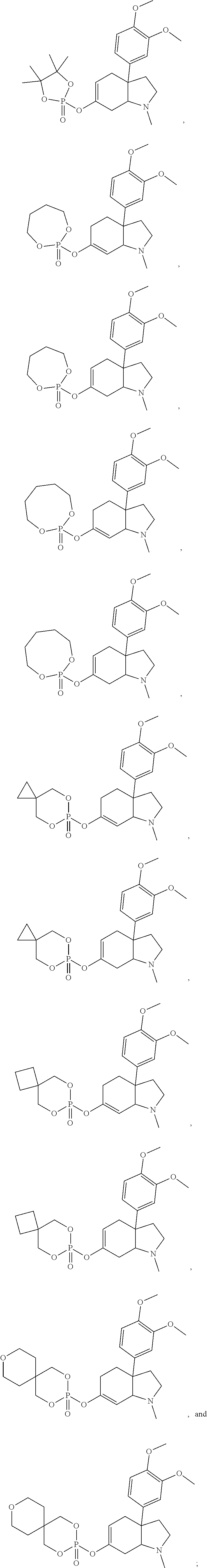

or a pharmaceutically acceptable salt thereof.

24. The compound of embodiment 1, selected from:

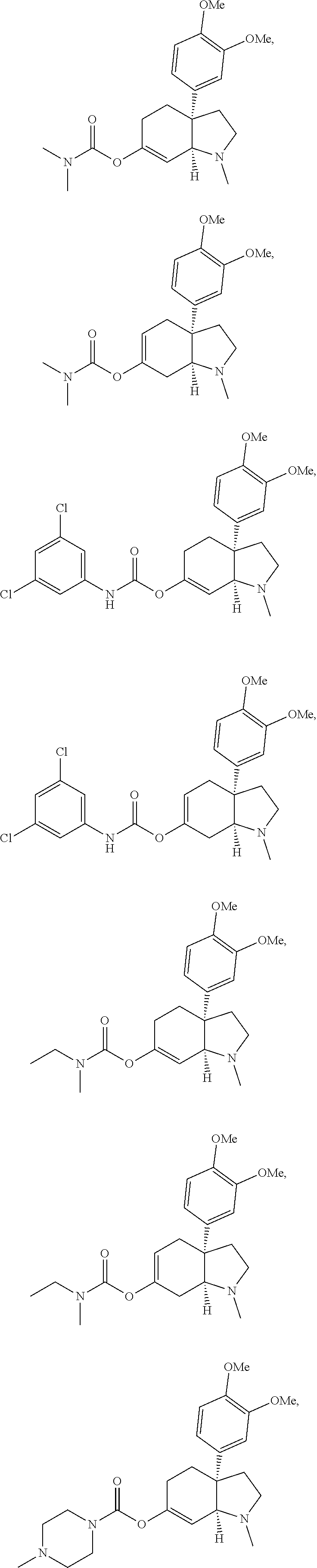

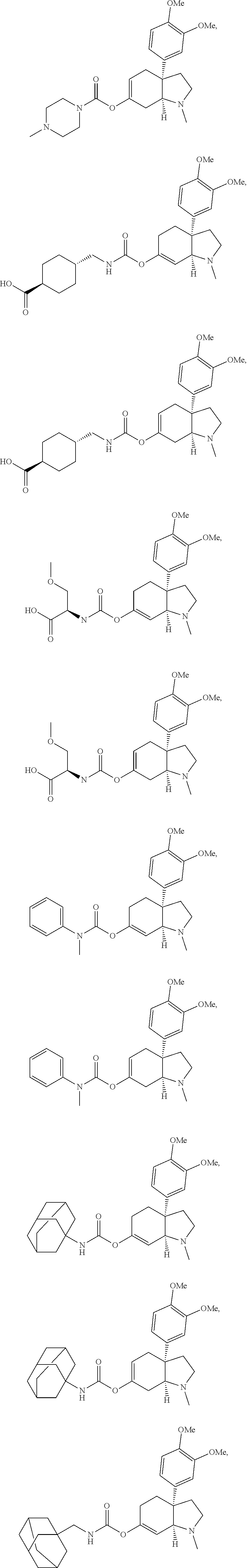

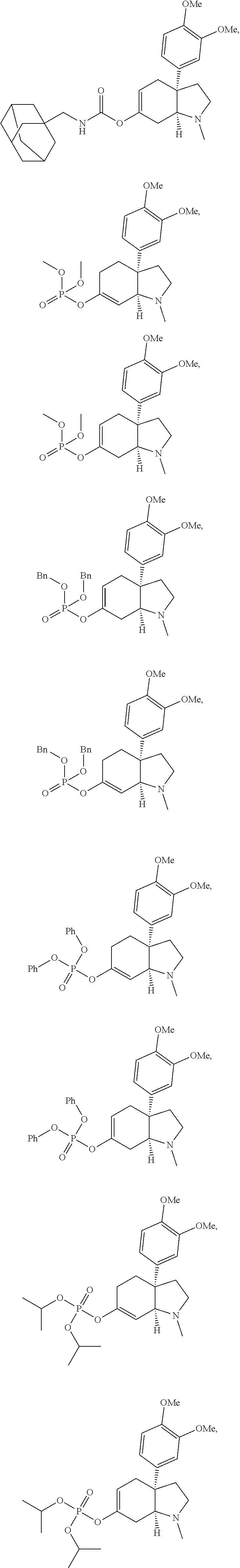

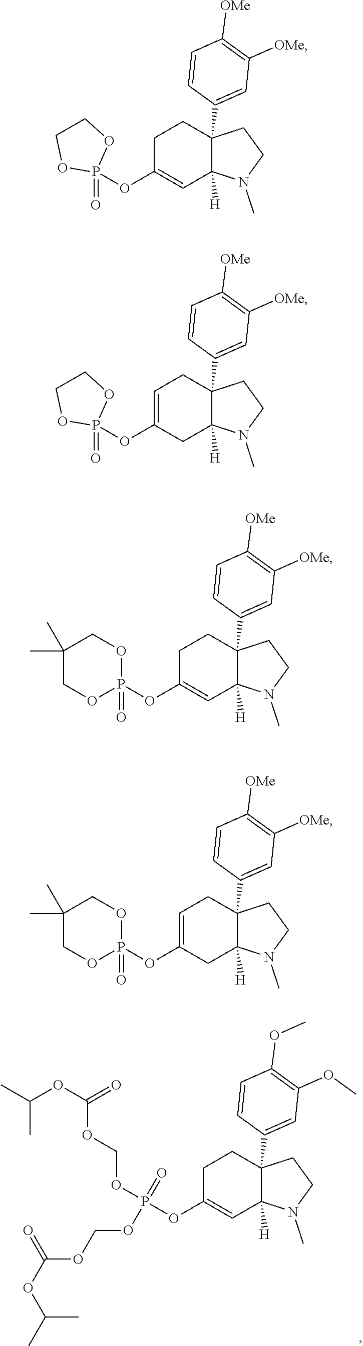

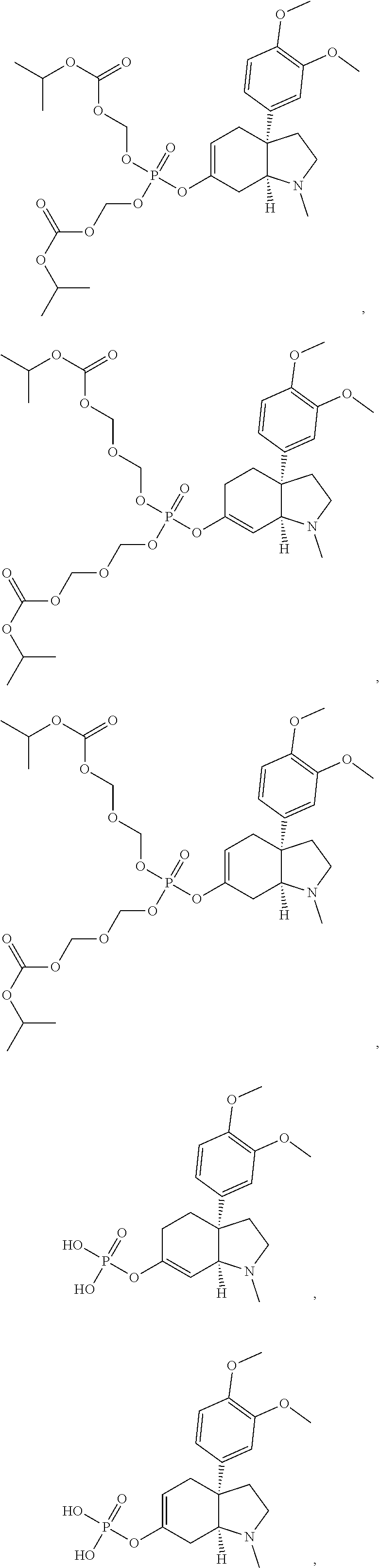

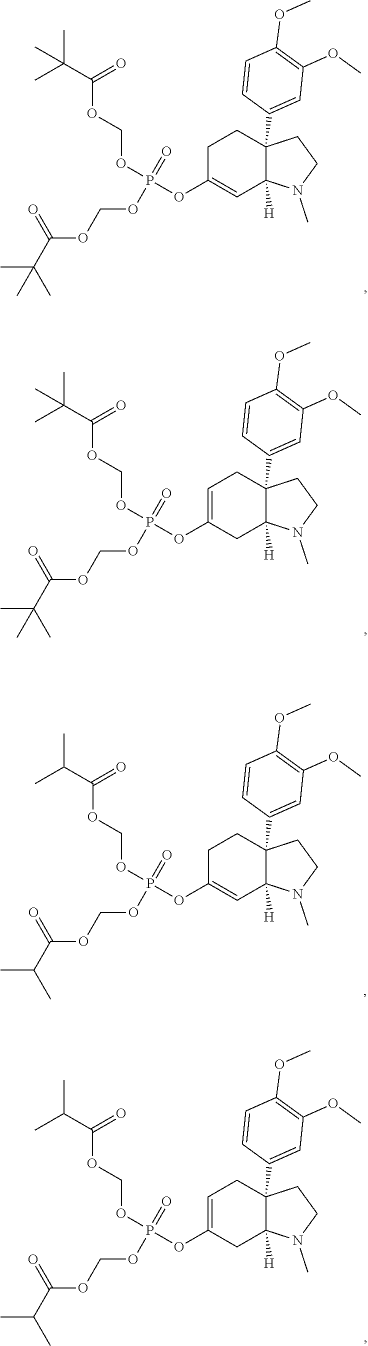

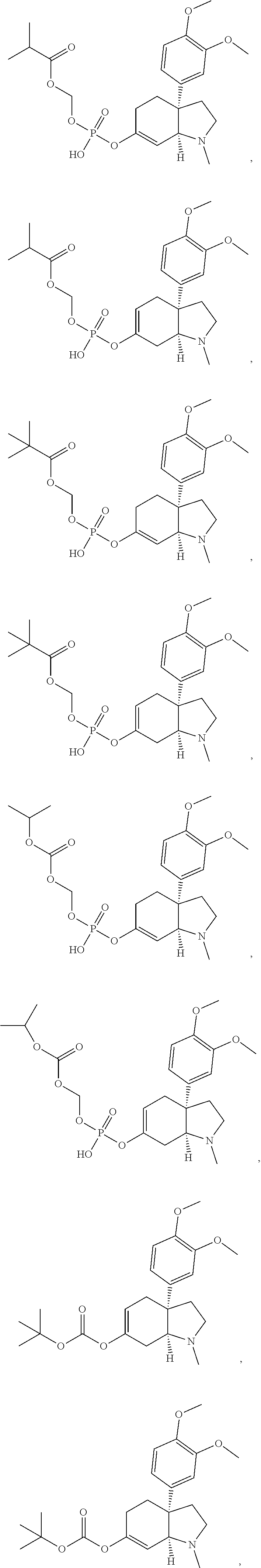

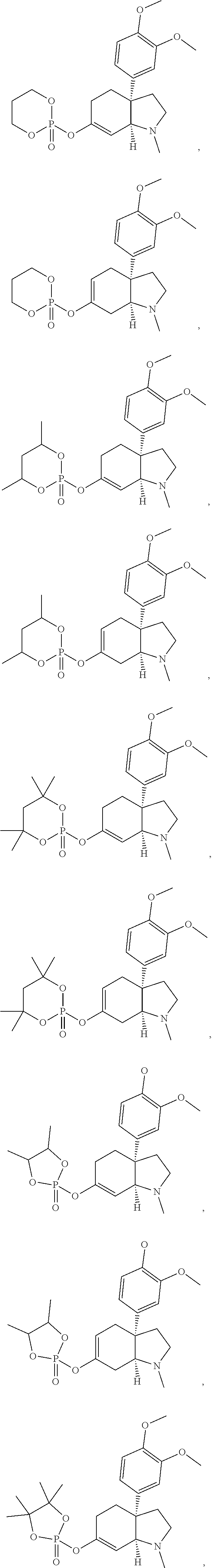

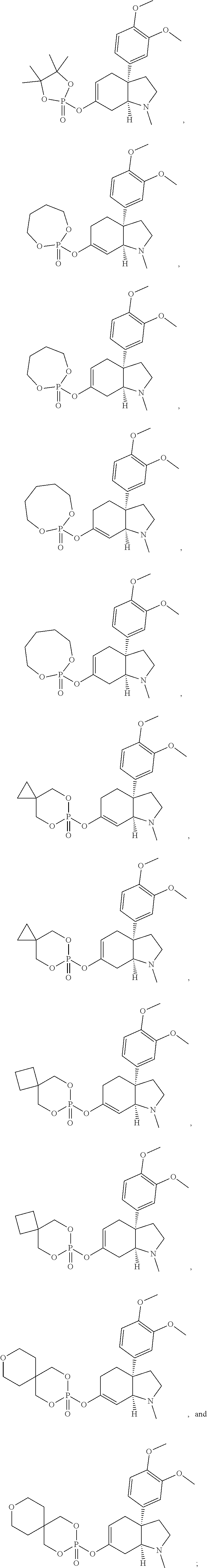

or a pharmaceutically acceptable salt thereof.

25. The compound of embodiment 1, selected from:

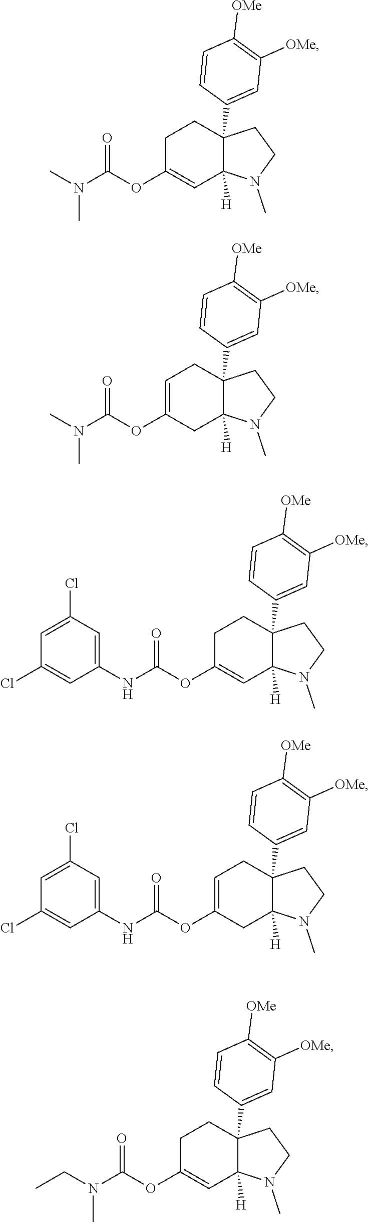

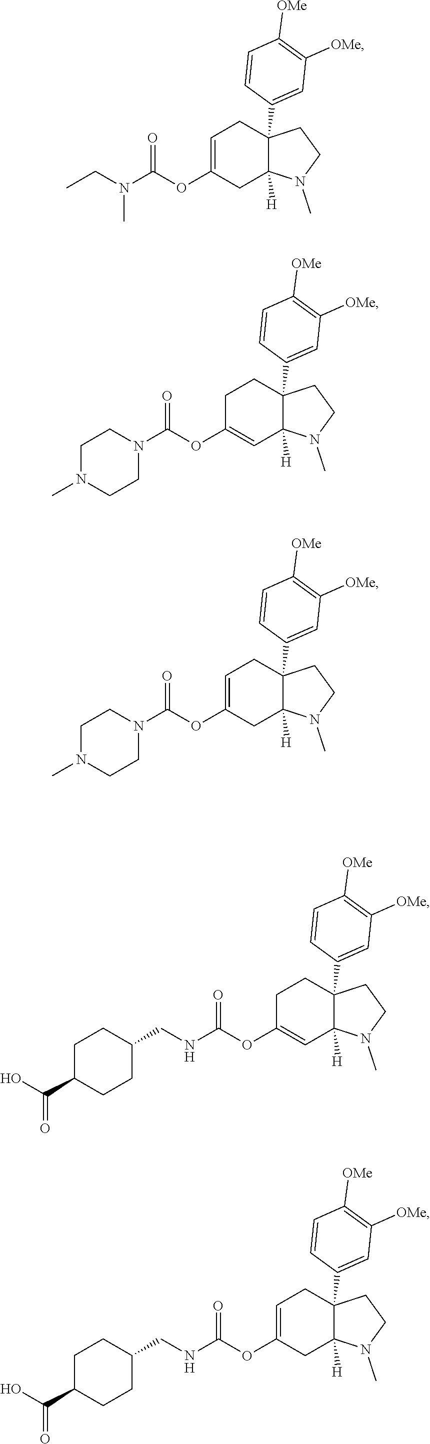 [SMILES: CCN(C)C(=O)OC1=CC[C@@]2(c3ccc(OC)c(OC)c3)CCN(C)[C@H]2C1 ; COc1ccc([C@@]23CCC(OC(=O)N4CCN(C)CC4)=C[C@@H]2N(C)CC3)cc1OC ; COc1ccc([C@@]23CC=C(OC(=O)N4CCN(C)CC4)C[C@@H]2N(C)CC3)cc1OC ; COc1ccc([C@@]23CCC(OC(=O)NC[C@H]4CC[C@H](C(=O)O)CC4)=C[C@@H]2N(C)CC3)cc1OC ; COc1ccc([C@@]23CC=C(OC(=O)NC[C@H]4CC[C@H](C(=O)O)CC4)C[C@@H]2N(C)CC3)cc1OC]

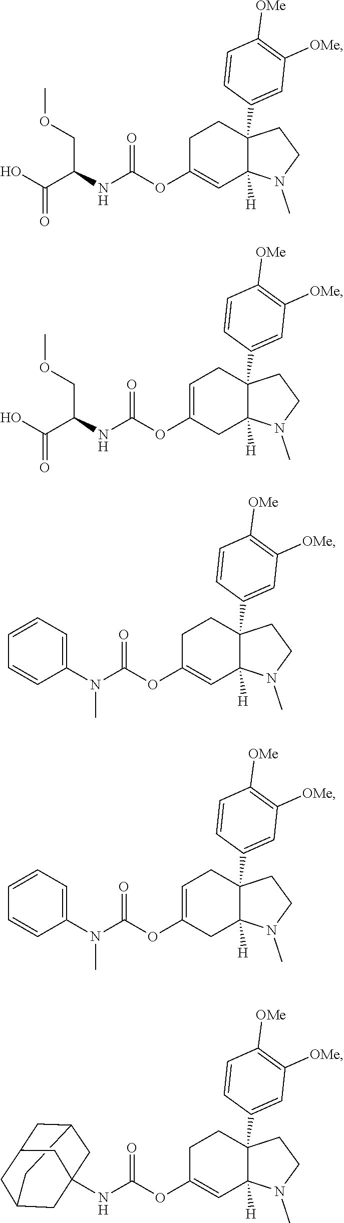 [SMILES: COC[C@@H](NC(=O)OC1=C[C@@H]2N(C)CC[C@]2(c2ccc(OC)c(OC)c2)CC1)C(=O)O ; COC[C@@H](NC(=O)OC1=CC[C@@]2(c3ccc(OC)c(OC)c3)CCN(C)[C@H]2C1)C(=O)O ; COc1ccc([C@@]23CCC(OC(=O)N(C)c4ccccc4)=C[C@@H]2N(C)CC3)cc1OC ; COc1ccc([C@@]23CC=C(OC(=O)N(C)c4ccccc4)C[C@@H]2N(C)CC3)cc1OC ; COc1ccc([C@@]23CCC(OC(=O)NC45CC6CC(CC(C6)C4)C5)=C[C@@H]2N(C)CC3)cc1OC]

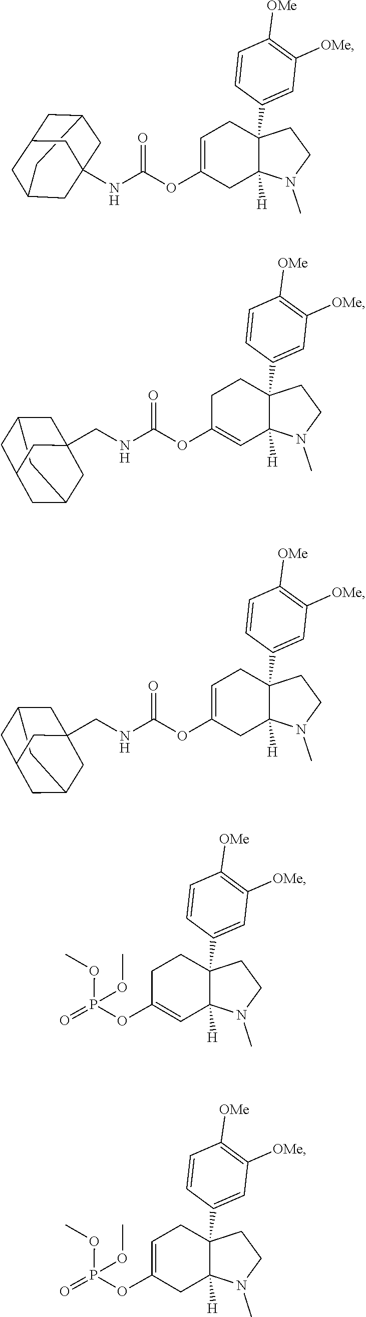 [SMILES: COc1ccc([C@@]23CC=C(OC(=O)NC45CC6CC(CC(C6)C4)C5)C[C@@H]2N(C)CC3)cc1OC ; COc1ccc([C@@]23CCC(OC(=O)NCC45CC6CC(CC(C6)C4)C5)=C[C@@H]2N(C)CC3)cc1OC ; COc1ccc([C@@]23CC=C(OC(=O)NCC45CC6CC(CC(C6)C4)C5)C[C@@H]2N(C)CC3)cc1OC ; COc1ccc([C@@]23CCC(OP(=O)(OC)OC)=C[C@@H]2N(C)CC3)cc1OC ; COc1ccc([C@@]23CC=C(OP(=O)(OC)OC)C[C@@H]2N(C)CC3)cc1OC]

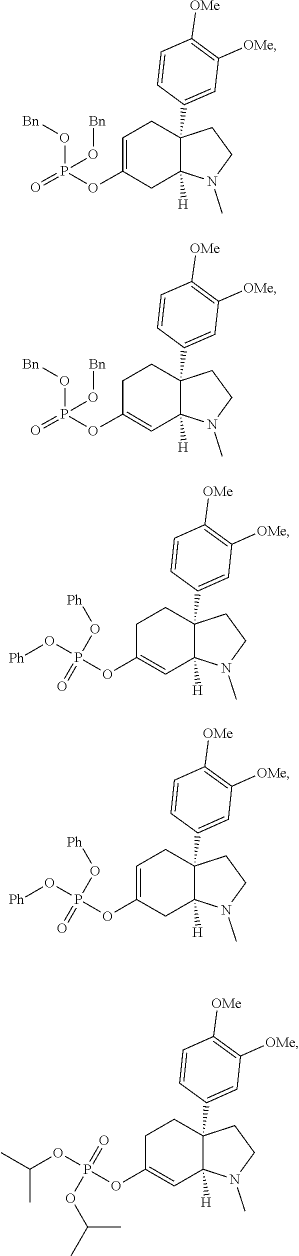

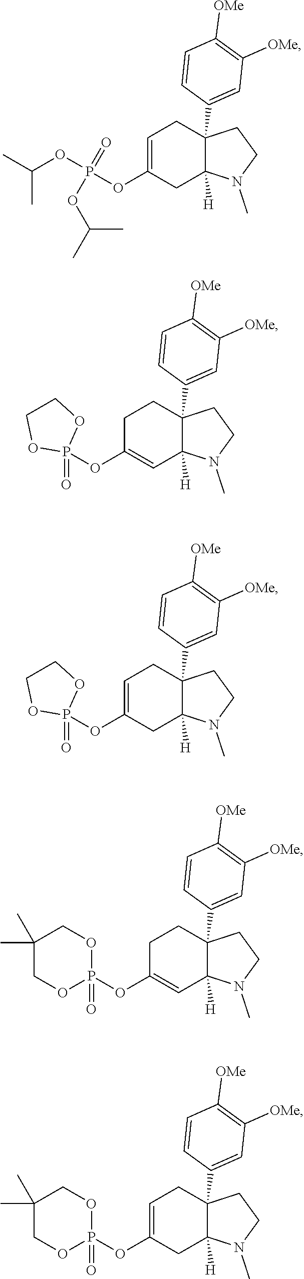 [SMILES: COc1ccc([C@@]23CC=C(OP(=O)(OC(C)C)OC(C)C)C[C@@H]2N(C)CC3)cc1OC ; COc1ccc([C@@]23CCC(OP4(=O)OCCO4)=C[C@@H]2N(C)CC3)cc1OC ; COc1ccc([C@@]23CC=C(OP4(=O)OCCO4)C[C@@H]2N(C)CC3)cc1OC ; COc1ccc([C@@]23CCC(OP4(=O)OCC(C)(C)CO4)=C[C@@H]2N(C)CC3)cc1OC ; COc1ccc([C@@]23CC=C(OP4(=O)OCC(C)(C)CO4)C[C@@H]2N(C)CC3)cc1OC]

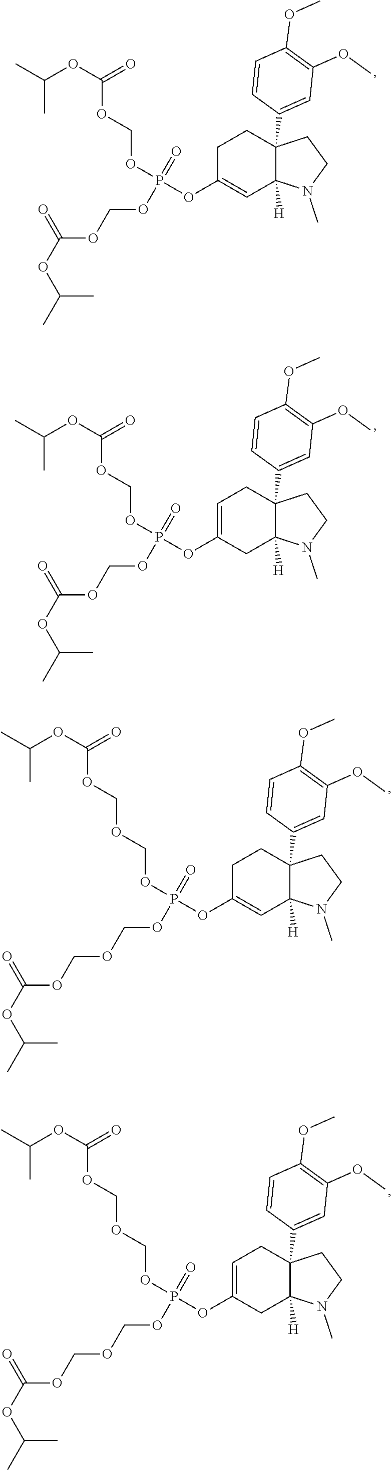

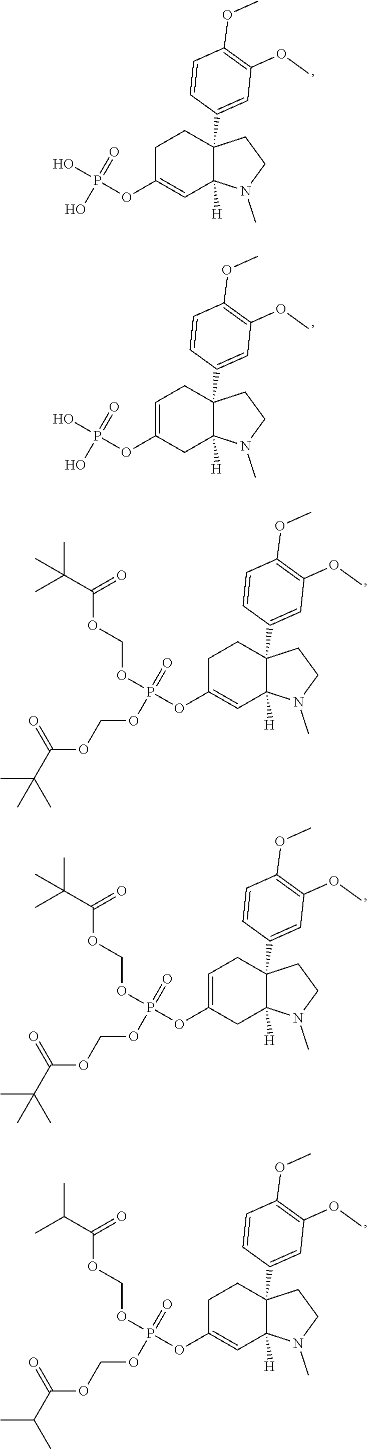

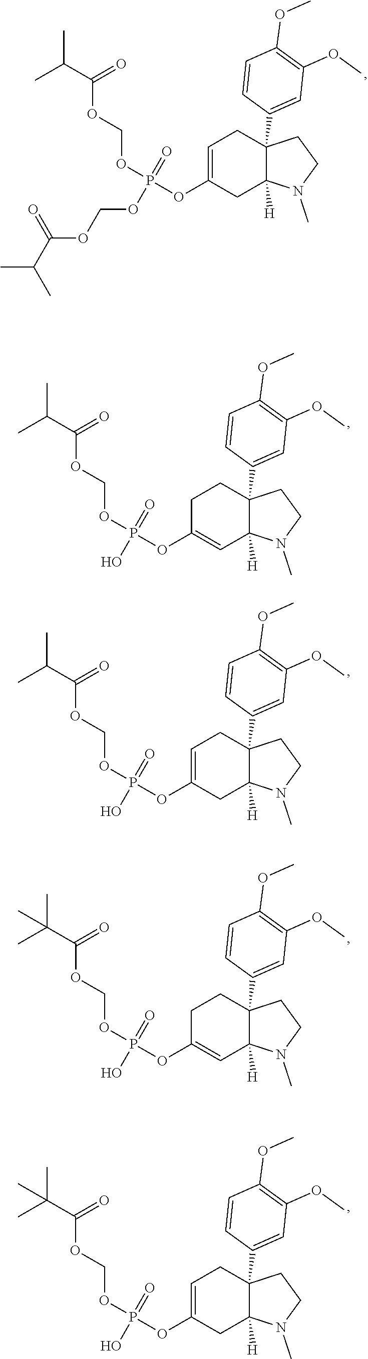

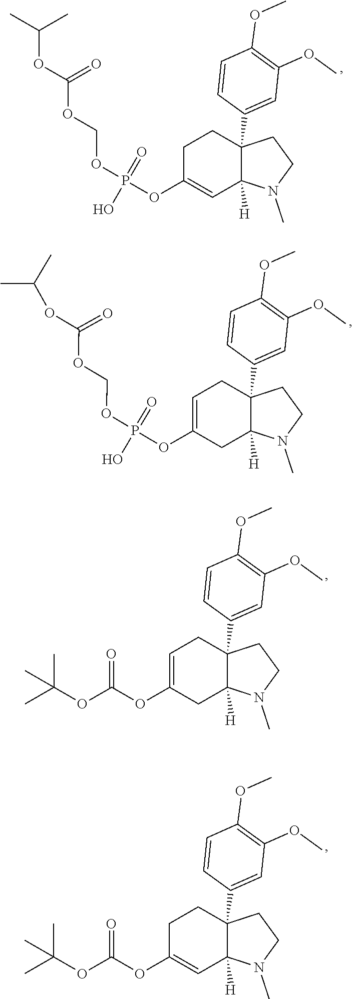

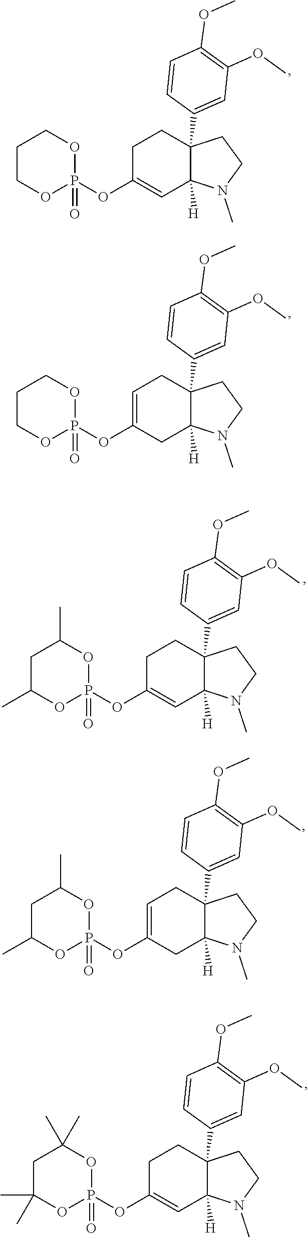

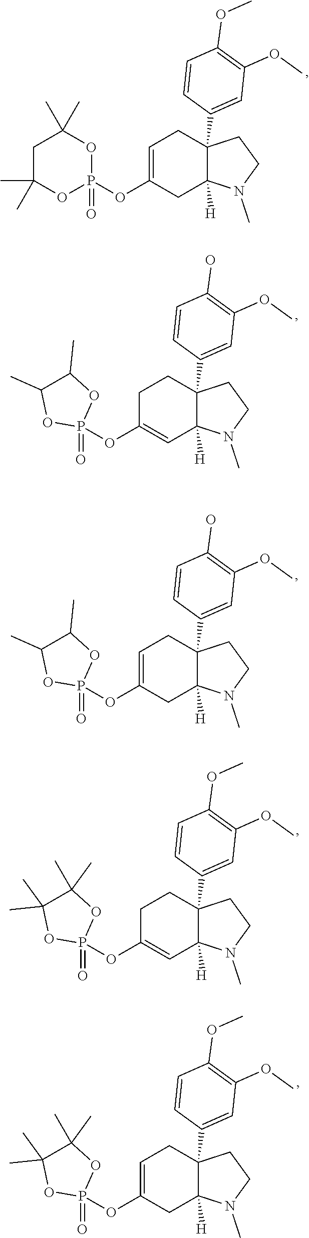

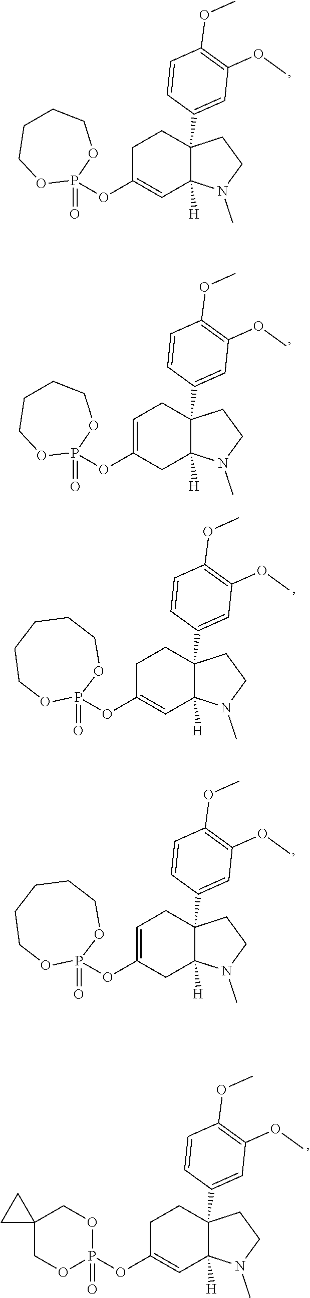

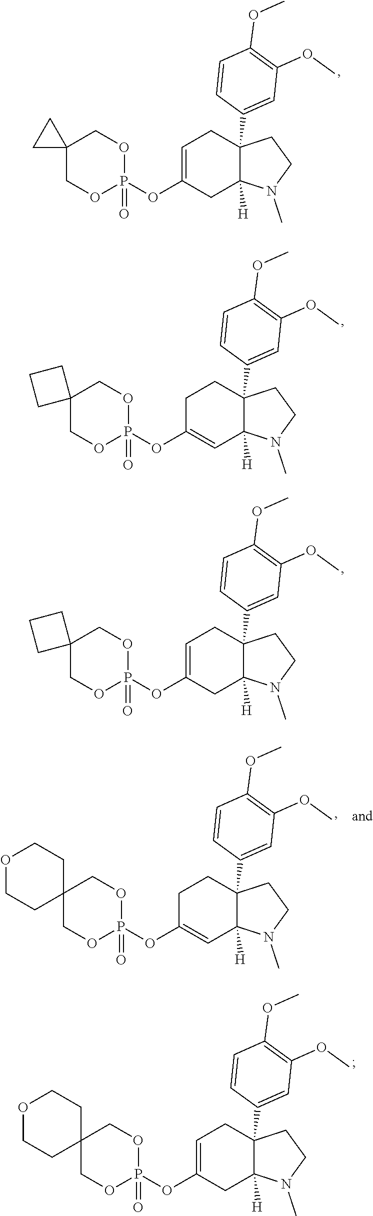

or a pharmaceutically acceptable salt thereof, and the compound has the absolute stereochemistry shown.

26. The compound of embodiment 1, selected from:

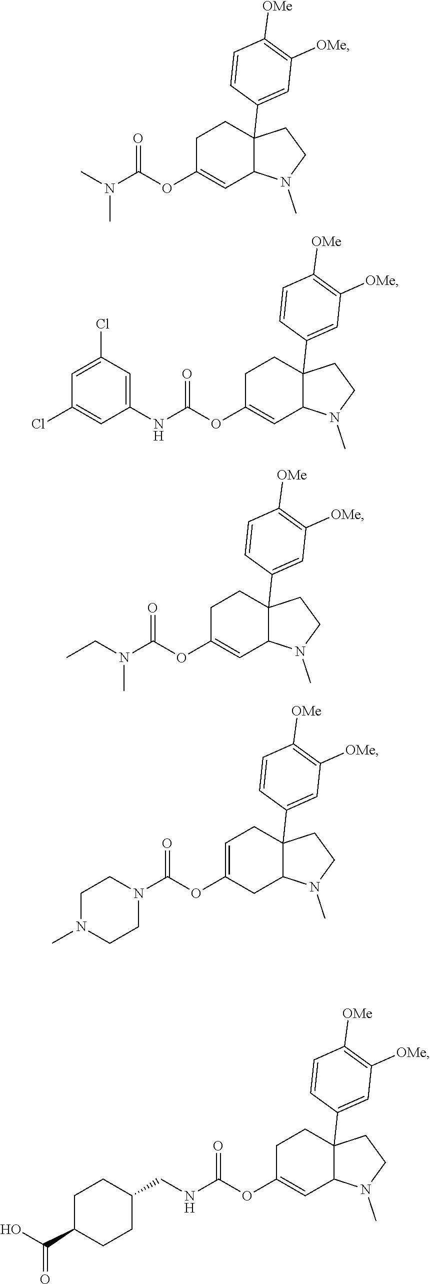

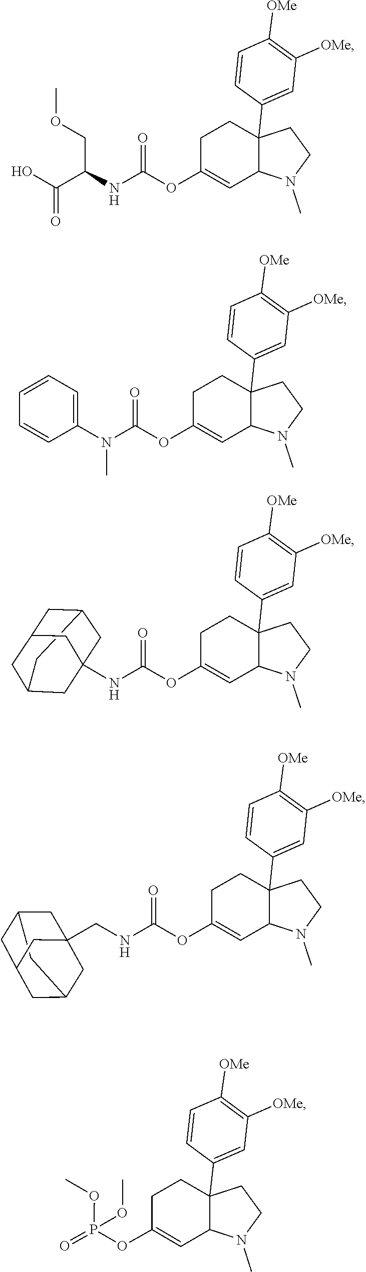

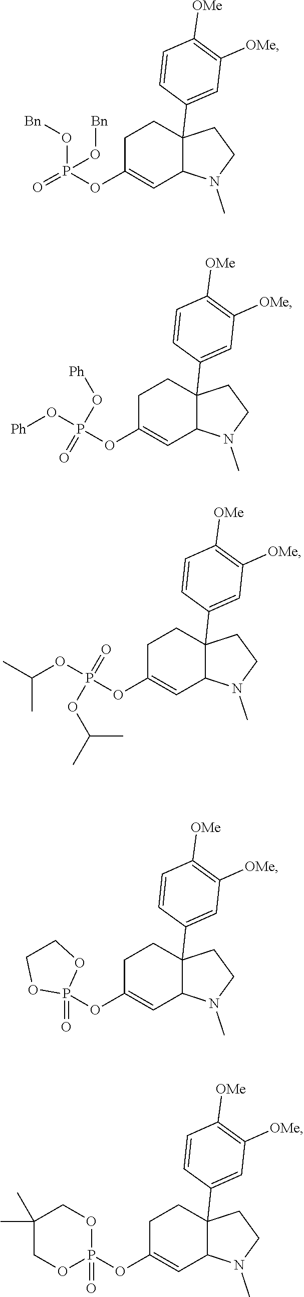

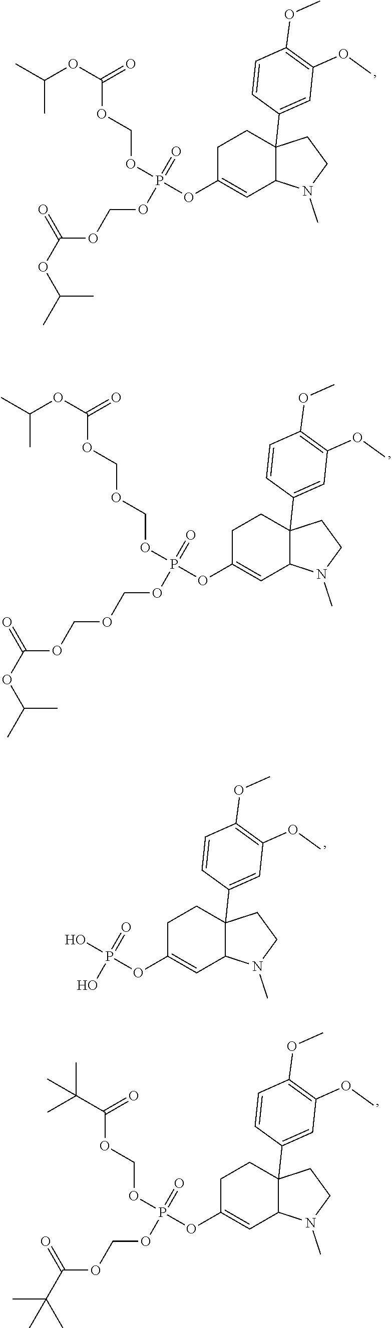

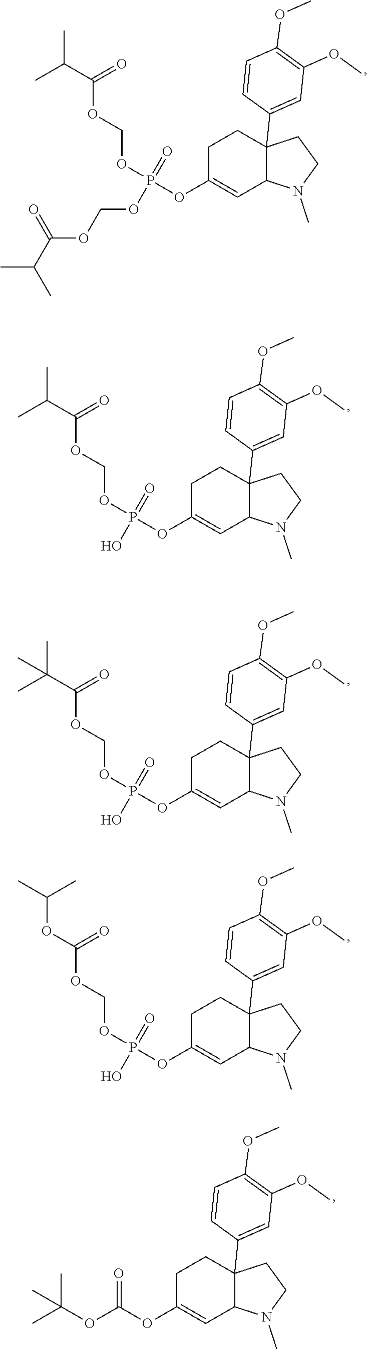

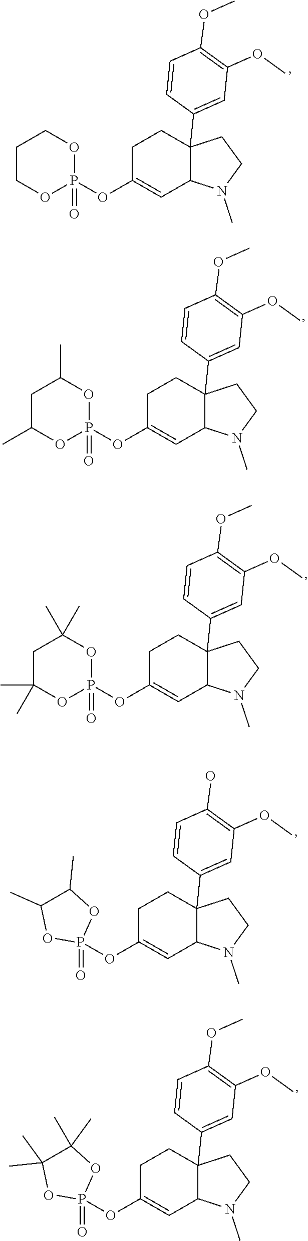

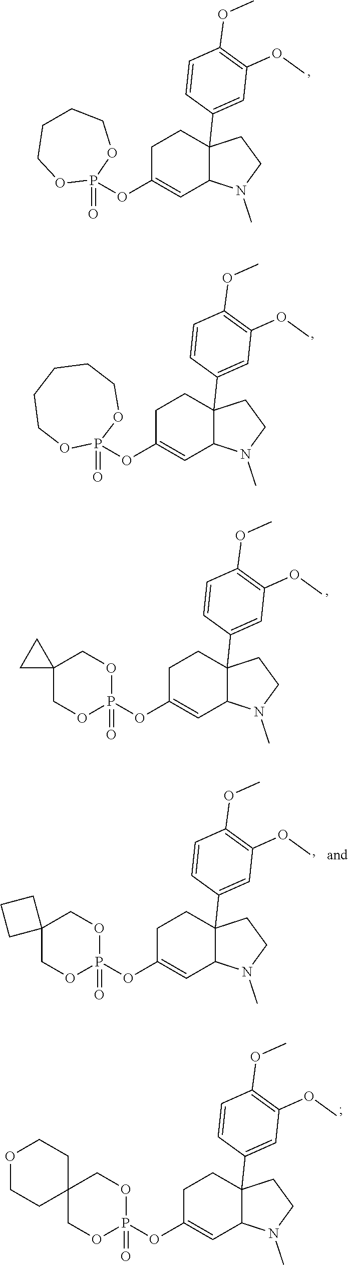

or a pharmaceutically acceptable salt thereof.

27. The compound of embodiment 1, selected from:

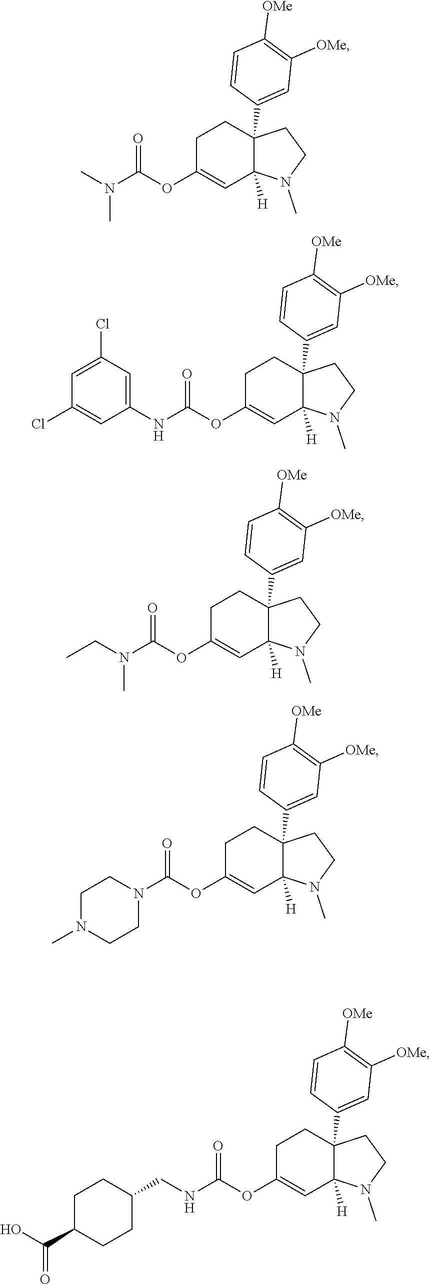

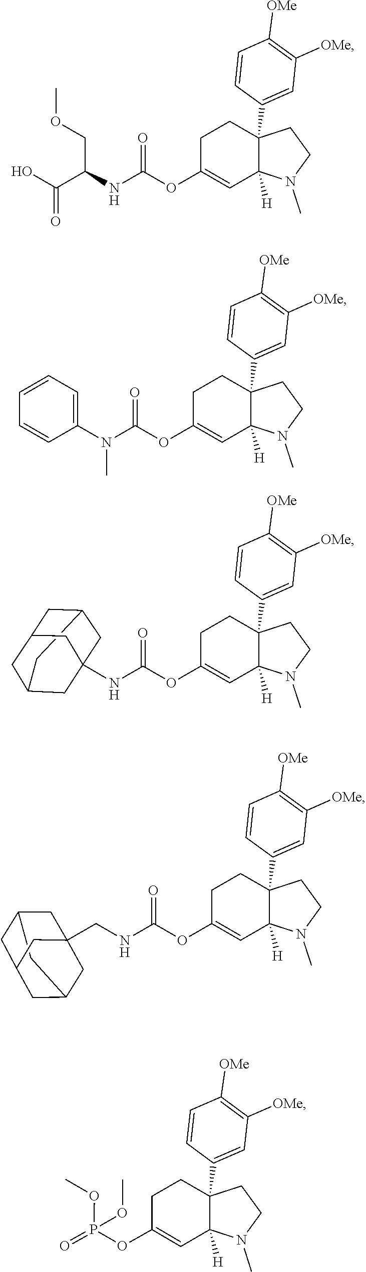

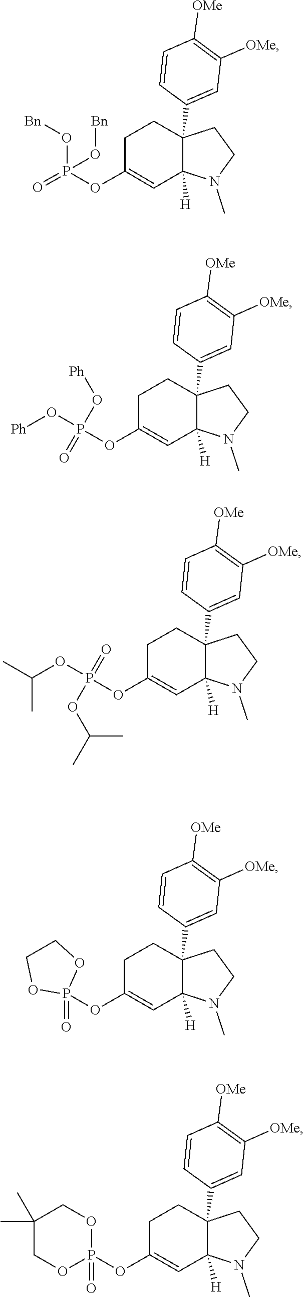

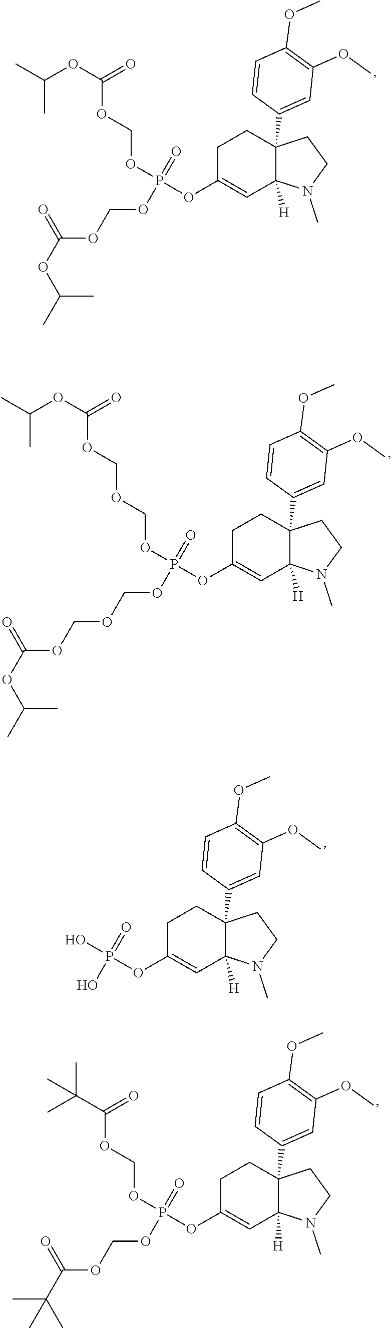

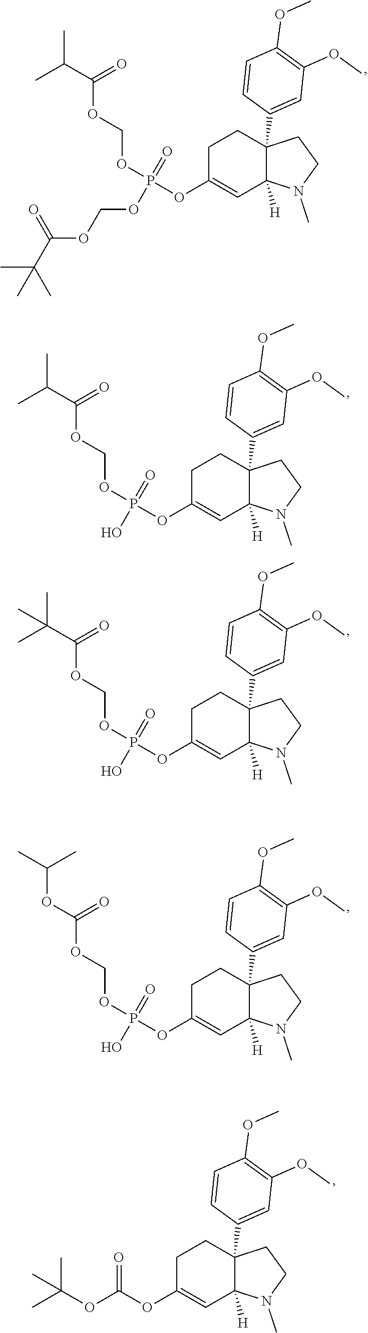

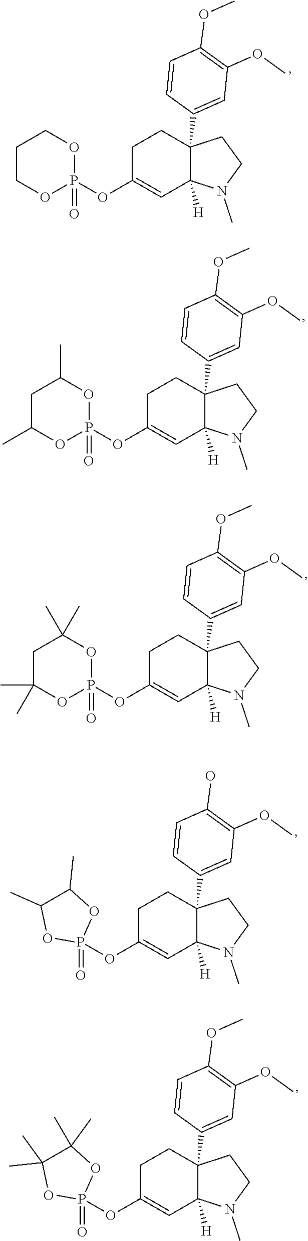

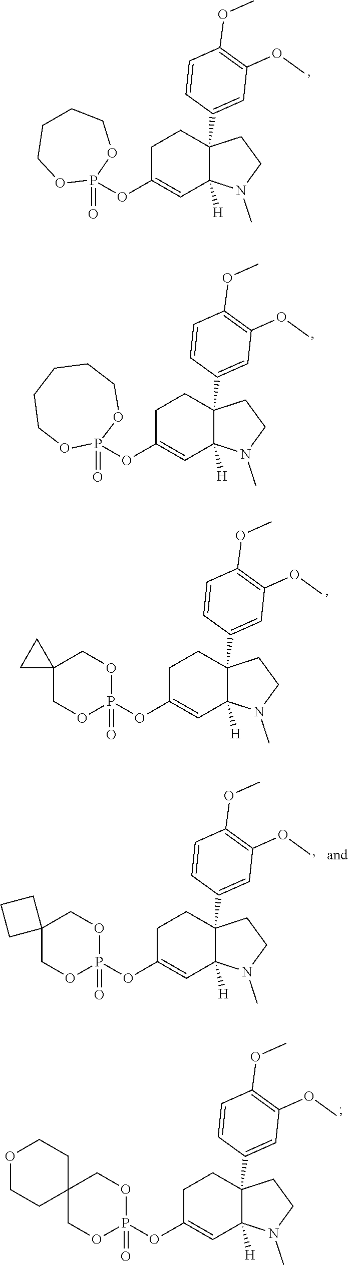

or a pharmaceutically acceptable salt thereof.

28. The compound of embodiment 1, selected from:

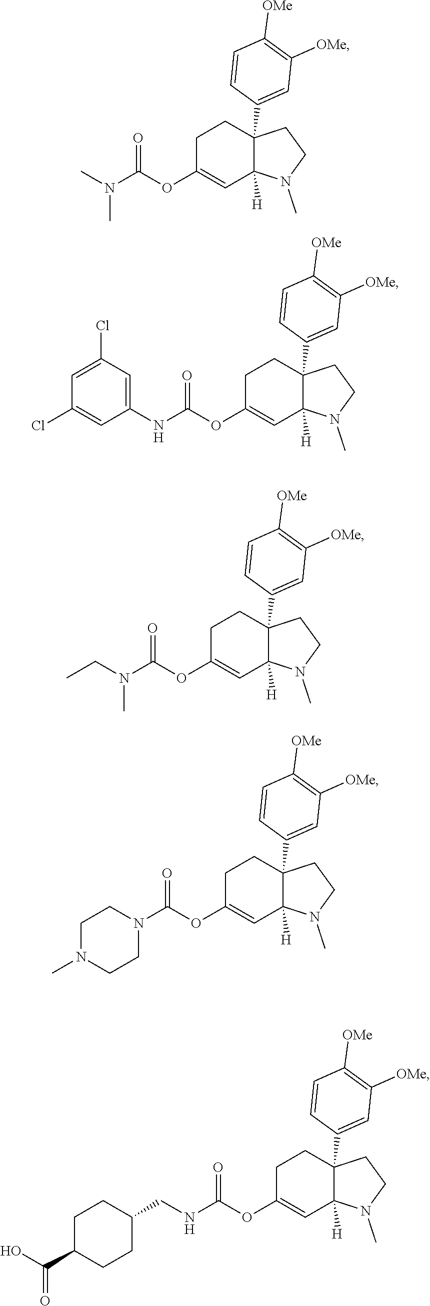

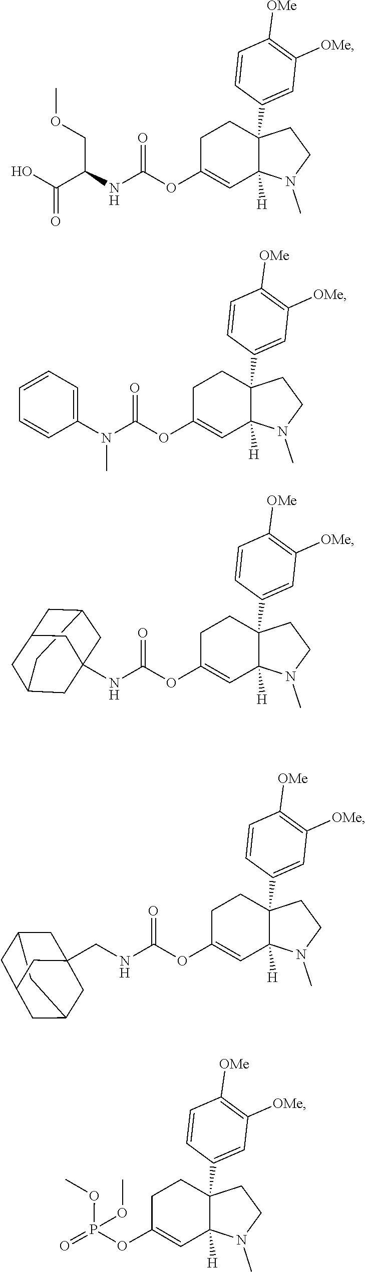

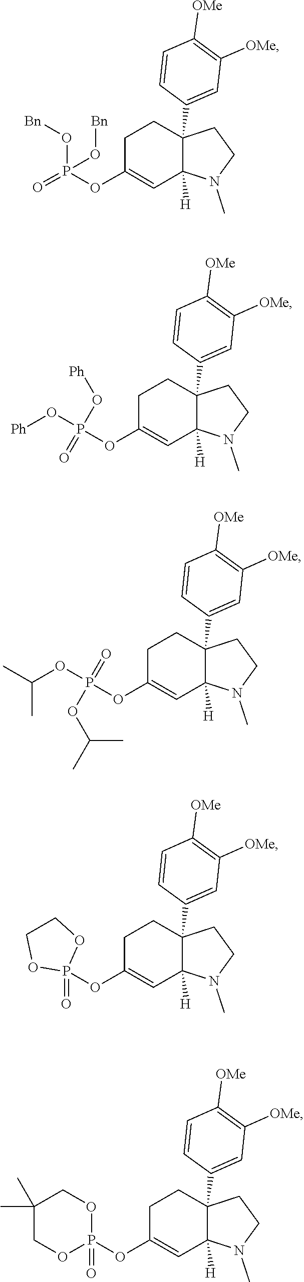 [SMILES: COc1ccc([C@@]23CCC(OP(=O)(OCc4ccccc4)OCc4ccccc4)=C[C@@H]2N(C)CC3)cc1OC ; COc1ccc([C@@]23CCC(OP(=O)(Oc4ccccc4)Oc4ccccc4)=C[C@@H]2N(C)CC3)cc1OC ; COc1ccc([C@@]23CCC(OP(=O)(OC(C)C)OC(C)C)=C[C@@H]2N(C)CC3)cc1OC ; COc1ccc([C@@]23CCC(OP4(=O)OCCO4)=C[C@@H]2N(C)CC3)cc1OC ; COc1ccc([C@@]23CCC(OP4(=O)OCC(C)(C)CO4)=C[C@@H]2N(C)CC3)cc1OC]

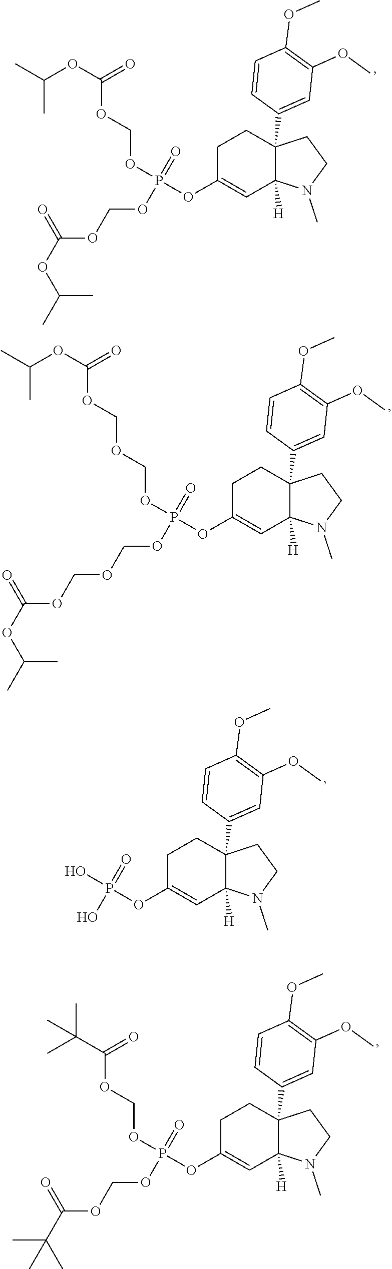

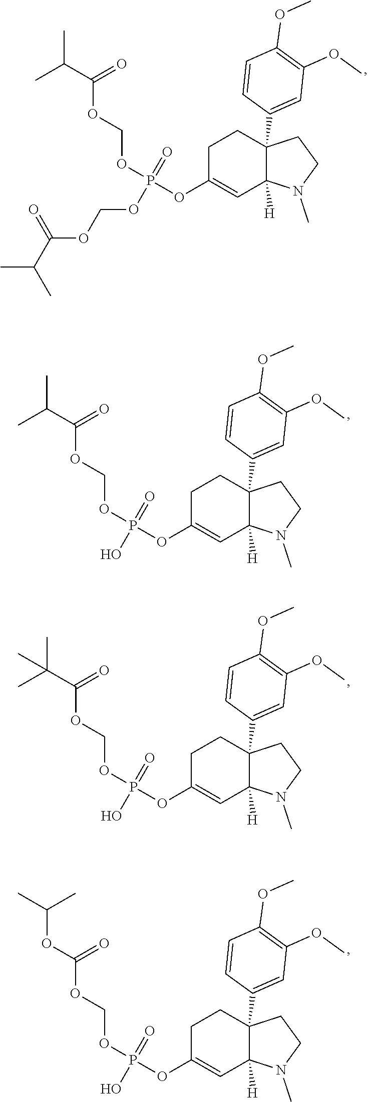

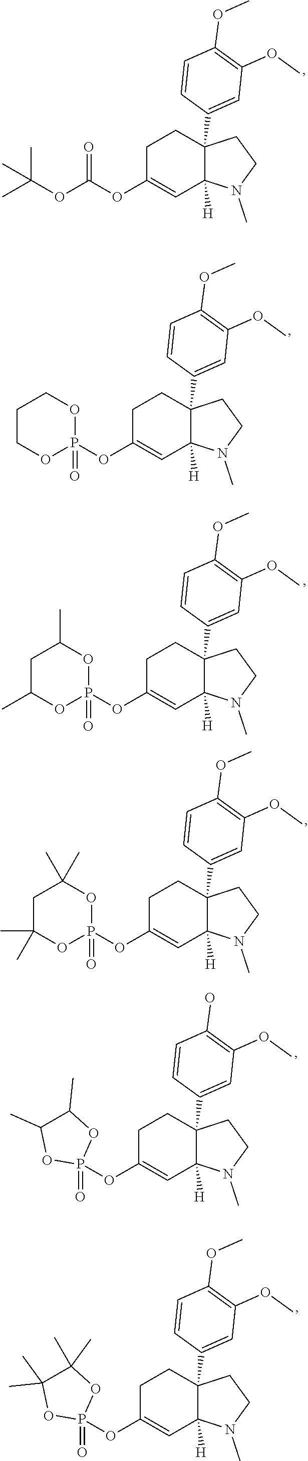

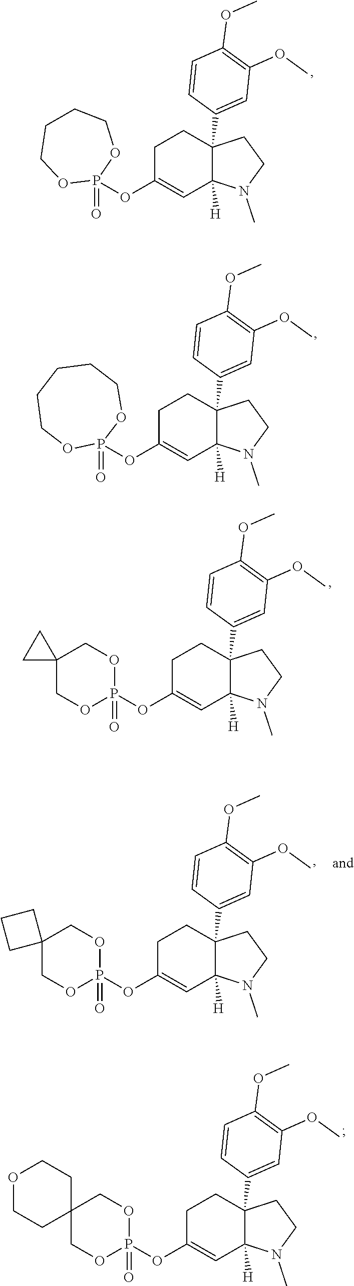

or a pharmaceutically acceptable salt thereof, and the compound has the absolute stereochemistry shown.

29. The compound of embodiment 1, represented by:

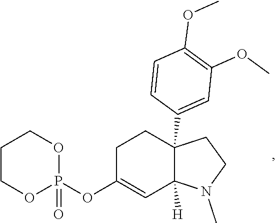 [SMILES: COc1ccc([C@@]23CCC(OP4(=O)OCCCO4)=C[C@@H]2N(C)CC3)cc1OC]

or a pharmaceutically acceptable salt thereof.

30. The compound of embodiment 1, represented by:

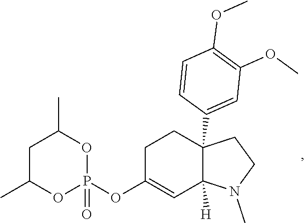

or a pharmaceutically acceptable salt thereof.

31. A pharmaceutical composition, comprising a compound of any one of embodiments 1-30; and a pharmaceutically acceptable excipient.

32. A method of treating a mental health disorder, comprising administering to a mammal in need thereof an effective amount of a compound of any one of embodiments 1-30.

33. The method of embodiment 32, wherein the mental health disorder is anxiety, stress, or depression.

34. The method of embodiment 33, wherein the mental health disorder is anxiety.

35. The method of embodiment 33, wherein the mental health disorder is stress.

36. The method of embodiment 33, wherein the mental health disorder is depression.

37. The method of any one of embodiments 32-36, wherein the mammal is a human.

### EXAMPLES

LC/MS spectra were obtained using Agilent 1200G1956A or SHIMADZU LCMS-2020. Standard LC/MS conditions were as follows (running time 1.55 minutes).

Acidic condition: Mobile Phase A: 0.037500 TEA in water (v/v). Mobile Phase B: 0.01875% TFA in acetonitrile (v/v); Column: Kinetex EVO C18 30*2.1 mm, 5 μm.

Basic condition: Mobile Phase A: 0.025% NH₃·H₂O in water (v/v). Mobile Phase B: Acetonitrile; Column: Kinetex EVO C18 2.1×30 mm, 5 μm.

5-95AB_0.8min

| Instrument | | SHIMADZU LCMS-2020; | | |
|---|---|---|---|---|
| Software | | LabSolution Version 5.97SP1 | | |
| HPLC | Column | Kinetex ® EVO C18 2.1 × 30 mm 5 um | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 5.0 | 2.0 |
| | | 0.60 | 95.0 | 2.0 |
| | | 0.78 | 95.0 | 2.0 |
| | | 0.79 | 5.0 | 2.0 |
| | | 0.80 | 5.0 | 2.0 |

| | | |
|---|---|---|
| | Column Temp | 50° C. |
| | Detector | PDA (220 nm&254 nm) |
| MS | Ionization source | ESI |
| | Drying Gas | N2 |
| | Drying Gas Flow | 15(L/min) |
| | DL Voltage | 120(v) |
| | Qarray DC Voltage | 20(V) |
| | MS Polarity | Positive |
| | MS Mode | Scan |
| | Mass range | 100-1000 |

### Table of Abbreviations

| | |
|---|---|
| Ac | Acetyl |
| ACN | Acentonitrile |
| Bn | Benzyl |
| C | Celsius |
| δ | Chemical shift |
| d | Doublet |
| DCM | Dichloroethane |
| DIBAL-H | Diisobutyl aluminum hydride |
| DMF | N,N-Dimethyformamide |
| ESI | Electrospray ionization |
| Et | Ethyl |
| FA | Formic acid |
| HPLC | High performance liquid chromatography |
| hr | Hours |
| Hz | Hertz |
| J | Coupling constant |
| LC-MS | Liquid chromatography-mass spectrometry |
| m | Multiplet |
| M | Mass |
| m/z | Mass to charge ratio |
| Me | Methyl |
| min | Minutes |
| mol | Moles |
| NMP | N-Methyl-2-pyrrolidone |
| NMR | Nuclear magnetic resonance |
| Ph | Phenyl |
| q | Quartet |
| s | Singlet |
| t-Bu | Tertiary butyl |
| TEA | Triethylamine |
| THF | Tetrahydrofuran |
| TMS | Trimethylsilyl |

### Example 1: Synthesis of ((+/−)-Mesembrine

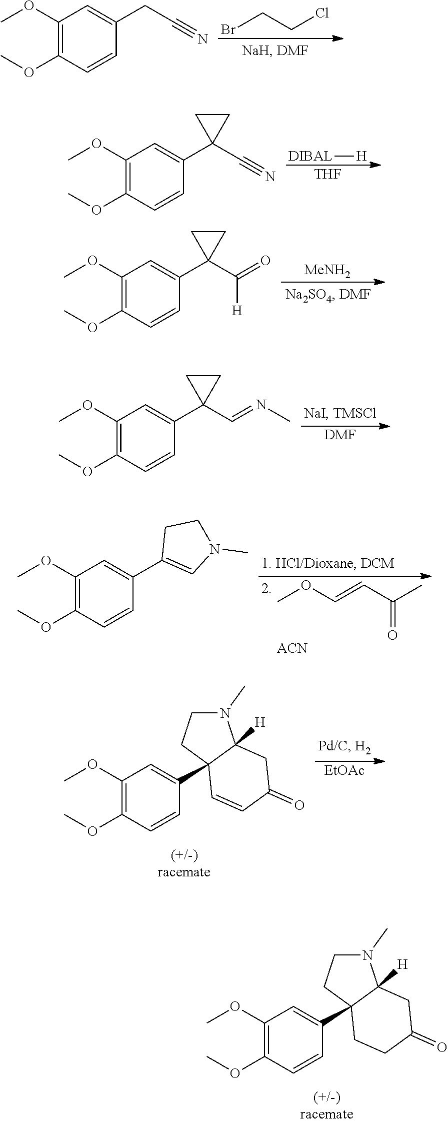

### Step 1: Synthesis of 1-(3,4-dimethoxyphenyl)cyclopropanecarbonitrile

To a solution of 2-(3,4-dimethoxyphenyl)acetonitrile (20 g, 112 mmol) in DMF (93 mL) was added NaH (18.0 g, 451 mmol, 60% purity) in portions. The mixture was allowed to stir at 25° C. for 20 min. 1-bromo-2-chloro-ethane (16.1 g, 112 mmol) was added, and the mixture was allowed to stir at 25° C. for 16 hr. The reaction was quenched by the addition of a MeOH/water mixture (1: 1, 1000 mL) and the resulting solution was extracted with EtOAc (3×500 mL). The organic solutions were combined, washed with water (4×500 mL) and brine (1×200 mL) and dried over (Na₂SO₄). The solution was filtered and the solvent was evaporated under reduced pressure. The resulting solid was purified by column chromatography (SiO₂, Petroleum ether/EtOAc=10/1 to 3/1) (15 g, 65%) as yellow oil. ¹H NMR (400 MHz, CHLOROFORM-d) δ 6.88 (s, 1H), 6.82 (d, J=1.2 Hz, 2H), 3.91 (s, 3H), 3.88 (s, 3H), 1.68-1.65 (m, 2H), 1.35 (d, J=2.4 Hz, 2H).

### Step 2: Synthesis of 1-(3,4-dimethoxyphenyl)cyclopropanecarbaldehyde

To a solution of 1-(3,4-dimethoxyphenyl)cyclopropanecarbonitrile (11 g, 54.1 mmol) in THF (160 mL) was added DIBAL-H (1 M in toluene, 81.2 mL). The mixture was allowed to stir at 25° C. for 3 hr and then the reaction was cautiously quenched by addition of aqueous 2 M HCl. The solution was extracted with DCM (3×200 mL). The organic solutions were combined, washed with water (2×200 mL) and brine (2×200 mL), and then dried over Na₂SO₄ to give the titled compound (9.6 g, 85%) as yellow oil. LC-MS (ESI+) m/z 207.0 (M+H)⁺. ¹H NMR (400 MHz, CDCl₃) δ 9.26 (s, 1H), 6.94-6.61 (m, 3H), 3.89 (d, J=2.8 Hz, 6H), 1.61-1.52 (m, 2H), 1.42-1.37 (m, 2H)

### Step 3: Synthesis of (Z)-1-[1-(3,4-dimethoxyphenyl)cyclopropyl]-N-methyl-methanimine

To a solution of 1-(3,4-dimethoxyphenyl)-cyclopropanecarbaldehyde (5.0 g, 24.2 mmol) in DCM (50 mL) was added MeNH₂ (2 M, 121 mL) and Na₂SO₄ (15.5 g, 109 mmol, 11.0 mL). The mixture was allowed to stir at 25° C. for 16 hr. The reaction mixture was filtered and the filtrate was concentrated under reduced pressure to give the titled compound (5.1 g, 99%) as white solid. LC-MS (ESI⁺) m/z 219.9 (M+H)⁺; ¹H NMR (400 MHz, CDCl₃) δ 7.55 (q, J=1.2 Hz, 1H), 6.93-6.77 (m, 3H), 3.88 (d, J=7.2 Hz, 6H), 3.24 (d, J=1.6 Hz, 3H), 1.29-1.23 (m, 2H), 1.18-1.12 (m, 2H).

### Step 4: Synthesis of 4-(3,4-dimethoxyphenyl)-1-methyl-2,3-dihydropyrrole

To a solution of (Z)-1-[1-(3,4-dimethoxyphenyl)cyclopropyl]-N-methyl-methanimine (5.4 g, 24.6 mmol) in DMF (19 mL) was added NaI (366 mg, 2.44 mmol) and TMSCI (267 mg, 2.46 mmol). The mixture was allowed to stir at 90° C. for 3 hr. The reaction mixture was diluted with water (100 mL) and extracted with EtOAc (100 mL×3). The organic solutions were combined, washed with water and brine, dried over Na₂SO₄, and filtered. The filtrate was concentrated under reduced pressure to give the titled compound (6.25 g, 80%) as yellow oil. LC-MS (ESI⁺) m/z 220.0 (M+H)⁺. ¹H NMR (400 MHz, CDCl₃) δ 6.90-6.66 (m, 3H), 6.31 (t, J=1.6 Hz, 1H), 3.95-3.80 (m, 6H), 3.18-3.11 (m, 2H), 2.79 (dt, J=1.2, 9.0 Hz, 2H), 2.65 (s, 3H).

### Step 5: Synthesis of rac-3a-(3,4-dimethoxyphenyl)-1-methyl-2,3,7,7a-tetrahydroindol-6-one

4-(3,4-dimethoxyphenyl)-1-methyl-2,3-dihydropyrrole (6.25 g, 28.5 mmol) was dissolved in DCM (100 mL). To this solution was added HCl (1 M in dioxane, 25 mL, 100 mmol). The mixture was evaporated to dryness and then dissolved in ACN (90 mL). To this solution was added (E)-4-methoxybut-3-en-2-one (4.28 g, 42.7 mmol). The reaction mixture was allowed to stir at 90° C. for 16 hr. The reaction mixture was filtered and the filtrate was concentrated under reduced pressure. The residue was purified by HPLC (column: Phenomenex luna C18 (250*70 mm, 10 um); mobile phase: [water (NH₄HCO₃)-ACN]; B %: 22%-52%, 20 min). The eluent was acidified with aq. HCl to give the titled compound (3.0 g, 30%) as a white solid. LC-MS (ESI⁺) m/z 288.3 (M+H)⁺. ¹H NMR (400 MHz, CDCl₃) δ 6.90-6.88 (m, 1H), 6.87-6.83 (m, 2H), 6.74 (dd, J=2.0, 10.1 Hz, 1H), 6.11 (d, J=10.0 Hz, 1H), 3.89 (d, J=4.0 Hz, 6H), 3.33 (dt, J=2.4, 8.8 Hz, 1H), 2.69-2.66 (m, 1H), 2.58-2.51 (m, 2H), 2.50-2.41 (m, 2H), 2.33 (s, 3H), 2.27-2.18 (m, 1H)

### Step 6: Synthesis of rac-(3a, 7a)-3a-(3,4-dimethoxyphenyl)-1-methyloctahydro-6H-indol-6-one

A mixture of 3a-(3,4-dimethoxyphenyl)-2,3,7,7a-tetrahydro-1H-indol-6-one (12.0 g, 43.9 mmol) and 10% Pd/C (300 mg) in EtOAc (120 mL) was degassed and then purged with H₂ for 3 times. The mixture was allowed to stir at 25° C. for 2 hr under 15 psi H₂. The reaction mixture was filtered and the filtrate was concentrated in vacuo to give the titled compound (10 g, 80%) as brown oil. LC-MS (ESI⁺) m/z 290.4 (M+H)⁺. ¹H NMR (400 MHz, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

### Example 2 Chiral resolution of rac-(3a, 7a)-3a-(3,4-dimethoxyphenyl)-1-nethyloctahydro-6H-indol-6-one to give (−) Mesembrine 001

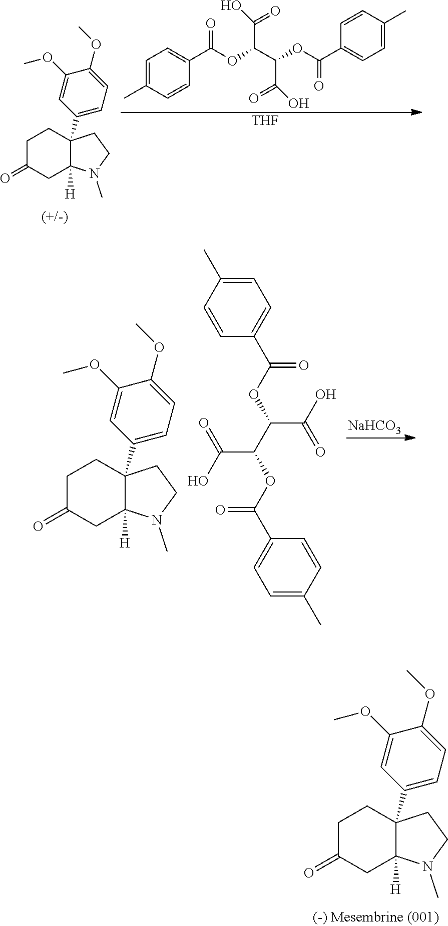

To a solution of 3a-(3,4-dimethoxyphenyl)-1-methyl-2,3,4,5,7,7a-hexahydroindol-6-one (28.0 g, 85.1 mmol) in THF (1400 mL) was added (2S,3S)-2,3-bis[(4-methylbenzoyl)oxy]butanedioic acid (19.7 g, 51.1 mmol). The suspension was allowed to stir at 25° C. for 16 hr and then filtered. The solid was dried in vacuo to give (3aS,7aS)-3a-(3,4-dimethoxyphenyl)-1-methyl-2,3,4,5,7,7a-hexahydroindol-6-one as a 1:1 complex with (2S,3S)-2,3-bis[(4-methylbenzoyl)oxy] butanedioic acid (25.0 g, 72% de). The salt was triturated 3 times with THF at 25° C. for 16 hr to give (3aS,7aS)-3a-(3,4-dimethoxyphenyl)-1-methyl-2,3,4,5,7,7a-hexahydroindol-6-one as a 1:1 complex with (2S,3S)-2,3-bis[(4-methylbenzoyl)oxy]butanedioic acid (22 g, 95% de) as a white solid.

The 1:1 complex of (3aS,7aS)-3a-(3,4-dimethoxyphenyl)-1-methyl-2,3,4,5,7,7a-hexahydroindol-6-one with (2S,3S)-2,3-bis[(4-methylbenzoyl)oxy]butanedioic acid (22 g) was added to a saturated sodium bicarbonate solution (500 mL). The mixture was extracted with ethyl acetate (500 mL). The organic solutions were combined, dried over sodium sulfate, and filtered. The filtrate was concentrated in vacuo to give (3aS,7aS)-3a-(3,4-dimethoxyphenyl)-1-methyl-2,3,4,5,7,7a-hexahydroindol-6-one (001) (7.50 g, 95% ee) as a yellow gum. LC-MS (ESI⁺) m/z 290.6 (M+H)⁺. ¹H NMR (400 MHz, CDCl₃) δ 6.89-6.81 (m, 2H), 6.80-6.75 (m, 1H), 3.82 (d, J=8.0 Hz, 6H), 3.11-3.03 (m, 1H), 2.88 (t, J=3.6 Hz, 1H), 2.59-2.48 (m, 2H), 2.43-2.32 (m, 1H), 2.31-2.21 (m, 4H), 2.20-2.09 (m, 3H), 2.08 (br s, 2H).

### Example 3: Synthesis of [(3aS,7aS)-3a-(3,4-dimethoxyphenyl)-1-methyl-3,4,5,7a-tetrahy dro-2H-indol-6-yl]dibenzyl phosphate (319)

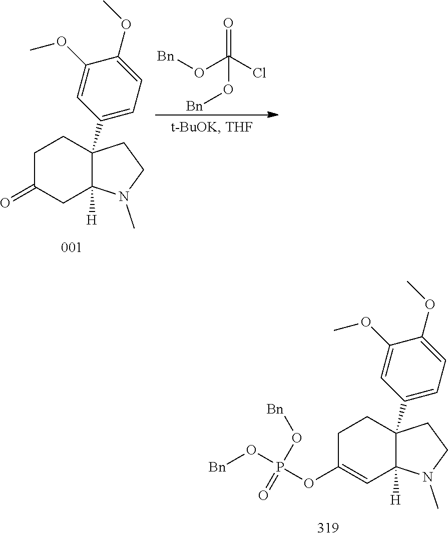

To a solution of (3aS,7aS)-3a-(3,4-dimethoxyphenyl)-1-methyl-2,3,4,5,7,7a-hexahydroindol-6-one (100 mg, 345 umol) in THF (2 mL) was added t-BuOK (1 M in THF, 691 uL). To this mixture was added [benzyloxy(chloro)phosphoryl]oxymethylbenzene (410 mg, 1.38 mmol). The mixture was allowed to stir at 25° C. for 2 hr and then filtered. The filtrate was concentra ted in vacuo and purified by HPLC (column: Phenomenex luna C18 150*25 mm*10 um; mob ile phase: [water(FA)-ACN]; B %: 20%-50%, 8 min) to give [(3aS,7aS)-3a-(3,4-dimethoxyphen yl)-1-methyl-3,4,5,7a-tetrahydro-2H-indol-6-yl] dibenzyl phosphate 319 (24.6 mg 16%) as a white solid. LC-MS (ESI⁺) m/z 550.0 (M+H). ¹H NMR (400 MHz, CDCl₃) δ=7.42-7.28 (m, 10H), 6.82-6.63 (m, 3H), 5.85-5.77 (m, 1H), 5.14-4.98 (m, 4H), 3.84 (d, J=13.4 Hz, 6H), 3.26-3.10 (m, 1H), 3.22-3.05 (m, 3H), 2.99-2.88 (s, 1H), 2.75-2.49 (m, 1H), 2.49-2.2 5 (m, 2H), 2.25-1.73 (m, 4H).

### Example 4: Synthesis of [(3aS,7aS)-3a-(3,4-dimethoxyphenyl)-1-methyl-3,4,5,7a-tetrahy dro-2H-indol-6-yl]diphenyl phosphate (320)

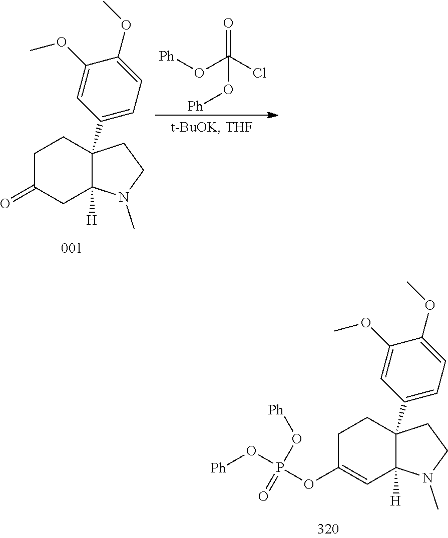

To a solution of (3aS,7aS)-3a-(3,4-dimethoxyphenyl)-1-methyl-2,3,4,5,7,7a-hexahydroindol-6-one (100 mg, 345 umol) in THF (1.0 mL) was added t-BuOK (1 M in THF, 691 uL) and [chloro(phenoxy)phosphoryloxy]benzene (142 uL, 691 umol) at 0° C. The mixture was allowed to stir at 25° C. for 2 hr. The reaction mixture was concentrated in vacuo and the residue was purified by HPLC (column: Waters xbridge 150*25 mm 10 um; mobile phase: [water(NH4HCO3)-ACN]; B %: 44%-74%, 8 min) to give [(3aS,7aS)-3a-(3,4-dimethoxyphenyl)-1-methyl-3,4,5,7a-tetrahydro-2H-indol-6-yl]diphenyl phosphate 320 (60.4 mg, 57%) as a white gum. LC-MS (ESI⁺) m/z 522.2 (M+H)⁺. ¹H NMR (400 MHz, CDCl₃) δ 7.39-7.29 (m, 4H), 7.26-7.13 (m, 6H), 6.81-6.75 (m, 2H), 6.75-6.71 (m, 1H), 5.94-5.89 (m, 1H), 3.84 (d, J=4.0 Hz, 6H), 3.33-3.12 (m, 1H), 2.98-2.81 (m, 1H), 2.38 (br s, 3H), 2.33-2.20 (m, 2H), 2.19-2.10 (m, 2H), 2.07-1.97 (m, 1H), 1.86-1.73 (m, 2H).

### Example 5: Synthesis of [(3aS,7aS)-3a-(3,4-dimethoxyphenyl)-1-methyl-3,4,5,7a-tetrahydro-2H-indol-6-yl]diisopropyl phosphate (321)

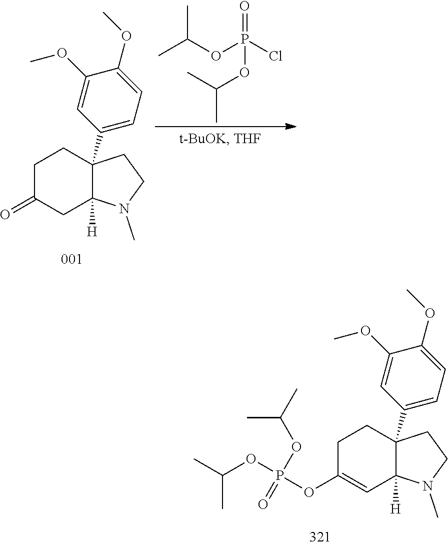

To a mixture of (3aS,7aS)-3a-(3,4-dimethoxyphenyl)-1-methyl-2,3,4,5,7,7a-hexahydroindol-6-one (100 mg, 345 umol), 2-[chloro(isopropoxy)phosphoryl]oxypropane (139 mg, 691 umol) in THE (2 mL) was added t-BuOK (1 M in THF, 691 uL). The reaction mixture was degassed and purged with N₂ 3 times, then the mixture was allowed to stir and warm from 0-25° C. over a period of 1 hr under an atmosphere of N₂. The reaction mixture was concentrated in vacuo and the residue was purified by HPLC (column: Phenomenex luna C18 150*25 mm*10 um; mobile phase: [water(FA)-ACN]; B %: 11%-41%, min) to give 321 (90 mg, 54%) as white gum. LC-MS (ESI+) m/z 454.4 (M+H)⁺. ¹H NMR (400 MHz, CDCl₃) δ 6.89-6.62 (m, 3H), 5.89-5.69 (m, 1H), 4.77-4.58 (m, 2H), 3.87 (d, J=12.0 Hz, 6H), 3.62-3.19 (m, 3H), 2.58 (s, 3H), 2.39-2.22 (m, 3H), 2.08 (dd, J=4.0, 16.0 Hz, 1H), 1.87-1.71 (m, 2H), 1.39-1.23 (m, 12H)

### Example 6: Synthesis of 2-[[(3aS,7aS)-3a-(3,4-dimethoxyphenyl)-1-methyl-3,4,5,7a-tetrahydro-2H-indol-6-yl]oxy]-5,5-dimethyl-1,3,2λ⁵-dioxaphosphinane 2-oxide (323)

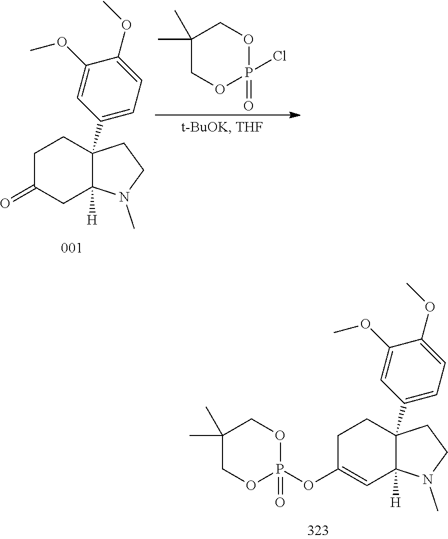

To a solution of (3aS,7aS)-3a-(3,4-dimethoxyphenyl)-1-methyl-2,3,4,5,7,7a-hexahydroindol-6-one (100 mg, 345 umol) in THF (3 mL) was added t-BuOK (38.8 mg, 345 umol) and 2-chloro-5,5-dimethyl-1,3,2λ⁵-dioxaphosphinane 2-oxide (127 mg, 691 umol). The reaction mixture was allowed to stir at 0° C. for 16 hr and then filtered. The filtrate was concentrated in vacuo. The residue was purified by neutral HPLC (column: Welch Ultimate XB—NH2 250*50*10 um; mobile phase: [Heptane-EtOH]; B %: 0%-30%, 15 min). to give 2-[[(3aS,7aS)-3a-(3,4-dimethoxyphenyl)-1-methyl-3,4,5,7a-tetrahydro-2H-indol-6-yl]oxy]-5,5-dimethyl-1,3,2λ⁵-dioxaphosphinane 2-oxide 323 (29.3 mg, 19%) as a white solid. LC-MS (ESI⁺) m/z 438.3 (M+H). ¹H NMR (400 MHz, CDCl₃) δ=6.86-6.74 (m, 3H), 5.90 (br d, J=4.1 Hz, 1H), 4.23-4.15 (m, 1H), 4.07 (br d, J=10.8 Hz, 1H), 3.99-3.83 (m, 10H), 2.65-2.44 (m, 3H), 2.27-2.03 (m, 3H), 1.84-1.68 (m, 4H), 1.30 (s, 3H), 0.86 (s, 3H).

### Example 7: Synthesis of [(3aS,7aS)-3a-(3,4-dimethoxyphenyl)-1-methyl-3,4,5,7a-tetrahydro-2H-indol-6-yl] 3-methylbutanoate (408)

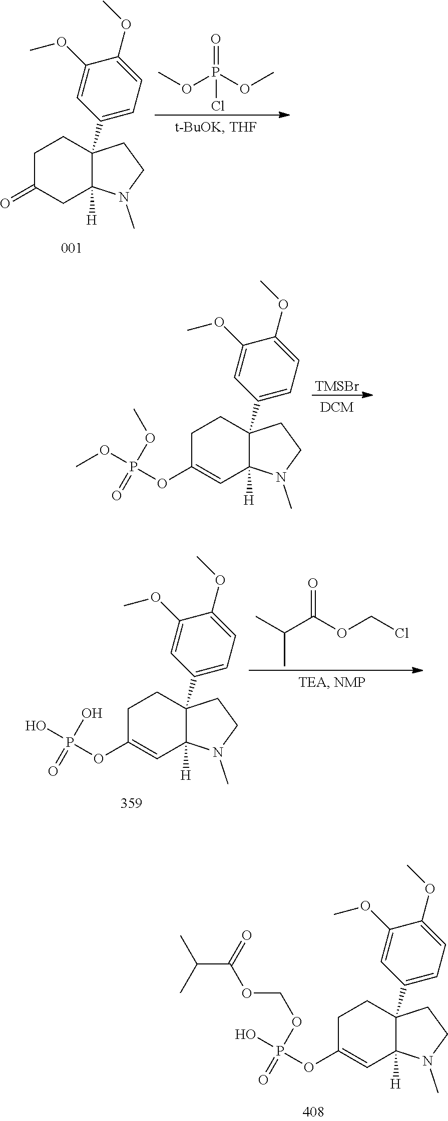

### Step 1: Synthesis of [(3aS,7aS)-3a-(3,4-dimethoxyphenyl)-1-methyl-3,4,5,7a-tetrahydro-2H-indol-6-yl]3-methylbutanoate (int.3)

To a solution of (3aS,7aS)-3a-(3,4-dimethoxyphenyl)-1-methyl-2,3,4,5,7,7a-hexahydroindol-6-one (300 mg, 1.04 mmol) in THE (1 mL) was added t-BuOK (1 M in THF, 2.07 mL). To this mixture was added [chloro(methoxy)phosphoryl]oxymethane (300 mg, 2.07 mmol, 224 uL) The reaction mixture was allowed to stir at 0° C. for 2 hr and then filtered. The filtrate was concentrated in vacuo to give the [(3aS,7aS)-3a-(3,4-dimethoxyphenyl)-1-methyl-3,4,5,7a-tetrahydro-2H-indol-6-yl] dimethyl phosphate (400 mg, 68%) as a yellow oil. LC-MS (ESI+) m/z 397.8 (M+H)+.

### Step 2: Synthesis of [(3aS,7aS)-3a-(3,4-dimethoxyphenyl)-1-methyl-3,4,5,7a-tetrahydro-2H-indol-6-yl]dihydrogen phosphate (359)

To a solution of [(3aS,7aS)-3a-(3,4-dimethoxyphenyl)-1-methyl-3,4,5,7a-tetrahydro-2H-indol-6-yl] dimethyl phosphate (400 mg, 1.01 mmol) in DCM (4 mL) was added TMSBr (462 mg, 3.02 mmol, 391 uL). The reaction mixture was allowed to stir at 25° C. for 16 hr and then filtered. The filtrate was concentrated in vacuo and the residue was purified by prep-HPLC (column: Waters xbridge 150*25 mm 10 um; mobile phase: [water(NH₄HCO₃)-ACN]; B %: 1%-18%, 8 min) to give [(3aS,7aS)-3a-(3,4-dimethoxyphenyl)-1-methyl-3,4,5,7a-tetrahydro-2H-indol-6-yl] dihydrogen phosphate 359 (60 mg, 15%) as white solid. LC-MS (ESI+) m/z 370.2 (M+H)+.

### Step 3: Synthesis of [(3aS,7aS)-3a-(3,4-dimethoxyphenyl)-1-methyl-3,4,5,7a-tetrahydro-2H-indol-6-yl]3-methylbutanoate (408)

To a solution of [(3aS,7aS)-3a-(3,4-dimethoxyphenyl)-1-methyl-3,4,5,7a-tetrahydro-2H-indol-6-yl] dihydrogen phosphate (60 mg, 162 umol) in NMP (2 mL) was added TEA (65.8 mg, 650 umol, 90.4 uL) and chloromethyl 2-methylpropanoate (89 mg, 650 umol). The reaction mixture was allowed to stir at 60° C. for 16 hr and then filtered. The filtrate was concentrated in vacuo to give the residue. The residue was purified by prep-HPLC (column: Waters xbridge 150*25 mm 10 um; mobile phase: [water(NH₄HCO₃)-ACN]; B %: 10%-40%, 8 min) to give the [(3aS,7aS)-3a-(3,4-dimethoxyphenyl)-1-methyl-3,4,5,7a-tetrahydro-2H-indol-6-yl] dihydrogen phosphate [[(3aS,7aS)-3a-(3,4-dimethoxyphenyl)-1-methyl-3,4,5,7a-tetrahydro-2H-indol-6-yl]oxy-hydroxy-phosphoryl]oxymethyl 2-methylpropanoate 408 (9.33 mg, 12%) as an off-white solid. LC-MS (ESI+) m/z 469.9 (M+H)+. ¹H NMR (400 MHz, CDCl₃) δ=12.53-11.70 (m, 1H), 6.87-6.63 (m, 3H), 5.91 (d, J=4.4 Hz, 1H), 5.74-5.46 (m, 2H), 4.17-4.03 (m, 1H), 3.88 (d, J=4.0 Hz, 6H), 3.75 (d, J=2.8 Hz, 1H), 2.95 (s, 4H), 2.62-2.23 (m, 4H), 2.10 (d, J=4.0 Hz, 1H), 1.79 (s, 2H), 1.26 (s, 1H), 1.13 (d, J=7.2 Hz, 5H).

### Example 8: Synthesis of [[(3aS,7aS)-3a-(3,4-dimethoxyphenyl)-1-methyl-3,4,5,7a-tetrahydro-2H-indol-6-yl]oxy-hydroxy-phosphoryl]oxymethyl 2,2-dimethylpropanoate (409)

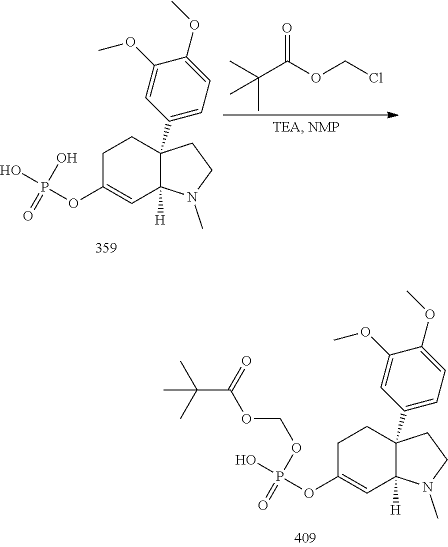

To a solution of [(3aS,7aS)-3a-(3,4-dimethoxyphenyl)-1-methyl-3,4,5,7a-tetrahydro-2H-indol-6-yl] dihydrogen phosphate 359 (200 mg, 541 umol) and chloromethyl 2,2-dimethylpropanoate (407 mg, 2.71 mmol, 392 uL) in NMP (3.0 mL) was added TEA (164 mg, 1.62 mmol, 226 uL). The mixture was allowed to stir at 60° C. for 16 hr. The reaction mixture was concentrated in vacuo and the residue was purified by prep-HPLC (FA condition: column: Phenomenex luna C18 150*25 mm*10 um; mobile phase: [water(FA)-ACN]; B %: 15%-45%, 8 min) to give [[(3aS,7aS)-3a-(3,4-dimethoxyphenyl)-1-methyl-3,4,5,7a-tetrahydro-2H-indol-6-yl]oxy-hydroxy-phosphoryl]oxymethyl 2,2-dimethylpropanoate 409 (17.48 mg, 6%) as a pink solid. LC-MS (ESI⁺) m/z 484.1 (M+H)⁺. ¹H NMR (400 MHz, CDCl₃) δ 12.10-11.72 (m, 1H), 6.87-6.80 (m, 1H), 6.80-6.70 (m, 2H), 5.91 (d, J=4.8 Hz, 1H), 5.69-5.54 (m, 2H), 4.18-4.05 (m, 1H), 3.89 (d, J=2.8 Hz, 6H), 3.77 (d, J=3.2 Hz, 1H), 2.97 (s, 4H), 2.65-2.34 (m, 3H), 2.13 (d, J=3.6 Hz, 1H), 1.89-1.69 (m, 2H), 1.26-1.12 (m, 9H).

### Example 9: Synthesis of [(3aS,7aS)-3a-(3,4-dimethoxyphenyl)-1-methyl-3,4,5,7a-tetrahydro-2H-indol-6-yl] N,N-dimethylcarbamate (324)

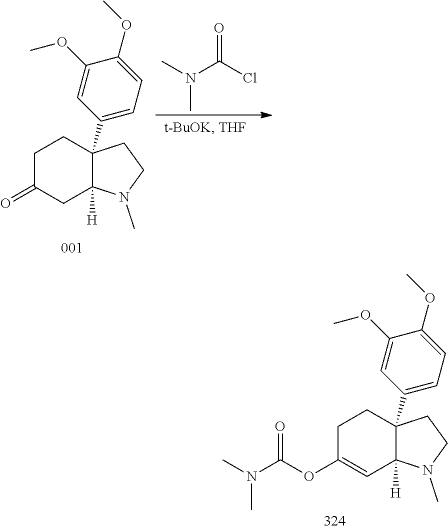

To a solution of (3aS,7aS)-3a-(3,4-dimethoxyphenyl)-1-methyl-2,3,4,5,7,7a-hexahydroindol-6-one (50.0 mg, 172 umol, 001) and N,N-dimethylcarbamoyl chloride (27.8 mg, 259 umol) in THF (2 mL) was added potassium 2-methylpropan-2-olate (100 mg, 864 umol), the mixture was allowed to stir at 0° C. for 2 hr and then filtered. The filtrate was concentrated and the residue was purified by reversed-phase HPLC (column: Phenomenex Luna C18 150*25 mm*10 um; mobile phase: [water(FA)-ACN]B %: 7%-37%, 58 min) to give [(3aS, 7aS)-3a-(3,4-dimethoxyphenyl)-1-methyl-3,4,5,7a-tetrahydro-2H-indol-6-yl]N,N-dimethylcarbamate 324 (21.2 mg, 99%) as a white gum. LC-MS (ESI⁺) m/z 361.1 (M+H)⁺. ¹H NMR (400 MHz, CDCl₃) δ 6.83 (s, 3H), 5.74 (br d, J=3.6 Hz, 1H), 3.88 (d, J=12.8 Hz, 6H), 3.72-3.48 (m, 2H), 2.94 (d, J=12.4 Hz, 6H), 2.85-2.74 (m, 1H), 2.67 (s, 3H), 2.40-2.37 (m, 3H), 2.06-1.97 (m, 1H), 1.95-1.86 (m, 1H), 1.85-1.77 (m, 1H).

### Example 10: Synthesis of [(3aS,7aS)-3a-(3,4-dimethoxyphenyl)-1-methyl-3,4,5,7a-tetrahydro-2H-indol-6-yl] N-ethyl-N-methyl-carbamate (326)

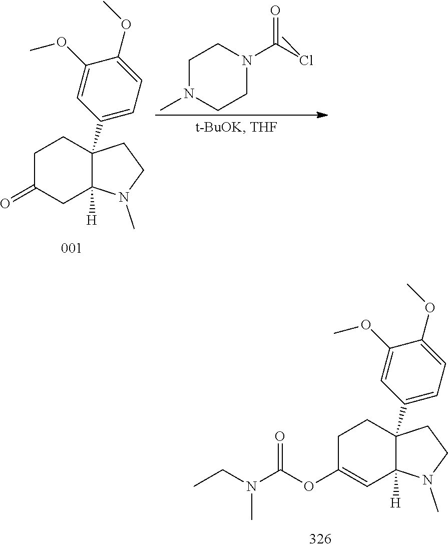

To a solution of (3aS,7aS)-3a-(3,4-dimethoxyphenyl)-1-methyl-2,3,4,5,7,7a-hexahydroindol-6-one 001 (150 mg, 518 umol) in THE (1 mL) was added t-BuOK (1 M in THF, 1.04 mL) at 0° C. and then the reaction mixture was allowed to stir for 30 min. To this mixture was added N-ethyl-N-methyl-carbamoyl chloride (126 mg, 1.04 mmol). The reaction mixture was allowed to stir at 25° C. for 16 hr and then filtered. The filtrate was concentrated in vacuo and the residue was purified by prep-HPLC (column: Waters xbridge 150*25 mm 10 um; mobile phase: [water(NH₄HCO₃)-ACN]; B %: 22%-52%, 11 min) to give [(3aS,7aS)-3a-(3,4-dimethoxyphenyl)-1-methyl-3,4,5,7a-tetrahydro-2H-indol-6-yl] N-ethyl-N-methyl-carbamate 326 (46.0 mg, 30%) as a white gum. LC-MS (ESI⁺) m/z 375.1 (M+H). ¹H NMR (400 MHz, CDCl₃) δ=6.9 7-6.85 (m, 2H), 6.85-6.77 (m, 1H), 5.71 (d, J=4.0 Hz, 1H), 3.88 (d, J=16.0 Hz, 6H), 3.4 3-3.26 (m, 2H), 3.25-3.15 (m, 1H), 3.01-2.83 (m, 4H), 2.39 (s, 3H), 2.34-2.09 (m, 4H), 1.93-1.84 (m, 2H), 1.76-1.69 (m, 1H), 1.12 (t, J=7.2 Hz, 3H).

### Example 11: Synthesis of [(3aS,7aS)-3a-(3,4-dimethoxyphenyl)-1-methyl-3,4,5,7a-tetrahydro-2H-indol-6-yl]4-methylpiperazine-1-carboxylate (327)

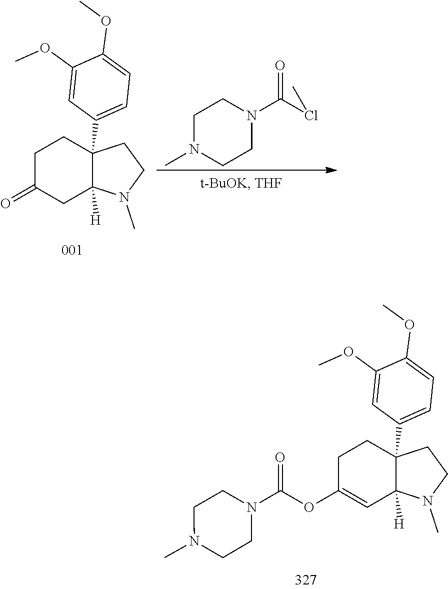

To a solution of (3aS,7aS)-3a-(3,4-dimethoxyphenyl)-1-methyl-2,3,4,5,7,7a-hexahydroindol-6-one (100 mg, 345 umol) in THF (1.0 mL) was added t-BuOK (1 M in THF, 1.38 mL) and 4-methylpiperazine-1-carbonyl chloride hydrochloride (137 mg, 691 umol) at 0° C. The reaction mixture was allowed to stir at 25° C. for 2 hr and then concentrated in vacuo. The resulting residue was purified by prep-HPLC (column: Welch Xtimate C18 150*25 mm*5 um; mobile phase: [water (ammonia hydroxide v/v)-ACN]; B %: 23%-53%, 2 min) to give the [(3aS,7aS)-3a-(3,4-dimethoxyphenyl)-1-methyl-3,4,5,7a-tetrahydro-2H-indol-6-yl]4-methylpiperazine-1-carboxylate 327 (31.5 mg, 32%) as a white gum. LC-MS (ESI⁺) m/z 416.4 (M+H)⁺.

¹H NMR (400 MHz, CDCl₃) δ 6.90-6.85 (m, 2H), 6.84-6.80 (m, 1H), 5.74-5.69 (m, 1H), 3.88 (d, J=15.4 Hz, 6H), 3.51 (br s, 4H), 3.21 (br t, J=7.8 Hz, 1H), 2.87 (br s, 1H), 2.39 (s, 7H), 2.34-2.26 (m, 4H), 2.26-2.18 (m, 2H), 2.17-2.09 (m, 1H), 1.93-1.82 (m, 2H), 1.73 (br d, J=3.6 Hz, 1H).

### Example 12: Synthesis of [(3aS,7aS)-3a-(3,4-dimethoxyphenyl)-1-methyl-3,4,5,7a-tetrahydro-2H-indol-6-yl]N-methyl-N-phenyl-carbamate (331)

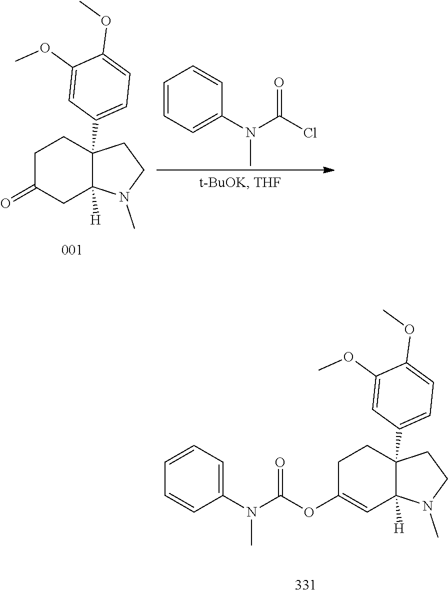

A mixture of (3aS,7aS)-3a-(3,4-dimethoxyphenyl)-1-methyl-2,3,4,5,7,7a-hexahydroindol-6-one (100 mg, 346 umol), N-methyl-N-phenyl-carbamoyl chloride (293 mg, 1.73 mmol) and t-BuOK (1 M in THF, 691 uL) in THF (2.00 mL) was degassed and purged with N₂ 3 times, and then the mixture was allowed to stir at 25° C. for 1 hr under an atmosphere of N₂. The reaction mixture was concentrated in vacuo and the residue was purified by prep-HPLC (neutral condition: column: Waters xbridge 150*25 mm 10 um; mobile phase: [water (NH₄HCO₃)-ACN]; B %: 30%-60%, 11 min) to give [(3aS,7aS)-3a-(3,4-dimethoxyphenyl)-1-methyl-3,4,5,7a-tetrahydro-2H-indol-6-yl]N-methyl-N-phenyl-carbamate 331 (17.3 mg, 6.7%) as yellow gum. LC-MS (ESI+) m/z 423.3 (M+H)+. ¹H NMR (400 MHz, CDCl₃) δ 7.39-7.30 (m, 2H), 7.27-7.18 (m, 3H), 6.91-6.76 (m, 3H), 5.73 (s, 1H), 3.87 (d, J=11.2 Hz, 6H), 3.32 (s, 3H), 3.24-3.14 (m, 1H), 2.87 (s, 1H), 2.38 (s, 3H), 2.31-2.11 (m, 4H), 1.92-1.82 (m, 2H), 1.74 (s, 1H).

### Example 13: Synthesis of N-[[(3aS,7aS)-3a-(3,4-dimethoxyphenyl)-1-methyl-3,4,5,7a-tetrahydro-2H-indol-6-yl]tert-butylcarbonate (358)

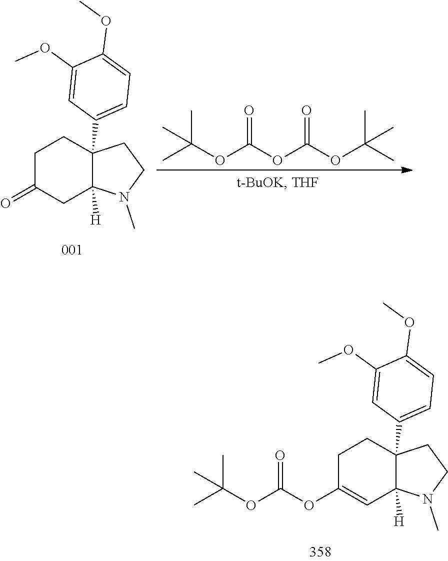

To a solution of (3aS,7aS)-3a-(3,4-dimethoxyphenyl)-1-methyl-2,3,4,5,7,7a-hexahydroindol-6-one (100 mg, 346 umol) tert-butoxycarbonyl tert-butyl carbonate (75.4 mg, 346 umol) in THE (2 mL) was added t-BuOK (1 M, 691 uL). The mixture was allowed to stir at 0-25° C. for 1 hr. The reaction mixture was concentrated in vacuo and the residue was purified by prep-HPLC (column: Phenomenex luna C18 150*25 mm*10 um; mobile phase: [water (FA)-ACN]; B %: 11%-41%, 10 min) to give [(3aS,7aS)-3a-(3,4-dimethoxyphenyl)-1-methyl-3,4,5,7a-tetrahydro-2H-indol-6-yl] tert-butyl carbonate 358 (38 mg, 27%) as a yellow gum. LC-MS (ESI⁺) m/z 431.9 (M+H). ¹H NMR (400 MHz, CDCl₃) δ 6.95-6.73 (m, 3H), 5.91-5.82 (m, 1H), 3.88 (d, J=6 Hz, 6H), 3.71-3.48 (m, 2H), 2.93-2.77 (m, 1H), 2.71-2.61 (m, 3H), 2.42-2.34 (m, 2H), 2.29-2.21 (m, 1H), 2.11-2.00 (m, 1H), 1.94-1.85 (m, 1H), 1.85-1.77 (m, 1H), 1.49 (s, 8H), 1.37-1.21 (m, 1H).

### Example 14: Synthesis of [[(3aS,7aS)-3a-(3,4-dimethoxyphenyl)-1-methyl-3,4,5,7a-tetrahydro-2H-indol-6-yl]oxy-hydroxy-phosphoryl]oxymethyl isopropyl carbonate (410)

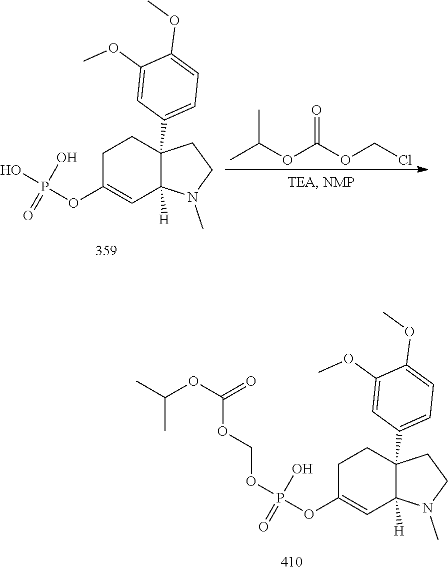

A mixture of [(3aS,7aS)-3a-(3,4-dimethoxyphenyl)-1-methyl-3,4,5,7a-tetrahydro-2H-indol-6-yl] dihydrogen phosphate 359 (300 mg, 812 umol), chloromethyl isopropyl carbonate (619 mg, 4.06 mmol), and TEA (246 mg, 2.44 mmol, 339 uL) in NMP (1 mL) was degassed and purged with nitrogen gas 3 times. The mixture was allowed to stir at 25° C. for 1 hr under an atmosphere of nitrogen and then concentrated in vacuo. The residue was purified by prep-HPLC (column: Waters xbridge 150*25 mm 10 um; mobile phase: [water(NH₄HCO₃)-ACN]; B %: 10%-40%, 10 min) to give [[(3aS,7aS)-3a-(3,4-dimethoxyphenyl)-1-methyl-3,4,5,7a-tetrahydro-2H-indol-6-yl]oxy-hydroxy-phosphoryl]oxymethyl isopropyl carbonate 410 (4.31 mg, 14%) as a white oil. LC-MS (ESI⁺) m/z 486.18 (M+H)⁺. ¹H NMR (400 MHz, CDCl₃) δ=12.11-11.75 (m, 1H), 6.85-6.80 (m, 1H), 6.77-6.69 (m, 2H), 5.95 (br d, J=4.4 Hz, 1H), 5.60 (d, J=12.4 Hz, 2H), 4.84 (td, J=6.4, 12.4 Hz, 1H), 4.30-3.99 (m, 1H), 3.87 (d, J=3.2 Hz, 6H), 3.77 (s, 1H), 2.98 (s, 3H), 2.93-2.84 (m, 1H), 2.64-2.34 (m, 3H), 2.22-2.02 (m, 1H), 1.90-1.80 (m, 2H), 1.26 (s, 6H).

### Example 15: Synthesis of 2-(((3aS,7aS)-3a-(3,4-dimethoxyphenyl)-1-methyl-2,3,3a, 4,5,7a-hexahydro-1H-indol-6-yl)oxy)-1,3,2-dioxaphosphinane 2-oxide (422)

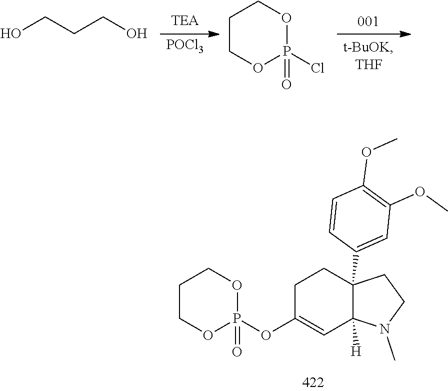

### Step 1: Synthesis of 2-chloro-1,3,2-dioxaphosphinane 2-oxide

To a solution of propane-1,3-diol (500 mg, 6.57 mmol, 476 uL) in DCM (10 mL) was added TEA (1.33 g, 13.1 mmol, 1.83 mL) and POCl₃ (1.01 g, 6.57 mmol, 610 uL) at 0° C. The reaction mixture was allowed to stir at 25° C. for 2 hr and then concentrated in vacuo to give 2-chloro-1,3,2-dioxaphosphinane 2-oxide (1 g, 73%) as a white solid.

### Step 2: Synthesis of 2-(((3aS,7aS)-3a-(3,4-dimethoxyphenyl)-1-methyl-2,3,3a, 4,5,7a-hexahydro-1H-indol-6-yl)oxy)-1,3,2-dioxaphosphinane 2-oxide (422)

To a solution of 001 (150 mg, 518 umol) in THF (1.0 mL) was added t-BuOK (1M in THF, 1.04 mL). The reaction mixture was allowed to stir at 0° C. for 30 min, then 2-chloro-1,3,2-dioxaphosphinane 2-oxide (405 mg, 2.59 mmol) was added at 0° C. The reaction mixture was allowed to stir at 25° C. for 2 hr and then concentrated in vacuo. The residue was purified by prep-HPLC (column: Welch Ultimate XB-Diol 250*50*10 um; mobile phase: [Hexane-EtOH]; B %: 10%-50%, 15 min) to give 422 (23.7 mg, 15%) as a yellow gum. LC-MS (ESI⁺) m/z 410.1 (M+H)⁺. ¹H NMR (400 MHz, CDCl₃) δ 7.09-6.52 (m, 3H), 5.90 (br d, J=4.8 Hz, 1H), 4.55-4.42 (m, 2H), 4.40-4.34 (m, 2H), 3.88 (d, J=8.8 Hz, 6H), 3.67-3.52 (m, 1H), 3.41-3.21 (m, 1H), 2.63 (s, 3H), 2.41-2.25 (m, 3H), 2.18-2.06 (m, 1H), 1.89-1.80 (m, 3H), 1.79-1.73 (m, 2H).

### Example 16: Synthesis of 2-(((3aS,7aS)-3a-(3,4-dimethoxyphenyl)-1-methyl-2,3,3a, 4,5,7a-hexahydro-1H-indol-6-yl)oxy)-4,6-dimethyl-1,3,2-dioxaphosphinane 2-oxide (423)

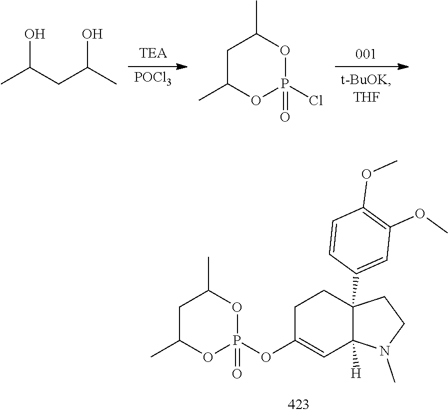

### Step 1: Synthesis of 2-chloro-4,6-dimethyl-1,3,2-dioxaphosphinane 2-oxide

To a solution of pentane-2,4-diol (1 g, 9.60 mmol) and TEA (1.94 g, 19.2 mmol, 2.67 mL) in DCM (6 mL) was added POCl₃ (1.77 g, 11.5 mmol, 1.07 mL) at 0° C. The reaction mixture was allowed to stir at 25° C. for 2 hr under an atmosphere of N₂. The reaction mixture was concentrated in vacuo to give 2-chloro-4,6-dimethyl-1,3,2-dioxaphosphinane 2-oxide (1 g) as a white solid which was used without purification.

### Step 2: Synthesis of 2-(((3aS,7aS)-3a-(3,4-dimethoxyphenyl)-1-methyl-2,3,3a, 4,5,7a-hexahydro-1H-indol-6-yl)oxy)-4,6-dimethyl-1,3,2-dioxaphosphinane 2-oxide (423)

To a solution of (3aS,7aS)-3a-(3,4-dimethoxyphenyl)-1-methyl-2,3,4,5,7,7a-hexahydroindol-6-one 001 (200 mg, 691 umol) in THF (4 mL) was added t-BuOK (1 M in THF, 1.38 mL) at 0° C. over a time period of 30 min. 2-chloro-4,6-dimethyl-1,3,2-dioxaphosphinane 2-oxide (510 mg, 2.76 mmol) in THF (4 mL) was added and the reaction mixture was allowed to stir at 25° C. for 2 hr. The reaction mixture was concentrated in vacuo and the residue was purified by prep-HPLC (column: Welch Ultimate XB—SiOH 250*50*10 um; mobile phase: [EtOH+MeOH (4:1, neutral)]; B %: 20%-70%, 16 min) to give 423 (30 mg, 15%) as a white solid. LC-MS (ESI⁺) m/z 438.4 (M+H)⁺¹H NMR (400 MHz, CDCl₃) δ=6.77-6.65 (m, 3H), 5.79-5.73 (m, 1H), 4.72-4.57 (m, 2H), 3.80 (d, J=11.6 Hz, 6H), 3.53-3.27 (m, 1H), 3.26-3.02 (m, 1H), 2.66-2.36 (m, 4H), 2.29-2.15 (m, 2H), 2.09-1.97 (m, 1H), 1.84-1.68 (m, 5H), 1.33 (br d, J=6.2 Hz, 3H), 1.31-1.27 (m, 3H).

### Example 17: Synthesis of 2-(((3aS,7aS)-3a-(3,4-dimethoxyphenyl)-1-methyl-2,3,3a, 4,5,7a-hexahydro-1H-indol-6-yl)oxy)-4,4,6,6-tetramethyl-1,3,2-dioxaphosphinane 2-oxide (435)

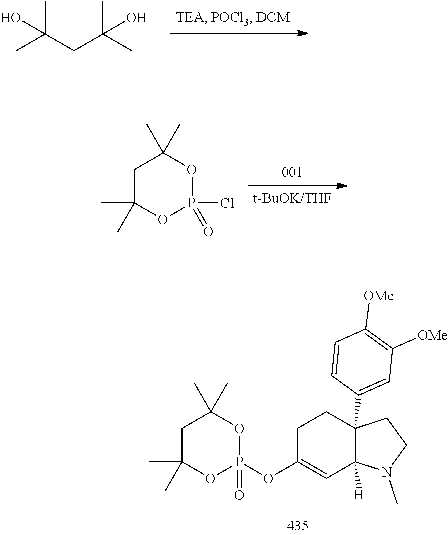

### Step 1-Synthesis of 2-chloro-4,4,6,6-tetramethyl-1,3,2dioxaphosphinane2-oxide

To a solution of 2,4-dimethylpentane-2,4-diol (500 mg, 3.78 mmol) in DCM (10 mL) was added TEA (765 mg, 7.56 mmol, 1.05 mL). Then the mixture was added dropwise POCl₃ (579 mg, 3.78 mmol, 352 μL) over 10 minutes at 0° C. The mixture was stirred at 0° C. for 1 hour. On completion, The reaction mixture was concentrated in vacuo to give 2-chloro-4,4,6,6-tetramethyl-1,3,2 dioxaphosphinane 2-oxide (550 mg, 34% yield) as a brown gum.

### Step 2-Synthesis of 2-[[(3aS,7aS)-3a-(3,4-dimethoxyphenyl)-1-methyl-3,4,5,7a-tetrahydro-2H-indol-6-yl]oxy]-4,4,6,6-tetramethyl-1,3,2dioxaphosphinane2-oxide2-oxide (435)

To a solution of (3aS,7aS)-3a-(3,4-dimethoxyphenyl)-1-methyl-2,3,4,5,7,7a-hexahydroindol-6-one (250 mg, 863 μmol) in THF (1.0 mL) was added t-BuOK (1 M, 1.73 mL) was stirred at 0° C. for 10 minutes. Then 2-chloro-4,4,6,6-tetramethyl-1,3,2dioxaphosphinane 2-oxide (459 mg, 2.16 mmol) was added in the mixture. The mixture was stirred at 25° C. for 2 hours. On completion, the reaction mixture was concentrated in vacuo to give the residue. The residue was purified by prep-HPLC (column: Agela DuraShell C18 150*25 mm*5 um; mobile phase: [water(NH₃H₂O)-ACN]; B %: 32%-62%, min) to give 2-[[(3aS,7aS)-3a-(3,4-dimethoxyphenyl)-1-methyl-3,4,5,7a-tetrahydro-2H-indol-6-yl]oxy]-4,4,6,6-tetramethyl-1,3,2dioxaphosphinane 2-oxide (38.4 mg, 9% yield) as a red gum.

LC-MS (ESI⁺) m/z 466.4 (M+H)⁺;

¹H NMR (400 MHz, CDCl₃) δ 6.82-6.65 (m, 3H), 5.87-5.73 (m, 1H), 3.91-3.71 (m, 6H), 3.17-3.04 (m, 1H), 2.79 (s, 1H), 2.32 (s, 3H), 2.08 (s, 4H), 2.02-1.86 (m, 3H), 1.74 (s, 2H), 1.49 (d, J=5.2 Hz, 6H), 1.43 (s, 3H), 1.36 (s, 3H).

### Example 18: Synthesis of 2-(((3aS,7aS)-3a-(3,4-dimethoxyphenyl)-1-methyl-2,3,3a, 4,5,7a-hexahydro-1H-indol-6-yl)oxy)-1,3,2-dioxaphosphepane 2-oxide (450)

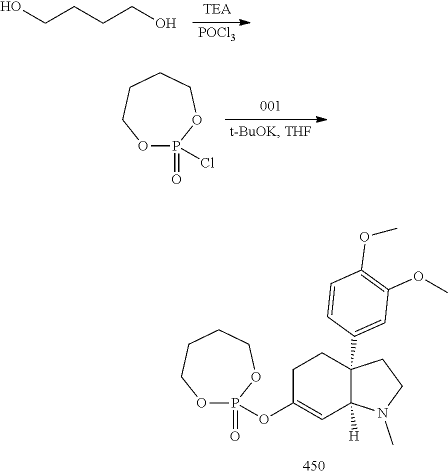

2-chloro-1,3,2-dioxaphosphepane 2-oxide, which could be prepared by reaction of butane-1,4-diol with POCl₃ and a base such as TEA in a solvent, such as DCM, could be reacted with 001 in the presence of a base, such as t-BuOK, in a solvent, such as THF, to give 450.

### Example 19: Synthesis of 2-(((3aS,7aS)-3a-(3,4-dimethoxyphenyl)-1-methyl-2,3,3a, 4,5,7a-hexahydro-1H-indol-6-yl)oxy)-4,5-dimethyl-1,3,2-dioxaphospholane 2-oxide (424)

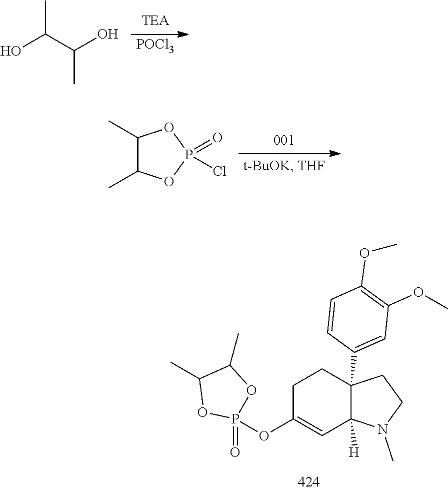

2-chloro-4,5-dimethyl-1,3,2-dioxaphospholane 2-oxide, which could be prepared by reaction of butane-2,3-diol with POCl₃ and a base such as TEA in a solvent, such as DCM, could be reacted with 001 in the presence of a base, such as t-BuOK, in a solvent, such as THF, to give 424.

### Example 20: Synthesis of 2-(((3aS,7aS)-3a-(3,4-dimethoxyphenyl)-1-methyl-2,3,3a, 4,5,7a-hexahydro-1H-indol-6-yl)oxy)-1,3,2-dioxaphosphocane 2-oxide (451)

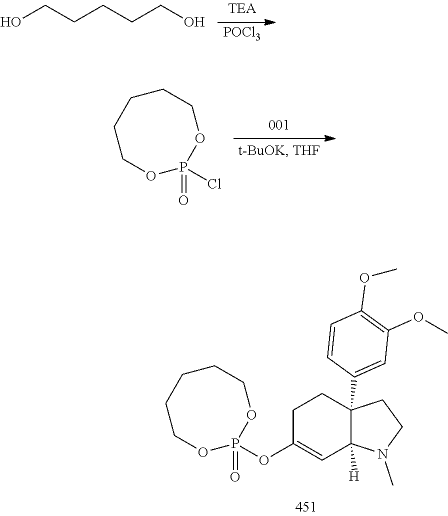

2-chloro-1,3,2-dioxaphosphocane 2-oxide, which could be prepared by reaction of pentane-1,5-diol with POCl₃ and a base such as TEA in a solvent such as DCM, could be reacted with 001 in the presence of a base such as t-BuOK in a solvent such as THF to give 451.

### Example 21: Synthesis of 6-(((3aS,7aS)-3a-(3,4-dimethoxyphenyl)-1-methyl-2,3,3a, 4,5,7a-hexahydro-1H-indol-6-yl)oxy)-5,7-dioxa-6-phosphaspiro[2.5]octane 6-oxide (453)

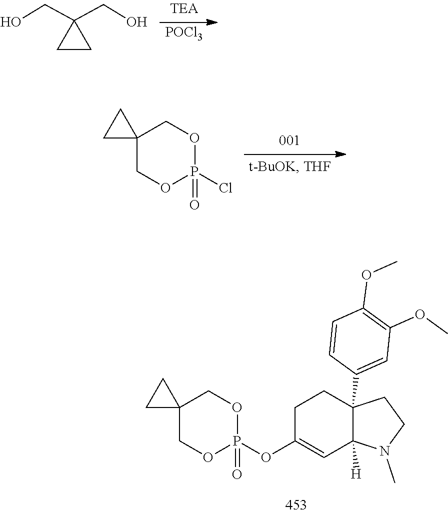

### Step 1: Synthesis of 6-chloro-5,7-dioxa-6-phosphaspiro[2.5]octane 6-oxide

To a solution of [1-(hydroxymethyl)cyclopropyl]methanol (200 mg, 1.96 mmol) in DCM (4 mL) was added POCl₃ (360 mg, 2.35 mmol, 218 uL) and TEA (476 mg, 4.70 mmol, 654 uL). The mixture was stirred at 25° C. for 2 hours. On completion, the reaction mixture was filtered, concentrated in vacuo to give 6-chloro-5,7-dioxa-6-phosphaspiro[2.5]octane 6-oxide (280 mg, 63%) as a yellow solid.

### Step 2: Synthesis of 6-(((3aS,7aS)-3a-(3,4-dimethoxyphenyl)-1-methyl-2,3,3a, 4,5,7a-hexahydro-1H-indol-6-yl)oxy)-5,7-dioxa-6-phosphaspiro[2.5]octane 6-oxide (453)

To a solution of 001 (200 mg, 691 umol) in THF (4 mL) was added t-BuOK (1M in THF, 1.38 mL) at 0° C. over 10 min. The mixture was added 6-chloro-5,7-dioxa-6-phosphaspiro[2.5]octane 6-oxide (252 mg, 1.38 mmol). The reaction mixture was allowed to stir at 25° C. for 2 hr and then filtered. The eluent was concentrated in vacuo and the residue was purified by prep-HPLC (column: Phenomenex luna C₁₈ 150*25 mm*10 um; mobile phase: [water(FA)-ACN]; B %: 3%-33%, 8 min) to 453 (55.0 mg, 18%) as a yellow gum. LC-MS (ESI+) m/z 436.1 (M+H)+. ¹H NMR (400 MHz, CDCl₃) δ=6.94-6.68 (m, 3H), 5.93 (d, J=2.8 Hz, 1H), 4.73 (d, J=11.2 Hz, 1H), 4.63 (d, J=11.2 Hz, 1H), 3.88 (d, J=8.8 Hz, 6H), 3.83-3.73 (m, 1H), 3.71-3.48 (m, 3H), 2.79-2.66 (m, 4H), 2.41-2.34 (m, 3H), 2.18 (d, J=17.6 Hz, 1H), 1.99-1.72 (m, 2H), 1.00-0.81 (m, 2H), 0.77-0.49 (m, 2H).

### Example 22: Synthesis of 7-(((3aS,7aS)-3a-(3,4-dimethoxyphenyl)-1-methyl-2,3,3a, 4,5,7a-hexahydro-1H-indol-6-yl)oxy)-6,8-dioxa-7-phosphaspiro[3.5]nonane 7-oxide (454)

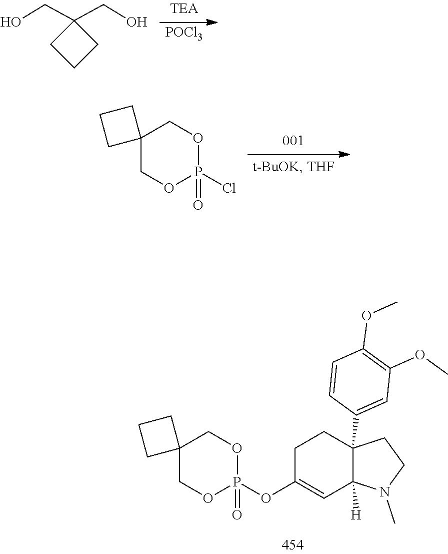

### Step 1: Synthesis of 7-chloro-6,8-dioxa-7-phosphaspiro[3.5]nonane 7-oxide

To a solution of [1-(hydroxymethyl)cyclobutyl]methanol (200 mg, 1.72 mmol) in DCM (4 mL) was added POCl₃ (317 mg, 2.07 mmol, 192 uL) and TEA (418 mg, 4.13 mmol, 575 uL). The reaction mixture was allowed to stir at 25° C. for 0.5 hr and then was filtered. The eluent was concentrated in vacuo to give 7-chloro-6,8-dioxa-7-phosphaspiro[3.5]nonane 7-oxide (300 mg, 71%) as a yellow liquid.

### Step 2: Synthesis of 7-(((3aS,7aS)-3a-(3,4-dimethoxyphenyl)-1-methyl-2,3,3a, 4,5,7a-hexahydro-1H-indol-6-yl)oxy)-6,8-dioxa-7-phosphaspiro[3.5]nonane 7-oxide (454)

To a solution of 001 (200 mg, 691 umol) in THF (4 mL) was added t-BuOK (1M in THF, 1.38 mL) at 0° C. over 30 minutes. To this mixture was then added 7-chloro-6,8-dioxa-7-phosphaspiro[3.5]nonane 7-oxide (272 mg, 1.38 mmol). The reaction mixture was allowed to stir at 25° C. for 2 hr and was then filtered. The eluent was concentrated in vacuo and the resulting residue was purified by prep-HPLC (column: Phenomenex luna C₁₈ 150*25 mm*10 um; mobile phase: [water(FA)-ACN]; B %: 5%-35%, 8 min)) to 454 (42 mg, 15%) as a yellow gum. LC-MS (ESI⁺) m/z 450.1 (M+H)⁺. ¹H NMR (400 MHz, CDCl₃-d) 6=6.96-6.62 (m, 3H), 5.89 (d, J=4.4 Hz, 1H), 4.43-4.11 (m, 4H), 3.87 (d, J=8.0 Hz, 6H), 3.83-3.72 (m, 1H), 3.55 (d, J=2.4 Hz, 1H), 2.90-2.62 (m, 4H), 2.50-2.30 (m, 3H), 2.28-1.95 (m, 5H), 1.94-1.60 (m, 4H).

### Example 23: Synthesis of 3-(((3aS,7aS)-3a-(3,4-dimethoxyphenyl)-1-methyl-2,3,3a, 4,5,7a-hexahydro-1H-indol-6-yl)oxy)-2,4,9-trioxa-3-phosphaspiro[5.5]undecane 3-oxide (455)

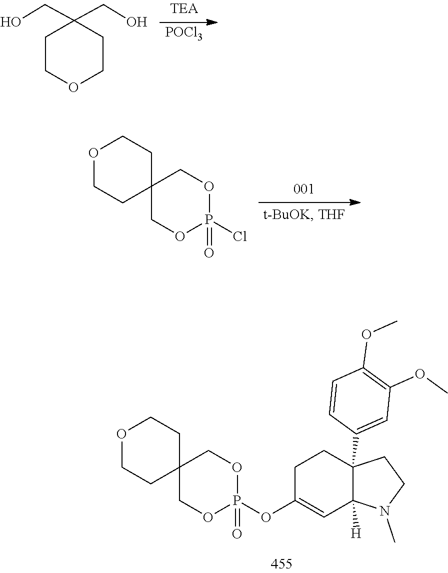

3-chloro-2,4,9-trioxa-3-phosphaspiro[5.5]undecane 3-oxide, which could be prepared by reaction of (tetrahydro-2H-pyran-4,4-diyl)dimethanol with POCl₃ and a base such as TEA in a solvent such as DCM, could be reacted with 001 in the presence of a base such as t-BuOK in a solvent such as THF to give 455.

### Example 24: Synthesis of 2-(((3aS,7aS)-3a-(3,4-dimethoxyphenyl)-1-methyl-2,3,3a, 4,5,7a-hexahydro-1H-indol-6-yl)oxy)-4,4,5,5-tetramethyl-1,3,2-dioxaphospholane 2-oxide (437)

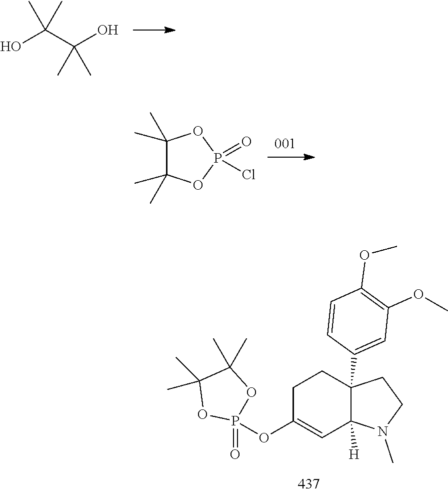

2-chloro-4,4,5,5-tetramethyl-1,3,2-dioxaphospholane 2-oxide, which could be prepared by reaction of 2,3-dimethylbutane-2,3-diol with POCl₃ and a base, could be reacted with 001 in the presence of a base to give 437.

### Example A1: Hydrolysis Assay

### General Method Description of Hydrolysis Assay—Condition 1 (SGF)

50 μL of compound (10 mM) in DMSO was diluted into 950 μL of Simulated Gastric Fluid (SGF) with pepsin (pH 1.5) to a final concentration of 0.5 mM. The hydrolysis kinetics were conducted at 37° C. and measured by LCMS at a certain time interval.

### General Method Description of Hydrolysis Assay—Condition 2 (DI Water)

50 μL of compound (10 mM) in DMSO was diluted into 950 μL of DI water to a final concentration of 0.5 mM. The hydrolysis kinetics were conducted at 25° C. and measured by LCMS at a certain time interval.

The corresponding LCMS chromatogram were recorded and the conversions were calculated by integration of each peak.

### Note:

1) LCMS spectra were obtained using Agilent 1200G1956A or SHIMADZU LCMS-2020. Standard LCMS conditions were as follows (running time 1.55 minutes):
   Acidic condition: Mobile Phase A: 0.0375% TFA in water (v/v). Mobile Phase B: 0.01875% TFA in acetonitrile (v/v); Column: Kinetex EVO C18 30*2.1 mm, 5 μm.
   Basic condition: Mobile Phase A: 0.025% NH₃·H₂O in water (v/v). Mobile Phase B: Acetonitrile; Column: Kinetex EVO C18 2.1×30 mm, 5 μm.
2) Conversion (%)=peak area 1/(peak area 1+peak area 2)*100%, peak area 1 is the integration of hydrolyzed product, peak area 2 is the integration of starting material.

TABLE 1
Carbonates

| | Time | Condition 1 | Condition 2 |
|---|---|---|---|
| Compound | (min) | Conversion (%) | Conversion (%) |
| 358 | 1 | 0 | 0 |
| | 5 | 0 | 0 |
| | 30 | 0 | 0 |

| | | | |
|---|---|---|---|
| 1 | (h) | 5 | 0 |
| 4 | (h) | 100 | 83.46 |
| 24 | (h) | 100 | 100 |

TABLE 2
Phosphate Esters

| | Time | Condition 1 | Condition 2 |
|---|---|---|---|
| Compound | (min) | Conversion (%) | Conversion (%) |
| 321 | 1 | 0 | 0 |
| | 5 | 0 | 0 |
| | 30 | 0 | 0 |

| | | | |
|---|---|---|---|
| 1 | (h) | 0 | 0 |
| 4 | (h) | 0 | 0 |
| 24 | (h) | 0 | 0 |

| | | | |
|---|---|---|---|
| 320 | 1 | 0 | 0 |
| | 5 | 0 | 0 |
| | 30 | 0 | 0 |

| | | | |
|---|---|---|---|
| 1 | (h) | 0 | 0 |
| 4 | (h) | 0 | 0 |
| 24 | (h) | 0 | 0 |

| | | | |
|---|---|---|---|
| 323 | 1 | 0 | 0 |
| | 5 | 0 | 0 |
| | 30 | 0 | 0 |

| | | | |
|---|---|---|---|
| 1 | (h) | 0 | 0 |
| 4 | (h) | 0 | 0 |
| 24 | (h) | 0 | 0 |

| | | | |
|---|---|---|---|
| 319 | 1 | 2 | 2 |
| | 5 | 5 | 4 |
| | 30 | 25 | 5 |

| | | | |
|---|---|---|---|
| 1 | (h) | 26 | 10 |
| 4 | (h) | 60 | 20 |
| 24 | (h) | 61 | 59 |

TABLE 3
Carbamates

| | Time | Condition 1 | Condition 2 |
|---|---|---|---|
| Compound | (min) | Conversion (%) | Conversion (%) |
| 324 | 1 | 0 | 0 |
| | 5 | 0 | 0 |
| | 30 | 0 | 0 |

| | | | |
|---|---|---|---|
| 1 | (h) | 0 | 0 |
| 4 | (h) | 0 | 0 |
| 24 | (h) | 0 | 0 |

| | | | |
|---|---|---|---|
| 326 | 1 | 0 | 0 |
| | 5 | 0 | 0 |
| | 30 | 0 | 0 |

| | | | |
|---|---|---|---|
| 1 | (h) | 0 | 0 |
| 4 | (h) | 0 | 0 |
| 24 | (h) | 0 | 0 |

| | | | |
|---|---|---|---|
| 327 | 1 | 0 | 0 |
| | 5 | 0 | 0 |
| | 30 | 0 | 0 |

| | | | |
|---|---|---|---|
| 1 | (h) | 0 | 0 |
| 4 | (h) | 0 | 0 |
| 24 | (h) | 0 | 0 |

### Example A2: Plasma Stability Assay

Compound stability in mouse and human plasma was measured by incubating 5 μM of compound with mouse or human plasma at 37° C. in a microwell plate for sequential time points, monitoring pro-drug disappearance and metabolite appearance (mesembrine). Each test was carried out in duplicate for each time point; 0 min, 15 min, 30 min, 60 min, 120, 240 min. Samples were analyzed via LC-MS/MS for known peaks, and results reported as 00 of prodrug compound remaining at time point, and 00 of metabolite (mesembrine) accumulated at time point. The data is represented in the tables below.

TABLE 4
Carbonates

| | | Mouse Plasma | | Human Plasma | |
|---|---|---|---|---|---|
| | Time | Prodrug | Metabolite | Prodrug | Metabolite |
| Compound | (min) | (%) | (%) | (%) | (%) |
| 358 | 0 | 100 | 58.8 | 100 | 0.6 |
| | 15 | 14.8 | 65.4 | 75.4 | 38.5 |
| | 30 | 10.3 | 73.2 | 58.1 | 45.8 |
| | 60 | 5.4 | 76.9 | 35.5 | 66.9 |
| | 120 | 0.0 | 61.8 | 11.7 | 76.4 |
| | 240 | 0.0 | 62.2 | 0.7 | 86.4 |

TABLE 5
Phosphate Esters

| | | Mouse Plasma | | Human Plasma | |
|---|---|---|---|---|---|
| | Time | Prodrug | Metabolite | Prodrug | Metabolite |
| Compound | (min) | (%) | (%) | (%) | (%) |
| 321 | 0 | 100 | 11.9 | 100 | 0.08 |
| | 15 | 77.3 | 28.4 | >99 | 0.29 |
| | 30 | 74.5 | 38.3 | >99 | 0.40 |
| | 60 | 58.4 | 51.1 | >99 | 0.49 |
| | 120 | 43.1 | 53.7 | >99 | 0.67 |
| | 240 | 31.7 | 55.2 | 78.2 | 0.61 |
| 323 | 0 | 100.0 | 18 | 100 | 1.8 |
| | 15 | >99 | 5.6 | 76.6 | 18.5 |
| | 30 | >99 | 8.3 | 64.5 | 29.7 |
| | 60 | 94.1 | 13.7 | 38.1 | 43.9 |
| | 120 | 71.9 | 24.0 | 12.8 | 61.5 |
| | 240 | 50.3 | 32.0 | 1.60 | 576 |
| 320 | 0 | 100 | 11.8 | 100 | 7.6 |
| | 15 | 58.5 | 15.9 | 95.7 | 8.0 |
| | 30 | 46.5 | 16.9 | 98.5 | 8.5 |
| | 60 | 30.7 | 18.6 | 95.7 | 8.6 |
| | 120 | 19.4 | 19.1 | 98.2 | 9.4 |
| | 240 | 11.7 | 18.9 | 95.3 | 10.4 |
| 359 | 0 | 100 | 0.0 | 100 | 0.0 |
| | 15 | >99 | 0.0 | 69.0 | 0.0 |
| | 30 | 82.7 | 0.0 | 65.1 | 0.0 |
| | 60 | 79.0 | 0.0 | 50.4 | 0.0 |
| | 120 | 50.7 | 0.0 | 32.8 | 15.7 |
| | 240 | 59.1 | 0.0 | 25.6 | 23.0 |

TABLE 6
Carbamates

| | | Mouse Plasma | | Human Plasma | |
|---|---|---|---|---|---|
| | Time | Prodrug | Metabolite | Prodrug | Metabolite |
| Compound | (min) | (%) | (%) | (%) | (%) |
| 327 | 0 | 100 | 0.37 | 100 | 0.18 |
| | 15 | >99 | 0.47 | >99 | 0.39 |
| | 30 | >99 | 0.52 | 98.9 | 0.33 |
| | 60 | >99 | 0.70 | >99 | 0.28 |
| | 120 | >99 | 0.91 | >99 | 0.42 |
| | 240 | >99 | 2.0 | 94.4 | 0.43 |
| 324 | 0 | 100.0 | 0.37 | 100.0 | 0.39 |
| | 15 | 97.4 | 0.57 | 98.2 | 0.33 |
| | 30 | 94.4 | 0.58 | 98.7 | 0.45 |
| | 60 | 94.9 | 0.73 | >99 | 0.41 |
| | 120 | 92.6 | 0.85 | >99 | 0.62 |
| | 240 | 88.9 | 1.15 | 96.5 | 0.69 |
| 326 | 0 | 100.0 | 0 | 100.0 | 0.0 |
| | 15 | >99 | 0.08 | 98.2 | 0.0 |
| | 30 | 96.6 | 0.13 | 99.9 | 0.0 |
| | 60 | >99 | 0.21 | 98.4 | 0.0 |
| | 120 | >99 | 0.32 | >99 | 0.07 |
| | 240 | 98.5 | 0.54 | 93.9 | 0.15 |

### Example A3: Plasma Stability Assay

Compound stability in rat and human plasma was measured by incubating compound with rat or human plasma at 37° C. in a microwell plate for sequential time points, monitoring pro-drug disappearance and metabolite appearance (mesembrine, 001). A 1 mM stock solution of test compounds were prepared by dissolving appropriate amount of compounds in DMSO. The 1 mM stock was further diluted 200-folds in rat or human plasma to attain a final concentration of 10 μM respectively (0.5% DMSO). 50 μL aliquots of positive controls and test compounds spiked into rat or human plasma (n=2) were added to a pre-warmed plate (37° C.) and shaken at 50 rpm. At each respective time point, the reaction was stopped by adding 500 μL of acetonitrile containing internal standards (100 nM aprozolam, 200 nM caffeine, 100 nM tolbutamide). All samples were vortexed for 10 minutes, followed by centrifugation at 3,220 g for 30 minutes to precipitate proteins. 100 μL of the supernatant is transferred to a new plate. The supernatant will be diluted with ultrapure water according to the LC-MS signal response and peak shape. The concentrations of test compounds and positive control were quantified in the test samples using LC-MS/MS. Results reported as % of prodrug compound remaining at time point, and 00 of metabolite (mesembrine, 001) accumulated at time point. The data is represented in the table below.

TABLE 7
Phosphate Esters

| | | Rat Plasma | | Human Plasma | |
|---|---|---|---|---|---|
| | Time | Prodrug | Metabolite | Prodrug | Metabolite |
| Compound | (min) | (%) | (%) | (%) | (%) |
| 321 | 0 | 100.0 | 23.9 | 100.0 | 0.40 |
| | 10 | 74.0 | 53.7 | >99 | 0.50 |
| | 30 | 69.8 | 56.0 | >99 | 0.57 |
| | 60 | 69.2 | 59.1 | 98.1 | 0.65 |
| | 120 | 68.4 | 59.3 | 94.5 | 0.82 |
| | 240 | 65.7 | 56.7 | 96.3 | 0.96 |
| 323 | 0 | 100.00 | 0.34 | 100.00 | 2.2 |
| | 10 | >99 | 3.5 | 86.9 | 23.4 |
| | 30 | 95.5 | 11.3 | 70.8 | 50.0 |
| | 60 | 82.0 | 24.7 | 52.7 | 82.1 |
| | 120 | 58.8 | 52.0 | 27.0 | >99 |
| | 240 | 34.0 | 91.0 | 9.3 | >99 |
| 408 | 0.0 | 100.0 | 0.4 | 100.0 | 0.67 |
| | 10 | 63.8 | 2.0 | 94.2 | 0.79 |
| | 30 | 29.2 | 4.7 | 82.6 | 1.1 |
| | 60 | 8.2 | 7.7 | 69.1 | 1.2 |
| | 120 | 0.8 | 11.8 | 45.4 | 1.2 |
| | 240 | 0.0 | 18.9 | 23.5 | 1.9 |
| 409 | 0.0 | 100.0 | 2.2 | 100.0 | 0.16 |
| | 10 | 89.9 | 6.4 | >99 | 0.20 |
| | 30 | 81.2 | 10.9 | 95.2 | 0.30 |
| | 60 | 71.0 | 13.7 | 93.0 | 0.33 |
| | 120 | 44.5 | 14.3 | 87.3 | 0.45 |
| | 240 | 22.7 | 13.9 | 75.8 | 0.65 |
| 410 | 0.0 | 100.0 | 1.3 | 100.0 | 0.02 |
| | 10 | 63.0 | 7.1 | 96.3 | 0.09 |
| | 30 | 25.4 | 12.0 | 98.8 | 0.15 |
| | 60 | 7.1 | 16.3 | 92.7 | 0.21 |
| | 120 | 0.52 | 15.8 | 88.2 | 0.44 |
| | 240 | 0.01 | 12.2 | 75.5 | 0.71 |
| 422 | 0.0 | 100.0 | 1.1 | 100.0 | 2.6 |
| | 10 | >99 | 1.9 | 89.4 | 11.2 |
| | 30 | >99 | 4.2 | 73.1 | 26.6 |
| | 60 | >99 | 7.6 | 62.4 | 38.9 |
| | 120 | 91.0 | 16.2 | 39.2 | 57.7 |
| | 240 | 76.8 | 31.3 | 19.6 | 68.3 |
| 423 | 0.0 | 100.00 | 1.02 | 100.00 | 0.76 |
| | 10 | >99 | 2.7 | >99 | 0.83 |
| | 30 | >99 | 5.8 | >99 | 0.85 |
| | 60 | 91.05 | 10.3 | 98.77 | 0.84 |
| | 120 | 83.29 | 19.3 | 97.80 | 0.84 |
| | 240 | 67.32 | 33.7 | 89.67 | 1.0 |

### Example A4: SERT Inhibition Assay

SERT inhibition was measured using a Neruotransmitter Transportation Fluorescence assay. Briefly, stable 5HTT cells were prepared in a 384 microwell plate. Compounds were prepared by in assay buffer (20 mM HEPES, 0.1% BSA). The compounds were added to the plated cells and incubated for 30 minutes at 37° C. 25 μL of dye solution (Molecular Devices Neurotransmitter Transporter Uptake Assay Kit) was added per well and incubated for 30 minutes at 37° C. The plates were then read on a plate reader.

The in vitro SERT inhibition was measured for the compounds listed in Table 7.

TABLE 8

| Compound | SERT IC₅₀ (nM) |
|---|---|
| 323 | 62.7 |

### Example A5: Metabolic Stability in Human Hepatocytes

A 2.5 uL aliquot of 100 μM test compound was incubated with 247.5 uL of cryopreserved hepatocytes suspended in serum-free incubation medium at 5×10⁵ viable cells/mL. The mixture was incubated at 37° C. and shaken at 500 rpm for the designated time points (0.5, 15, 30, 60, 90, or 120 min). At each time point, 25 μL aliquot of incubation mixture was transferred to 125 μL of cold acetonitrile containing internal standard, followed by centrifugation for 30 min at 3,220 g. 100 μL of supernatant was mixed with 100 μL of distilled water for analysis by LC-MS/MS. Peak areas were determined from extracted ion chromatograms and in vitro half-life (t_{1/2}) was determined from the concentration vs time regression slope, in vitro t_{1/2}=0.693/k. Conversion of the in vitro t_{1/2} (in min) into the in vitro intrinsic clearance (in vitro CLᵢₙₜ, in μL/min/10⁶ cells) is done using: in vitro CLᵢₙₜ=kV/N.

TABLE 9

| Compound | t_{1/2} (min) | Clᵢₙₜ (μL/min/10⁶ cells) |
|---|---|---|
| 001 | 17 | 40 |
| 321 | 23 | 59.4 |
| 323 | 327 | 4.2 |
| 408 | 220 | 6.4 |
| 409 | 1540 | 1.1 |
| 422 | 366 | 3.9 |
| 423 | 57 | 24.4 |

### Example B1: Pharmacokinetics of 321

Total fifteen male mice were used in this study with n=3 mice/time point with sparse sampling study design. Animals were administered through intravenous route with solution formulation of 321 at 2 mg/kg dose. The formulation vehicle used was 1% NMP and 99% normal saline. Blood samples (approximately 60 μL) were collected under light isoflurane anesthesia from a set of three mice at 0.083, 0.25, 0.50, 1 and 2 h. The blood samples were collected at each time point in labeled micro centrifuge tube containing K₂EDTA as anticoagulant and phenyl methyl sulfonyl fluoride [PMSF (100 mM; 10 μL/mL of blood)] as a stabilizer. Plasma samples were separated by centrifugation of whole blood and stored below −70° C. until bioanalysis. Following blood collection, immediately animals were sacrificed, and vena-cava was cut open and blood was drained. Whole body was perfused from heart using 10 mL of normal saline. Brain samples were collected from set of three mice at 0.083, 0.25, 0.50, 1 and 2 h. After isolation, brain samples were rinsed three times in ice cold normal saline (for 5-10 seconds/rinsed using ˜10-20 mL normal saline in disposable petri dish for each rinse), dried on blotting paper and weighed. Brain samples were homogenized using 2 volumes of ice-cold PBS (pH−7.4) of brain weight. Total homogenate volume was three times of the brain weight. Brain homogenates were stored below −70±10° C. until analysis.

The extraction procedure for plasma/brain samples and the spiked plasma/brain calibration standards were identical (PMSF Stabilized Plasma and Brain used for Preparation of calibration standards): A 20 μL of study sample plasma/brain or spiked plasma/brain calibration standard was added to individual pre-labeled micro-centrifuge tubes followed by 200 μL of internal standard prepared in acetonitrile (Rosuvastatin, 50 ng/mL) was added except for blank, where 200 μL of acetonitrile was added. Samples were vortexed for 5 minutes. Samples were centrifuged for 10 minutes at a speed of 4000 rpm at 4° C. Following centrifugation, 200 μL of clear supernatant was transferred in 96 well plates and analyzed using LC-MS/MS.

Brain samples were diluted (1-part of tissue: 2-part of buffer) and homogenized. The homogenate was submitted for bioanalysis and the concentrations (ng/mL) received were corrected with dilution factor (3×) and the final reported concentrations were represented in ng/g.

The plasma and brain concentration-time data of 321 and 001 were used for the pharmacokinetic analysis. Plasma and brain samples were quantified by fit-for-purpose LC-MS/MS method.

LLOQ: 321: 1.02 ng/mL for plasma and brain
001: 1.01 ng/mL for plasma and brain

### Formulation Preparation

Stock of strength 40 mg/mL was prepared in NMP.
IV (0.4 mg/mL) 0.060 mL (2.4 mg of compound) of stock of 321 for IV dosing was added in a labeled bottle. Individual excipient volumes were calculated to prepare solution formulation of 321 at strength of 0.4 mg/mL. The volume of 5.940 mL of normal saline was added to bottle (containing 0.060 mL of NMP stock) and vortexed for 2 minutes to get clear solution. The amount weighed and calculation details are in Table 9.

TABLE 10

| Ingredients | % Content | mg or mL |
|---|---|---|
| 321 | — | 0.060 mL (2.4 mg of |
| | | compound) of stock |
| NMP | 1 | 0.060 mL (From stock) |
| Normal saline | 99 | 5.940 mL |

Note:
The volume of NMP stock (0.06 mL) was equivalent to 1% of total formulation volume.

### Formulation Analysis Results

After preparation of formulation, a volume of 200 μL was taken for analysis. The formulation was analyzed and found to be within the acceptance criteria (in-house acceptance criteria is ±20% from the nominal value). Formulation was prepared freshly prior to dosing.

TABLE 11

| | Theoretical Conc. | Conc. Found | |
|---|---|---|---|
| Compound | (mg/mL) | (mg/mL) | % Change |
| 321 | 0.40 | 0.41 | 2.50 |

### Results:

Following a single intravenous administration of 321 in male C57BL/6 mice at 2 mg/kg dose, plasma and brain concentrations were quantifiable up to 2 and 0.5 h, respectively. Brain exposure Kp was 0.28.
Concentrations of 001 were quantifiable up to 1 h in plasma and brain. Brain exposure Kp was 1.21.

TABLE 12
Pharmacokinetic data of 321 (Prodrug) and 001 (metabolite) in male C57BL/6
mice following a single intravenous administration (Dose: 2 mg/kg, IV)

| | | Dose | | ^{a}C₀ | AUCₗₐₛₜ | T_{1/2} | CL | Vₛₛ | |
|---|---|---|---|---|---|---|---|---|---|
| Route | Compound | (mg/kg) | Matrix | (ng/mL) | (hr*ng/mL) | (hr) | (mL/min/kg) | (L/kg) | Brain-Kp |
| IV | 321 | 2 | Plasma | 503.64 | 97.14 | 0.36 | 340.73 | 4.80 | — |
| | | | ^{#}Brain | 158.45 | 27.57 | 0.11 | NR (1154.07) | 10.34 | 0.28 |
| IV | 001 | — | Plasma | 64.05 | 10.52 | — | — | | — |
| | | | ^{#}Brain | 59.10 | 12.72 | | | | 1.21 |

^{a}Back extrapolated concentration in IV group
^{#}Brain exposure was expressed as hr*ng/g
NR—Clearance values are not reportable since very high compared to normal Liver blood flow in mice (90 mL/min/kg).

TABLE 13
Plasma concentrations of 321 in male C57BL/6 mice following
a single intravenous administration (Dose: 2 mg/kg)

| | Plasma concentrations (ng/mL) | | | | |
|---|---|---|---|---|---|
| | Time (h) | | | | |
| | 0.08 | 0.25 | 0.5 | 1 | 2 |
| Mean | 304.66 | 104.69 | 26.16 | 8.57 | 1.38^{d} |
| SD | 70.38 | 36.56 | 8.00 | 6.47 | NA |
| % CV | 23.10 | 34.92 | 30.56 | 75.56 | NA |

LLOQ: 1.02 ng/mL; BLQ—Below limit of quantitation; NA—Not applicable,
^{d}Average of two values reported, considered for data analysis and graphical presentation

TABLE 14
Brain concentrations of 321 in male C57BL/6 mice following
a single intravenous administration (Dose: 2 mg/kg)

| | Brain concentrations (ng/g) | | | | |
|---|---|---|---|---|---|
| | Time (h) | | | | |
| | 0.08 | 0.25 | 0.5 | 1 | 2 |
| Mean | 99.58 | 37.11 | 7.89 | NA | NA |
| SD | 30.34 | 5.83 | 2.98 | NA | NA |
| % CV | 30.47 | 15.71 | 37.73 | NA | NA |

LLOQ: 1.02 ng/mL; BLQ—Below limit of quantitation; NA—Not applicable

TABLE 15
Plasma concentrations of 001 in male C57BL/6 mice following a
single intravenous administration of 321 at a dose of 2 mg/kg

| | Plasma concentrations (ng/mL) | | | | |
|---|---|---|---|---|---|
| | Time (h) | | | | |
| Animal ID | 0.08 | 0.25 | 0.5 | 1 | 2 |
| Mean | 37.90 | 12.43 | 4.85 | 6.39^{c} | NA |
| SD | 5.37 | 5.10 | 1.23 | NA | NA |
| % CV | 14.17 | 41.02 | 25.36 | NA | NA |

LLOQ: 1.01 ng/mL for plasma; BLQ—Below limit of quantitation; NA—Not applicable,
^{c}Single value reported and excluded from data analysis and graphical presentation

TABLE 16
Brain concentrations of 001 in male C57BL/6 mice following a
single intravenous administration of 321 at a dose of 2 mg/kg

| | Brain concentrations (ng/g) | | | | |
|---|---|---|---|---|---|
| | Time (h) | | | | |
| | 0.08 | 0.25 | 0.5 | 1 | 2 |
| Mean | 40.03 | 17.49 | 13.40 | 4.05^{c} | NA |
| SD | 12.49 | 5.18 | 1.49 | NA | NA |
| % CV | 31.20 | 29.61 | 11.09 | NA | NA |

LLOQ: 1.01 ng/ml; BLQ—Below limit of quantitation; NA—Not applicable,
^{c}Single value reported and excluded from data analysis and graphical presentation

Bioanalytical Summary

| | |
|---|---|
| Instrument: | XEVO TQ-XS (Waters 003 for 321) |
| | XEVO TQ-XS (Waters 002 for 001) |
| Mobile Phase | A: 0.1% Formic acid in Acetonitrile |
| | B: 0.1% Formic acid in Water |
| Column: | Acquity BEH C18, 50 × 2.1 mm, 1.7 μm (For 001) |
| | Kinetex XB, C18, 100A, 50 × 2.1 mm, 1.7 μm (For 358F) |
| Injection Volume (μL): | 1 |
| Column Oven Temperature (° C.): | 45 |
| Retention Time (in min): | Analyte: 001: 1.07 |
| | 321: 1.00 |
| | IS: Rosuvastatin: 1.08, Rosuvastatin: 1.27 |

TABLE 17
LC Gradient Used for 321

| Time | Flow Rate | PUMP A | PUMP B |
|---|---|---|---|
| (Minutes) | (mL/min) | (% Conc) | ( % Conc) |
| Initial | 0.4 | 2 | 98 |
| 0.30 | 0.4 | 2 | 98 |
| 0.60 | 0.4 | 98 | 2 |
| 1.40 | 0.4 | 98 | 2 |
| 1.80 | 0.4 | 2 | 98 |
| 2.20 | 0.4 | 2 | 98 |

### Example B2: Pharmacokinetics of 323

Total fifteen male mice were used in this study with n=3 mice/time point with sparse sampling study design. Animals were administered through intravenous route with solution formulation of 323 at 2 mg/kg dose. The formulation vehicle used was 1% NMP and 99% normal saline. Blood samples (approximately 60 μL) were collected under light isoflurane anesthesia from a set of three mice at 0.083, 0.25, 0.50, 1 and 2 h. The blood samples were collected at each time point in labeled micro centrifuge tube containing K₂EDTA as anticoagulant and phenyl methyl sulfonyl fluoride [PMSF (100 mM; 10 μL/mL of blood)] as a stabilizer. Plasma samples were separated by centrifugation of whole blood and stored below −70° C. until bioanalysis. Following blood collection, immediately animals were sacrificed, and vena-cava was cut open and blood was drained. Whole body was perfused from heart using 10 mL of normal saline. Brain samples were collected from set of three mice at 0.083, 0.25, 0.50, 1 and 2 h. After isolation, brain samples were rinsed three times in ice cold normal saline (for 5-10 seconds/rinsed using ˜10-20 mL normal saline in disposable petri dish for each rinse), dried on blotting paper and weighed. Brain samples were homogenized using 2 volumes of ice-cold PBS (pH−7.4) of brain weight. Total homogenate volume was three times of the brain weight. Brain homogenates were stored below −70±10° C. until analysis.

The extraction procedure for plasma/brain samples and the spiked plasma/brain calibration standards were identical (PMSF Stabilized Plasma and Brain used for Preparation of calibration standards): A 20 μL of study sample plasma/brain or spiked plasma/brain calibration standard was added to individual pre-labeled micro-centrifuge tubes followed by 200 μL of internal standard prepared in acetonitrile (Rosuvastatin+Cetrizine, 50 ng/mL) was added except for blank, where 200 μL of acetonitrile was added. Samples were vortexed for 5 minutes. Samples were centrifuged for 10 minutes at a speed of 4000 rpm at 4° C. Following centrifugation, 200 μL of clear supernatant was transferred in 96 well plates and analyzed using LC-MS/MS.

Brain samples were diluted (1-part of tissue: 2-part of buffer) and homogenized. The homogenate was submitted for bioanalysis and the concentrations (ng/mL) received were corrected with dilution factor (3×) and the final reported concentrations were represented in ng/g.

The plasma and brain concentration-time data of 323 and 001 were used for the pharmacokinetic analysis. Plasma and brain samples were quantified by fit-for-purpose LC-MS/MS method.

LLOQ: 323: 1.02 ng/mL for plasma and brain
001: 1.01 ng/mL for plasma and brain

### Formulation Preparation

Stock of strength 40 mg/mL was prepared in NMP.
IV (0.4 mg/mL): Accurately 0.050 mL (˜2 mg of compound) of stock of 323 for IV dosing was added in a labeled bottle. Individual excipient volumes were calculated to prepare solution formulation of 323 at strength of 0.4 mg/mL. The volume of 4.950 mL of normal saline was added to bottle (containing 0.050 mL of NMP stock) and vortexed for 2 minutes to get clear solution. The amount weighed and calculation details are in Table 17.

TABLE 18

| Ingredients | % Content | mg or mL |
|---|---|---|
| 323 | — | 0.050 mL (~2 mg of |
| | | compound) of stock |
| NMP | 1 | 0.050 mL (From stock) |
| Normal saline | 99 | 4.950 mL |

Note:
The volume of NMP stock (().05 mL) was equivalent to 1% of total formulation volume.

### Formulation Analysis Results

After preparation of formulation, a volume of 200 μL was taken for analysis. The formulation was analyzed and found to be within the acceptance criteria (in-house acceptance criteria is ±20% from the nominal value). Formulation was prepared freshly prior to dosing.

TABLE 19

| | Theoretical Conc. | Conc. Found | |
|---|---|---|---|
| Compound | (mg/mL) | (mg/mL) | % Change |
| 323 | 0.40 | 0.42 | 5.00 |

### Results:

Following a single intravenous administration of 323 in male C57BL/6 mice at 2 mg/kg dose, plasma and brain concentrations were quantifiable up to 2 and 0.5 h, respectively. Brain exposure Kp was 0.07.
Concentrations of 001 were quantifiable up to 1 h in plasma and up to 2 h in brain. Brain exposure Kp was ˜23.

TABLE 20
Pharmacokinetic data of 323 (Prodrug) and 001 (metabolite) in male C57BL/6
mice following a single intravenous administration (Dose: 2 mg/kg, IV)

| | | Dose | | ^{a}C₀ | AUCₗₐₛₜ | T_{1/2} | CL | Vₛₛ | |
|---|---|---|---|---|---|---|---|---|---|
| Route | Compound | (mg/kg) | Matrix | (ng/mL) | (hr*ng/mL) | (hr) | (mL/min/kg) | (L/kg) | Brain-Kp |
| IV | 323 | 2 | Plasma | 609.01 | 96.00 | 0.17 | NR (343.84) | 3.25 | — |
| | | | ^{#}Brain | 33.89 | 6.56 | 0.18 | NR (4350.55) | 61.73 | 0.07 |
| IV | 001 | — | Plasma | 121.37 | 17.60 | — | — | — | — |
| | | | ^{#}Brain | 278.48 | 411.95 | — | — | — | 23.40 |

^{a}Back extrapolated concentration in IV group
^{#}Brain exposure was expressed as hr*ng/g
NR— Clearance values are not reportable since very high compared to normal iver blood flow in mice (90 mL/min/kg).

TABLE 21
Plasma concentrations of 323 in male C57BL/6 mice following
a single intravenous administration (Dose: 2 mg/kg)

| | Plasma concentrations (ng/mL) | | | | |
|---|---|---|---|---|---|
| | Time (h) | | | | |
| | 0.08 | 0.25 | 0.5 | 1 | 2 |
| Mean | 332.15 | 91.59 | 26.34 | 4.05 | 1.03^{c} |
| SD | 104.54 | 19.84 | 8.89 | 1.54 | NA |
| % CV | 31.48 | 21.66 | 33.74 | 38.01 | NA |

LLOQ: 1.02 ng/mL; BLQ—Below limit of quantitation; NA—Not applicable,
^{c}Single value reported and excluded from data analysis and graphical presentation

TABLE 22
Brain concentrations of 323 in male C57BL/6 mice following
a single intravenous administration (Dose: 2 mg/kg)

| | Brain concentrations (ng/g) | | | | |
|---|---|---|---|---|---|
| | Time (h) | | | | |
| | 0.08 | 0.25 | 0.5 | 1 | 2 |
| Mean | 22.15 | 8.97 | 4.43^{d} | NA | NA |
| SD | 2.51 | 1.69 | NA | NA | NA |
| % CV | 11.33 | 18.81 | NA | NA | NA |

LLOQ: 1.02 ng/mL; BLQ—Below limit of quantitation; NA—Not applicable,
^{d}Average of two values reported, considered for data analysis and graphical presentation

TABLE 23
Plasma concentrations of 001 in male C57BL/6 mice following a
single intravenous administration of 323 at a dose of 2 mg/kg

| | Plasma concentrations (ng/ml) | | | | |
|---|---|---|---|---|---|
| | Time (h) | | | | |
| | 0.08 | 0.25 | 0.5 | 1 | 2 |
| Mean | 60.71 | 13.93 | 4.79 | 1.75 | NA |
| SD | 28.43 | 9.51 | 2.45 | 0.51 | NA |
| % CV | 46.82 | 68.26 | 51.10 | 29.07 | NA |

LLOQ: 1.01 ng/mL for plasma; BLQ—Below limit of quantitation; NA—Not applicable,

TABLE 24
Brain concentrations of 001 in male C57BL/6 mice following a
single intravenous administration of 323 at a dose of 2 mg/kg

Brain concentrations (ng/g)
Time (h)

| | 0.08 | 0.25 | 0.5 | 1 | 2 |
|---|---|---|---|---|---|
| Mean | 276.23 | 271.50 | 196.29 | 190.53 | 185.52 |
| SD | 123.64 | 31.37 | 9.32 | 10.42 | 8.03 |
| % CV | 44.76 | 11.55 | 4.75 | 5.47 | 4.33 |

LLOQ: 1.01 ng/ml

| | |
|---|---|
| Instrument ID: | Parent: XEVO TQ-XS (Waters-001) |
| | 001: XEVO TQ-XS (Waters-002) |
| Mobile Phase | A: 0.1% Formic acid in Acetonitrile |
| | B: 0.1% Formic acid in Water |
| Column: | Acquity BEH C18, 50 × 2.1 mm, 1.7 μm |
| Injection Volume (μL): | 1 |
| Column Oven | 45 |
| Temperature (° C.): | |
| Retention Time (in min): | Analyte: 001: 1.06 |
| | 323 0.98 |
| | IS: Rosuvastatin: 1.03 |
| | Cetrizine: 1.05 |

TABLE 25
LC Gradient Used for 323

| Time | Flow Rate | PUMP A | PUMP B |
|---|---|---|---|
| (Minutes) | (mL/min) | (% Conc) | (% Conc) |
| Initial | 0.4 | 0 | 100 |
| 0.30 | 0.4 | 0 | 100 |
| 0.50 | 0.4 | 95 | 5 |
| 1.40 | 0.4 | 95 | 5 |
| 1.80 | 0.4 | 0 | 100 |
| 2.20 | 0.4 | 0 | 100 |

TABLE 26
LC Gradient Used for 001

| Time | Flow Rate | PUMP A | PUMP B |
|---|---|---|---|
| (Minutes) | (mL/min) | (% Conc) | (% Conc) |
| Initial | 0.4 | 0 | 100 |
| 0.40 | 0.4 | 0 | 100 |
| 0.60 | 0.4 | 95 | 5 |
| 1.80 | 0.4 | 95 | 5 |
| 2.00 | 0.4 | 0 | 100 |
| 2.20 | 0.4 | 0 | 100 |

### Example B3: Pharmacokinetics of 358

Total fifteen male mice were used in this study with n=3 mice/time point with sparse sampling study design. Animals were administered through intravenous route with solution formulation of 358 at 2 mg/kg dose. The formulation vehicle used was 1% NMP and 99% normal saline. Blood samples (approximately 60 μL) were collected under light isoflurane anesthesia from a set of three mice at 0.083, 0.25, 0.50, 1 and 2 h. The blood samples were collected at each time point in labeled micro centrifuge tube containing K₂EDTA as anticoagulant and phenyl methyl sulfonyl fluoride [PMSF (100 mM; 10 μL/mL of blood)] as a stabilizer. Plasma samples were separated by centrifugation of whole blood and stored below −70° C. until bioanalysis. Following blood collection, immediately animals were sacrificed, and vena-cava was cut open and blood was drained. Whole body was perfused from heart using 10 mL of normal saline. Brain samples were collected from set of three mice at 0.083, 0.25, 0.50, 1 and 2 h. After isolation, brain samples were rinsed three times in ice cold normal saline (for 5-10 seconds/rinsed using ˜10-20 mL normal saline in disposable petri dish for each rinse), dried on blotting paper and weighed. Brain samples were homogenized using 2 volumes of ice-cold PBS (pH-7.4) of brain weight. Total homogenate volume was three times of the brain weight. Brain homogenates were stored below −70±10° C. until analysis.

The extraction procedure for plasma/brain samples and the spiked plasma/brain calibration standards were identical (PMSF Stabilized Plasma and Brain used for Preparation of calibration standards):

A 20 μL of study sample plasma/brain or spiked plasma/brain calibration standard was added to individual pre-labeled micro-centrifuge tubes followed by 200 μL of internal standard prepared in acetonitrile (Rosuvastatin+Cetrizine, 50 ng/mL) was added except for blank, where 200 μL of acetonitrile was added. Samples were vortexed for 5 minutes. Samples were centrifuged for 10 minutes at a speed of 4000 rpm at 4° C. Following centrifugation, 200 μL of clear supernatant was transferred in 96 well plates and analyzed using LC-MS/MS.

Note: Brain samples were diluted (1-part of tissue: 2-part of buffer) and homogenized. The homogenate was submitted for bioanalysis and the concentrations (ng/mL) received were corrected with dilution factor (3×) and the final reported concentrations were represented in ng/g.

The plasma and brain concentration-time data of 358 and 001 were used for the pharmacokinetic analysis. Plasma and brain samples were quantified by fit-for-purpose LC-MS/MS method.

LLOQ: 358: 1.02 ng/mL for plasma and brain
001: 1.01 ng/mL for plasma and brain

### Formulation Preparation

Stock of strength 40 mg/mL (with salt and purity) correction was prepared in NMP.
IV (0.4 mg/mL): 0.060 mL (2.4 mg of compound equivalent to free base) of stock of 358 for IV dosing was added in a labeled bottle. Individual excipient volumes were calculated to prepare solution formulation of 358 at strength of 0.4 mg/mL. The volume of 5.940 mL of normal saline was added to bottle (containing 0.060 mL of NMP stock) and vortexed for 2 minutes to get clear solution. The amount weighed and calculation details are in Table 26.

TABLE 27

| Ingredients | % Content | mg or mL |
|---|---|---|
| 358 | — | 0.060 mL (2.4 mg of |
| | | compound) of stock |
| NMP | 1 | 0.060 mL (From stock) |
| Normal saline | 99 | 5.940 mL |

Note:
The volume of NMP stock (0.06 mL) was equivalent to 1% of total formulation volume.

### Formulation Analysis Results

After preparation of formulation, a volume of 200 μL was taken for analysis. The formulation was analyzed and found to be within the acceptance criteria (in-house acceptance criteria is +20% from the nominal value). Formulation was prepared freshly prior to dosing.

TABLE 28

| | Theoretical | Conc. | |
|---|---|---|---|
| | Conc. | Found | |
| Compound | (mg/mL) | (mg/mL) | % Change |
| 358 | 0.40 | 0.33 | −17.50 |

### Results:

Following a single intravenous administration of 358 in male C57BL/6 mice at 2 mg/kg dose, plasma and brain concentrations were quantifiable up to 0.25 and 0.08 h, respectively.
Concentrations of 001 were quantifiable up to 2 h in plasma and brain.

TABLE 29
Pharmacokinetic data of 358 (Prodrug) and 001 (metabolite) in male C57BL/6
mice following a single intravenous administration (Dose: 2 mg/kg, IV)

| | | Dose | | ^{a}C₀ | AUCₗₐₛₜ | T_{1/2} | CL | Vₛₛ | |
|---|---|---|---|---|---|---|---|---|---|
| Route | Compound | (mg/kg) | Matrix | (ng/mL) | (hr*ng/mL) | (hr) | (mL/min/kg) | (L/kg) | Brain-Kp |
| IV | 358 | 2 | Plasma | | | NC | | | |
| | | | ^{#}Brain | | | NC | | | |
| IV | 001 | | Plasma | 288.49 | 36.64 | — | — | — | — |
| | | | ^{#}Brain | 1567.60 | 190.88 | — | — | — | — |

^{a} Back extrapolated concentration in IV group
^{#}Brain exposure was expressed as hr*ng/g
NC—Not calculated since, concentrations were not quantifiable at sufficient time points

TABLE 30
Plasma concentrations of 358 in male C57BL/6 mice following
a single intravenous administration (Dose: 2 mg/kg)

| | Plasma concentrations (ng/mL) | | | | |
|---|---|---|---|---|---|
| | Time (h) | | | | |
| | 0.08 | 0.25 | 0.5 | 1 | 2 |
| Mean | 14.29 | 1.51^{d} | NA | NA | NA |
| SD | 4.94 | NA | NA | NA | NA |
| % CV | 34.59 | NA | NA | NA | NA |

LLOQ: 1.02 ng/ml; BLQ—Below limit of quantitation; NA—Not applicable,
^{d}Average of two values reported

TABLE 31
Brain concentrations of 358 in male C57BL/6 mice following
a single intravenous administration (Dose: 2 mg/kg)

| | Brain concentrations (ng/g) | | | | |
|---|---|---|---|---|---|
| | Timc (h) | | | | |
| | 0.08 | 0.25 | 0.5 | 1 | 2 |
| Mean | 18.27 | NA | NA | NA | NA |
| SD | 2.09 | NA | NA | NA | NA |
| % CV | 11.46 | NA | NA | NA | NA |

LLOQ: 1.02 ng/mL; BLQ—Below limit of quantitation; NA—Not applicable

TABLE 32
Plasma concentrations of 001 in male C57BL/6 mice following a
single intravenous administration of 358 at a dose of 2 mg/kg

| | Plasma concentrations (ng/mL) | | | | |
|---|---|---|---|---|---|
| | Time (h) | | | | |
| | 0.08 | 0.25 | 0.5 | 1 | 2 |
| Mean | 141.64 | 31.24 | 6.66 | 1.13^{c} | 1.11^{c} |
| SD | 44.68 | 1.16 | 3.04 | NA | NA |
| % CV | 31.54 | 3.72 | 45.71 | NA | NA |

LLOQ: 1.01 ng/mL for plasma; BLQ—Below limit of quantitation; NA—Not applicable,
^{c}Single value reported and excluded from data analysis

TABLE 33
Brain concentrations of 001 in male C57BL/6 mice following a
single intravenous administration of 358 at a dose of 2 mg/kg

| | Brain concentrations (ng/g) | | | | |
|---|---|---|---|---|---|
| | Time (h) | | | | |
| | 0.08 | 0.25 | 0.5 | 1 | 2 |
| Mean | 685.15 | 118.02 | 39.51 | 11.72^{d} | 20.01^{c} |
| SD | 198.04 | 16.30 | 21.11 | NA | NA |
| % CV | 28.90 | 13.81 | 53.43 | NA | NA |

LLOQ: 1.01 ng/mL, BLQ—Below limit of quantitation; NA—Not applicable,
^{d}Average of two values reported, considered for data analysis and graphical presentation,
^{c}Single value reported and excluded from data analysis

Bioanalytical Summary

| | |
|---|---|
| Instrument: | XEVO TQ-XS (Waters 003 for 358) |
| | XEVO TQ-XS (Waters 002 for 001) |
| Mobile Phase | A: 0.1% Formic acid in Acetonitrile |
| | B: 0.1% Formic acid in Water |
| Column: | Acquity BEH C18, 50 × 2.1 mm, 1.7 μm (For 001) |
| | Kinetex XB, C18, 100A, 50 × 2.1 mm, 1.7 μm (For 358) |
| Injection Volume (μL): | 1 |
| Column Oven Temperature (° C.): | 45 |
| Retention Time (in min): | |
| Analyte: | 001: 1.07 |
| | 358: 1.01 |
| | IS: Rosuvastatin: 1.08, Rosuvastatin: 1.27 |

TABLE 34
LC Gradient Used for 358

| Time | Flow Rate | PUMP A | PUMP B |
|---|---|---|---|
| (Minutes) | (mL/min) | (% Conc) | ( % Conc) |
| Initial | 0.4 | 2 | 98 |
| 0.30 | 0.4 | 2 | 98 |
| 0.60 | 0.4 | 98 | 2 |
| 1.40 | 0.4 | 98 | 2 |
| 1.80 | 0.4 | 2 | 98 |
| 2.20 | 0.4 | 2 | 98 |

TABLE 35
LC Gradient Used for 001

| Time | Flow Rate | PUMP A | PUMP B |
|---|---|---|---|
| (Minutes) | (mL/min) | (% Conc) | ( % Conc) |
| Initial | 0.4 | 0 | 100 |
| 0.40 | 0.4 | 0 | 100 |
| 0.60 | 0.4 | 95 | 5 |
| 1.80 | 0.4 | 95 | 5 |
| 2.00 | 0.4 | 0 | 100 |
| 2.20 | 0.4 | 0 | 100 |

1-37. (canceled)

38. A compound of formula (I): 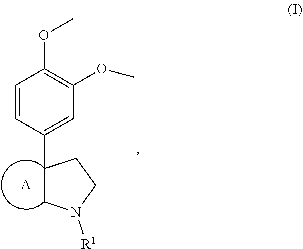
or a pharmaceutically acceptable salt thereof,
wherein
R¹ is H or C₁-C₇ alkyl;
ring A is 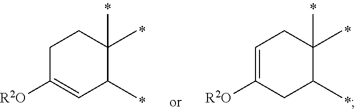
wherein * denotes the attachment points of ring A to the compound of formula (I),
R² is —P(O)OR⁴OR⁵, —C(O)NR³R⁴, or —C(O)OR⁶;
R³ is H, C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein C₁-C₆ alkyl, C₂-C₆ alkenyl, C₃-C₁₀ cycloalkyl, phenyl, and 5- to 7-membered heteroaryl are optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein C₁-C₆ alkyl is optionally substituted by phenyl;
or R³ and R⁴ combine to form a 4- to 7-membered heterocycle, wherein the 4- to 7-membered heterocycle is optionally substituted by halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁵ is H, C₁-C₆ alkyl, phenyl, —(CH₂O)ₙ—C(O)OC₁-C₆ alkyl, or —(CH₂O)ₙ—C(O)C₁-C₆ alkyl, wherein C₁-C₆ alkyl is optionally substituted by phenyl;
or R⁴ and R⁵ combine to form a 4- to 8-membered heterocycle, wherein the 4- to 8-membered heterocycle is optionally substituted by halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein C₁-C₆ alkyl, C₂-C₆ alkenyl, C₃-C₁₀ cycloalkyl, phenyl, and 5- to 7-membered heteroaryl are optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.

39. The compound of claim 38, wherein R¹ is methyl.

40. The compound of claim 39, wherein R² is —C(O)NR³R⁴.

41. The compound of claim 40, wherein:
a. R³ and R⁴ are each independently H or C₁-C₆ alkyl; or
b. R³ is phenyl optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and R⁴ is H or C₁-C₆ alkyl; or
c. R³ and R⁴ combine to form a 4- to 7-membered heterocycle, wherein the 4- to 7-membered heterocycle is optionally substituted by halo, hydroxy, C₁-C₃ alkyl, C₁-C₃ alkoxy, nitro, —N(C₁-C₃ alkyl)₂, —NH₂, —N(H)C₁-C₃ alkyl, C₁-C₃ haloalkyl, —COOH, or cyano; or
d. R³ is C₃-C₁₀ cycloalkyl optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, or cyano; and R⁴ is H or C₁-C₆ alkyl; or
e. R³ is C₁-C₆ alkyl optionally substituted by C₁-C₃ alkoxy, and —COOH; and R⁴ is H or C₁-C₆ alkyl.

42. The compound of claim 41, wherein ring A is

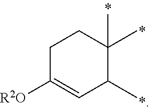

43. The compound of claim 40, wherein the compound is selected from: 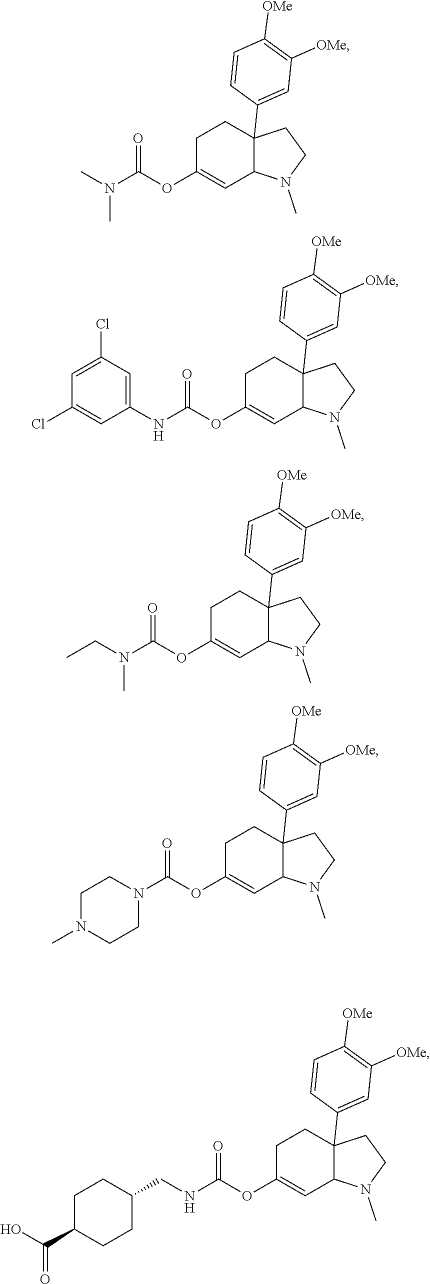 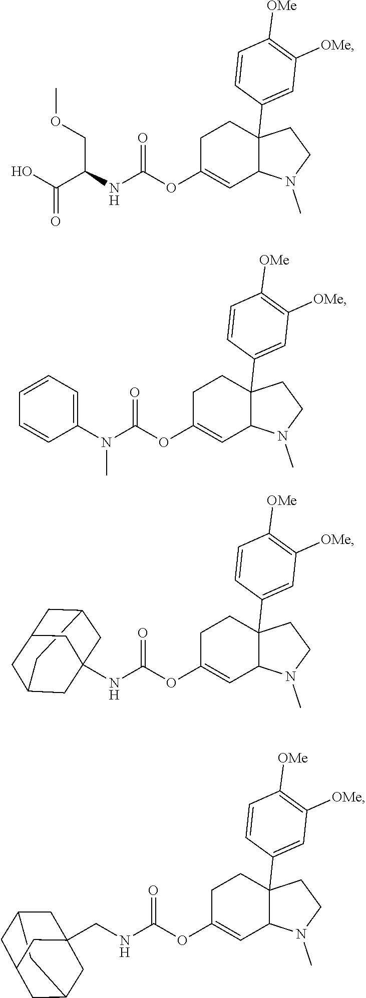
or a pharmaceutically acceptable salt thereof.

44. The compound of claim 39, wherein R² is —C(O)OR⁶.

45. The compound of claim 44, wherein R⁶ is C₁-C₆ alkyl.

46. The compound of claim 45, wherein ring A is

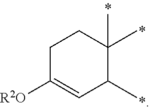

47. The compound of claim 44, wherein the compound is 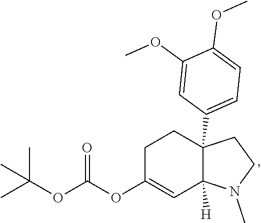 [SMILES: COc1ccc([C@@]23CCC(OC(=O)OC(C)(C)C)=C[C@@H]2N(C)CC3)cc1OC]
or a pharmaceutically acceptable salt thereof.

48. The compound of claim 39, wherein R² is —P(O)OR⁴OR⁵.

49. The compound of claim 48, wherein R⁴ and R⁵ combine to form a 4- to 8-membered heterocycle, wherein the 4- to 8-membered heterocycle is optionally substituted by halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

50. The compound of claim 48, wherein
f. R⁴ and R⁵ are each phenyl;
g. R⁴ and R⁵ are each C₁-C₆ alkyl;
h. R⁴ is H and R⁵ is —(CH₂O)ₙ—C(O)OC₁-C₆ alkyl, or —(CH₂O)ₙ—C(O)C₁-C₆ alkyl; or
i. R⁴ and R⁵ combine to form a 6-membered heterocycle, wherein the 6-membered heterocycle is optionally substituted by C₁-C₃ alkyl.

51. The compound of claim 50, wherein ring A is

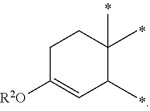

52. The compound of claim 51, wherein R² is

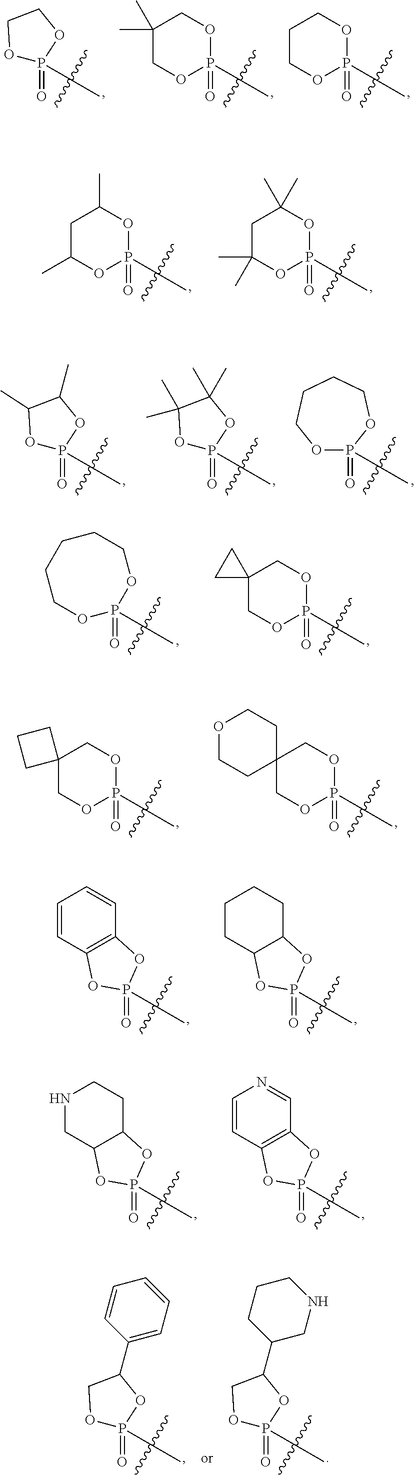

53. The compound of claim 51, wherein the compound is: 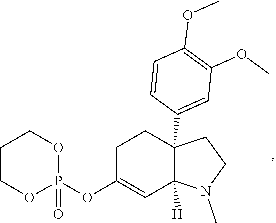 [SMILES: COc1ccc([C@@]23CCC(OP4(=O)OCCCO4)=C[C@@H]2N(C)CC3)cc1OC]
or a pharmaceutically acceptable salt thereof.

54. The compound of claim 51, represented by: 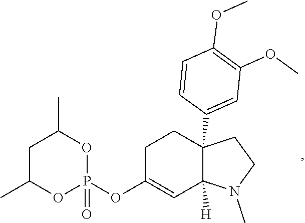
or a pharmaceutically acceptable salt thereof.

55. A compound of formula (I): 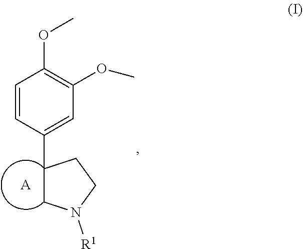
or a pharmaceutically acceptable salt thereof,
wherein
R¹ is methyl;
ring A is 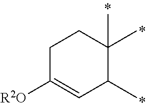
wherein * denotes the attachment points of ring A to the compound of formula (I),
R² is —P(O)OR⁴OR⁵ or —C(O)OR⁶;
R⁴ and R⁵ combine to form a 4- to 8-membered heterocycle, wherein the 4- to 8-membered heterocycle is optionally substituted by C₁-C₃ alkyl; and
R⁶ is C₁-C₆ alkyl.

56. A pharmaceutical composition, comprising a compound of claim 38, and a pharmaceutically acceptable excipient.

57. A method of treating social anxiety disorder, generalized anxiety disorder or depression, the method comprising administering to a mammal in need thereof an effective amount of a compound according to claim 38.
